SEQUENCE LISTING (6 sequences)

SEQ ID NO: 1 - RNA, 100 nt, Artificial Sequence
  source: /note=“Description of Artificial Sequence Synthetic polynucleotide”
uuacagccacgucuacagcaguuuuagagcuagaaauagcaaguuaaaauaaggcuaguccguuaucaacuugaaaaaguggcaccgagucggugcuuuu

SEQ ID NO: 2 - RNA, 100 nt, Artificial Sequence
  source: /note=“Description of Artificial Sequence Synthetic polynucleotide”
gacaagcaccagaaagaccaguuuuagagcuagaaauagcaaguuaaaauaaggcuaguccguuaucaacuugaaaaaguggcaccgagucggugcuuuu

SEQ ID NO: 3 - RNA, 4516 nt, Artificial Sequence
  source: /note=“Description of Artificial Sequence Synthetic polynucleotide”
gggucccgcagucggcguccagcggcucugcuuguucgugugugugucguugcaggccuuauucggauccgccaccauggacaagaaguacagcaucggacuggacaucggaacaaacagcgucggaugggcagucaucacagacgaauacaaggucccgagcaagaaguucaagguccugggaaacacagacagacacagcaucaagaagaaccugaucggagcacugcuguucgacagcggagaaacagcagaagcaacaagacugaagagaacagcaagaagaagauacacaagaagaaagaacagaaucugcuaccugcaggaaaucuucagcaacgaaauggcaaaggucgacgacagcuucuuccacagacuggaagaaagcuuccuggucgaagaagacaagaagcacgaaagacacccgaucuucggaaacaucgucgacgaagucgcauaccacgaaaaguacccgacaaucuaccaccugagaaagaagcuggucgacagcacagacaaggcagaccugagacugaucuaccuggcacuggcacacaugaucaaguucagaggacacuuccugaucgaaggagaccugaacccggacaacagcgacgucgacaagcuguucauccagcugguccagacauacaaccagcuguucgaagaaaacccgaucaacgcaagcggagucgacgcaaaggcaauccugagcgcaagacugagcaagagcagaagacuggaaaaccugaucgcacagcugccgggagaaaagaagaacggacuguucggaaaccugaucgcacugagccugggacugacaccgaacuucaagagcaacuucgaccuggcagaagacgcaaagcugcagcugagcaaggacacauacgacgacgaccuggacaaccugcuggcacagaucggagaccaguacgcagaccuguuccuggcagcaaagaaccugagcgacgcaauccugcugagcgacauccugagagucaacacagaaaucacaaaggcaccgcugagcgcaagcaugaucaagagauacgacgaacaccaccaggaccugacacugcugaaggcacuggucagacagcagcugccggaaaaguacaaggaaaucuucuucgaccagagcaagaacggauacgcaggauacaucgacggaggagcaagccaggaagaauucuacaaguucaucaagccgauccuggaaaagauggacggaacagaagaacugcuggucaagcugaacagagaagaccugcugagaaagcagagaacauucgacaacggaagcaucccgcaccagauccaccugggagaacugcacgcaauccugagaagacaggaagacuucuacccguuccugaaggacaacagagaaaagaucgaaaagauccugacauucagaaucccguacuacgucggaccgcuggcaagaggaaacagcagauucgcauggaugacaagaaagagcgaagaaacaaucacaccguggaacuucgaagaagucgucgacaagggagcaagcgcacagagcuucaucgaaagaaugacaaacuucgacaagaaccugccgaacgaaaagguccugccgaagcacagccugcuguacgaauacuucacagucuacaacgaacugacaaaggucaaguacgucacagaaggaaugagaaagccggcauuccugagcggagaacagaagaaggcaaucgucgaccugcuguucaagacaaacagaaaggucacagucaagcagcugaaggaagacuacuucaagaagaucgaaugcuucgacagcgucgaaaucagcggagucgaagacagauucaacgcaagccugggaacauaccacgaccugcugaagaucaucaaggacaaggacuuccuggacaacgaagaaaacgaagacauccuggaagacaucguccugacacugacacuguucgaagacagagaaaugaucgaagaaagacugaagacauacgcacaccuguucgacgacaaggucaugaagcagcugaagagaagaagauacacaggauggggaagacugagcagaaagcugaucaacggaaucagagacaagcagagcggaaagacaauccuggacuuccugaagagcgacggauucgcaaacagaaacuucaugcagcugauccacgacgacagccugacauucaaggaagacauccagaaggcacaggucagcggacagggagacagccugcacgaacacaucgcaaaccuggcaggaagcccggcaaucaagaagggaauccugcagacagucaaggucgucgacgaacuggucaaggucaugggaagacacaagccggaaaacaucgucaucgaaauggcaagagaaaaccagacaacacagaagggacagaagaacagcagagaaagaaugaagagaaucgaagaaggaaucaaggaacugggaagccagauccugaaggaacacccggucgaaaacacacagcugcagaacgaaaagcuguaccuguacuaccugcagaacggaagagacauguacgucgaccaggaacuggacaucaacagacugagcgacuacgacgucgaccacaucgucccgcagagcuuccugaaggacgacagcaucgacaacaagguccugacaagaagcgacaagaacagaggaaagagcgacaacgucccgagcgaagaagucgucaagaagaugaagaacuacuggagacagcugcugaacgcaaagcugaucacacagagaaaguucgacaaccugacaaaggcagagagaggaggacugagcgaacuggacaaggcaggauucaucaagagacagcuggucgaaacaagacagaucacaaagcacgucgcacagauccuggacagcagaaugaacacaaaguacgacgaaaacgacaagcugaucagagaagucaaggucaucacacugaagagcaagcuggucagcgacuucagaaaggacuuccaguucuacaaggucagagaaaucaacaacuaccaccacgcacacgacgcauaccugaacgcagucgucggaacagcacugaucaagaaguacccgaagcuggaaagcgaauucgucuacggagacuacaaggucuacgacgucagaaagaugaucgcaaagagcgaacaggaaaucggaaaggcaacagcaaaguacuucuucuacagcaacaucaugaacuucuucaagacagaaaucacacuggcaaacggagaaaucagaaagagaccgcugaucgaaacaaacggagaaacaggagaaaucgucugggacaagggaagagacuucgcaacagucagaaagguccugagcaugccgcaggucaacaucgucaagaagacagaaguccagacaggaggauucagcaaggaaagcauccugccgaagagaaacagcgacaagcugaucgcaagaaagaaggacugggacccgaagaaguacggaggauucgacagcccgacagucgcauacagcguccuggucgucgcaaaggucgaaaagggaaagagcaagaagcugaagagcgucaaggaacugcugggaaucacaaucauggaaagaagcagcuucgaaaagaacccgaucgacuuccuggaagcaaagggauacaaggaagucaagaaggaccugaucaucaagcugccgaaguacagccuguucgaacuggaaaacggaagaaagagaaugcuggcaagcgcaggagaacugcagaagggaaacgaacuggcacugccgagcaaguacgucaacuuccuguaccuggcaagccacuacgaaaagcugaagggaagcccggaagacaacgaacagaagcagcuguucgucgaacagcacaagcacuaccuggacgaaaucaucgaacagaucagcgaauucagcaagagagucauccuggcagacgcaaaccuggacaagguccugagcgcauacaacaagcacagagacaagccgaucagagaacaggcagaaaacaucauccaccuguucacacugacaaaccugggagcaccggcagcauucaaguacuucgacacaacaaucgacagaaagagauacacaagcacaaaggaaguccuggacgcaacacugauccaccagagcaucacaggacuguacgaaacaagaaucgaccugagccagcugggaggagacggaggaggaagcccgaagaagaagagaaaggucuagcuagccaucacauuuaaaagcaucucagccuaccaugagaauaagagaaagaaaaugaagaucaauagcuuauucaucucuuuuucuuuuucguugguguaaagccaacacccugucuaaaaaacauaaauuucuuuaaucauuuugccucuuuucucugugcuucaauuaauaaaaaauggaaagaaccucgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuag

SEQ ID NO: 4 - RNA, 100 nt, Artificial Sequence
  source: /note=“Description of Artificial Sequence Synthetic polynucleotide”
acacaaauaccaguccagcgguuuuagagcuagaaauagcaaguuaaaauaaggcuaguccguuaucaacuugaaaaaguggcaccgagucggugcuuuu

SEQ ID NO: 5 - RNA, 100 nt, Artificial Sequence
  source: /note=“Description of Artificial Sequence Synthetic polynucleotide”
acgcaaauaucaguccagcgguuuuagagcuagaaauagcaaguuaaaauaaggcuaguccguuaucaacuugaaaaaguggcaccgagucggugcuuuu

SEQ ID NO: 6 - RNA, 4140 nt, Artificial Sequence
  source: /note=“Description of Artificial Sequence Synthetic polynucleotide”
auggacaagaaguacuccaucggccuggacaucggcaccaacuccgugggcugggccgugaucaccgacgaguacaaggugcccuccaagaaguucaaggugcugggcaacaccgaccggcacuccaucaagaagaaccugaucggcgcccugcuguucgacuccggcgagaccgccgaggccacccggcugaagcggaccgcccggcggcgguacacccggcggaagaaccggaucugcuaccugcaggagaucuucuccaacgagauggccaagguggacgacuccuucuuccaccggcuggaggaguccuuccugguggaggaggacaagaagcacgagcggcaccccaucuucggcaacaucguggacgagguggccuaccacgagaaguaccccaccaucuaccaccugcggaagaagcugguggacuccaccgacaaggccgaccugcggcugaucuaccuggcccuggcccacaugaucaaguuccggggccacuuccugaucgagggcgaccugaaccccgacaacuccgacguggacaagcuguucauccagcuggugcagaccuacaaccagcuguucgaggagaaccccaucaacgccuccggcguggacgccaaggccauccuguccgcccggcuguccaagucccggcggcuggagaaccugaucgcccagcugcccggcgagaagaagaacggccuguucggcaaccugaucgcccugucccugggccugacccccaacuucaaguccaacuucgaccuggccgaggacgccaagcugcagcuguccaaggacaccuacgacgacgaccuggacaaccugcuggcccagaucggcgaccaguacgccgaccuguuccuggccgccaagaaccuguccgacgccauccugcuguccgacauccugcgggugaacaccgagaucaccaaggccccccuguccgccuccaugaucaagcgguacgacgagcaccaccaggaccugacccugcugaaggcccuggugcggcagcagcugcccgagaaguacaaggagaucuucuucgaccaguccaagaacggcuacgccggcuacaucgacggcggcgccucccaggaggaguucuacaaguucaucaagcccauccuggagaagauggacggcaccgaggagcugcuggugaagcugaaccgggaggaccugcugcggaagcagcggaccuucgacaacggcuccaucccccaccagauccaccugggcgagcugcacgccauccugcggcggcaggaggacuucuaccccuuccugaaggacaaccgggagaagaucgagaagauccugaccuuccggauccccuacuacgugggcccccuggcccggggcaacucccgguucgccuggaugacccggaaguccgaggagaccaucacccccuggaacuucgaggaggugguggacaagggcgccuccgcccaguccuucaucgagcggaugaccaacuucgacaagaaccugcccaacgagaaggugcugcccaagcacucccugcuguacgaguacuucaccguguacaacgagcugaccaaggugaaguacgugaccgagggcaugcggaagcccgccuuccuguccggcgagcagaagaaggccaucguggaccugcuguucaagaccaaccggaaggugaccgugaagcagcugaaggaggacuacuucaagaagaucgagugcuucgacuccguggagaucuccggcguggaggaccgguucaacgccucccugggcaccuaccacgaccugcugaagaucaucaaggacaaggacuuccuggacaacgaggagaacgaggacauccuggaggacaucgugcugacccugacccuguucgaggaccgggagaugaucgaggagcggcugaagaccuacgcccaccuguucgacgacaaggugaugaagcagcugaagcggcggcgguacaccggcuggggccggcugucccggaagcugaucaacggcauccgggacaagcaguccggcaagaccauccuggacuuccugaaguccgacggcuucgccaaccggaacuucaugcagcugauccacgacgacucccugaccuucaaggaggacauccagaaggcccagguguccggccagggcgacucccugcacgagcacaucgccaaccuggccggcucccccgccaucaagaagggcauccugcagaccgugaaggugguggacgagcuggugaaggugaugggccggcacaagcccgagaacaucgugaucgagauggcccgggagaaccagaccacccagaagggccagaagaacucccgggagcggaugaagcggaucgaggagggcaucaaggagcugggcucccagauccugaaggagcaccccguggagaacacccagcugcagaacgagaagcuguaccuguacuaccugcagaacggccgggacauguacguggaccaggagcuggacaucaaccggcuguccgacuacgacguggaccacaucgugccccaguccuuccugaaggacgacuccaucgacaacaaggugcugacccgguccgacaagaaccggggcaaguccgacaacgugcccuccgaggagguggugaagaagaugaagaacuacuggcggcagcugcugaacgccaagcugaucacccagcggaaguucgacaaccugaccaaggccgagcggggcggccuguccgagcuggacaaggccggcuucaucaagcggcagcugguggagacccggcagaucaccaagcacguggcccagauccuggacucccggaugaacaccaaguacgacgagaacgacaagcugauccgggaggugaaggugaucacccugaaguccaagcugguguccgacuuccggaaggacuuccaguucuacaaggugcgggagaucaacaacuaccaccacgcccacgacgccuaccugaacgccguggugggcaccgcccugaucaagaaguaccccaagcuggaguccgaguucguguacggcgacuacaagguguacgacgugcggaagaugaucgccaaguccgagcaggagaucggcaaggccaccgccaaguacuucuucuacuccaacaucaugaacuucuucaagaccgagaucacccuggccaacggcgagauccggaagcggccccugaucgagaccaacggcgagaccggcgagaucgugugggacaagggccgggacuucgccaccgugcggaaggugcuguccaugccccaggugaacaucgugaagaagaccgaggugcagaccggcggcuucuccaaggaguccauccugcccaagcggaacuccgacaagcugaucgcccggaagaaggacugggaccccaagaaguacggcggcuucgacucccccaccguggccuacuccgugcuggugguggccaagguggagaagggcaaguccaagaagcugaaguccgugaaggagcugcugggcaucaccaucauggagcgguccuccuucgagaagaaccccaucgacuuccuggaggccaagggcuacaaggaggugaagaaggaccugaucaucaagcugcccaaguacucccuguucgagcuggagaacggccggaagcggaugcuggccuccgccggcgagcugcagaagggcaacgagcuggcccugcccuccaaguacgugaacuuccuguaccuggccucccacuacgagaagcugaagggcucccccgaggacaacgagcagaagcagcuguucguggagcagcacaagcacuaccuggacgagaucaucgagcagaucuccgaguucuccaagcgggugauccuggccgacgccaaccuggacaaggugcuguccgccuacaacaagcaccgggacaagcccauccgggagcaggccgagaacaucauccaccuguucacccugaccaaccugggcgcccccgccgccuucaaguacuucgacaccaccaucgaccggaagcgguacaccuccaccaaggaggugcuggacgccacccugauccaccaguccaucaccggccuguacgagacccggaucgaccugucccagcugggcggcgacggcggcggcucccccaagaagaagcggaagguguga

(19) United States
(12) Patent Application Publication
(10) Pub. No.: US 2022/0402862 A1
(43) Pub. Date: Dec. 22, 2022

(54) IONIZABLE AMINE LIPIDS AND LIPID NANOPARTICLES

(71) Applicant: Intellia Therapeutics, Inc., Cambridge, MA (US)

(72) Inventors: Stephen S. Scully, Cambridge, MA (US); Derek LaPlaca, Cambridge, MA (US); Rachel Pelly, Cambridge, MA (US); Rubina Giare Parmar, Cambridge, MA (US); Micah Maatani, Cambridge, MA (US)

(21) Appl. No.: 17/605,780

(22) PCT Filed: Apr. 24, 2020
(86) PCT No.: PCT/US20/29812

§ 371 Date: Oct. 22, 2021

Related U.S. Application Data
(60) Provisional application No. 62/843,854, filed on May 6, 2019.
(60) Provisional application No. 62/838,551, filed on Apr. 25, 2019.

(51) Int. Cl. C07C 219/16; C07C 229/12; C07C 271/20; C07D 207/08; C07D 211/06; C12N 15/11

(52) U.S. Cl. CPC C07C 219/16; C07C 229/12; C07C 271/20; C07D 207/08; C07D 211/06; C12N 15/11

(57) ABSTRACT

The disclosure provides ionizable amine lipids and salts thereof (e.g., pharmaceutically acceptable salts thereof) useful for the delivery of biologically active agents, for example delivering biologically active agents to cells to prepare engineered cells. The ionizable amine lipids disclosed herein are useful as ionizable lipids in the formulation of lipid nanoparticle-based compositions.

### CROSS-REFERENCE TO RELATED APPLICATIONS 4

This application claims the benefit of priority to U.S. Provisional Patent Application No. 62/838,551, filed Apr. 25, 2019, and U.S. Provisional Patent Application No. 62/843,854 filed May 6, 2019, the entire contents of each of which are incorporated herein by reference.

### BACKGROUND

Lipid nanoparticles formulated with ionizable amine-containing lipids can serve as cargo vehicles for delivery of biologically active agents, in particular polynucleotides, such as RNAs, mRNAs, and guide RNAs into cells. The LNP compositions containing ionizable lipids can facilitate delivery of oligonucleotide agents across cell membranes, and can be used to introduce components and compositions for gene editing into living cells. Biologically active agents that are particularly difficult to deliver to cells include proteins, nucleic acid-based drugs, and derivatives thereof, particularly drugs that include relatively large oligonucleotides, such as mRNA. Compositions for delivery of promising gene editing technologies into cells, such as for delivery of CRISPR/Cas9 system components, are of particular interest (e.g., mRNA encoding a nuclease and associated guide RNA (gRNA)).

Compositions for delivery of the protein and nucleic acid components of CRISPR/Cas to a cell, such as a cell in a patient, are needed. In particular, compositions for delivering mRNA encoding the CRISPR protein component, and for delivering CRISPR gRNAs are of particular interest. Compositions with useful properties for in vitro and in vivo delivery that can stabilize and deliver RNA components, are also of particular interest.

### BRIEF SUMMARY

The present disclosure provides amine-containing lipids useful for the formulation of lipid nanoparticle (LNP) compositions. Such LNP compositions may have properties advantageous for delivery of nucleic acid cargo, such as CRISPR/Cas gene editing components, to cells.

In some embodiments, the lipid is a compound having a structure of Formula II

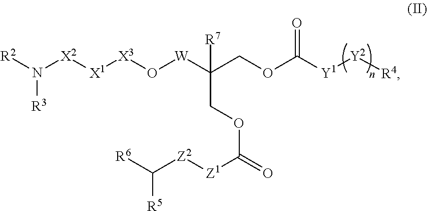

wherein
X¹ is O, NR¹, or a direct bond,
X² is C₂₋₅ alkylene,
X³ is C(═O) or a direct bond,
R¹ is H or Me,
R³ is C₁₋₃ alkyl,
R² is C₁₋₃ alkyl, or
R² taken together with the nitrogen atom to which it is attached and 1-3 carbon atoms of X² form a 4-, 5-, or 6-membered ring, or
X¹ is NR¹, R¹ and R² taken together with the nitrogen atoms to which they are attached form a 5- or 6-membered ring, or
R² taken together with R³ and the nitrogen atom to which they are attached form a 5-, 6-, or 7-membered ring,
Y¹ is C₂₋₁₂ alkylene,

Y² is selected from

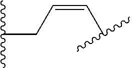 [SMILES: */C=C\C*]

(in either orientation),

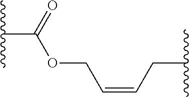 [SMILES: *C/C=C\COC(*)=O]

(in either orientation), and

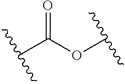 [SMILES: *OC(*)=O]

(in either orientation),
n is 0 to 3,
R⁴ is C₁₋₁₅ alkyl,
Z¹ is C₁₋₆ alkylene or a direct bond,
Z² is

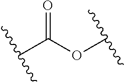 [SMILES: *OC(*)=O]

(in either orientation) or absent, provided that if Z¹ is a direct bond, Z² is absent;
R⁵ is C₅₋₉ alkyl or C₆₋₁₀ alkoxy,
R⁶ is C₅₋₉ alkyl or C₆₋₁₀ alkoxy,
W is methylene or a direct bond, and
R⁷ is H or Me,
or a salt thereof,

provided that if R³ and R² are C₂ alkyls, X¹ is O, X² is linear C₃ alkylene, X³ is C(═O), Y¹ is linear C₆ alkylene, (Y²)ₙ—R⁴ is

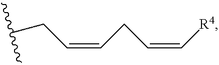

R⁴ is linear C₅ alkyl, Z¹ is C₂ alkylene, Z² is absent, W is methylene, and R⁷ is H, then R⁵ and R⁶ are not C₈ alkoxy.

In certain embodiments, the lipid is a compound having a structure of Formula (I):

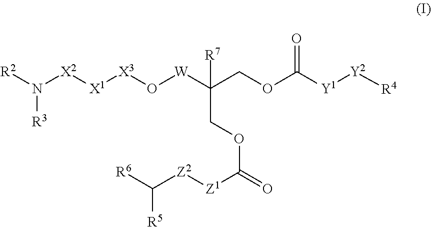

X¹ is O, NR¹, or a direct bond,
X² is C₂₋₅ alkylene,
X³ is C(═O) or a direct bond,
R¹ is H or Me,
R³ is C₁₋₃ alkyl,
R² is C₁₋₃ alkyl, or
R² taken together with the nitrogen atom to which it is attached and 1-3 carbon atoms of X² form a 4-, 5-, or 6-membered ring, or
X¹ is NR¹, R¹ and R² taken together with the nitrogen atoms to which they are attached form a 5- or 6-membered ring, or
R² taken together with R³ and the nitrogen atom to which they are attached form a 5-, 6-, or 7-membered ring,
Y¹ is C₂₋₁₂ alkylene,
Y² is selected from

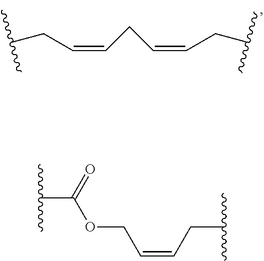 [SMILES: *C/C=C\C/C=C\C* ; *C/C=C\COC(*)=O]

(in either orientation), and

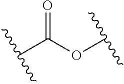 [SMILES: *OC(*)=O]

(in either orientation),
R⁴ is C₃₋₁₅ alkyl,

Z¹ is C₁₋₆ alkylene or a direct bond,
Z² is

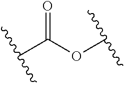 [SMILES: *OC(*)=O]

(in either orientation) or absent, provided that if Z¹ is a direct bond, Z² is absent,
R⁵ is C₅₋₉ alkyl or C₆₋₁₀ alkoxy,
R⁶ is C₅₋₉ alkyl or C₆₋₁₀ alkoxy,
W is methylene or a direct bond, and
R⁷ is H or Me,
or a salt thereof,

provided that if R³ and R² are C₂ alkyls, X¹ is O, X² is linear C₃ alkylene, X³ is C(═O), Y¹ is linear C₆ alkylene, Y² is

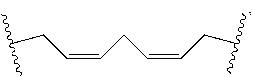 [SMILES: *C/C=C\C/C=C\C*]

R⁴ is linear C₄ alkyl, Z¹ is C₂ alkylene, Z² is absent, W is methylene, and R⁷ is H, then R⁵ and R⁶ are not C₈ alkoxy.

In certain embodiments, the invention relates to any compound described herein, wherein the pKa of the protonated form of the compound is from about 5.1 to about 9.0, for example from about 5.7 to about 7.6, or from about 6 to about 7.5.

In certain embodiments, the invention relates to a composition comprising any compound described herein and a lipid component, for example comprising about 50% (for example, about 50% of the lipid component) of a compound described herein and a lipid component, for example, an amine lipid, preferably a compound of Formula (I) or Formula (II).

In certain embodiments, the invention relates to any composition described herein, wherein the composition is an LNP composition. For example, the invention relates to an LNP composition comprising any compound described herein and a lipid component. In certain embodiments, the invention relates to any LNP composition described herein, wherein the lipid component comprises a helper lipid and a PEG lipid. In certain embodiments, the invention relates to any LNP composition described herein, wherein the lipid component comprises a helper lipid, a PEG lipid, and a neutral lipid. In certain embodiments, the invention relates to any LNP composition described herein, further comprising a cryoprotectant. In certain embodiments, the invention relates to any LNP composition described herein, further comprising a buffer.

In certain embodiments, the invention relates to any LNP composition described herein, further comprising a nucleic acid component. In certain embodiments, the invention relates to any LNP composition described herein, further comprising an RNA or DNA component. In certain embodiments, the invention relates to any LNP composition described herein, wherein the LNP composition has an N/P ratio of about 3-10, for example the N/P ratio is about 6±1, or the N/P ratio is about 6±0.5. In certain embodiments, the invention relates to any LNP composition described herein, wherein the LNP composition has an N/P ratio of about 6.

In certain embodiments, the invention relates to any LNP composition described herein, wherein the RNA component comprises an mRNA. In certain embodiments, the invention relates to any LNP composition described herein, wherein the RNA component comprises an RNA-guided DNA-binding agent, for example a Cas nuclease mRNA, such as a Class 2 Cas nuclease mRNA, or a Cas9 nuclease mRNA.

In certain embodiments, the invention relates to any LNP composition described herein, wherein the mRNA is a modified mRNA. In certain embodiments, the invention relates to any LNP composition described herein, wherein the RNA component comprises a gRNA nucleic acid. In certain embodiments, the invention relates to any LNP composition described herein, wherein the gRNA nucleic acid is a gRNA.

In certain embodiments, the invention relates to an LNP composition described herein, wherein the RNA component comprises a Class 2 Cas nuclease mRNA and a gRNA. In certain embodiments, the invention relates to any LNP composition described herein, wherein the gRNA nucleic acid is or encodes a dual-guide RNA (dgRNA). In certain embodiments, the invention relates to any LNP composition described herein, wherein the gRNA nucleic acid is or encodes a single-guide RNA (sgRNA).

In certain embodiments, the invention relates to any LNP composition described herein, wherein the gRNA is a modified gRNA. In certain embodiments, the invention relates to any LNP composition described herein, wherein the modified gRNA comprises a modification at one or more of the first five nucleotides at a 5′ end. In certain embodiments, the invention relates to any LNP composition described herein, wherein the modified gRNA comprises a modification at one or more of the last five nucleotides at a 3′ end.

In certain embodiments, the invention relates to any LNP composition described herein, further comprising at least one template nucleic acid.

In certain embodiments, the invention relates to a method of gene editing, comprising contacting a cell with an LNP. In certain embodiments, the invention relates to any method of gene editing described herein, comprising cleaving DNA.

In certain embodiments, the invention relates to a method of cleaving DNA, comprising contacting a cell with an LNP composition. In certain embodiments, the invention relates to any method of cleaving DNA described herein, wherein the cleaving step comprises introducing a single stranded DNA nick. In certain embodiments, the invention relates to any method of cleaving DNA described herein, wherein the cleaving step comprises introducing a double-stranded DNA break. In certain embodiments, the invention relates to any method of cleaving DNA described herein, wherein the LNP composition comprises a Class 2 Cas mRNA and a gRNA nucleic acid. In certain embodiments, the invention relates to any method of cleaving DNA described herein, further comprising introducing at least one template nucleic acid into the cell. In certain embodiments, the invention relates to any method of cleaving DNA described herein, comprising contacting the cell with an LNP composition comprising a template nucleic acid.

In certain embodiments, the invention relates to any a method of gene editing described herein, wherein the method comprises administering the LNP composition to an animal, for example a human. In certain embodiments, the invention relates to any method of gene editing described herein, wherein the method comprises administering the LNP composition to a cell, such as a eukaryotic cell.

In certain embodiments, the invention relates to any method of gene editing described herein, wherein the method comprises administering the mRNA formulated in a first LNP composition and a second LNP composition comprising one or more of an mRNA, a gRNA, a gRNA nucleic acid, and a template nucleic acid. In certain embodiments, the invention relates to any method of gene editing described herein, wherein the first and second LNP compositions are administered simultaneously. In certain embodiments, the invention relates to any method of gene editing described herein, wherein the first and second LNP compositions are administered sequentially. In certain embodiments, the invention relates to any method of gene editing described herein, wherein the method comprises administering the mRNA and the gRNA nucleic acid formulated in a single LNP composition.

In certain embodiments, the invention relates to any method of gene editing described herein, wherein the gene editing results in a gene knockout.

In certain embodiments, the invention relates to any method of gene editing described herein, wherein the gene editing results in a gene correction.

### BRIEF DESCRIPTION OF DRAWINGS

FIG. 1A is a graph showing percentage of editing of TTR in mouse liver cells after delivery using LNPs comprising Compound 1, Compound 29, Compound 31, or Compound 32. Dose response data are also shown. See Example 122.

FIG. 1B is a graph showing percentage of editing of TTR in mouse liver cells after delivery using LNPs comprising Compound 1, Compound 40, Compound 53, or Compound 54. Dose response data are also shown. See Example 122.

FIG. 1C is a graph showing percentage of editing of B2M and TTR in mouse liver cells after delivery using LNPs comprising Compound 1 or Compound 59. Dose response data are also shown. See Example 122.

FIG. 1D is a graph showing percentage of editing of TTR in mouse liver cells after delivery using LNPs comprising Compound 1, Compound 24, Compound 59, Compound 61, or Compound 94. Dose response data are also shown. See Example 122.

FIG. 2 is a graph showing the percentage of editing of TTR in mouse liver cells after delivery using LNPs comprising Compound 1, Compound 100, Compound 101, Compound 102, or Compound 103. See Example 123.

FIG. 3 is a graph showing the percentage of editing of TTR in mouse liver cells after delivery using LNPs comprising Compound 1, Compound 114, Compound 115, or Compound 116. See Example 123.

FIG. 4 is a graph showing the percentage of editing of TTR in rat liver cells after delivery using LNPs comprising Compound 1, Compound 12, Compound 59, or Compound 94. Dose response data are shown. See Example 125.

### DETAILED DESCRIPTION

The present disclosure provides lipids, particularly ionizable lipids, useful for delivering biologically active agents, including nucleic acids, such as CRISPR/Cas component RNAs (the “cargo”), to a cell, and methods for preparing and using such compositions. The lipids and pharmaceutically acceptable salts thereof are provided, optionally as compositions comprising the lipids, including LNP compositions. In certain embodiments, the LNP composition may comprise a biologically active agent, e.g. an RNA component, and a lipid component that includes a compound of Formula (II) or (I), as defined herein. In certain embodiments, the RNA component includes an RNA. In some embodiments, the lipids are used to deliver a biologically active agent, e.g. an mRNA to a cell such as a liver cell. In certain embodiments, the RNA component includes a gRNA and optionally an mRNA encoding a Class 2 Cas nuclease. Methods of gene editing and methods of making engineered cells using these compositions are also provided.

### Lipid Nanoparticle Compositions

Disclosed herein are various LNP compositions for delivering biologically active agents, such as nucleic acids, e.g., mRNAs and gRNAs, including CRISPR/Cas cargoes. Such LNP compositions include an “ionizable amine lipid”, along with a neutral lipid, a PEG lipid, and a helper lipid. “Lipid nanoparticle” or “LNP” refers to, without limiting the meaning, a particle that comprises a plurality of (i.e., more than one) LNP components physically associated with each other by intermolecular forces.

### Lipids

The disclosure provides lipids that can be used in LNP compositions. In some embodiments, the lipid is a compound having a structure of Formula II

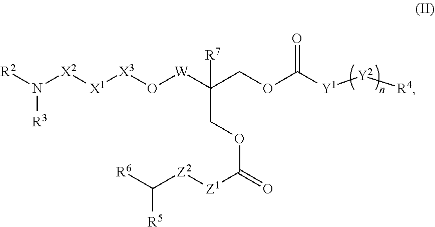

wherein
X¹ is O, NR¹, or a direct bond,
X² is C₂₋₅ alkylene,
X³ is C(═O) or a direct bond,
R¹ is H or Me,
R³ is C₁₋₃ alkyl,
R² is C₁₋₃ alkyl, or
R² taken together with the nitrogen atom to which it is attached and 1-3 carbon atoms of X² form a 4-, 5-, or 6-membered ring, or
X¹ is NR¹, R¹ and R² taken together with the nitrogen atoms to which they are attached form a 5- or 6-membered ring, or
R² taken together with R³ and the nitrogen atom to which they are attached form a 5-, 6-, or 7-membered ring,
Y¹ is C₂₋₁₂ alkylene,
Y² is selected from

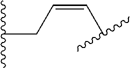 [SMILES: */C=C\C*]

(in either orientation),

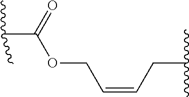 [SMILES: *C/C=C\COC(*)=O]

(in either orientation), and

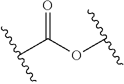 [SMILES: *OC(*)=O]

(in either orientation),
n is 0 to 3,
R⁴ is C₁₋₁₅ alkyl,
Z¹ is C₁₋₆ alkylene or a direct bond,
Z² is

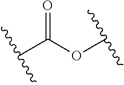 [SMILES: *OC(*)=O]

(in either orientation) or absent, provided that if Z¹ is a direct bond, Z² is absent;
R⁵ is C₅₋₉ alkyl or C₆₋₁₀ alkoxy,
R⁶ is C₅₋₉ alkyl or C₆₋₁₀ alkoxy,
W is methylene or a direct bond, and
R⁷ is H or Me,
   or a salt thereof,

provided that if R³ and R² are C₂ alkyls, X¹ is O, X² is linear C₃ alkylene, X³ is C(═O), Y¹ is linear C₆ alkylene, (Y²)ₙ—R⁴ is

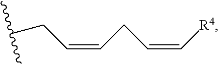

R⁴ is linear C₅ alkyl, Z¹ is C₂ alkylene, Z² is absent, W is methylene, and R⁷ is H, then R⁵ and R⁶ are not C₈ alkoxy.

In some embodiments n is 1 to 3, for example, n is 1. In certain embodiments, n is 2. In some embodiments, n is 3.

In certain embodiments, the lipid is a compound having a structure of Formula (I):

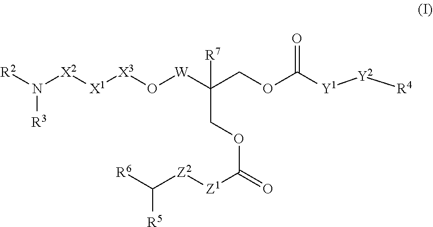

X¹ is O, NR¹, or a direct bond,
X² is C₂₋₅ alkylene,
X³ is C(═O) or a direct bond,
R¹ is H or Me,
R³ is C₁₋₃ alkyl,
R² is C₁₋₃ alkyl, or
R² taken together with the nitrogen atom to which it is attached and 1-3 carbon atoms of X² form a 4-, 5-, or 6-membered ring, or
X¹ is NR¹, R¹ and R² taken together with the nitrogen atoms to which they are attached form a 5- or 6-membered ring, or
R² taken together with R³ and the nitrogen atom to which they are attached form a 5-, 6-, or 7-membered ring,
Y¹ is C₂₋₁₂ alkylene,
Y² is selected from

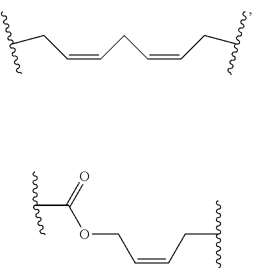 [SMILES: *C/C=C\C/C=C\C* ; *C/C=C\COC(*)=O]

(in either orientation), and

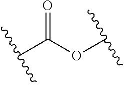 [SMILES: *OC(*)=O]

(in either orientation),
R⁴ is C₃₋₁₅ alkyl,
Z¹ is C₁₋₆ alkylene or a direct bond,
Z² is

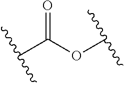 [SMILES: *OC(*)=O]

(in either orientation) or absent, provided that if Z¹ is a direct bond, Z² is absent,
R⁵ is C₅₋₉ alkyl or C₆₋₁₀ alkoxy,
R⁶ is C₅₋₉ alkyl or C₆₋₁₀ alkoxy,
W is methylene or a direct bond, and
R⁷ is H or Me,
or a salt thereof,

provided that if R³ and R² are C₂ alkyls, X¹ is O, X² is linear C₃ alkylene, X³ is C(═O), Y¹ is linear C₆ alkylene, Y² is

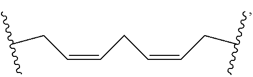 [SMILES: *C/C=C\C/C=C\C*]

R⁴ is linear C₄ alkyl, Z¹ is C₂ alkylene, Z² is absent, W is methylene, and R⁷ is H, then R⁵ and R⁶ are not C₈ alkoxy (e.g., the compound is not Compound 1).

Preferably, the compound is a compound having a structure of Formula (I) provided that if R³ and R² are C₂ alkyls, X¹ is O, X² is linear C₃ alkylene, X³ is C(═O), Y¹ is linear C₆ alkylene, Y² is

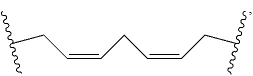 [SMILES: *C/C=C\C/C=C\C*]

R⁴ is linear C₄ alkyl, Z¹ is C₂ alkylene, Z² is absent, W is methylene, and R⁷ is H, then R⁵ and R⁶ are not C₆₋₁₀ alkoxy.

In some embodiments, the compound is a compound of Formula (Ia):

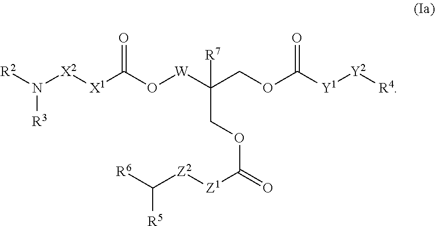

In some embodiments,

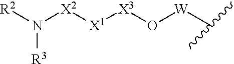

is selected from

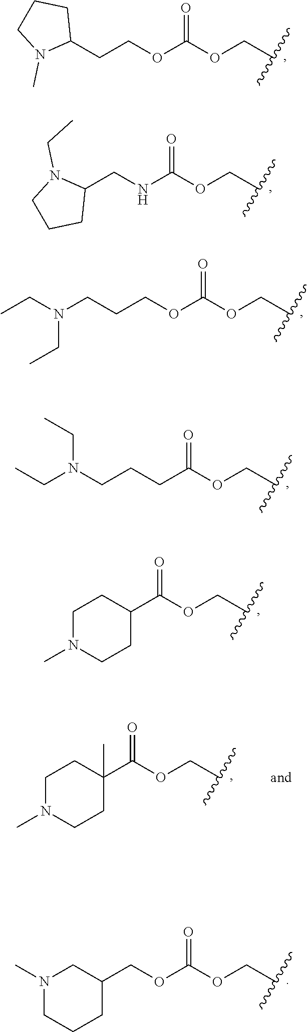

In some embodiments, X² is linear C₂ alkylene, or linear C₃ alkylene, or linear C₄ alkylene.

In other embodiments, R³ is C₁ alkyl or C₂ alkyl.

In certain embodiments, R² is C₁ alkyl or C₂ alkyl. In some other embodiments, R² taken together with the nitrogen atom and 1-2 carbon atoms of X² form a 5-membered ring. Alternatively, R² taken together with the nitrogen atom and 1-3 carbon atoms of X² may form a 6-membered ring.

In yet other embodiments, R² and R³ taken together with the nitrogen atom form a 5-membered ring. In certain embodiments, X¹ is NH or is a direct bond.

In some embodiments, Y¹ is linear C₃₋₁₀ alkylene, such as a linear C₄₋₈ alkylene, for example, a linear C_{5 -7} alkylene.

In certain embodiments, R⁴ is linear C₄₋₁₄ alkyl, preferably a linear C₆₋₁₂ alkyl.

In some embodiments, Z¹ is linear C₂₋₄ alkylene.

In certain embodiments, R⁵ and R⁶ are each independently linear C₅₋₉ alkyl, such as a linear C₆₋₈ alkyl.

In some embodiments, R⁵ and R⁶ are each independently linear C₇₋₉ alkoxy.

In certain embodiments, R⁵ and R⁶ are identical. Alternatively, R⁵ and R⁶ are different.

In some embodiments, Y² is

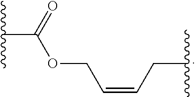 [SMILES: *C/C=C\COC(*)=O]

In other embodiments, Y² is

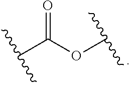 [SMILES: *OC(*)=O]

In some embodiments, Y¹ is linear C₇ alkylene, Y²

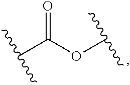 [SMILES: *OC(*)=O]

is n is 1, and R⁴ is linear C₁₀ alkyl.

In certain embodiments, Z² is

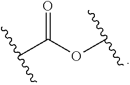 [SMILES: *OC(*)=O]

In some embodiments, Z¹, Z², and R⁵ are selected to form a linear chain of 6-18 atoms, including the carbon and oxygen atoms of the ester and the acetal.

In some embodiments, Y¹, Y², and R⁴ are selected to form a linear chain of 14-24 atoms, including the carbon and oxygen atoms of the ester.

Representative compounds of Formula (II) include:

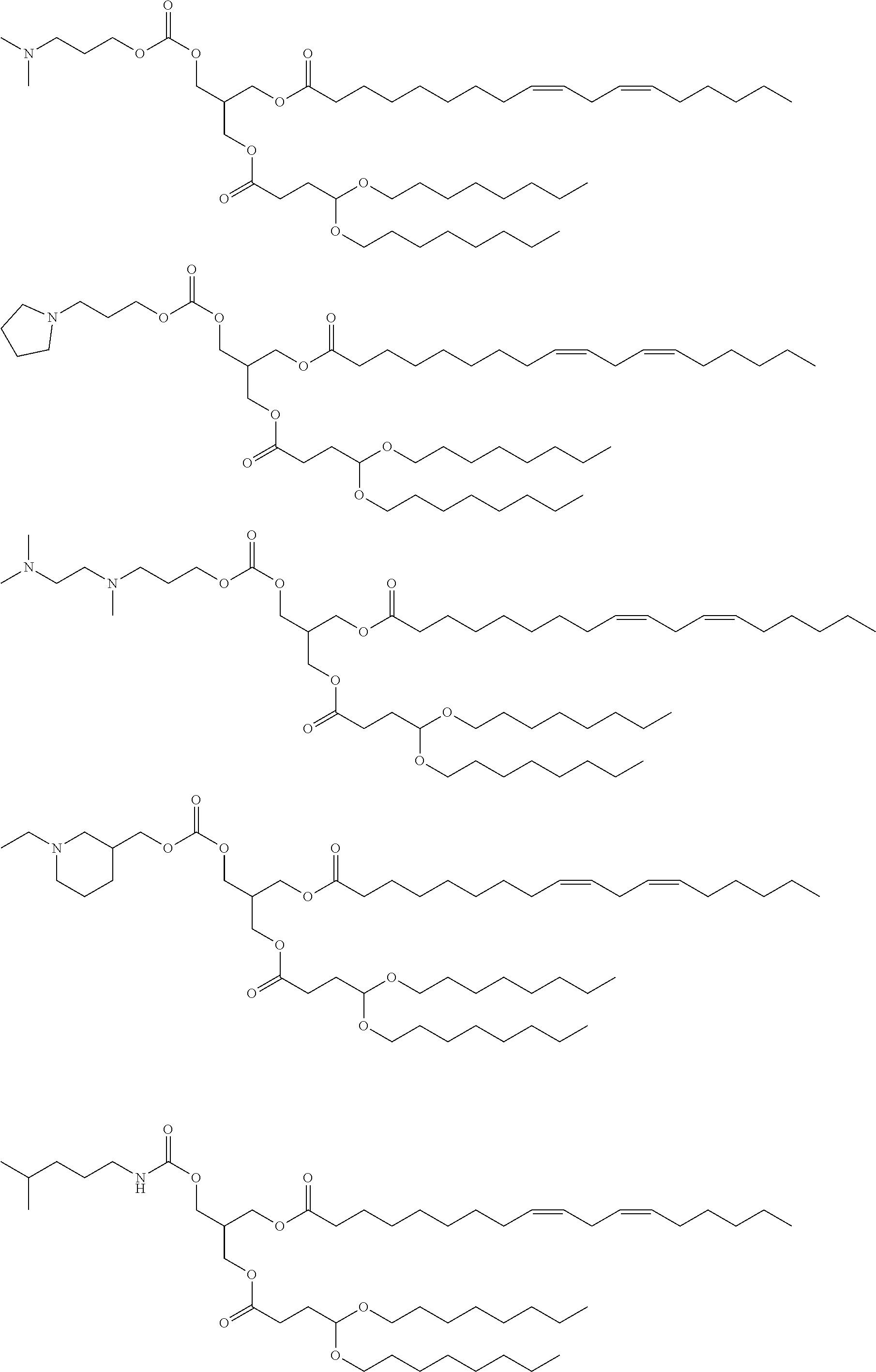

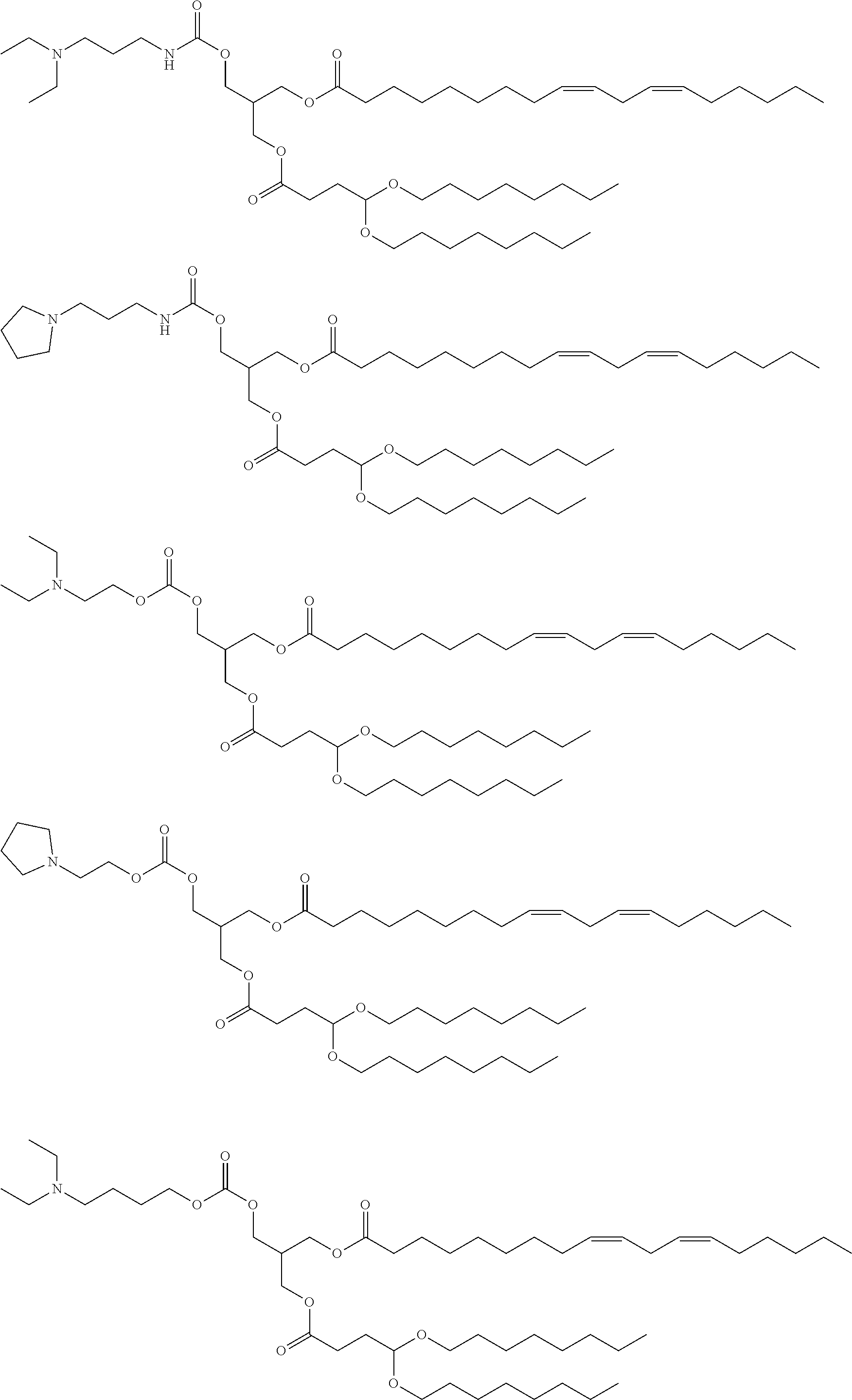

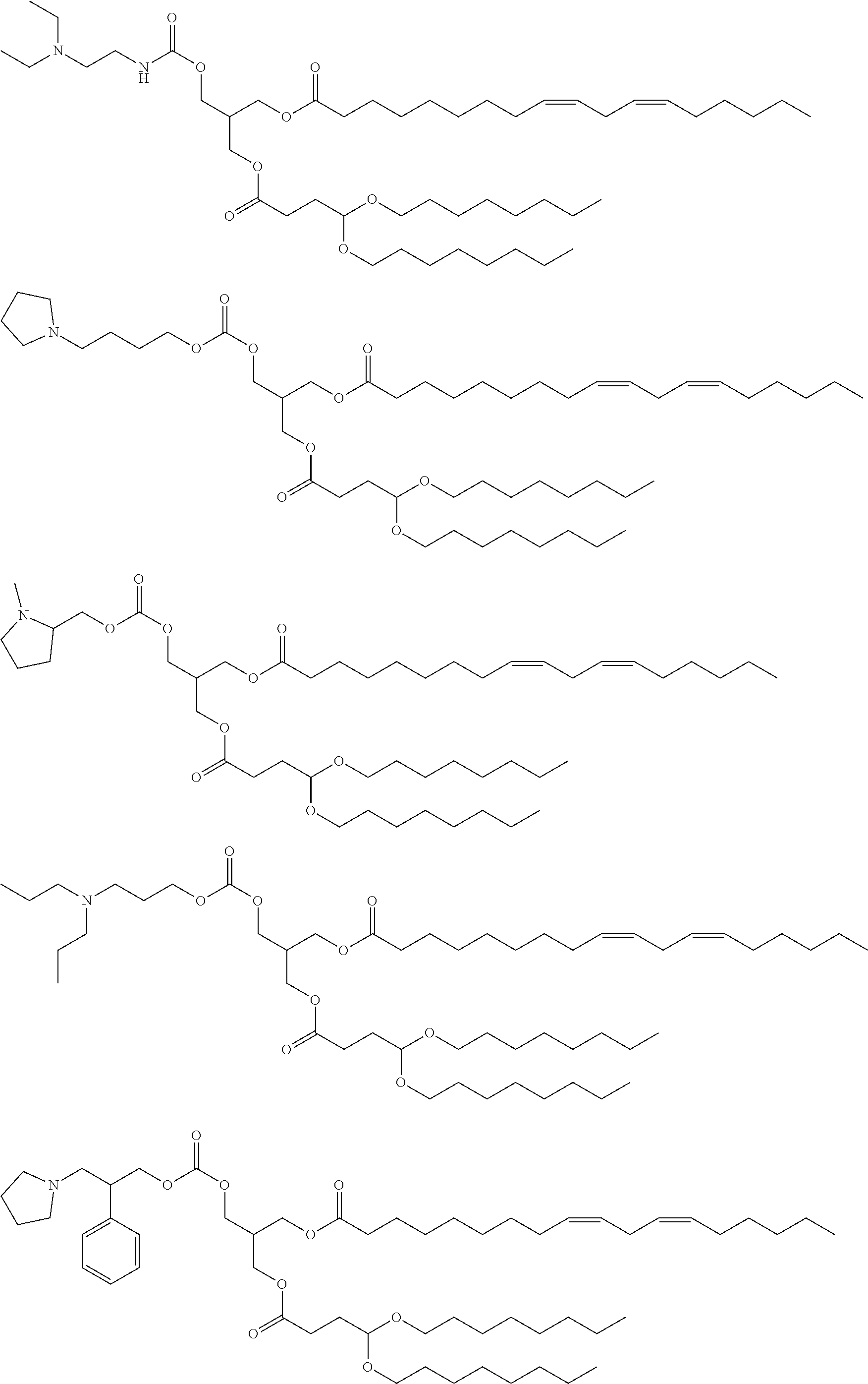

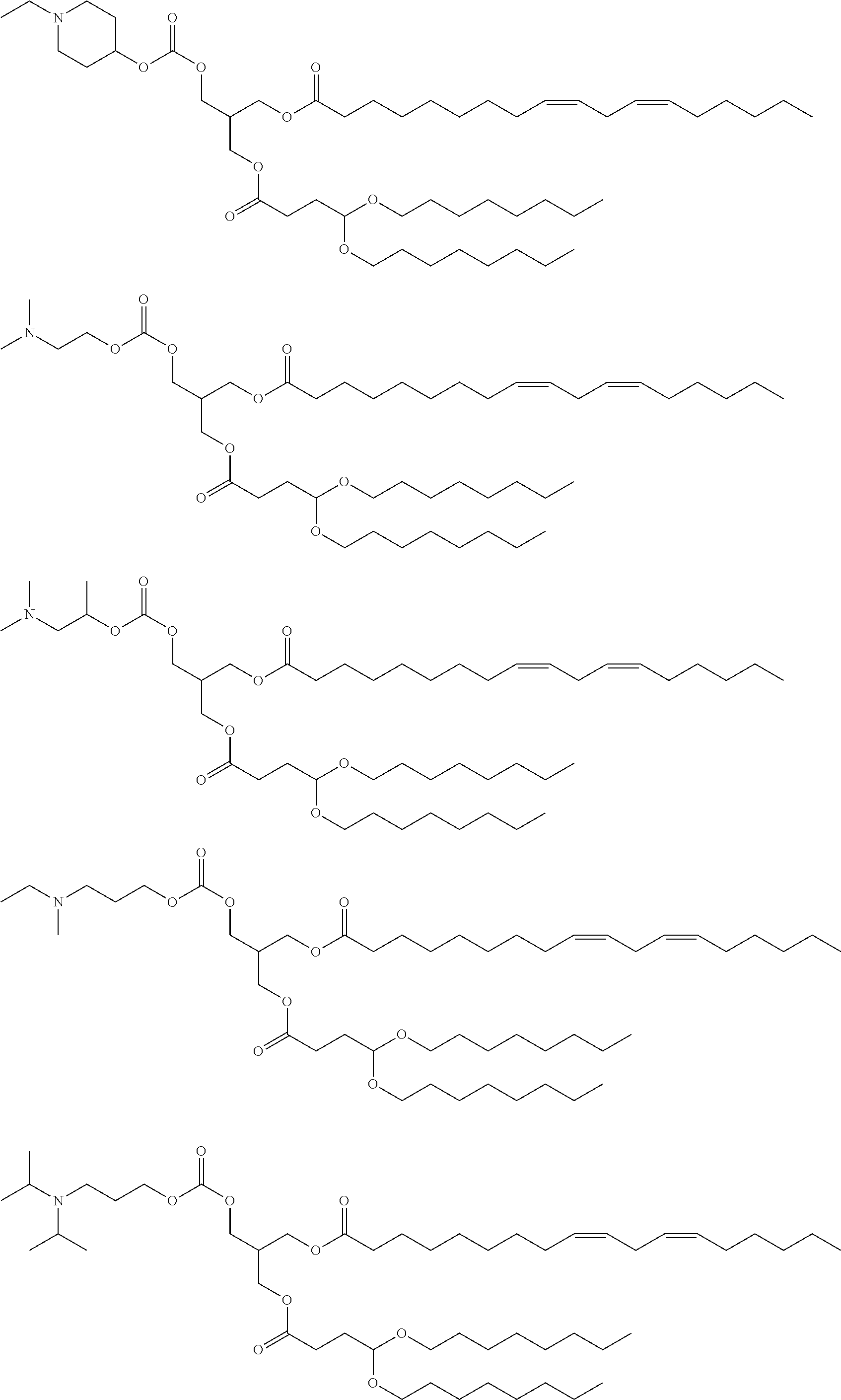

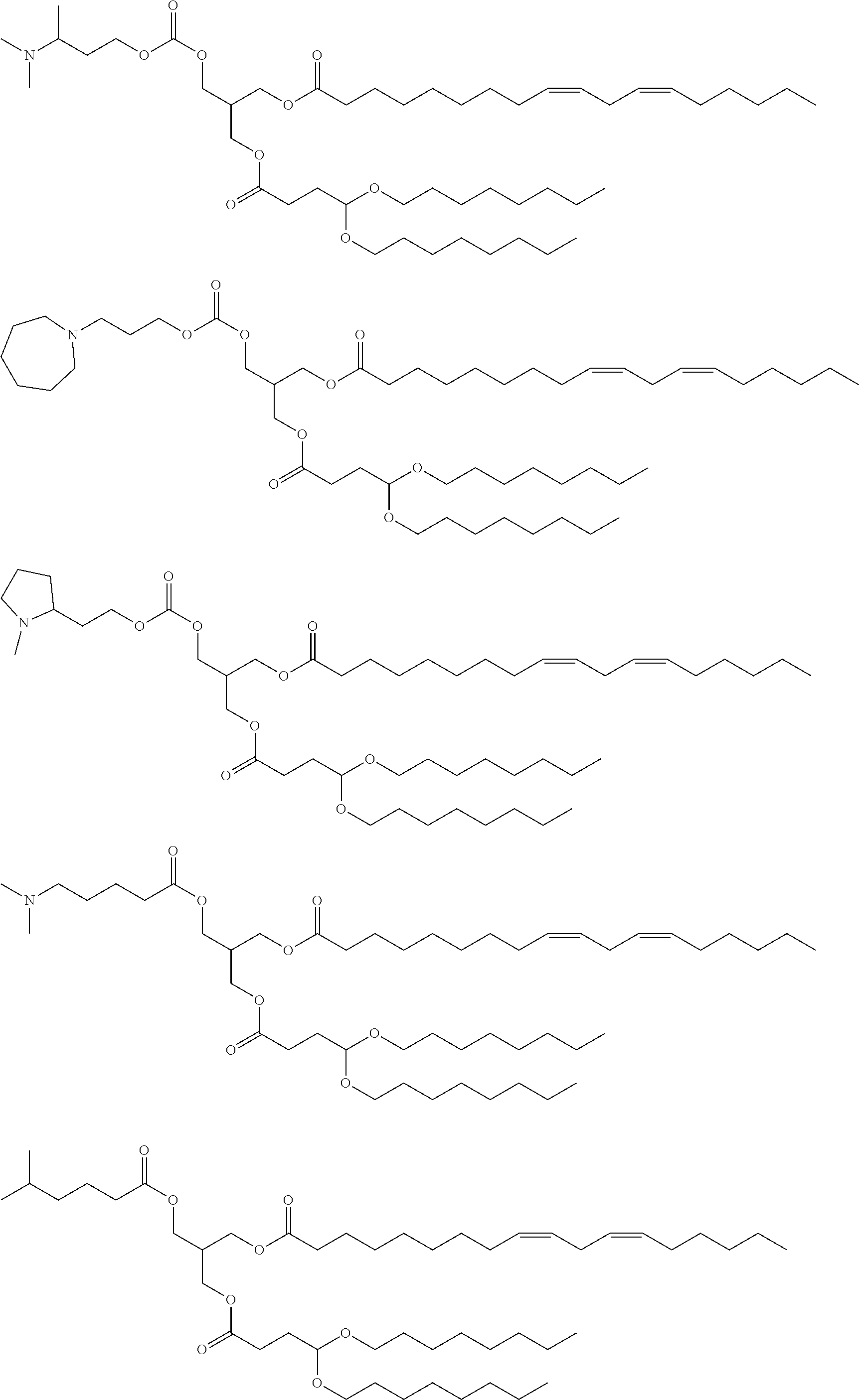

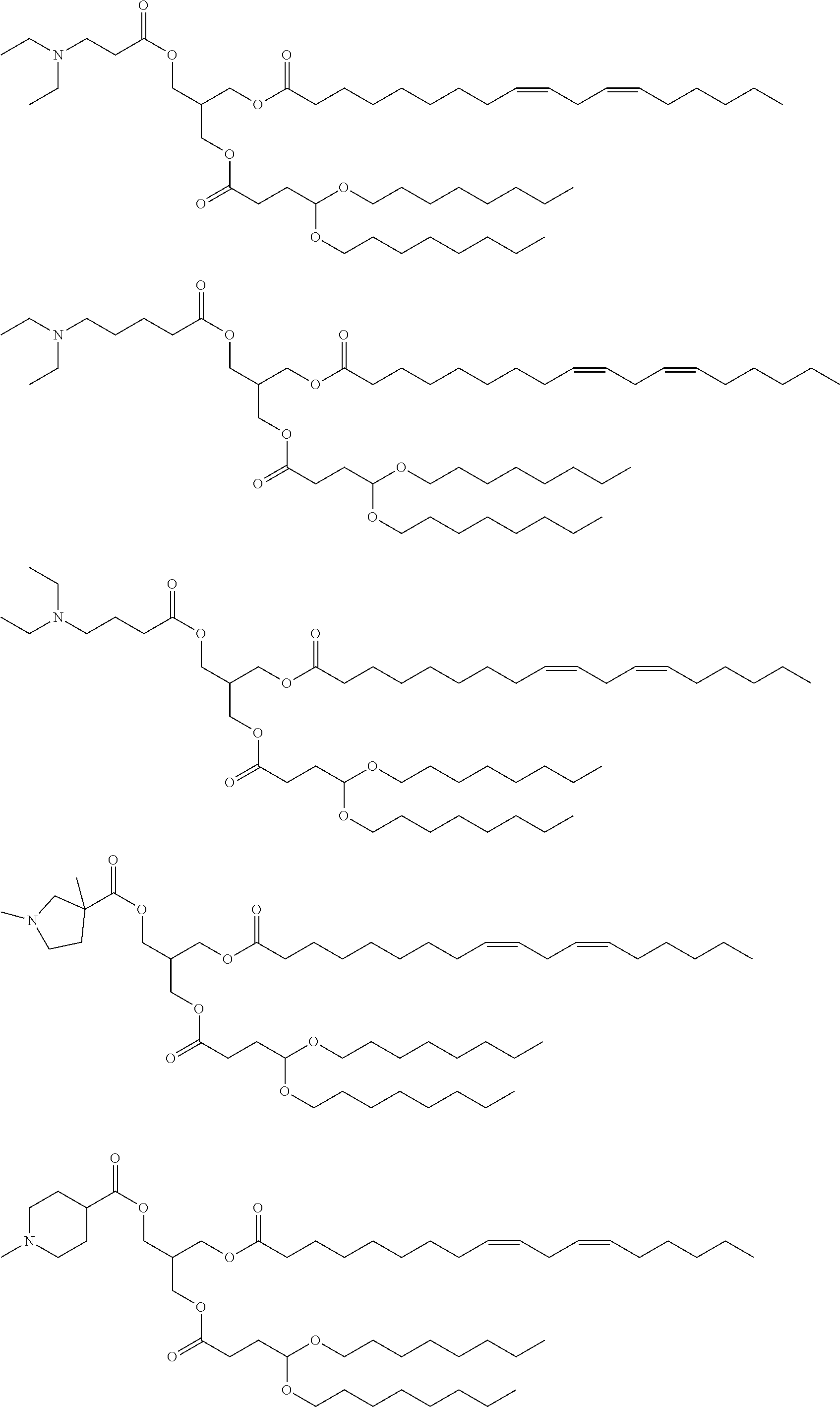

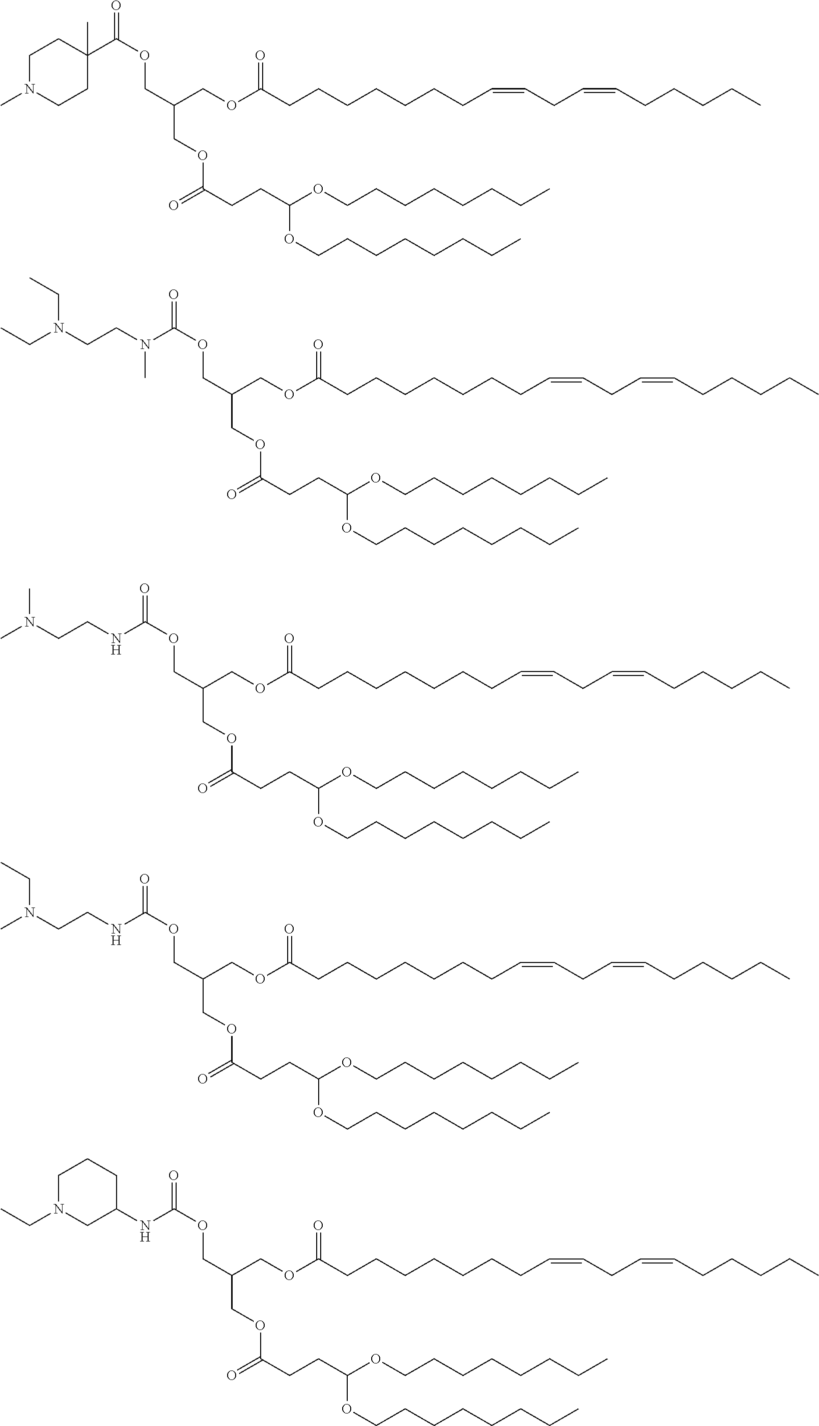

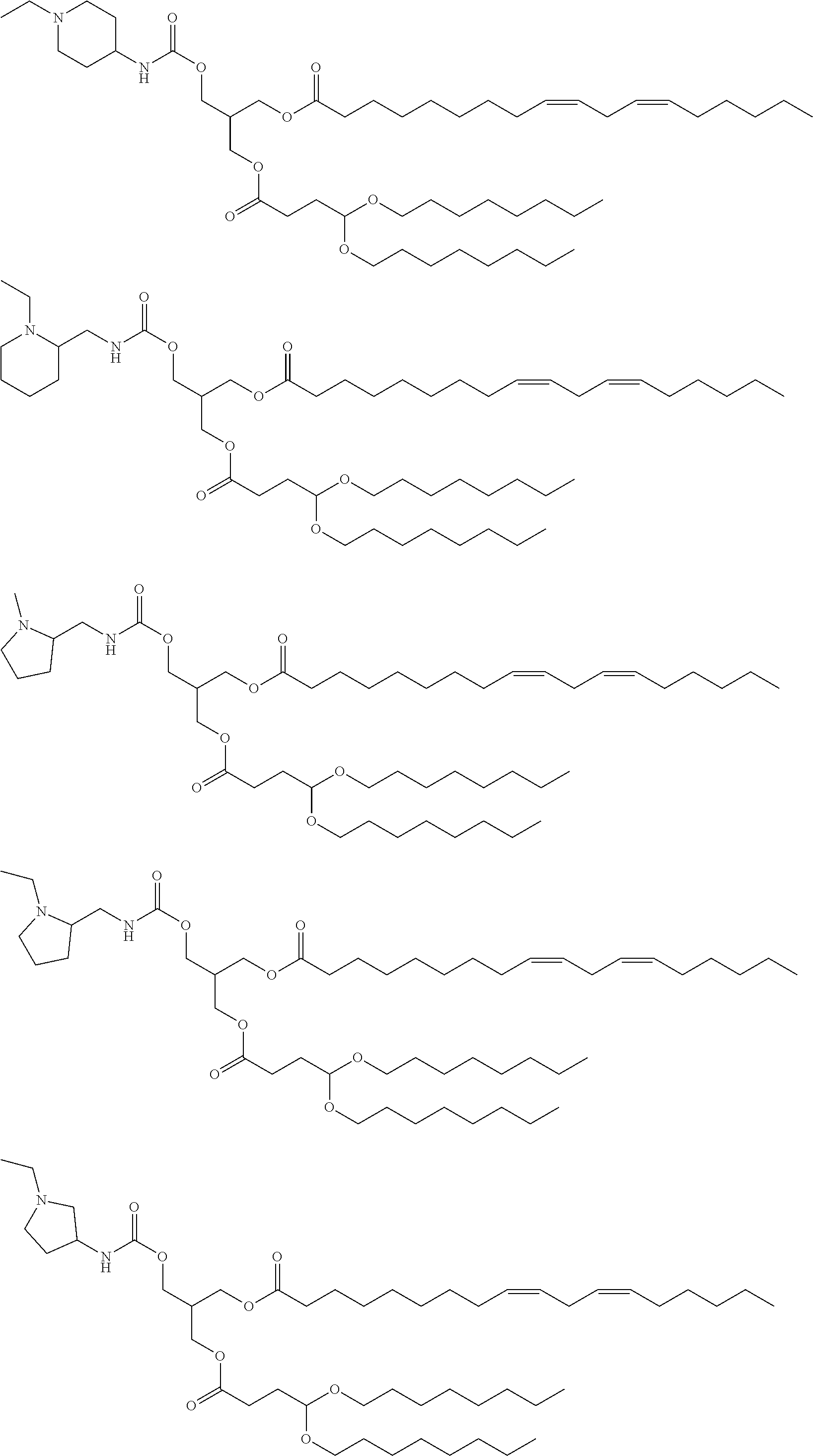

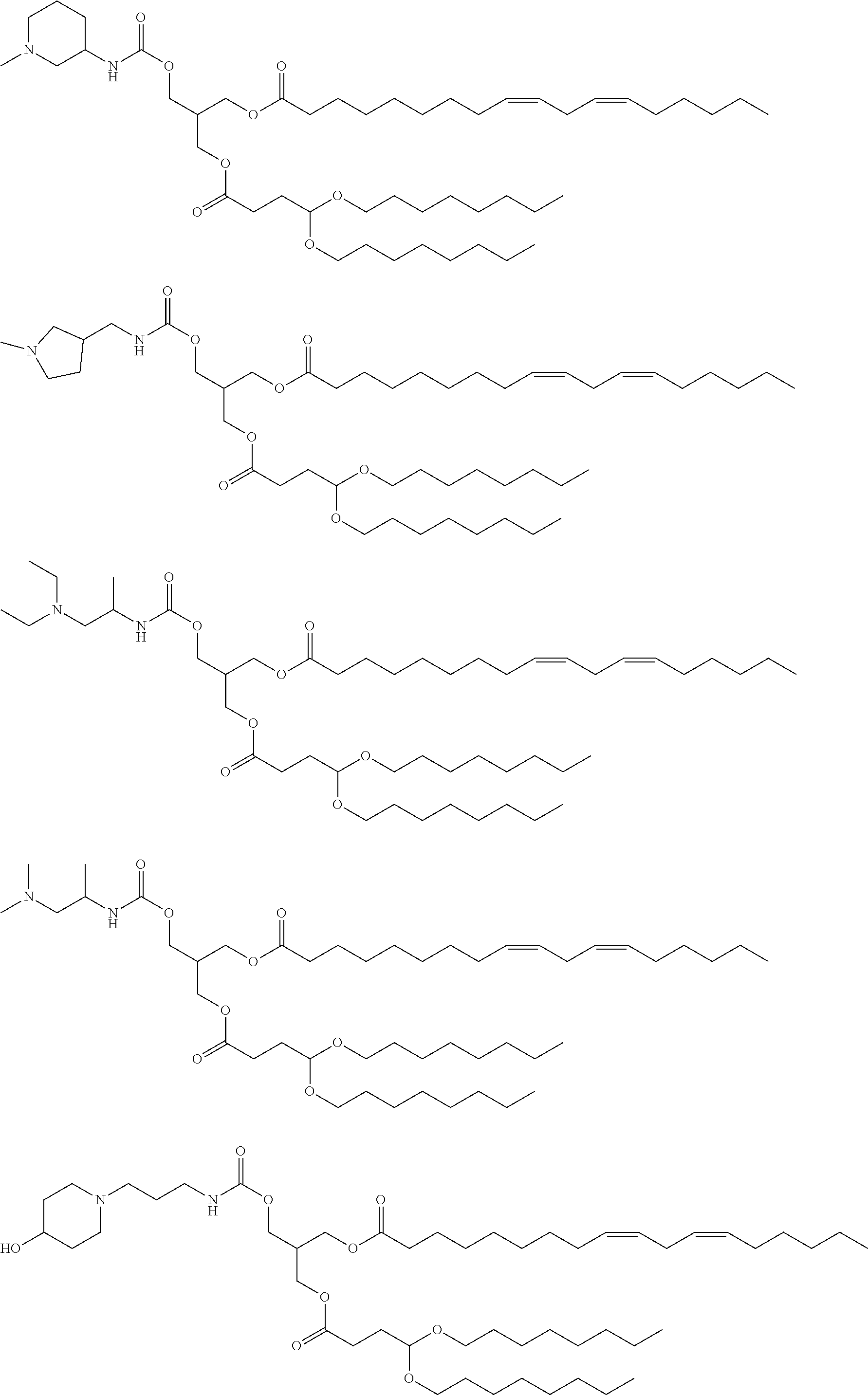

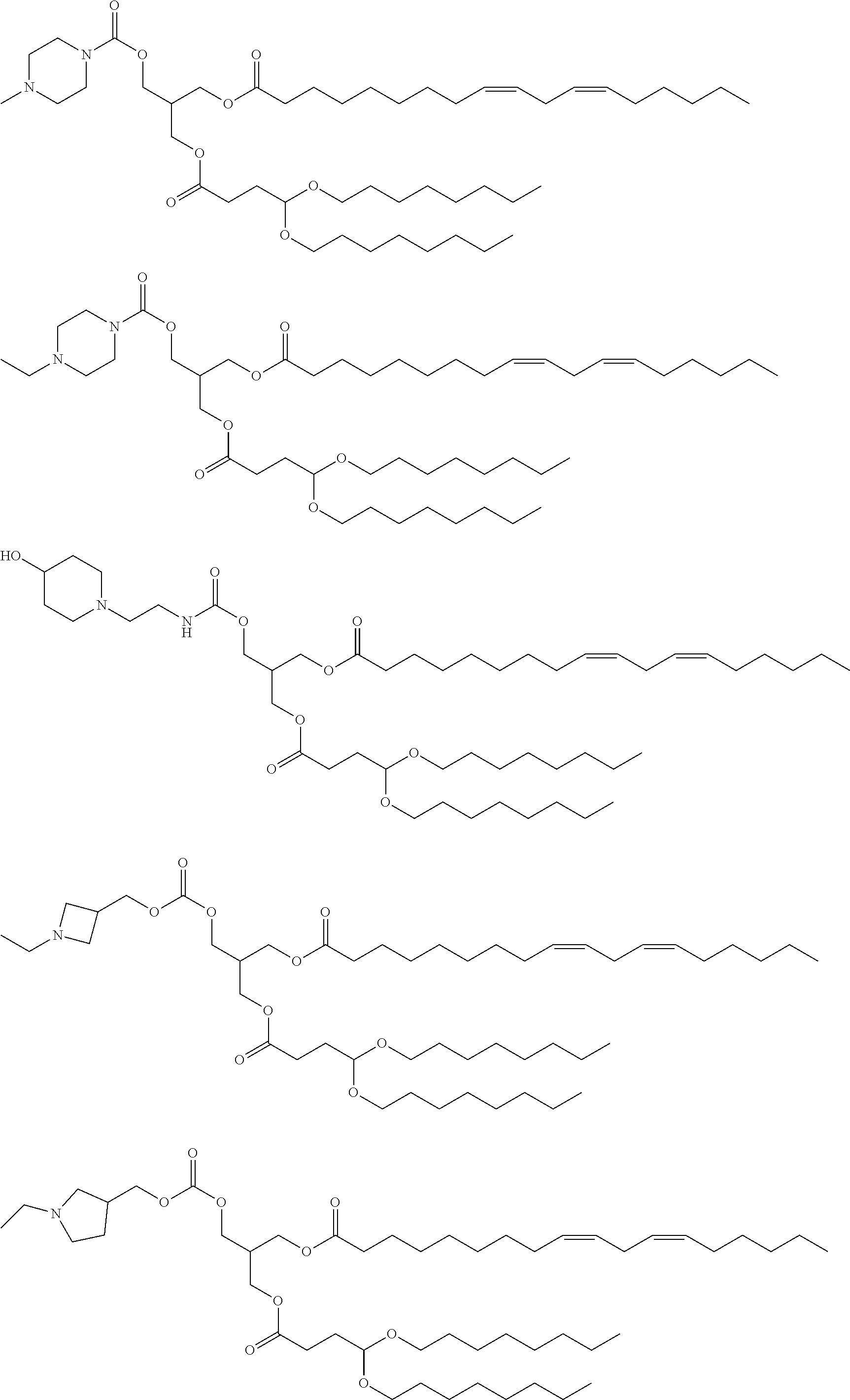

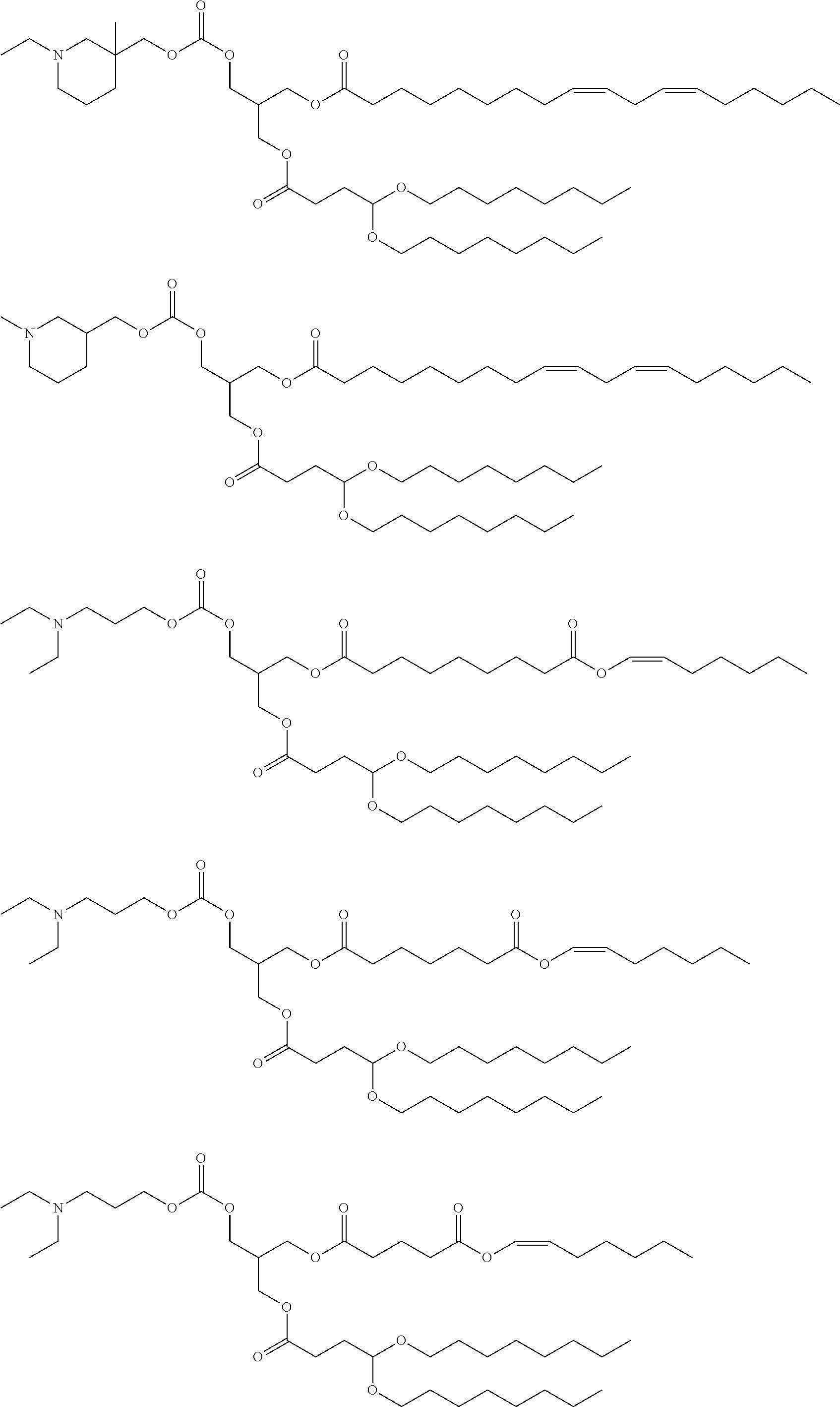

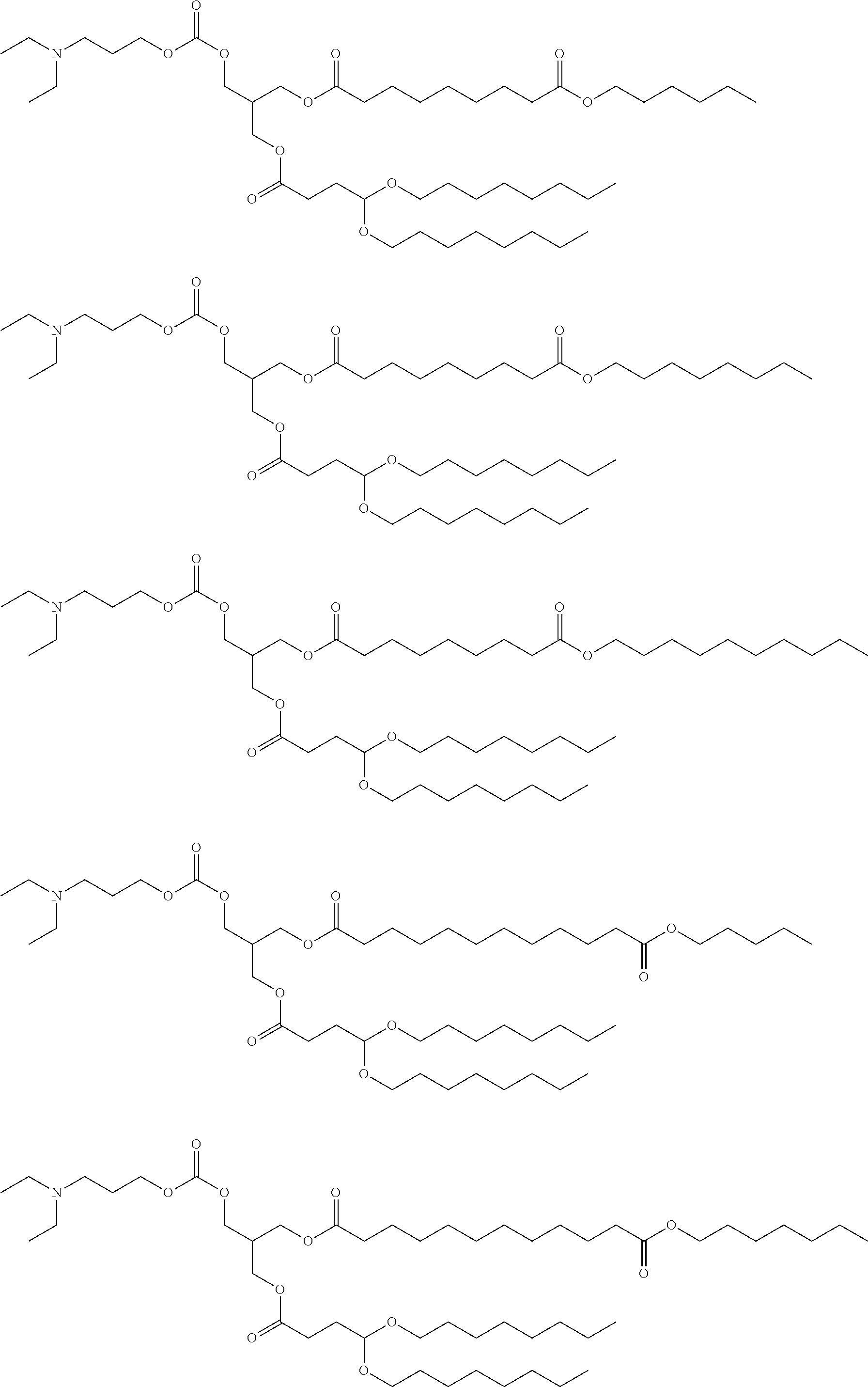

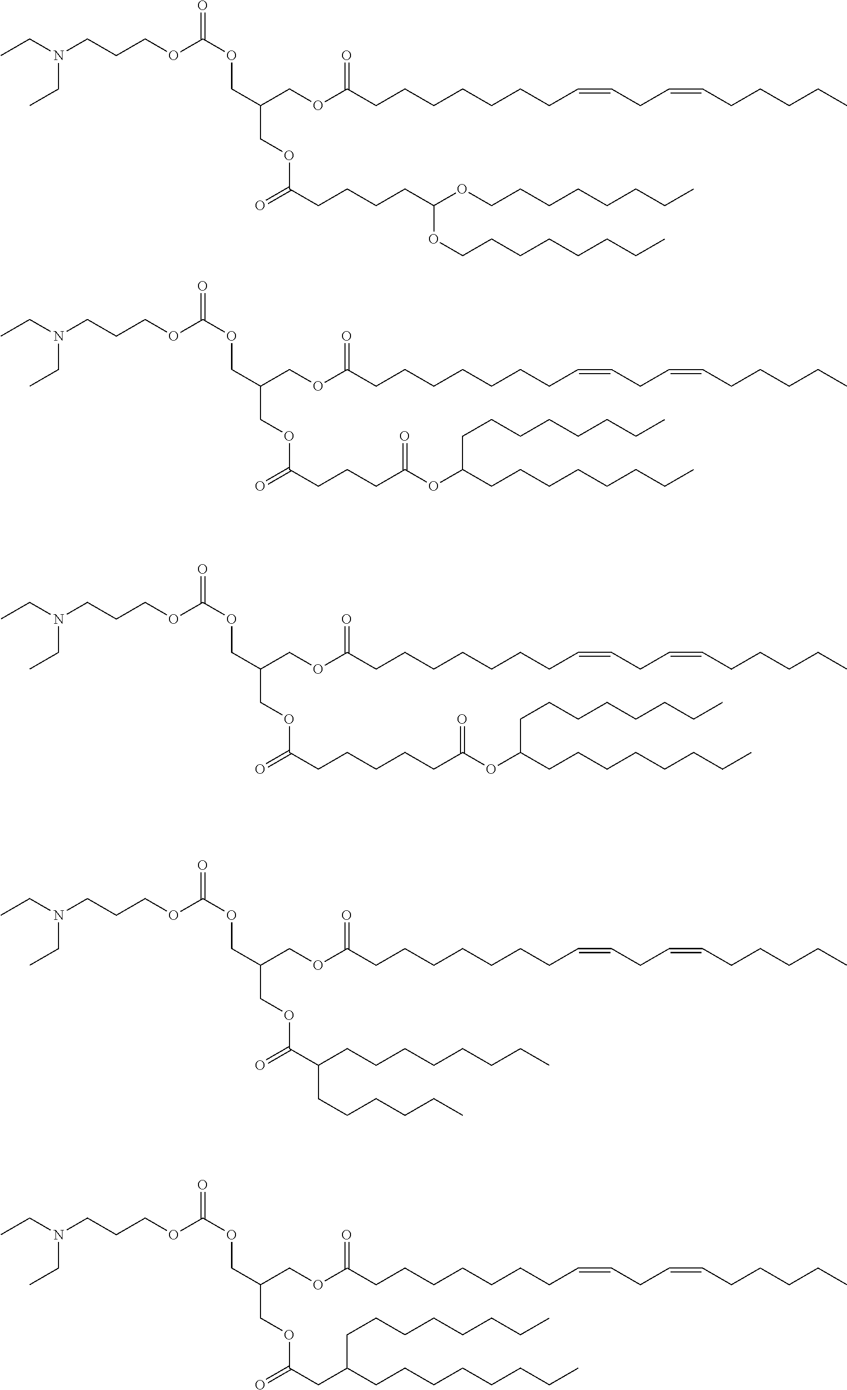

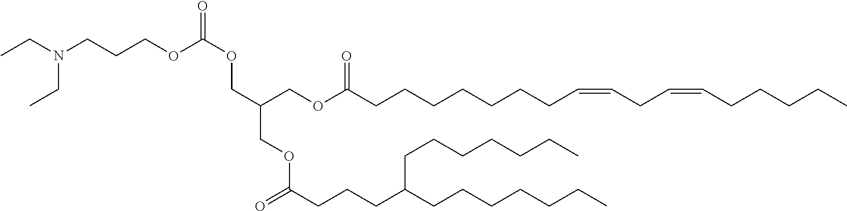 [SMILES: CCCCC/C=C\C/C=C\CCCCCCCC(=O)OCC(COC(=O)CCCC(CCCCCCC)CCCCCCC)COC(=O)OCCCN(CC)CC]

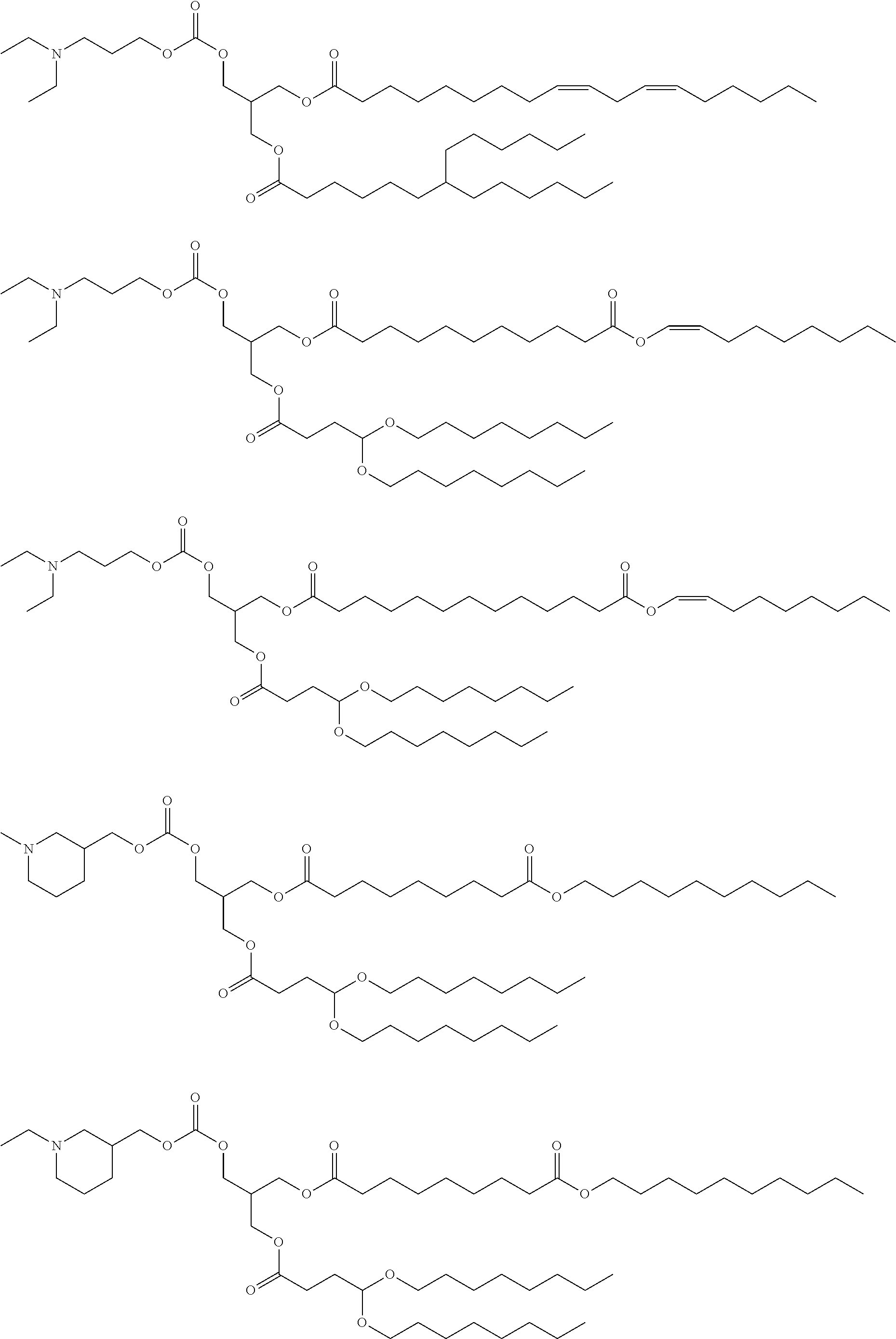

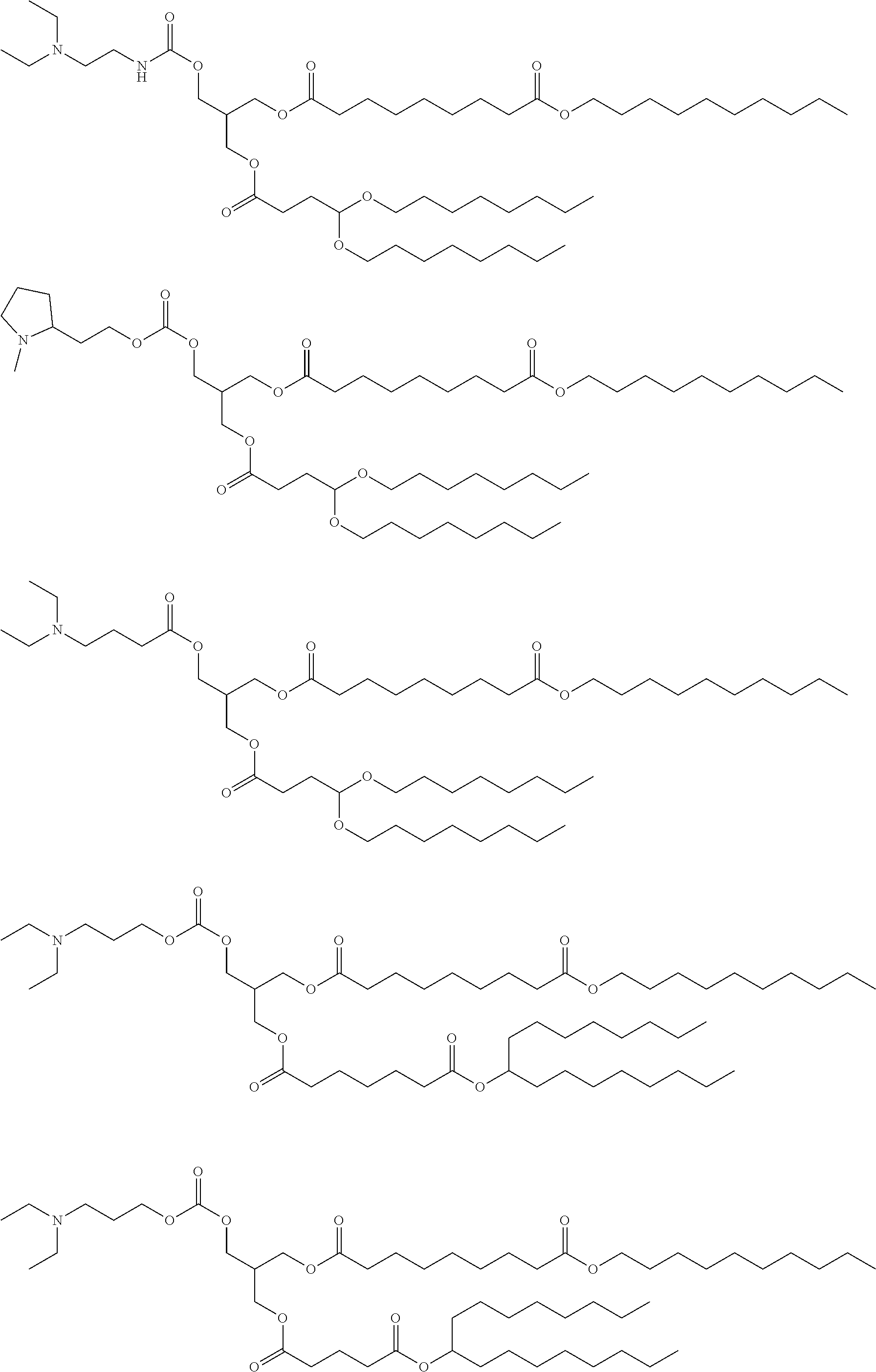

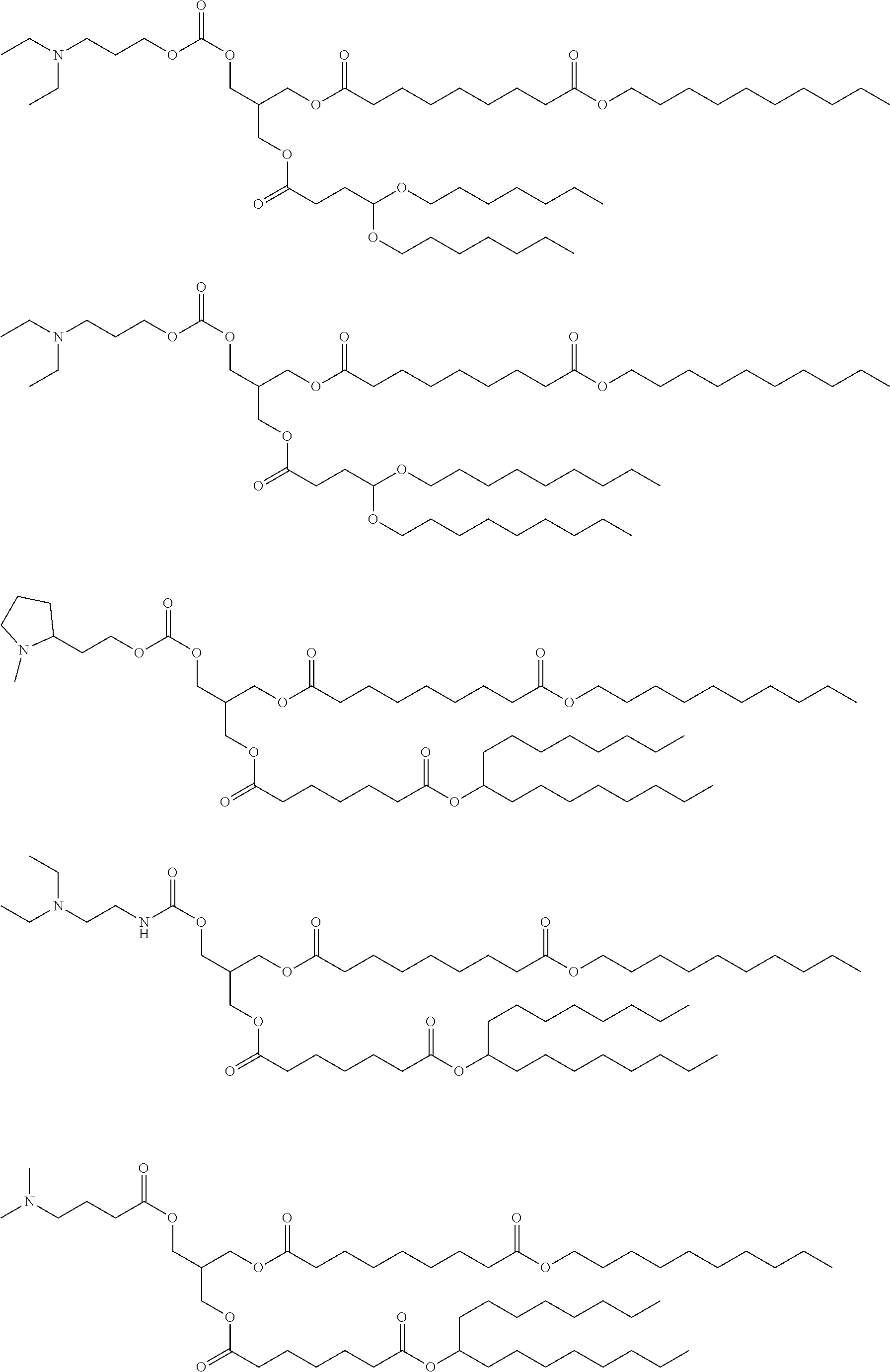

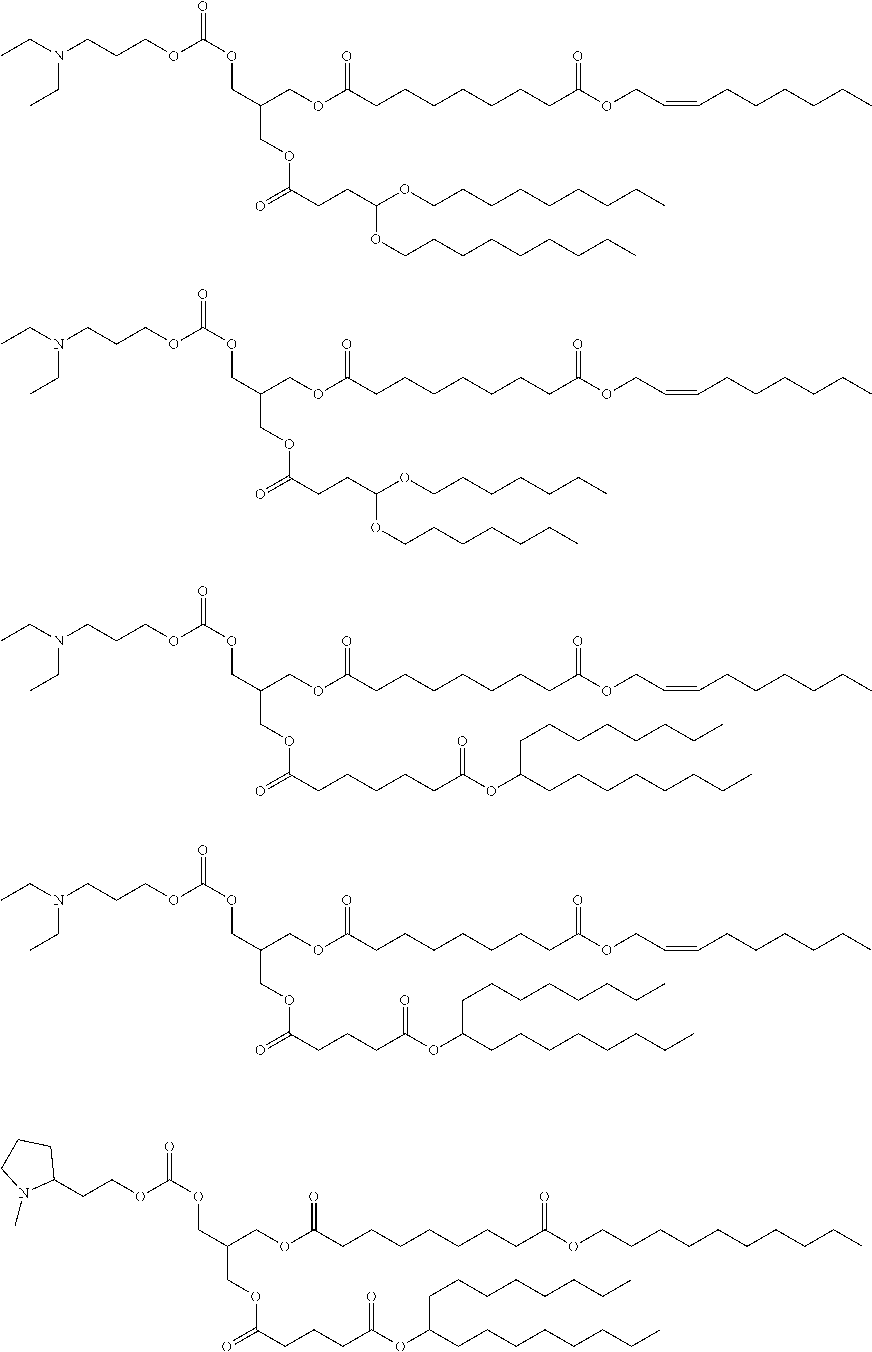

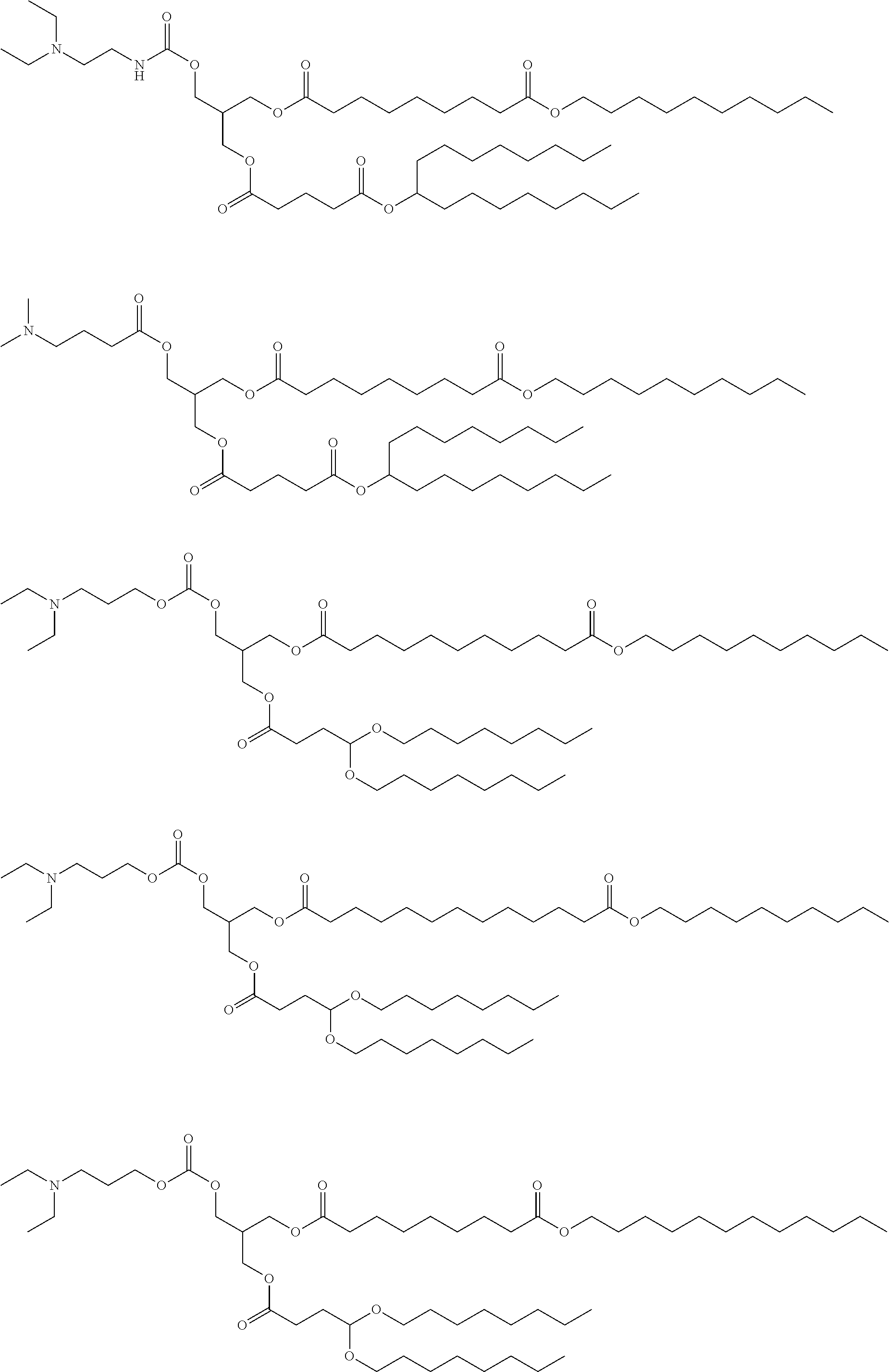

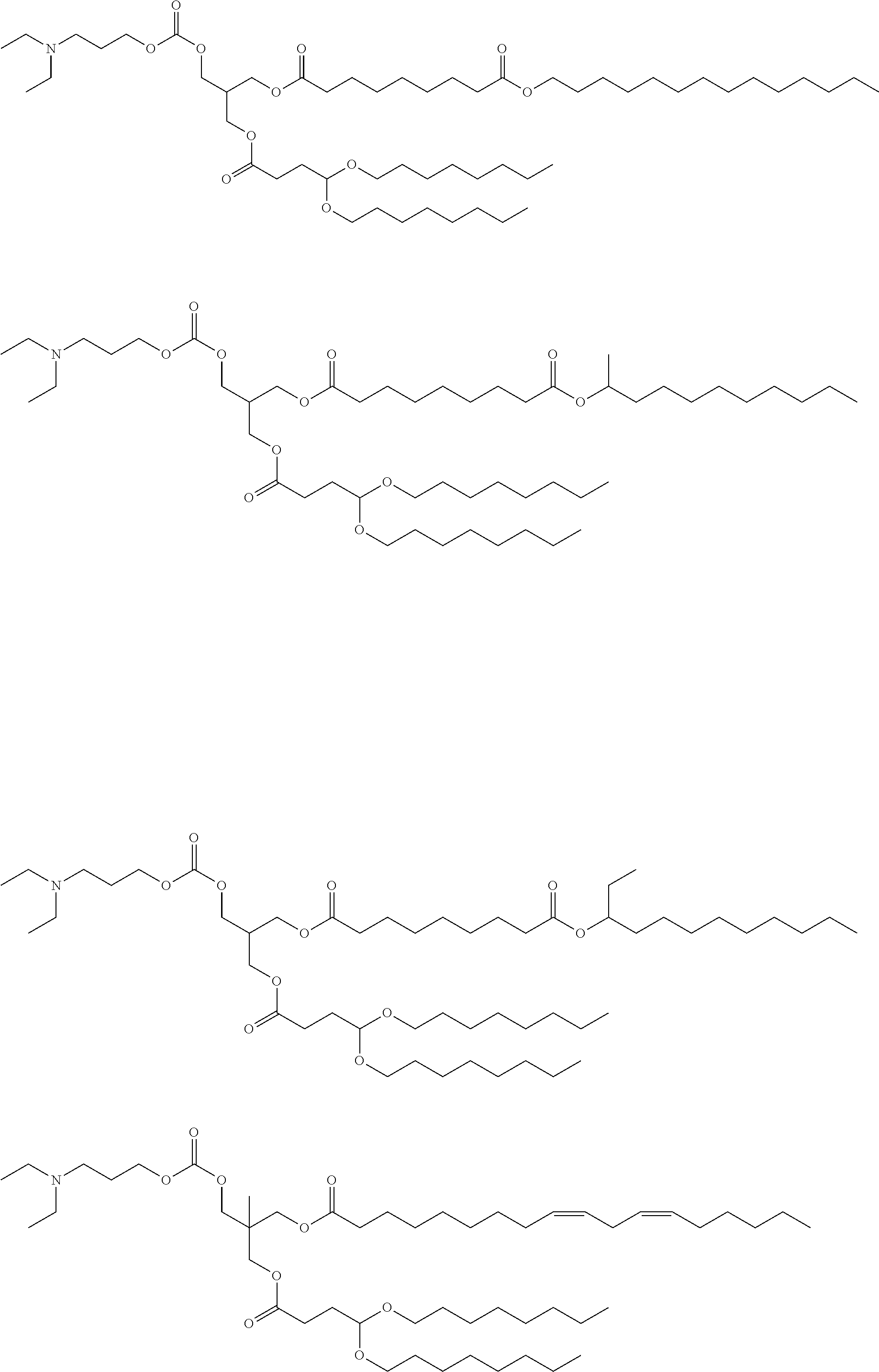

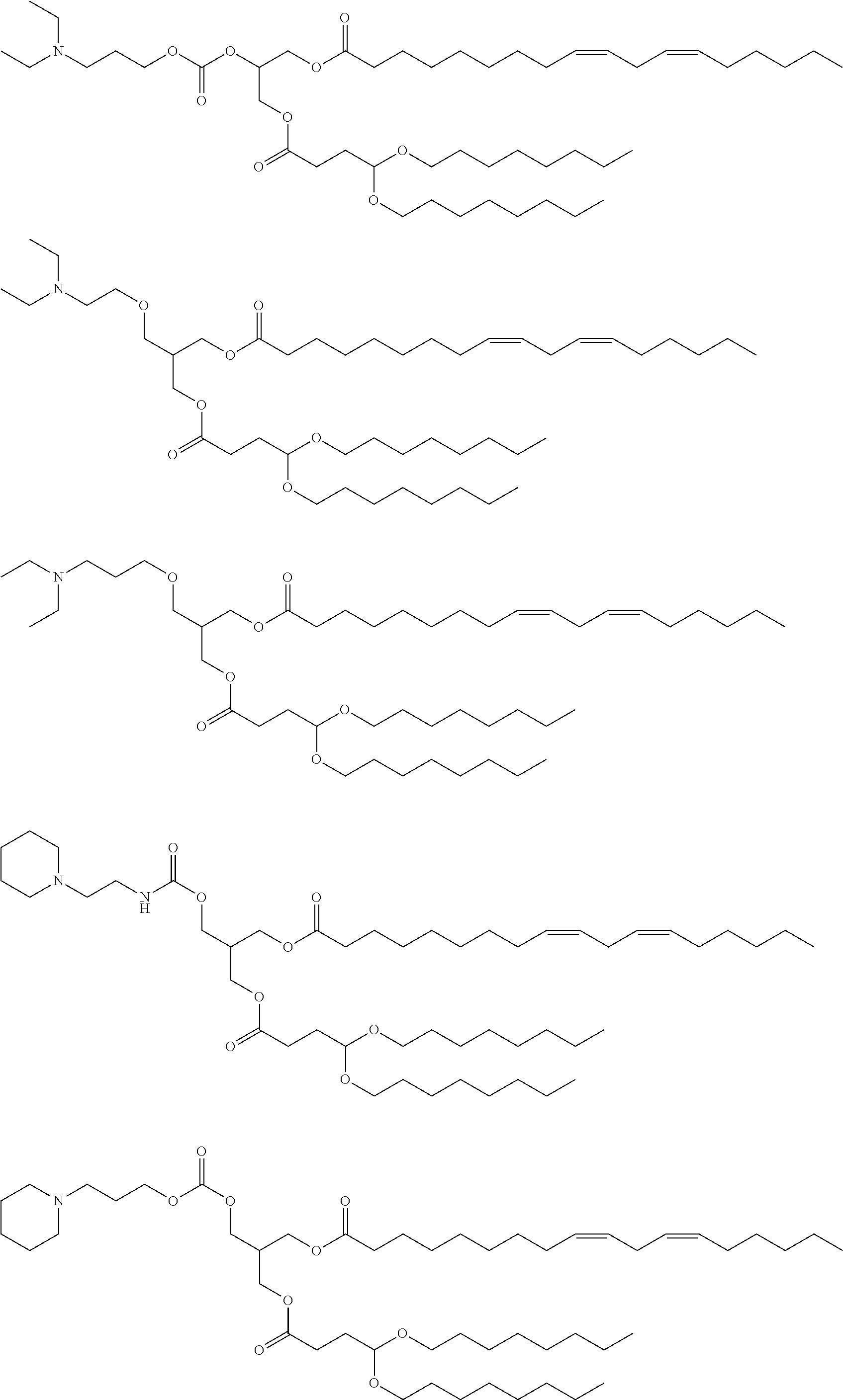

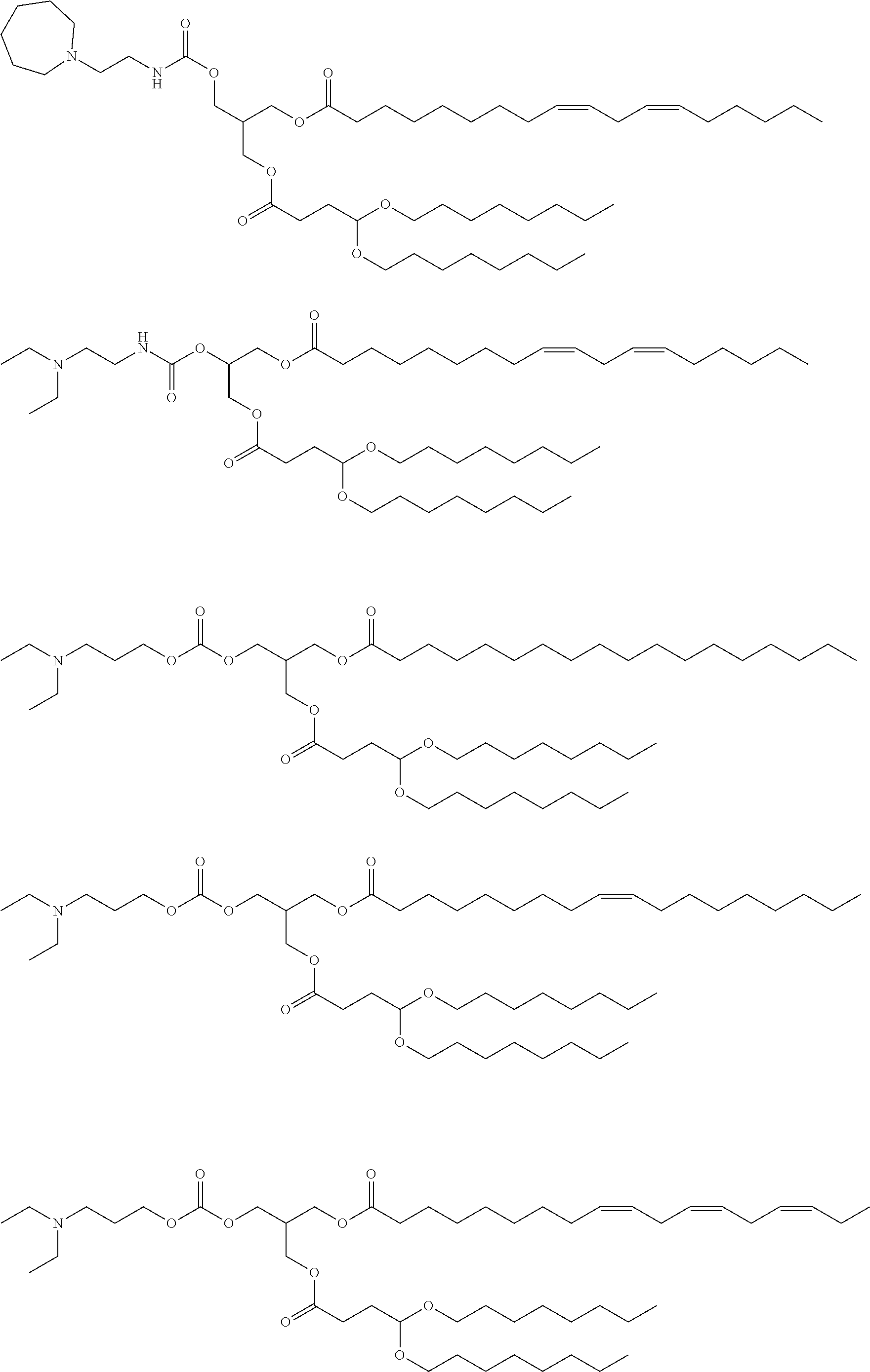

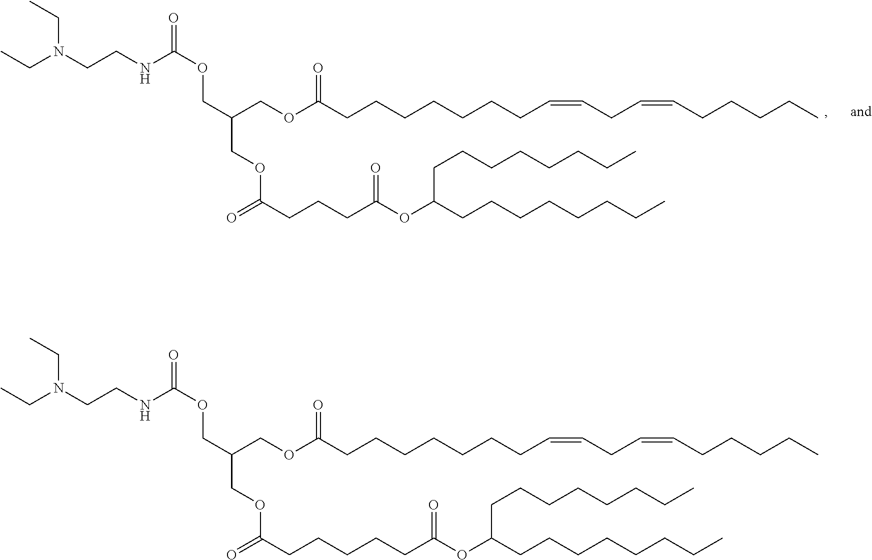

or a salt thereof, such as a pharmaceutically acceptable salt thereof. In certain embodiments, at least 75% of the compound of Formula (II) or (I) of lipid compositions formulated as disclosed herein is cleared from the subject's plasma within 8, 10, 12, 24, or 48 hours, or 3, 4, 5, 6, 7, or 10 days after administration. In certain embodiments, at least 50% of the lipid compositions comprising a compound of Formula (II) or (I) as disclosed herein are cleared from the subject's plasma within 8, 10, 12, 24, or 48 hours, or 3, 4, 5, 6, 7, or 10 days after administration, which can be determined, for example, by measuring a lipid (e.g. a compound of Formula (II) or (I)), RNA (e.g. mRNA), or other component in the plasma. In certain embodiments, lipid-encapsulated versus free lipid, RNA, or nucleic acid component of the lipid composition is measured.

Lipid clearance may be measured as described in literature. See Maier, M. A., et al. Biodegradable Lipids Enabling Rapidly Eliminated Lipid Nanoparticles for Systemic Delivery of RNAi Therapeutics. Mol. Ther. 2013, 21(8), 1570-78 (“Maier”). For example, in Maier, LNP-siRNA systems containing luciferases-targeting siRNA were administered to six- to eight-week old male C57Bl/6 mice at 0.3 mg/kg by intravenous bolus injection via the lateral tail vein. Blood, liver, and spleen samples were collected at 0.083, 0.25, 0.5, 1, 2, 4, 8, 24, 48, 96, and 168 hours post-dose. Mice were perfused with saline before tissue collection and blood samples were processed to obtain plasma. All samples were processed and analyzed by LC-MS. Further, Maier describes a procedure for assessing toxicity after administration of LNP-siRNA compositions. For example, a luciferase-targeting siRNA was administered at 0, 1, 3, 5, and 10 mg/kg (5 animals/group) via single intravenous bolus injection at a dose volume of 5 mL/kg to male Sprague-Dawley rats. After 24 hours, about 1 mL of blood was obtained from the jugular vein of conscious animals and the serum was isolated. At 72 hours post-dose, all animals were euthanized for necropsy. Assessment of clinical signs, body weight, serum chemistry, organ weights and histopathology was performed. Although Maier describes methods for assessing siRNA-LNP compositions, these methods may be applied to assess clearance, pharmacokinetics, and toxicity of administration of lipid compositions, such as LNP compositions, of the present disclosure.

In certain embodiments, lipid compositions using the compounds of Formula (II) or (I) disclosed herein exhibit an increased clearance rate relative to alternative ionizable amine lipids. In some such embodiments, the clearance rate is a lipid clearance rate, for example the rate at which a compound of Formula (II) or (I) is cleared from the blood, serum, or plasma. In some embodiments, the clearance rate is a cargo (e.g. biologically active agent) clearance rate, for example the rate at which a cargo component is cleared from the blood, serum, or plasma. In some embodiments, the clearance rate is an RNA clearance rate, for example the rate at which an mRNA or a gRNA is cleared from the blood, serum, or plasma. In some embodiments, the clearance rate is the rate at which LNP is cleared from the blood, serum, or plasma. In some embodiments, the clearance rate is the rate at which LNP is cleared from a tissue, such as liver tissue or spleen tissue. Desirably, a high rate of clearance can result in a safety profile with no substantial adverse effects, and/or reduced LNP accumulation in circulation and/or in tissues.

The compounds of Formula (II) or (I) of the present disclosure may form salts depending upon the pH of the medium they are in. For example, in a slightly acidic medium, the compounds of Formula (II) or (I) may be protonated and thus bear a positive charge. Conversely, in a slightly basic medium, such as, for example, blood where pH is approximately 7.35, the compounds of Formula (II) or (I) may not be protonated and thus bear no charge. In some embodiments, the compounds of Formula (II) or (I) of the present disclosure may be predominantly protonated at a pH of at least about 9. In some embodiments, the compounds of Formula (II) or (I) of the present disclosure may be predominantly protonated at a pH of at least about 10.

The pH at which a compound of Formula (II) or (I) is predominantly protonated is related to its intrinsic pKa. In preferred embodiments, a salt of a compound of Formula (II) or (I) of the present disclosure has a pKa in the range of from about 5.1 to about 8.0, even more preferably from about 5.5 to about 7.6. In other embodiments, a salt of a compound of Formula (II) or (I) of the present disclosure has a pKa in the range of from about 5.7 to about 7.6, e.g., from about 6 to about 7.5. Alternatively, a salt of a compound of Formula (II) or (I) of the present disclosure has a pKa in the range of from about 6 to about 8. The pKa of a salt of a compound of Formula (II) or (I) can be an important consideration in formulating LNPs, as it has been found that LNPs formulated with certain lipids having a pKa ranging from about 5.5 to about 7.0 are effective for delivery of cargo in vivo, e.g. to the liver. Further, it has been found that LNPs formulated with certain lipids having a pKa ranging from about 5.3 to about 6.4 are effective for delivery in vivo, e.g. to tumors. See, e.g., WO 2014/136086.

### Additional Lipids

“Neutral lipids” suitable for use in a lipid composition of the disclosure include, for example, a variety of neutral, uncharged or zwitterionic lipids. Examples of neutral phospholipids suitable for use in the present disclosure include, but are not limited to, dipalmitoylphosphatidylcholine (DPPC), di stearoylphosphatidylcholine (DSPC), phosphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine di stearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof. In certain embodiments, the neutral phospholipid may be selected from di stearoylphosphatidylcholine (DSPC) and dimyristoyl phosphatidyl ethanolamine (DMPE), preferably di stearoylphosphatidylcholine (DSPC).

“Helper lipids” include steroids, sterols, and alkyl resorcinols. Helper lipids suitable for use in the present disclosure include, but are not limited to, cholesterol, 5-heptadecylresorcinol, and cholesterol hemisuccinate. In certain embodiments, the helper lipid may be cholesterol or a derivative thereof, such as cholesterol hemisuccinate.

PEG lipids can affect the length of time the nanoparticles can exist in vivo (e.g., in the blood). PEG lipids may assist in the formulation process by, for example, reducing particle aggregation and controlling particle size. PEG lipids used herein may modulate pharmacokinetic properties of the LNPs. Typically, the PEG lipid comprises a lipid moiety and a polymer moiety based on PEG (sometimes referred to as poly(ethylene oxide)) (a

PEG moiety). PEG lipids suitable for use in a lipid composition with a compound of Formula (II) or (I) of the present disclosure and information about the biochemistry of such lipids can be found in Romberg et al., Pharmaceutical Research 25(1), 2008, pp. 55-71 and Hoekstra et al., Biochimica et Biophysica Acta 1660 (2004) 41-52. Additional suitable PEG lipids are disclosed, e.g., in WO 2015/095340 (p. 31, line 14 top. 37, line 6), WO 2006/007712, and WO 2011/076807 (“stealth lipids”).

In some embodiments, the lipid moiety may be derived from diacylglycerol or diacylglycamide, including those comprising a dialkylglycerol or dialkylglycamide group having alkyl chain length independently comprising from about C₄ to about C₄₀ saturated or unsaturated carbon atoms, wherein the chain may comprise one or more functional groups such as, for example, an amide or ester. In some embodiments, the alkyl chain length comprises about C₁₀ to C_{20.} The dialkylglycerol or dialkylglycamide group can further comprise one or more substituted alkyl groups. The chain lengths may be symmetrical or asymmetric.

Unless otherwise indicated, the term “PEG” as used herein means any polyethylene glycol or other polyalkylene ether polymer, such as an optionally substituted linear or branched polymer of ethylene glycol or ethylene oxide. In certain embodiments, the PEG moiety is unsubstituted. Alternatively, the PEG moiety may be substituted, e.g., by one or more alkyl, alkoxy, acyl, hydroxy, or aryl groups. For example, the PEG moiety may comprise a PEG copolymer such as PEG-polyurethane or PEG-polypropylene (see, e.g., J. Milton Harris, Poly(ethylene glycol) chemistry: biotechnical and biomedical applications (1992)); alternatively, the PEG moiety may be a PEG homopolymer. In certain embodiments, the PEG moiety has a molecular weight of from about 130 to about 50,000, such as from about 150 to about 30,000, or even from about 150 to about 20,000. Similarly, the PEG moiety may have a molecular weight of from about 150 to about 15,000, from about 150 to about 10,000, from about 150 to about 6,000, or even from about 150 to about 5,000. In certain preferred embodiments, the PEG moiety has a molecular weight of from about 150 to about 4,000, from about 150 to about 3,000, from about 300 to about 3,000, from about 1,000 to about 3,000, or from about 1,500 to about 2,500.

In certain preferred embodiments, the PEG moiety is a “PEG-2K,” also termed “PEG 2000,” which has an average molecular weight of about 2,000 daltons. PEG-2K is represented herein by the following formula (II), wherein n is 45, meaning that the number averaged degree of polymerization comprises about 45 subunits

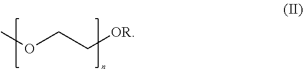

However, other PEG embodiments known in the art may be used, including, e.g., those where the number-averaged degree of polymerization comprises about 23 subunits (n=23), and/or 68 subunits (n=68). In some embodiments, n may range from about 30 to about 60. In some embodiments, n may range from about 35 to about 55. In some embodiments, n may range from about 40 to about 50. In some embodiments, n may range from about 42 to about 48. In some embodiments, n may be 45. In some embodiments, R may be selected from H, substituted alkyl, and unsubstituted alkyl. In some embodiments, R may be unsubstituted alkyl, such as methyl.

In any of the embodiments described herein, the PEG lipid may be selected from PEG-dilauroylglycerol, PEG-dimyristoylglycerol (PEG-DMG) (catalog #GM-020 from NOF, Tokyo, Japan), PEG-dipalmitoylglycerol, PEG-distearoylglycerol (PEG-DSPE) (catalog #DSPE-020CN, NOF, Tokyo, Japan), PEG-dilaurylglycamide, PEG-dimyristylglycamide, PEG-dipalmitoylglycamide, and PEG-di stearoylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol)ether), 1,2-dimyristoyl-sn-glycero-3-phosphoethanolamine-N-[methoxy(polyethylene glycol)-2000] (PEG2k-DMG), 1,2-distearoyl-sn-glycero-3-phosphoethanolamine-N-[methoxy(polyethylene glycol)-2000] (PEG2k-DSPE) (cat. #880120C from Avanti Polar Lipids, Alabaster, Ala., USA), 1,2-distearoyl-sn-glycerol, methoxypolyethylene glycol (PEG2k-DSG; GS-020, NOF Tokyo, Japan), poly(ethylene glycol)-2000-dimethacrylate (PEG2k-DMA), and 1,2-distearyloxypropyl-3-amine-N-[methoxy(polyethylene glycol)-2000] (PEG2k-DSA). In certain such embodiments, the PEG lipid may be PEG2k-DMG. In some embodiments, the PEG lipid may be PEG2k-DSG. In other embodiments, the PEG lipid may be PEG2k-DSPE. In some embodiments, the PEG lipid may be PEG2k-DMA. In yet other embodiments, the PEG lipid may be PEG2k-C-DMA. In certain embodiments, the PEG lipid may be compound S027, disclosed in WO2016/010840 (paragraphs [00240] to [00244]). In some embodiments, the PEG lipid may be PEG2k-DSA. In other embodiments, the PEG lipid may be PEG2k-C_{11.} In some embodiments, the PEG lipid may be PEG2k-C_{14.} In some embodiments, the PEG lipid may be PEG2k-C_{16.} In some embodiments, the PEG lipid may be PEG2k-C_{18.}

Cationic lipids suitable for use in a lipid composition of the invention include, but are not limited to, N,N-dioleyl-N,N-dimethylammonium chloride (DODAC),N,N-distearyl-N,N-dimethylammonium bromide (DDAB), N-(1-(2,3-dioleoyloxy)propyl)-N,N,N-trimethylammonium chloride (DOTAP), 1,2-Dioleoyl-3-Dimethylammonium -propane (DODAP), N-(1-(2,3-dioleyloxy)propyl)-N,N,N-trimethylammonium chloride (DOTMA), 1,2-Dioleoylcarbamyl-3-Dimethylammonium-propane (DOCDAP), 1,2-Dilineoyl-3-Dimethylammonium-propane (DLINDAP), dilauryl(C_{12:0}) trimethyl ammonium propane (DLTAP), Dioctadecylamidoglycyl spermine (DOGS), DC-Choi, Dioleoyloxy-N-[2-(sperminecarboxamido)ethyl]-N,N-dimethyl-1-propanaminiumtrifluoroacetate (DOSPA), 1,2-Dimyristyloxypropyl-3-dimethyl-hydroxyethyl ammonium bromide (DMRIE), 3-Dimethylamino-2-(Cholest-5-en-3-beta-oxybutan-4-oxy)-1-(cis,cis-9,12-octadecadienoxy)propane (CLinDMA), N,N-dimethyl-2,3-dioleyloxy)propylamine (DODMA), 2-[5′-(cholest-5-en-3[beta]-oxy)-3′-oxapentoxy)-3-dimethyl-1-(cis,cis-9′,1-2′-octadecadienoxy) propane (CpLinDMA), N,N-Dimethyl-3,4-dioleyloxybenzylamine (DMOBA), and 1,2-N,N′-Dioleylcarbamyl-3-dimethylaminopropane (DOcarbDAP). In one embodiment the cationic lipid is DOTAP or DLTAP.

Anionic lipids suitable for use in the present invention include, but are not limited to, phosphatidylglycerol, cardiolipin, diacylphosphatidylserine, diacylphosphatidic acid, N-dodecanoyl phosphatidyl ethanolamine, N-succinyl phosphatidylethanolamine, N-glutaryl phosphatidylethanolamine cholesterol hemisuccinate (CHEMS), and lysylphosphatidylglycerol.

### Lipid Compositions

The present invention provides a lipid composition comprising at least one compound of Formula (II) or (I) or a salt thereof (e.g., a pharmaceutically acceptable salt thereof) and at least one other lipid component. Such compositions can also contain a biologically active agent, optionally in combination with one or more other lipid components. In some embodiments, the lipid compositions comprise a lipid component and an aqueous component comprising a biologically active agent.

In one embodiment, the lipid composition comprises a compound of Formula (II) or (I), or a pharmaceutically acceptable salt thereof, and at least one other lipid component. In another embodiment, the lipid composition further comprises a biologically active agent, optionally in combination with one or more other lipid components. In another embodiment the lipid composition is in the form of a liposome. In another embodiment the lipid composition is in the form of a lipid nanoparticle (LNP). In another embodiment the lipid composition is suitable for delivery to the liver.

In one embodiment, the lipid composition comprises a compound of Formula (II) or (I), or a pharmaceutically acceptable salt thereof, and another lipid component. Such other lipid components include, but are not limited to, neutral lipids, helper lipids, PEG lipids, cationic lipids, and anionic lipids. In certain embodiments, the lipid composition comprises a compound of Formula (II) or (I), or a pharmaceutically acceptable salt thereof, and a neutral lipid, e.g. DSPC, optionally with one or more additional lipid components. In another embodiment, the lipid composition comprises a compound of Formula (II) or (I), or a pharmaceutically acceptable salt thereof, and a helper lipid, e.g. cholesterol, optionally with one or more additional lipid components. In further embodiment, the lipid composition comprises a compound of Formula (II) or (I), or a pharmaceutically acceptable salt thereof, and a PEG lipid, optionally with one or more additional lipid components. In further embodiment, the lipid composition comprises a compound of Formula (II) or (I), or a pharmaceutically acceptable salt thereof, and a cationic lipid, optionally with one or more additional lipid components. In further embodiment, the lipid composition comprises a compound of Formula (II) or (I), or a pharmaceutically acceptable salt thereof, and an anionic lipid, optionally with one or more additional lipid components. In a sub-embodiment, the lipid composition comprises a compound of Formula (II) or (I), or a pharmaceutically acceptable salt thereof, a helper lipid, and a PEG lipid, optionally with a neutral lipid. In a further sub-embodiment, the lipid composition comprises a compound of Formula (II) or (I), or a pharmaceutically acceptable salt thereof, a helper lipid, a PEG lipid, and a neutral lipid.

Compositions containing lipids of Formula (II) or (I), or a pharmaceutically acceptable salt thereof, or lipid compositions thereof may be in various forms, including, but not limited to, particle forming delivery agents including microparticles, nanoparticles and transfection agents that are useful for delivering various molecules to cells. Specific compositions are effective at transfecting or delivering biologically active agents. Preferred biologically active agents are RNAs and DNAs. In further embodiments, the biologically active agent is chosen from mRNA, gRNA, and DNA. The gRNA may be a dgRNA or an sgRNA. In certain embodiments, the cargo includes an mRNA encoding an RNA-guided DNA-binding agent (e.g. a Cas nuclease, a Class 2 Cas nuclease, or Cas9), a gRNA or a nucleic acid encoding a gRNA, or a combination of mRNA and gRNA.

Exemplary compounds of Formula (I) or (II) for use in the above lipid compositions are given in Examples 2-99, 100-103, and 113-118. In certain embodiments, the compound of Formula (I) is Compound 2. In certain embodiments, the compound of Formula (I) is Compound 3. In certain embodiments, the compound of Formula (I) is Compound 4. In certain embodiments, the compound of Formula (I) is Compound 5. In certain embodiments, the compound of Formula (I) is Compound 6. In certain embodiments, the compound of Formula (I) is Compound 7. In certain embodiments, the compound of Formula (I) is Compound 8. In certain embodiments, the compound of Formula (I) is Compound 9. In certain embodiments, the compound of Formula (I) is Compound 10. In certain embodiments, the compound of Formula (I) is Compound 11. In certain embodiments, the compound of Formula (I) is Compound 12. In certain embodiments, the compound of Formula (I) is Compound 13. In certain embodiments, the compound of Formula (I) is Compound 14. In certain embodiments, the compound of Formula (I) is Compound 15. In certain embodiments, the compound of Formula (I) is Compound 16. In certain embodiments, the compound of Formula (I) is Compound 17. In certain embodiments, the compound of Formula (I) is Compound 18. In certain embodiments, the compound of Formula (I) is Compound 19. In certain embodiments, the compound of Formula (I) is Compound 20. In certain embodiments, the compound of Formula (I) is Compound 21. In certain embodiments, the compound of Formula (I) is Compound 22. In certain embodiments, the compound of Formula (I) is Compound 23. In certain embodiments, the compound of Formula (I) is Compound 24. In certain embodiments, the compound of Formula (I) is Compound 25. In certain embodiments, the compound of Formula (I) is Compound 26. In certain embodiments, the compound of Formula (I) is Compound 27. In certain embodiments, the compound of Formula (I) is Compound 28. In certain embodiments, the compound of Formula (I) is Compound 29. In certain embodiments, the compound of Formula (I) is Compound 30. In certain embodiments, the compound of Formula (I) is Compound 31. In certain embodiments, the compound of Formula (I) is Compound 32. In certain embodiments, the compound of Formula (I) is Compound 33. In certain embodiments, the compound of Formula (I) is Compound 34. In certain embodiments, the compound of Formula (I) is Compound 35. In certain embodiments, the compound of Formula (I) is Compound 36. In certain embodiments, the compound of Formula (I) is Compound 37. In certain embodiments, the compound of Formula (I) is Compound 38. In certain embodiments, the compound of Formula (I) is

Compound 39. In certain embodiments, the compound of Formula (I) is Compound 40. In certain embodiments, the compound of Formula (I) is Compound 41. In certain embodiments, the compound of Formula (I) is Compound 42. In certain embodiments, the compound of Formula (I) is Compound 43. In certain embodiments, the compound of Formula (I) is Compound 44. In certain embodiments, the compound of Formula (I) is Compound 45. In certain embodiments, the compound of Formula (I) is Compound 46. In certain embodiments, the compound of Formula (I) is Compound 47. In certain embodiments, the compound of Formula (I) is Compound 48. In certain embodiments, the compound of Formula (I) is Compound 49. In certain embodiments, the compound of Formula (I) is Compound 50. In certain embodiments, the compound of Formula (I) is Compound 51. In certain embodiments, the compound of Formula (I) is Compound 52. In certain embodiments, the compound of Formula (I) is Compound 53. In certain embodiments, the compound of Formula (I) is Compound 54. In certain embodiments, the compound of Formula (I) is Compound 55. In certain embodiments, the compound of Formula (I) is Compound 56. In certain embodiments, the compound of Formula (I) is Compound 57. In certain embodiments, the compound of Formula (I) is Compound 58. In certain embodiments, the compound of Formula (I) is Compound 59. In certain embodiments, the compound of Formula (I) is Compound 60. In certain embodiments, the compound of Formula (I) is Compound 61. In certain embodiments, the compound of Formula (I) is Compound 62. In certain embodiments, the compound of Formula (I) is Compound 63. In certain embodiments, the compound of Formula (I) is Compound 64. In certain embodiments, the compound of Formula (I) is Compound 65. In certain embodiments, the compound of Formula (I) is Compound 66. In certain embodiments, the compound of Formula (I) is Compound 67. In certain embodiments, the compound of Formula (I) is Compound 68. In certain embodiments, the compound of Formula (I) is Compound 69. In certain embodiments, the compound of Formula (I) is Compound 70. In certain embodiments, the compound of Formula (I) is Compound 71. In certain embodiments, the compound of Formula (I) is Compound 72. In certain embodiments, the compound of Formula (I) is Compound 73. In certain embodiments, the compound of Formula (I) is Compound 74. In certain embodiments, the compound of Formula (I) is Compound 75. In certain embodiments, the compound of Formula (I) is Compound 76. In certain embodiments, the compound of Formula (I) is Compound 77. In certain embodiments, the compound of Formula (I) is Compound 78. In certain embodiments, the compound of Formula (I) is Compound 79. In certain embodiments, the compound of Formula (I) is Compound 80. In certain embodiments, the compound of Formula (I) is Compound 81. In certain embodiments, the compound of Formula (I) is Compound 82. In certain embodiments, the compound of Formula (I) is Compound 83. In certain embodiments, the compound of Formula (I) is Compound 84. In certain embodiments, the compound of Formula (I) is Compound 85. In certain embodiments, the compound of Formula (I) is Compound 86. In certain embodiments, the compound of Formula (I) is Compound 87. In certain embodiments, the compound of Formula (I) is Compound 88. In certain embodiments, the compound of Formula (I) is Compound 89. In certain embodiments, the compound of Formula (I) is Compound 90. In certain embodiments, the compound of Formula (I) is Compound 91. In certain embodiments, the compound of Formula (I) is Compound 92. In certain embodiments, the compound of Formula (I) is Compound 93. In certain embodiments, the compound of Formula (I) is Compound 94. In certain embodiments, the compound of Formula (I) is Compound 95. In certain embodiments, the compound of Formula (I) is Compound 96. In certain embodiments, the compound of Formula (I) is Compound 97. In certain embodiments, the compound of Formula (I) is Compound 98. In certain embodiments, the compound of Formula (I) is Compound 99. In certain embodiments, the compound is Compound 100. In certain embodiments, the compound is Compound 101. In certain embodiments, the compound is Compound 102. In certain embodiments, the compound is Compound 103. In certain embodiments, the compound is Compound 113. In certain embodiments, the compound is Compound 114. In certain embodiments, the compound is Compound 115. In certain embodiments, the compound is Compound 116. In certain embodiments, the compound is Compound 117. In certain embodiments, the compound is Compound 118.

### LNP Compositions

The lipid compositions may be provided as LNP compositions. Lipid nanoparticles may be, e.g., microspheres (including unilamellar and multilamellar vesicles, e.g. “liposomes”—lamellar phase lipid bilayers that, in some embodiments are substantially spherical, and, in more particular embodiments can comprise an aqueous core, e.g., comprising a substantial portion of RNA molecules), a dispersed phase in an emulsion, micelles or an internal phase in a suspension.

The LNPs have a size of about 1 to about 1,000 nm, about 10 to about 500 nm, about 20 to about 500 nm, in a sub-embodiment about 50 to about 400 nm, in a sub-embodiment about 50 to about 300 nm, in a sub-embodiment about 50 to about 200 nm, and in a sub-embodiment about 50 to about 150 nm, and in another sub-embodiment about 60 to about 120 nm. Preferably, the LNPs have a size from about 60 nm to about 100 nm. The average sizes (diameters) of the fully formed LNP, may be measured by dynamic light scattering on a Malvern Zetasizer or Wyatt NanoStar. The LNP sample is diluted in phosphate buffered saline (PBS) so that the count rate is approximately 200 — 400 kcps. The data is presented as a weighted average of the intensity measure.

Embodiments of the present disclosure provide lipid compositions described according to the respective molar ratios of the component lipids in the composition. All mol-% numbers are given as a fraction of the lipid component of the lipid composition or, more specifically, the LNP compositions. In certain embodiments, the mol-% of the compound of Formula (II) or (I) may be from about 30 mol-% to about 70 mol-%. In certain embodiments, the mol-% of the compound of Formula (II) or (I) may at least 30 mol-%, at least 40 mol-%, at least 50 mol-%, or at least 60 mol-%. In certain embodiments, the mol-% of the neutral lipid may be from about 0 mol-% to about 30 mol-%. In certain embodiments, the mol-% of the neutral lipid may be from about 0 mol-% to about 20 mol-%. In certain embodiments, the mol-% of the neutral lipid may be about 10 mol-%. In certain embodiments, the mol-% of the neutral lipid may be about 9 mol-%.

In certain embodiments, the mol-% of the helper lipid may be from about 0 mol-% to about 80 mol-%. In certain embodiments, the mol-% of the helper lipid may be from about 20 mol-% to about 60 mol-%. In certain embodiments, the mol-% of the helper lipid may be from about 30 mol-% to about 50 mol-%. In certain embodiments, the mol-% of the helper lipid may be from 30 mol-% to about 40 mol-% or from about 35% mol-% to about 45 mol-%. In certain embodiments, the mol-% of the helper lipid is adjusted based on compound of Formula (II) or (I), neutral lipid, and/or PEG lipid concentrations to bring the lipid component to 100 mol-%.

In certain embodiments, the mol-% of the PEG lipid may be from about 1 mol-% to about 10 mol-%. In certain embodiments, the mol-% of the PEG lipid may be from about 1 mol-% to about 4 mol-%. In certain embodiments, the mol-% of the PEG lipid may be about 1 mol-% to about 2 mol-%. In certain embodiments, the mol-% of the PEG lipid may be about 1.5 mol-%.

In various embodiments, an LNP composition comprises a compound of Formula (II) or (I) or a salt thereof (such as a pharmaceutically acceptable salt thereof (e.g., as disclosed herein)), a neutral lipid (e.g., DSPC), a helper lipid (e.g., cholesterol), and a PEG lipid (e.g., PEG2k-DMG). In some embodiments, an LNP composition comprises a compound of Formula (II) or (I) or a pharmaceutically acceptable salt thereof (e.g., as disclosed herein), DSPC, cholesterol, and a PEG lipid. In some such embodiments, the LNP composition comprises a PEG lipid comprising DMG, such as PEG2k-DMG. In certain preferred embodiments, an LNP composition comprises a compound of Formula (II) or (I) or a pharmaceutically acceptable salt thereof, cholesterol, DSPC, and PEG2k-DMG.

In certain embodiments, the lipid compositions, such as LNP compositions, comprise a lipid component and a nucleic acid component, e.g. an RNA component and the molar ratio of compound of Formula (II) or (I) to nucleic acid can be measured. Embodiments of the present disclosure also provide lipid compositions having a defined molar ratio between the positively charged amine groups of pharmaceutically acceptable salts of the compounds of Formula (II) or (I) (N) and the negatively charged phosphate groups (P) of the nucleic acid to be encapsulated. This may be mathematically represented by the equation N/P. In some embodiments, a lipid composition, such as an LNP composition, may comprise a lipid component that comprises a compound of Formula (II) or (I) or a pharmaceutically acceptable salt thereof; and a nucleic acid component, wherein the N/P ratio is about 3 to 10. In some embodiments, an LNP composition may comprise a lipid component that comprises a compound of Formula (II) or (I) or a pharmaceutically acceptable salt thereof; and an RNA component, wherein the N/P ratio is about 3 to 10. For example, the N/P ratio may be about 4-7. Alternatively, the N/P ratio may about 6, e.g., 6±1, or 6±0.5.

In some embodiments, the aqueous component comprises a biologically active agent. In some embodiments, the aqueous component comprises a polypeptide, optionally in combination with a nucleic acid. In some embodiments, the aqueous component comprises a nucleic acid, such as an RNA. In some embodiments, the aqueous component is a nucleic acid component. In some embodiments, the nucleic acid component comprises DNA and it can be called a DNA component. In some embodiments, the nucleic acid component comprises RNA. In some embodiments, the aqueous component, such as an RNA component may comprise an mRNA, such as an mRNA encoding an RNA-guided

DNA-binding agent. In some embodiments, the RNA-guided DNA-binding agent is a Cas nuclease. In certain embodiments, aqueous component may comprise an mRNA that encodes Cas9. In certain embodiments, the aqueous component may comprise a gRNA. In some compositions comprising an mRNA encoding an RNA-guided DNA-binding agent, the composition further comprises a gRNA nucleic acid, such as a gRNA. In some embodiments, the aqueous component comprises an RNA-guided DNA-binding agent and a gRNA. In some embodiments, the aqueous component comprises a Cas nuclease mRNA and a gRNA. In some embodiments, the aqueous component comprises a Class 2 Cas nuclease mRNA and a gRNA.

In certain embodiments, a lipid composition, such as an LNP composition, may comprise an mRNA encoding a Cas nuclease such as a Class 2 Cas nuclease, a compound of Formula (II) or (I) or a pharmaceutically acceptable salt thereof, a helper lipid, optionally a neutral lipid, and a PEG lipid. In certain compositions comprising an mRNA encoding a Cas nuclease such as a Class 2 Cas nuclease, the helper lipid is cholesterol. In other compositions comprising an mRNA encoding a Cas nuclease such as a Class 2 Cas nuclease, the neutral lipid is DSPC. In additional embodiments comprising an mRNA encoding a Cas nuclease such as a Class 2 Cas nuclease, e.g. Cas9, the PEG lipid is PEG2k-DMG. In specific compositions comprising an mRNA encoding a Cas nuclease such as a Class 2 Cas nuclease, and a compound of Formula (II) or (I) or a pharmaceutically acceptable salt thereof. In certain compositions, the composition further comprises a gRNA, such as a dgRNA or an sgRNA.

In some embodiments, a lipid composition, such as an LNP composition, may comprise a gRNA. In certain embodiments, a composition may comprise a compound of Formula (II) or (I) or a pharmaceutically acceptable salt thereof, a gRNA, a helper lipid, optionally a neutral lipid, and a PEG lipid. In certain LNP compositions comprising a gRNA, the helper lipid is cholesterol. In some compositions comprising a gRNA, the neutral lipid is DSPC. In additional embodiments comprising a gRNA, the PEG lipid is PEG2k-DMG. In certain compositions, the gRNA is selected from dgRNA and sgRNA.

In certain embodiments, a lipid composition, such as an LNP composition, comprises an mRNA encoding an RNA-guided DNA-binding agent and a gRNA, which may be an sgRNA, in an aqueous component and a compound of Formula (II) or (I) in a lipid component. For example, an LNP composition may comprise a compound of Formula (II) or (I) or a pharmaceutically acceptable salt thereof, an mRNA encoding a Cas nuclease, a gRNA, a helper lipid, a neutral lipid, and a PEG lipid. In certain compositions comprising an mRNA encoding a Cas nuclease and a gRNA, the helper lipid is cholesterol. In some compositions comprising an mRNA encoding a Cas nuclease and a gRNA, the neutral lipid is DSPC. In additional embodiments comprising an mRNA encoding a Cas nuclease and a gRNA, the PEG lipid is PEG2k-DMG.

In certain embodiments, the lipid compositions, such as LNP compositions include an RNA-guided DNA-binding agent, such as a Class 2 Cas mRNA and at least one gRNA. In certain embodiments, the LNP composition includes a ratio of gRNA to RNA-guided DNA-binding agent mRNA, such as Class 2 Cas nuclease mRNA of about 1:1 or about 1:2. In some embodiments, the ratio is from about 25:1 to about 1:25, from about 10:1 to about 1:10, from about 8:1 to about 1:8, from about 4:1 to about 1:4, or from about 2:1 to about 1:2.

The lipid compositions disclosed herein, such as LNP compositions, may include a template nucleic acid, e.g., a DNA template. The template nucleic acid may be delivered with, or separately from the lipid compositions comprising a compound of Formula (II) or

(I) or a pharmaceutically acceptable salt thereof, including as LNP compositions. In some embodiments, the template nucleic acid may be single- or double-stranded, depending on the desired repair mechanism. The template may have regions of homology to the target DNA, e.g. within the target DNA sequence, and/or to sequences adjacent to the target DNA.

In some embodiments, LNPs are formed by mixing an aqueous RNA solution with an organic solvent-based lipid solution. Suitable solutions or solvents include or may contain: water, PBS, Tris buffer, NaCl, citrate buffer, acetate buffer, ethanol, chloroform, diethylether, cyclohexane, tetrahydrofuran, methanol, isopropanol. For example, the organic solvent may be 100% ethanol. A pharmaceutically acceptable buffer, e.g., for in vivo administration of LNPs, may be used. In certain embodiments, a buffer is used to maintain the pH of the composition comprising LNPs at or above pH 6.5. In certain embodiments, a buffer is used to maintain the pH of the composition comprising LNPs at or above pH 7.0. In certain embodiments, the composition has a pH ranging from about 7.2 to about 7.7. In additional embodiments, the composition has a pH ranging from about 7.3 to about 7.7 or ranging from about 7.4 to about 7.6. In further embodiments, the composition has a pH of about 7.2, 7.3, 7.4, 7.5, 7.6, or 7.7. The pH of a composition may be measured with a micro pH probe. In certain embodiments, a cryoprotectant is included in the composition. Non-limiting examples of cryoprotectants include sucrose, trehalose, glycerol, DMSO, and ethylene glycol. Exemplary compositions may include up to 10% cryoprotectant, such as, for example, sucrose. In certain embodiments, the composition may comprise tris saline sucrose (TSS). In certain embodiments, the LNP composition may include about 1, 2, 3, 4, 5, 6, 7, 8, 9, or 10% cryoprotectant. In certain embodiments, the LNP composition may include about 1, 2, 3, 4, 5, 6, 7, 8, 9, or 10% sucrose. In some embodiments, the LNP composition may include a buffer. In some embodiments, the buffer may comprise a phosphate buffer (PBS), a Tris buffer, a citrate buffer, and mixtures thereof. In certain exemplary embodiments, the buffer comprises NaCl. In certain embodiments, the buffer lacks NaCl. Exemplary amounts of NaCl may range from about 20 mM to about 45 mM. Exemplary amounts of NaCl may range from about 40 mM to about 50 mM. In some embodiments, the amount of NaCl is about 45 mM. In some embodiments, the buffer is a Tris buffer. Exemplary amounts of Tris may range from about 20 mM to about 60 mM. Exemplary amounts of Tris may range from about 40 mM to about 60 mM. In some embodiments, the amount of Tris is about 50 mM. In some embodiments, the buffer comprises NaCl and Tris. Certain exemplary embodiments of the LNP compositions contain 5% sucrose and 45 mM NaCl in Tris buffer. In other exemplary embodiments, compositions contain sucrose in an amount of about 5% w/v, about 45 mM NaCl, and about 50 mM Tris at pH 7.5. The salt, buffer, and cryoprotectant amounts may be varied such that the osmolality of the overall composition is maintained. For example, the final osmolality may be maintained at less than 450 mOsm/L. In further embodiments, the osmolality is between 350 and 250 mOsm/L. Certain embodiments have a final osmolality of 300 +/− 20 mOsm/L or 310 +/− 40 mOsm/L.

In some embodiments, microfluidic mixing, T-mixing, or cross-mixing of the aqueous RNA solution and the lipid solution in an organic solvent is used. In certain aspects, flow rates, junction size, junction geometry, junction shape, tube diameter, solutions, and/or RNA and lipid concentrations may be varied. LNPs or LNP compositions may be concentrated or purified, e.g., via dialysis, centrifugal filter, tangential flow filtration, or chromatography. The LNPs may be stored as a suspension, an emulsion, or a lyophilized powder, for example. In some embodiments, an LNP composition is stored at 2-8° C., in certain aspects, the LNP compositions are stored at room temperature. In additional embodiments, an LNP composition is stored frozen, for example at −20° C. or −80° C. In other embodiments, an LNP composition is stored at a temperature ranging from about 0° C. to about −80° C. Frozen LNP compositions may be thawed before use, for example on ice, at room temperature, or at 25° C.

The LNPs may be, e.g., microspheres (including unilamellar and multilamellar vesicles, e.g., “liposomes”—lamellar phase lipid bilayers that, in some embodiments, are substantially spherical—and, in more particular embodiments, can comprise an aqueous core, e.g., comprising a substantial portion of RNA molecules), a dispersed phase in an emulsion, micelles, or an internal phase in a suspension.

Preferred lipid compositions, such as LNP compositions, are biodegradable, in that they do not accumulate to cytotoxic levels in vivo at a therapeutically effective dose. In some embodiments, the compositions do not cause an innate immune response that leads to substantial adverse effects at a therapeutic dose level. In some embodiments, the compositions provided herein do not cause toxicity at a therapeutic dose level.

In some embodiments, the LNPs disclosed herein have a polydispersity index (PDI) that may range from about 0.005 to about 0.75. In some embodiments, the LNP have a PDI that may range from about 0.01 to about 0.5. In some embodiments, the LNP have a PDI that may range from about zero to about 0.4. In some embodiments, the LNP have a PDI that may range from about zero to about 0.35. In some embodiments, the LNP have a PDI that may range from about zero to about 0.35. In some embodiments, the LNP PDI may range from about zero to about 0.3. In some embodiments, the LNP have a PDI that may range from about zero to about 0.25. In some embodiments, the LNP PDI may range from about zero to about 0.2. In some embodiments, the LNP have a PDI that may be less than about 0.08, 0.1, 0.15, 0.2, or 0.4.

The LNPs disclosed herein have a size (e.g. Z-average diameter) of about 1 to about 250 nm. In some embodiments, the LNPs have a size of about 10 to about 200 nm. In further embodiments, the LNPs have a size of about 20 to about 150 nm. In some embodiments, the LNPs have a size of about 50 to about 150 nm. In some embodiments, the LNPs have a size of about 50 to about 100 nm. In some embodiments, the LNPs have a size of about 50 to about 120 nm. In some embodiments, the LNPs have a size of about 60 to about 100 nm. In some embodiments, the LNPs have a size of about 75 to about 150 nm. In some embodiments, the LNPs have a size of about 75 to about 120 nm. In some embodiments, the LNPs have a size of about 75 to about 100 nm. Unless indicated otherwise, all sizes referred to herein are the average sizes (diameters) of the fully formed nanoparticles, as measured by dynamic light scattering on a Malvern Zetasizer or Wyatt

NanoStar. The nanoparticle sample is diluted in phosphate buffered saline (PBS) so that the count rate is approximately 200-400 kcps. The data is presented as a weighted-average of the intensity measure (Z-average diameter).

In some embodiments, the LNPs are formed with an average encapsulation efficiency ranging from about 50% to about 100%. In some embodiments, the LNPs are formed with an average encapsulation efficiency ranging from about 50% to about 95%. In some embodiments, the LNPs are formed with an average encapsulation efficiency ranging from about 70% to about 90%. In some embodiments, the LNPs are formed with an average encapsulation efficiency ranging from about 90% to about 100%. In some embodiments, the LNPs are formed with an average encapsulation efficiency ranging from about 75% to about 95%.

### Cargo

The cargo delivered via LNP composition may be a biologically active agent. In certain embodiments, the cargo is or comprises one or more biologically active agent, such as mRNA, gRNA, expression vector, template nucleic acid, RNA-guided DNA-binding agent, antibody (e.g., monoclonal, chimeric, humanized, nanobody, and fragments thereof etc.), cholesterol, hormone, peptide, protein, chemotherapeutic and other types of antineoplastic agent, low molecular weight drug, vitamin, co-factor, nucleoside, nucleotide, oligonucleotide, enzymatic nucleic acid, antisense nucleic acid, triplex forming oligonucleotide, antisense DNA or RNA composition, chimeric DNA:RNA composition, allozyme, aptamer, ribozyme, decoys and analogs thereof, plasmid and other types of vectors, and small nucleic acid molecule, RNAi agent, short interfering nucleic acid (siNA), short interfering RNA (siRNA), double-stranded RNA (dsRNA), micro-RNA (miRNA), short hairpin RNA (shRNA) and “self-replicating RNA” (encoding a replicase enzyme activity and capable of directing its own replication or amplification in vivo) molecules, peptide nucleic acid (PNA), a locked nucleic acid ribonucleotide (LNA), morpholino nucleotide, threose nucleic acid (TNA), glycol nucleic acid (GNA), sisiRNA (small internally segmented interfering RNA), and iRNA (asymmetrical interfering RNA). The above list of biologically active agents is exemplary only, and is not intended to be limiting. Such compounds may be purified or partially purified, and may be naturally occurring or synthetic, and may be chemically modified.

The cargo delivered via LNP composition may be an RNA, such as an mRNA molecule encoding a protein of interest. For example, an mRNA for expressing a protein such as green fluorescent protein (GFP), an RNA-guided DNA-binding agent, or a Cas nuclease is included. LNP compositions that include a Cas nuclease mRNA, for example a Class 2 Cas nuclease mRNA that allows for expression in a cell of a Class 2 Cas nuclease such as a Cas9 or Cpf1 protein are provided. Further, the cargo may contain one or more gRNAs or nucleic acids encoding gRNAs. A template nucleic acid, e.g., for repair or recombination, may also be included in the composition or a template nucleic acid may be used in the methods described herein. In a sub-embodiment, the cargo comprises an mRNA that encodes a Streptococcus pyogenes Cas9, optionally and an S. pyogenes gRNA. In a further sub-embodiment, the cargo comprises an mRNA that encodes a Neisseria meningitidis Cas9, optionally and an Nme (Neisseria meningitidis) gRNA.

“mRNA” refers to a polynucleotide and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof. In general, mRNAs do not contain a substantial quantity of thymidine residues (e.g., 0 residues or fewer than 30, 20, 10, 5, 4, 3, or 2 thymidine residues; or less than 10%, 9%, 8%, 7%, 6%, 5%, 4%, 4%, 3%, 2%, 1%, 0.5%, 0.2%, or 0.1% thymidine content). An mRNA can contain modified uridines at some or all of its uridine positions.

### CRISPR/Cas Cargo

In certain embodiments, the disclosed compositions comprise an mRNA encoding an RNA-guided DNA-binding agent, such as a Cas nuclease. In particular embodiments, the disclosed compositions comprise an mRNA encoding a Class 2 Cas nuclease, such as S. pyogenes Cas9.

As used herein, an “RNA-guided DNA-binding agent” means a polypeptide or complex of polypeptides having RNA and DNA-binding activity, or a DNA-binding subunit of such a complex, wherein the DNA-binding activity is sequence-specific and depends on the sequence of the RNA. Exemplary RNA-guided DNA-binding agents include Cas cleavases/nickases and inactivated forms thereof (“dCas DNA-binding agents”). “Cas nuclease”, as used herein, encompasses Cas cleavases, Cas nickases, and dCas DNA-binding agents. Cas cleavases/nickases and dCas DNA-binding agents include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. As used herein, a “Class 2 Cas nuclease” is a single-chain polypeptide with RNA-guided DNA-binding activity. Class 2 Cas nucleases include Class 2 Cas cleavases/nickases (e.g., H840A, D10A, or N863A variants), which further have RNA-guided DNA cleavases or nickase activity, and Class 2 dCas DNA-binding agents, in which cleavase/nickase activity is inactivated. Class 2 Cas nucleases include, for example, Cas9, Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9(1.0) (e.g, K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015), is homologous to Cas9, and contains a RuvC-like nuclease domain. Cpf1 sequences of Zetsche are incorporated by reference in their entirety. See, e.g., Zetsche, Tables 2 and 4. See, e.g., Makarova et al., Nat Rev Microbiol, 13(11): 722-36 (2015); Shmakov et al., Molecular Cell, 60:385-397 (2015).

As used herein, “ribonucleoprotein” (RNP) or “RNP complex” refers to a gRNA together with an RNA-guided DNA-binding agent, such as a Cas nuclease, e.g., a Cas cleavase, Cas nickase, or dCas DNA-binding agent (e.g., Cas9). In some embodiments, the gRNA guides the RNA-guided DNA-binding agent such as Cas9 to a target sequence, and the gRNA hybridizes with and the agent binds to the target sequence; in cases where the agent is a cleavase or nickase, binding can be followed by cleaving or nicking.

In some embodiments of the present disclosure, the cargo for the LNP composition includes at least one gRNA comprising guide sequences that direct an RNA-guided DNA-binding agent, which can be a nuclease (e.g., a Cas nuclease such as Cas9), to a target DNA. The gRNA may guide the Cas nuclease or Class 2 Cas nuclease to a target sequence on a target nucleic acid molecule. In some embodiments, a gRNA binds with and provides specificity of cleavage by a Class 2 Cas nuclease. In some embodiments, the gRNA and the Cas nuclease may form a ribonucleoprotein (RNP), e.g., a CRISPR/Cas complex such as a CRISPR/Cas9 complex. In some embodiments, the CRISPR/Cas complex may be a Type-II CRISPR/Cas9 complex. In some embodiments, the CRISPR/Cas complex may be a Type-V CRISPR/Cas complex, such as a Cpf1/gRNA complex. Cas nucleases and cognate gRNAs may be paired. The gRNA scaffold structures that pair with each Class 2 Cas nuclease vary with the specific CRISPR/Cas system.

“Guide RNA”, “gRNA”, and simply “guide” are used herein interchangeably to refer to a cognate guide nucleic acid for an RNA-guided DNA-binding agent. Guide RNAs can include modified RNAs as described herein. A gRNA may be either a crRNA (also known as CRISPR RNA), or the combination of a crRNA and a trRNA (also known as tracrRNA). The crRNA and trRNA may be associated as a single RNA molecule (single guide RNA, sgRNA) or in two separate RNA molecules (dual guide RNA, dgRNA). “Guide RNA” or “gRNA” refers to each type. The trRNA may be a naturally-occurring sequence, or a trRNA sequence with modifications or variations compared to naturally-occurring sequences.

As used herein, a “guide sequence” refers to a sequence within a gRNA that is complementary to a target sequence and functions to direct a gRNA to a target sequence for binding or modification (e.g., cleavage) by an RNA-guided DNA-binding agent. A “guide sequence” may also be referred to as a “targeting sequence,” or a “spacer sequence.” A guide sequence can be 20 base pairs in length, e.g., in the case of Streptococcus pyogenes (i.e., Spy Cas9) and related Cas9 homologs/orthologs. Shorter or longer sequences can also be used as guides, e.g., 15-, 16-, 17-, 18-, 19-, 21-, 22-, 23-, 24-, or 25-nucleotides in length. In some embodiments, the target sequence is in a gene or on a chromosome, for example, and is complementary to the guide sequence. In some embodiments, the degree of complementarity or identity between a guide sequence and its corresponding target sequence may be about or at least 75%, 80%, 85%, 90%, 95%, 96%, 97%, 98%, 99%, or 100%. In some embodiments, the guide sequence and the target region may be 100% complementary or identical over a region of at least 15, 16, 17, 18, 19, or 20 contiguous nucleotides. In other embodiments, the guide sequence and the target region may contain at least one mismatch. For example, the guide sequence and the target sequence may contain 1, 2, 3, or 4 mismatches, where the total length of the target sequence is at least 17, 18, 19, 20 or more base pairs. In some embodiments, the guide sequence and the target region may contain 1-4 mismatches where the guide sequence comprises at least 17, 18, 19, 20 or more nucleotides. In some embodiments, the guide sequence and the target region may contain 1, 2, 3, or 4 mismatches where the guide sequence comprises 20 nucleotides.

Target sequences for RNA-guided DNA-binding proteins such as Cas proteins include both the positive and negative strands of genomic DNA (i.e., the sequence given and the sequence's reverse compliment), as a nucleic acid substrate for a Cas protein is a double stranded nucleic acid. Accordingly, where a guide sequence is said to be “complementary to a target sequence”, it is to be understood that the guide sequence may direct a gRNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

The length of the targeting sequence may depend on the CRISPR/Cas system and components used. For example, different Class 2 Cas nucleases from different bacterial species have varying optimal targeting sequence lengths. Accordingly, the targeting sequence may comprise 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the targeting sequence length is 0, 1, 2, 3, 4, or 5 nucleotides longer or shorter than the guide sequence of a naturally-occurring CRISPR/Cas system. In certain embodiments, the Cas nuclease and gRNA scaffold will be derived from the same

CRISPR/Cas system. In some embodiments, the targeting sequence may comprise or consist of 18-24 nucleotides. In some embodiments, the targeting sequence may comprise or consist of 19-21 nucleotides. In some embodiments, the targeting sequence may comprise or consist of 20 nucleotides. In some embodiments, the sgRNA is a “Cas9 sgRNA” capable of mediating RNA-guided DNA cleavage by a Cas9 protein. In some embodiments, the sgRNA is a “Cpf1 sgRNA” capable of mediating RNA-guided DNA cleavage by a Cpf1 protein. In certain embodiments, the gRNA comprises a crRNA and tracr RNA sufficient for forming an active complex with a Cas9 protein and mediating RNA-guided DNA cleavage. In certain embodiments, the gRNA comprises a crRNA sufficient for forming an active complex with a Cpf1 protein and mediating RNA-guided DNA cleavage. See Zetsche 2015.

Certain embodiments of the invention also provide nucleic acids, e.g., expression cassettes, encoding the gRNA described herein. A “guide RNA nucleic acid” is used herein to refer to a gRNA (e.g. an sgRNA or a dgRNA) and a gRNA expression cassette, which is a nucleic acid that encodes one or more gRNAs. Modified RNAs

In certain embodiments, the lipid compositions, such as LNP compositions comprise modified nucleic acids, including modified RNAs.

Modified nucleosides or nucleotides can be present in an RNA, for example a gRNA or mRNA. A gRNA or mRNA comprising one or more modified nucleosides or nucleotides, for example, is called a “modified” RNA to describe the presence of one or more non-naturally and/or naturally occurring components or configurations that are used instead of or in addition to the canonical A, G, C, and U residues. In some embodiments, a modified RNA is synthesized with a non-canonical nucleoside or nucleotide, here called “modified.”

Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the polynucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification). Certain embodiments comprise a 5′ end modification to an mRNA, gRNA, or nucleic acid. Certain embodiments comprise a modification to an mRNA, gRNA, or nucleic acid. Certain embodiments comprise a 3′ end modification to an mRNA, gRNA, or nucleic acid. A modified RNA can contain 5′ end and 3′ end modifications. A modified RNA can contain one or more modified residues at non-terminal locations. In certain embodiments, a gRNA includes at least one modified residue. In certain embodiments, an mRNA includes at least one modified residue.

Unmodified nucleic acids can be prone to degradation by, e.g., intracellular nucleases or those found in serum. For example, nucleases can hydrolyze nucleic acid phosphodiester bonds. Accordingly, in one aspect the RNAs (e.g. mRNAs, gRNAs) described herein can contain one or more modified nucleosides or nucleotides, e.g., to introduce stability toward intracellular or serum-based nucleases. In some embodiments, the modified RNA molecules described herein can exhibit a reduced innate immune response when introduced into a population of cells, both in vivo and ex vivo. The term “innate immune response” includes a cellular response to exogenous nucleic acids, including single stranded nucleic acids, which involves the induction of cytokine expression and release, particularly the interferons, and cell death.

Accordingly, in some embodiments, an RNA or nucleic acid comprises at least one modification which confers increased or enhanced stability to the nucleic acid, including, for example, improved resistance to nuclease digestion in vivo. As used herein, the terms “modification” and “modified” as such terms relate to the nucleic acids provided herein, include at least one alteration which preferably enhances stability and renders the RNA or nucleic acid more stable (e.g., resistant to nuclease digestion) than the wild-type or naturally occurring version of the RNA or nucleic acid. As used herein, the terms “stable” and “stability” as such terms relate to the nucleic acids of the present invention, and particularly with respect to the RNA, refer to increased or enhanced resistance to degradation by, for example nucleases (i.e., endonucleases or exonucleases) which are normally capable of degrading such RNA. Increased stability can include, for example, less sensitivity to hydrolysis or other destruction by endogenous enzymes (e.g., endonucleases or exonucleases) or conditions within the target cell or tissue, thereby increasing or enhancing the residence of such RNA or nucleic acid in the target cell, tissue, subject and/or cytoplasm. The stabilized RNA or nucleic acid molecules provided herein demonstrate longer half-lives relative to their naturally occurring, unmodified counterparts (e.g. the wild-type version of the molecule). Also contemplated by the terms “modification” and “modified” as such terms related to the mRNA of the LNP compositions disclosed herein are alterations which improve or enhance translation of mRNA nucleic acids, including for example, the inclusion of sequences which function in the initiation of protein translation (e.g., the Kozak consensus sequence). (Kozak, M., Nucleic Acids Res 15 (20): 8125-48 (1987)).

In some embodiments, the RNA or nucleic acid has undergone a chemical or biological modification to render it more stable. Exemplary modifications to an RNA or nucleic acid include the depletion of a base (e.g., by deletion or by the substitution of one nucleotide for another) or modification of a base, for example, the chemical modification of a base. The phrase “chemical modifications” as used herein, includes modifications which introduce chemistries which differ from those seen in naturally occurring RNA or nucleic acids, for example, covalent modifications such as the introduction of modified nucleotides, (e.g., nucleotide analogs, or the inclusion of pendant groups which are not naturally found in such RNA or nucleic acid molecules).

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens. The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino. mRNAs

In some embodiments, a composition or formulation disclosed herein comprises an mRNA comprising an open reading frame (ORF) encoding an RNA-guided DNA-binding agent, such as a Cas nuclease, or Class 2 Cas nuclease as described herein. In some embodiments, an mRNA comprising an ORF encoding an RNA-guided DNA-binding agent, such as a Cas nuclease or Class 2 Cas nuclease, is provided, used, or administered. An mRNA may comprise one or more of a 5′ cap, a 5′ untranslated region (UTR), a 3′ UTRs, and a polyadenine tail. The mRNA may comprise a modified open reading frame, for example to encode a nuclear localization sequence or to use alternate codons to encode the protein.

The mRNA in the disclosed LNP compositions may encode, for example, a secreted hormone, enzyme, receptor, polypeptide, peptide or other protein of interest that is normally secreted. In one embodiment of the invention, the mRNA may optionally have chemical or biological modifications which, for example, improve the stability and/or half-life of such mRNA or which improve or otherwise facilitate protein production.

In addition, suitable modifications include alterations in one or more nucleotides of a codon such that the codon encodes the same amino acid but is more stable than the codon found in the wild-type version of the mRNA. For example, an inverse relationship between the stability of RNA and a higher number cytidines (C's) and/or uridines (U's) residues has been demonstrated, and RNA devoid of C and U residues have been found to be stable to most RNases (Heidenreich, et al. J Biol Chem 269, 2131-8 (1994)). In some embodiments, the number of C and/or U residues in an mRNA sequence is reduced. In another embodiment, the number of C and/or U residues is reduced by substitution of one codon encoding a particular amino acid for another codon encoding the same or a related amino acid. Contemplated modifications to the mRNA nucleic acids of the present invention also include the incorporation of pseudouridines. The incorporation of pseudouridines into the mRNA nucleic acids of the present invention may enhance stability and translational capacity, as well as diminishing immunogenicity in vivo. See, e.g., Karikó, K., et al., Molecular Therapy 16 (11): 1833-1840 (2008). Substitutions and modifications to the mRNA of the present invention may be performed by methods readily known to one or ordinary skill in the art.

The constraints on reducing the number of C and U residues in a sequence will likely be greater within the coding region of an mRNA, compared to an untranslated region, (i.e., it will likely not be possible to eliminate all of the C and U residues present in the message while still retaining the ability of the message to encode the desired amino acid sequence). The degeneracy of the genetic code, however presents an opportunity to allow the number of C and/or U residues that are present in the sequence to be reduced, while maintaining the same coding capacity (i.e., depending on which amino acid is encoded by a codon, several different possibilities for modification of RNA sequences may be possible). The term modification also includes, for example, the incorporation of non-nucleotide linkages or modified nucleotides into the mRNA sequences of the present invention (e.g., modifications to one or both the 3′ and 5′ ends of an mRNA molecule encoding a functional secreted protein or enzyme). Such modifications include the addition of bases to an mRNA sequence (e.g., the inclusion of a poly A tail or a longer poly A tail), the alteration of the 3′ UTR or the 5′ UTR, complexing the mRNA with an agent (e.g., a protein or a complementary nucleic acid molecule), and inclusion of elements which change the structure of an mRNA molecule (e.g., which form secondary structures).

The poly A tail is thought to stabilize natural messengers. Therefore, in one embodiment a long poly A tail can be added to an mRNA molecule thus rendering the mRNA more stable. Poly A tails can be added using a variety of art-recognized techniques.

For example, long poly A tails can be added to synthetic or in vitro transcribed mRNA using poly A polymerase (Yokoe, et al. Nature Biotechnology. 1996; 14: 1252-1256). A transcription vector can also encode long poly A tails. In addition, poly A tails can be added by transcription directly from PCR products. In one embodiment, the length of the poly A tail is at least about 90, 200, 300, 400 at least 500 nucleotides. In one embodiment, the length of the poly A tail is adjusted to control the stability of a modified mRNA molecule of the invention and, thus, the transcription of protein. For example, since the length of the poly A tail can influence the half-life of an mRNA molecule, the length of the poly A tail can be adjusted to modify the level of resistance of the mRNA to nucleases and thereby control the time course of protein expression in a cell. In one embodiment, the stabilized mRNA molecules are sufficiently resistant to in vivo degradation (e.g., by nucleases), such that they may be delivered to the target cell without a transfer vehicle.

In one embodiment, an mRNA can be modified by the incorporation 3′ and/or 5′ untranslated (UTR) sequences which are not naturally found in the wild-type mRNA. In one embodiment, 3′ and/or 5′ flanking sequence which naturally flanks an mRNA and encodes a second, unrelated protein can be incorporated into the nucleotide sequence of an mRNA molecule encoding a therapeutic or functional protein in order to modify it. For example, 3′ or 5′ sequences from mRNA molecules which are stable (e.g., globin, actin, GAPDH, tubulin, histone, or citric acid cycle enzymes) can be incorporated into the 3′ and/or 5′ region of a sense mRNA nucleic acid molecule to increase the stability of the sense mRNA molecule. See, e.g., US2003/0083272.

More detailed descriptions of the mRNA modifications can be found in US2017/0210698A1, at pages 57-68, the contents of which are incorporated herein.

### Template Nucleic Acid

The compositions and methods disclosed herein may include a template nucleic acid. The template may be used to alter or insert a nucleic acid sequence at or near a target site for an RNA-guided DNA-binding protein such as a Cas nuclease, e.g., a Class 2 Cas nuclease. In some embodiments, the methods comprise introducing a template to the cell. In some embodiments, a single template may be provided. In other embodiments, two or more templates may be provided such that editing may occur at two or more target sites. For example, different templates may be provided to edit a single gene in a cell, or two different genes in a cell.

In some embodiments, the template may be used in homologous recombination. In some embodiments, the homologous recombination may result in the integration of the template sequence or a portion of the template sequence into the target nucleic acid molecule. In other embodiments, the template may be used in homology-directed repair, which involves DNA strand invasion at the site of the cleavage in the nucleic acid. In some embodiments, the homology-directed repair may result in including the template sequence in the edited target nucleic acid molecule. In yet other embodiments, the template may be used in gene editing mediated by non-homologous end joining. In some embodiments, the template sequence has no similarity to the nucleic acid sequence near the cleavage site. In some embodiments, the template or a portion of the template sequence is incorporated. In some embodiments, the template includes flanking inverted terminal repeat (ITR) sequences.

In some embodiments, the template sequence may correspond to, comprise, or consist of an endogenous sequence of a target cell. It may also or alternatively correspond to, comprise, or consist of an exogenous sequence of a target cell. As used herein, the term “endogenous sequence” refers to a sequence that is native to the cell. The term “exogenous sequence” refers to a sequence that is not native to a cell, or a sequence whose native location in the genome of the cell is in a different location. In some embodiments, the endogenous sequence may be a genomic sequence of the cell. In some embodiments, the endogenous sequence may be a chromosomal or extrachromosomal sequence. In some embodiments, the endogenous sequence may be a plasmid sequence of the cell.

In some embodiments, the template contains ssDNA or dsDNA containing flanking invert-terminal repeat (ITR) sequences. In some embodiments, the template is provided as a vector, plasmid, minicircle, nanocircle, or PCR product.

In some embodiments, the nucleic acid is purified. In some embodiments, the nucleic acid is purified using a precipitation method (e.g., LiCl precipitation, alcohol precipitation, or an equivalent method, e.g., as described herein). In some embodiments, the nucleic acid is purified using a chromatography-based method, such as an HPLC-based method or an equivalent method (e.g., as described herein). In some embodiments, the nucleic acid is purified using both a precipitation method (e.g., LiCl precipitation) and an HPLC-based method. In some embodiments, the nucleic acid is purified by tangential flow filtration (TFF).

The compounds or compositions will generally, but not necessarily, include one or more pharmaceutically acceptable excipients. The term “excipient” includes any ingredient other than the compound(s) of the disclosure, the other lipid component(s) and the biologically active agent. An excipient may impart either a functional (e.g. drug release rate controlling) and/or a non-functional (e.g. processing aid or diluent) characteristic to the compositions. The choice of excipient will to a large extent depend on factors such as the particular mode of administration, the effect of the excipient on solubility and stability, and the nature of the dosage form.

Parenteral formulations are typically aqueous or oily solutions or suspensions. Where the formulation is aqueous, excipients such as sugars (including but not restricted to glucose, mannitol, sorbitol, etc.) salts, carbohydrates and buffering agents (preferably to a pH of from 3 to 9), but, for some applications, they may be more suitably formulated with a sterile non-aqueous solution or as a dried form to be used in conjunction with a suitable vehicle such as sterile, pyrogen-free water (WFI).

While the invention is described in conjunction with the illustrated embodiments, it is understood that they are not intended to limit the invention to those embodiments. On the contrary, the invention is intended to cover all alternatives, modifications, and equivalents, including equivalents of specific features, which may be included within the invention as defined by the appended claims.

Both the foregoing general description and detailed description, as well as the following examples, are exemplary and explanatory only and are not restrictive of the teachings. The section headings used herein are for organizational purposes only and are not to be construed as limiting the desired subject matter in any way. In the event that any literature incorporated by reference contradicts any term defined in this specification, this specification controls. All ranges given in the application encompass the endpoints unless stated otherwise.

### Definitions

It should be noted that, as used in this application, the singular form “a”, “an” and “the” include plural references unless the context clearly dictates otherwise. Thus, for example, reference to “a composition” includes a plurality of compositions and reference to “a cell” includes a plurality of cells and the like. The use of “or” is inclusive and means “and/or” unless stated otherwise.

Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; embodiments in the specification that recite “about” various components are also contemplated as “at” the recited components; and embodiments in the specification that recite “consisting essentially of” various components are also contemplated as “consisting of” or “comprising” the recited components (this interchangeability does not apply to the use of these terms in the claims).

Numeric ranges are inclusive of the numbers defining the range. Measured and measureable values are understood to be approximate, taking into account significant digits and the error associated with the measurement. As used in this application, the terms “about” and “approximately” have their art-understood meanings; use of one vs the other does not necessarily imply different scope. Unless otherwise indicated, numerals used in this application, with or without a modifying term such as “about” or “approximately”, should be understood to encompass normal divergence and/or fluctuations as would be appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated reference value unless otherwise stated or otherwise evident from the context (except where such number would exceed 100% of a possible value).

As used herein, the term “contacting” means establishing a physical connection between two or more entities. For example, contacting a mammalian cell with a nanoparticle composition means that the mammalian cell and a nanoparticle are made to share a physical connection. Methods of contacting cells with external entities both in vivo and ex vivo are well known in the biological arts. For example, contacting a nanoparticle composition and a mammalian cell disposed within a mammal may be performed by varied routes of administration (e.g., intravenous, intramuscular, intradermal, and subcutaneous) and may involve varied amounts of nanoparticle compositions. Moreover, more than one mammalian cell may be contacted by a nanoparticle composition.

As used herein, the term “delivering” means providing an entity to a destination. For example, delivering a therapeutic and/or prophylactic to a subject may involve administering a nanoparticle composition including the therapeutic and/or prophylactic to the subject (e.g., by an intravenous, intramuscular, intradermal, or subcutaneous route). Administration of a nanoparticle composition to a mammal or mammalian cell may involve contacting one or more cells with the nanoparticle composition.

As used herein, “encapsulation efficiency” refers to the amount of a therapeutic and/or prophylactic that becomes part of a nanoparticle composition, relative to the initial total amount of therapeutic and/or prophylactic used in the preparation of a nanoparticle composition. For example, if 97 mg of therapeutic and/or prophylactic are encapsulated in a nanoparticle composition out of a total 100 mg of therapeutic and/or prophylactic initially provided to the composition, the encapsulation efficiency may be given as 97%. As used herein, “encapsulation” may refer to complete, substantial, or partial enclosure, confinement, surrounding, or encasement.

As used herein, the term “biodegradable” is used to refer to materials that, when introduced into cells, are broken down by cellular machinery (e.g., enzymatic degradation) or by hydrolysis into components that cells can either reuse or dispose of without significant toxic effect(s) on the cells. In certain embodiments, components generated by breakdown of a biodegradable material do not induce inflammation and/or other adverse effects in vivo. In some embodiments, biodegradable materials are enzymatically broken down. Alternatively or additionally, in some embodiments, biodegradable materials are broken down by hydrolysis.

As used herein, the “N/P ratio” is the molar ratio of ionizable nitrogen atom-containing lipid (e.g. Compound of Formula I) to phosphate groups in RNA, e.g., in a nanoparticle composition including a lipid component and an RNA.

Compositions may also include salts of one or more compounds. Salts may be pharmaceutically acceptable salts. As used herein, “pharmaceutically acceptable salts” refers to derivatives of the disclosed compounds wherein the parent compound is altered by converting an existing acid or base moiety to its salt form (e.g., by reacting a free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

As used herein, the “polydispersity index” is a ratio that describes the homogeneity of the particle size distribution of a system. A small value, e.g., less than 0.3, indicates a narrow particle size distribution. In some embodiments, the polydispersity index may be less than 0.1.

As used herein, “transfection” refers to the introduction of a species (e.g., an RNA) into a cell. Transfection may occur, for example, in vitro, ex vivo, or in vivo.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched (i.e., linear). The alkyl group can also be substituted or unsubstituted. For example, the alkyl group can be substituted with one or more groups including, but not limited to, alkyl, aryl, heteroaryl, cycloalkyl, alkoxy, amino, ether, halide, hydroxy, nitro, silyl, sulfoxo, sulfonate, carboxylate, or thiol, as described herein. A “lower alkyl” group is an alkyl group containing from one to six (e.g., from one to four) carbon atoms.

The term “alkenyl”, as used herein, refers to an aliphatic group containing at least one carbon-carbon double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, an alkenyl group may be substituted by one or more alkyl, carbocyclyl, aryl, heterocyclyl, or heteroaryl groups is contemplated. Exemplary alkenyl groups include, but are not limited to, vinyl (—CH═CH₂), allyl (—CH₂CH═CH₂), cyclopentenyl (—C₅H₇), and 5-hexenyl (—CH₂CH₂CH₂CH₂CH═CH₂).

An “alkylene” group refers to a divalent alkyl radical, which may be branched or unbranched (i.e., linear). Any of the above mentioned monovalent alkyl groups may be converted to an alkylene by abstraction of a second hydrogen atom from the alkyl. Representative alkylenes include C₂₋₄ alkylene and C₂₋₃ alkylene. Typical alkylene groups include, but are not limited to —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like. The alkylene group can also be substituted or unsubstituted. For example, the alkylene group can be substituted with one or more groups including, but not limited to, alkyl, aryl, heteroaryl, cycloalkyl, alkoxy, amino, ether, halide, hydroxy, nitro, silyl, sulfoxo, sulfonate, carboxylate, or thiol, as described herein.

The term “alkenylene” includes divalent, straight or branched, unsaturated, acyclic hydrocarbyl groups having at least one carbon-carbon double bond and, in one embodiment, no carbon-carbon triple bonds. Any of the above-mentioned monovalent alkenyl gorups may be converted to an alkenylene by abstraction of a second hydrogen atom from the alkenyl. Representative alkenylenes include C₂₋₆ alkenylenes.

The term “C_{x-y}” when used in conjunction with a chemical moiety, such as alkyl or alkylene, is meant to include groups that contain from x to y carbons in the chain. For example, the term “C_{x-y} alkyl” refers to substituted or unsubstituted saturated hydrocarbon groups, including straight-chain and branched-chain alkyl and alkylene groups that contain from x to y carbons in the chain.

### Incorporation by Reference

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicant reserves the right to physically incorporate into this application any and all materials and information from any such articles, patents, patent applications, or other physical and electronic documents.

### EXAMPLES

### General Information

All reagents and solvents were purchased and used as received from commercial vendors or synthesized according to cited procedures. All intermediates and final compounds were purified using flash column chromatography on silica gel. NMR spectra were recorded on a Bruker or Varian 400 MHz spectrometer, and NMR data were collected in CDCl₃ at ambient temperature. Chemical shifts are reported in parts per million (ppm) relative to CDCl₃ (7.26). Data for ¹H NMR are reported as follows: chemical shift, multiplicity (br=broad, s=singlet, d=doublet, t=triplet, dd=doublet of doublets, dt=doublet of triplets, q=quartet, m=multiplet, ddd=doublet of doublet of doublets, td=triplet of doublets, tt=triplet of triplets, tdd=triplet of doublet of doublets, dddd=doublet of doublet of doublet of doublets, etc.), coupling constant, and integration. MS data were recorded on a Waters SQD2 mass spectrometer with an electrospray ionization (ESI) source. Purity of the final compounds was determined by UPLC-MS-ELS using a Waters Acquity H-Class liquid chromatography instrument equipped with SQD2 mass spectrometer with photodiode array (PDA) and evaporative light scattering (ELS) detectors.

| Compound | Structure |
|---|---|
| 1 | 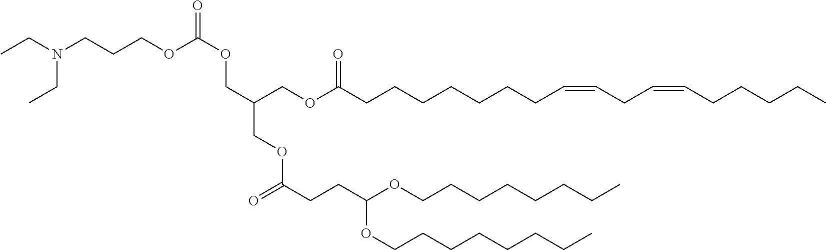 [SMILES: CCCCC/C=C\C/C=C\CCCCCCCC(=O)OCC(COC(=O)CCC(OCCCCCCCC)OCCCCCCCC)COC(=O)OCCCN(CC)CC] |
| 2 | 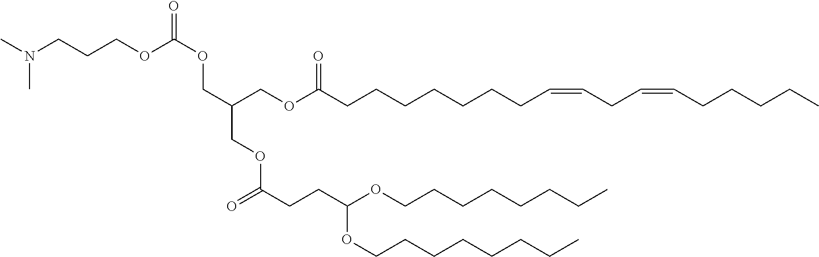 [SMILES: CCCCC/C=C\C/C=C\CCCCCCCC(=O)OCC(COC(=O)CCC(OCCCCCCCC)OCCCCCCCC)COC(=O)OCCCN(C)C] |
| 3 | 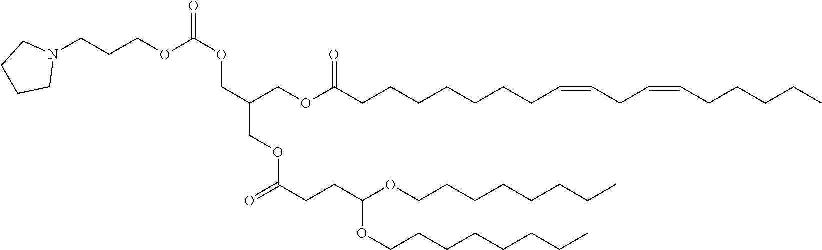 [SMILES: CCCCC/C=C\C/C=C\CCCCCCCC(=O)OCC(COC(=O)CCC(OCCCCCCCC)OCCCCCCCC)COC(=O)OCCCN1CCCC1] |
| 4 | 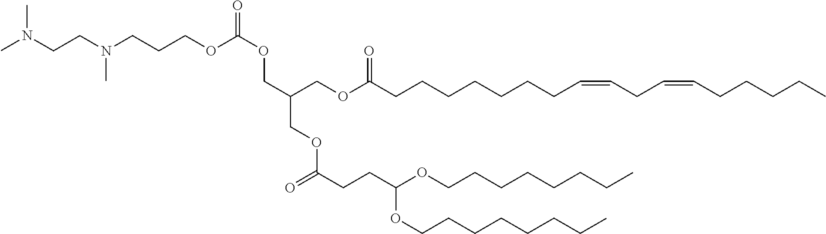 [SMILES: CCCCC/C=C\C/C=C\CCCCCCCC(=O)OCC(COC(=O)CCC(OCCCCCCCC)OCCCCCCCC)COC(=O)OCCCN(C)CCN(C)C] |
| 5 | 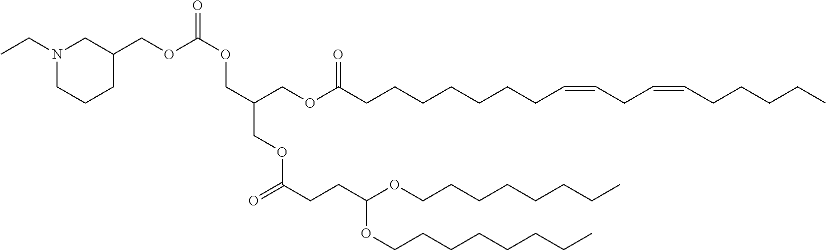 [SMILES: CCCCC/C=C\C/C=C\CCCCCCCC(=O)OCC(COC(=O)CCC(OCCCCCCCC)OCCCCCCCC)COC(=O)OCC1CCCN(CC)C1] |
| 6 | 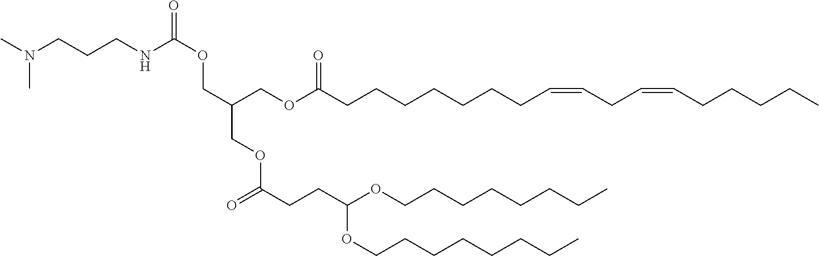 [SMILES: CCCCC/C=C\C/C=C\CCCCCCCC(=O)OCC(COC(=O)CCC(OCCCCCCCC)OCCCCCCCC)COC(=O)NCCCN(C)C] |
| 7 | 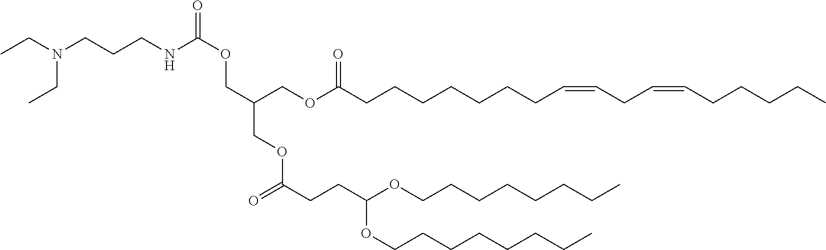 [SMILES: CCCCC/C=C\C/C=C\CCCCCCCC(=O)OCC(COC(=O)CCC(OCCCCCCCC)OCCCCCCCC)COC(=O)NCCCN(CC)CC] |
| 8 | 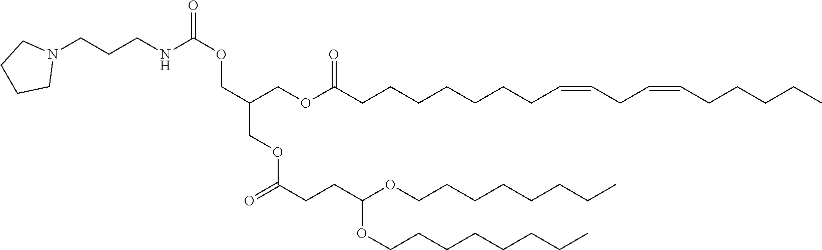 [SMILES: CCCCC/C=C\C/C=C\CCCCCCCC(=O)OCC(COC(=O)CCC(OCCCCCCCC)OCCCCCCCC)COC(=O)NCCCN1CCCC1] |
| 9 | 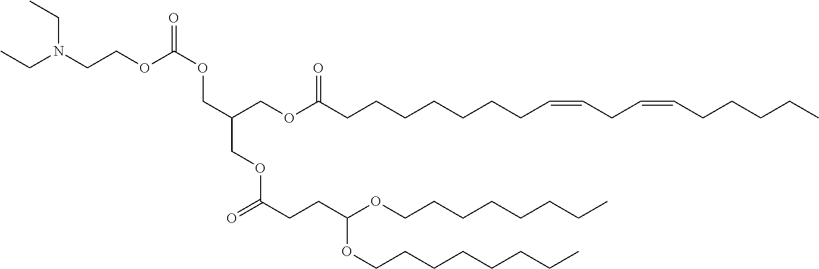 [SMILES: CCCCC/C=C\C/C=C\CCCCCCCC(=O)OCC(COC(=O)CCC(OCCCCCCCC)OCCCCCCCC)COC(=O)OCCN(CC)CC] |
| 10 | 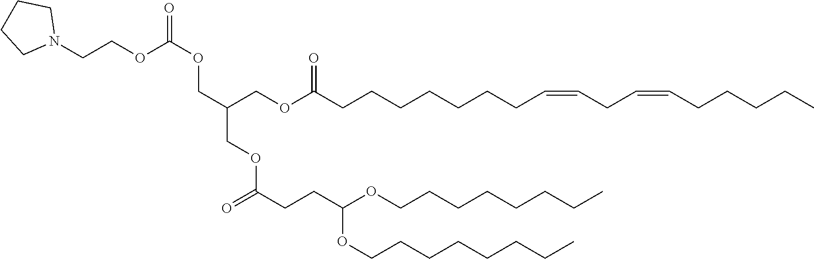 [SMILES: CCCCC/C=C\C/C=C\CCCCCCCC(=O)OCC(COC(=O)CCC(OCCCCCCCC)OCCCCCCCC)COC(=O)OCCN1CCCC1] |
| 11 | 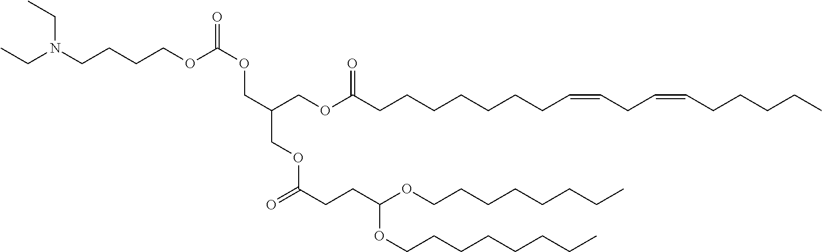 [SMILES: CCCCC/C=C\C/C=C\CCCCCCCC(=O)OCC(COC(=O)CCC(OCCCCCCCC)OCCCCCCCC)COC(=O)OCCCCN(CC)CC] |
| 12 | 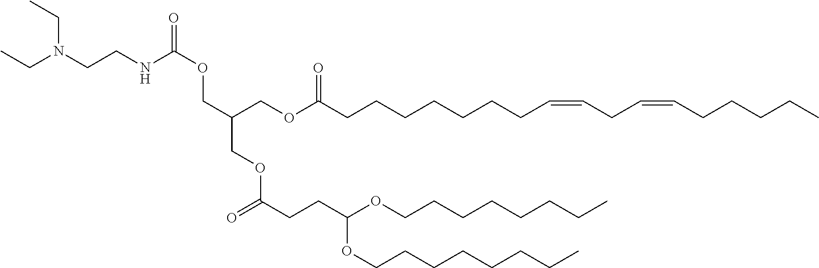 [SMILES: CCCCC/C=C\C/C=C\CCCCCCCC(=O)OCC(COC(=O)CCC(OCCCCCCCC)OCCCCCCCC)COC(=O)NCCN(CC)CC] |
| 13 | 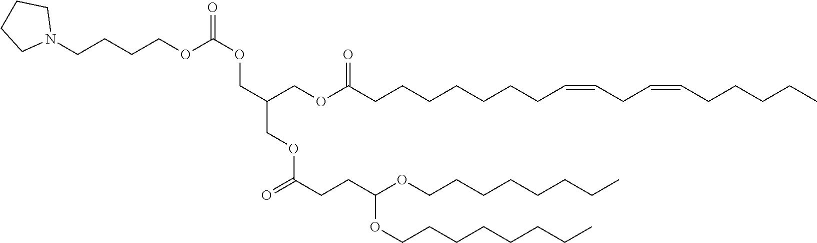 [SMILES: CCCCC/C=C\C/C=C\CCCCCCCC(=O)OCC(COC(=O)CCC(OCCCCCCCC)OCCCCCCCC)COC(=O)OCCCCN1CCCC1] |
| 14 | 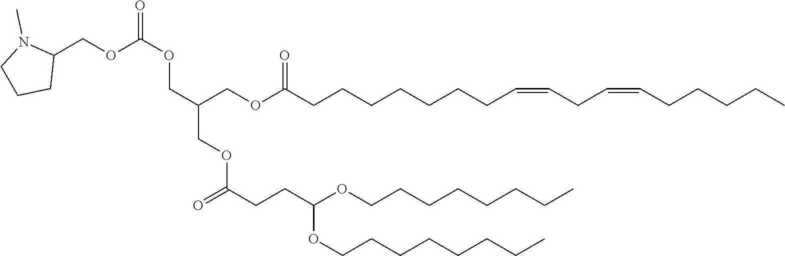 [SMILES: CCCCC/C=C\C/C=C\CCCCCCCC(=O)OCC(COC(=O)CCC(OCCCCCCCC)OCCCCCCCC)COC(=O)OCC1CCCN1C] |
| 15 | 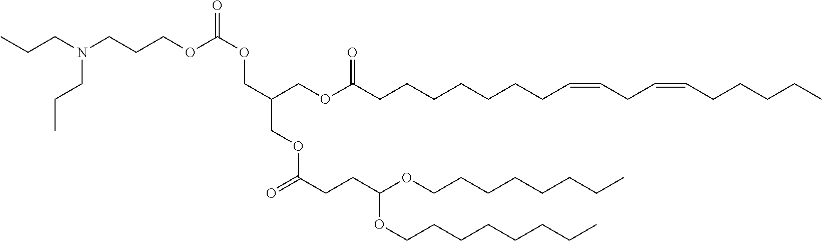 [SMILES: CCCCC/C=C\C/C=C\CCCCCCCC(=O)OCC(COC(=O)CCC(OCCCCCCCC)OCCCCCCCC)COC(=O)OCCCN(CCC)CCC] |
| 16 | 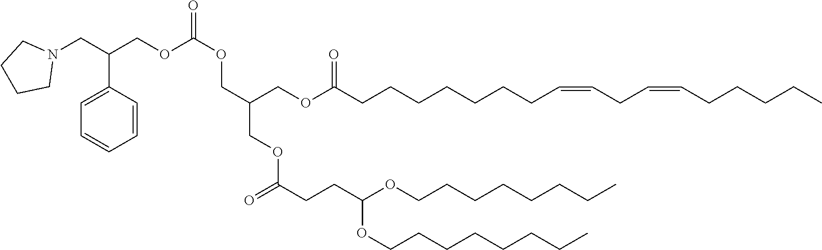 [SMILES: CCCCC/C=C\C/C=C\CCCCCCCC(=O)OCC(COC(=O)CCC(OCCCCCCCC)OCCCCCCCC)COC(=O)OCC(CN1CCCC1)c1ccccc1] |
| 17 | 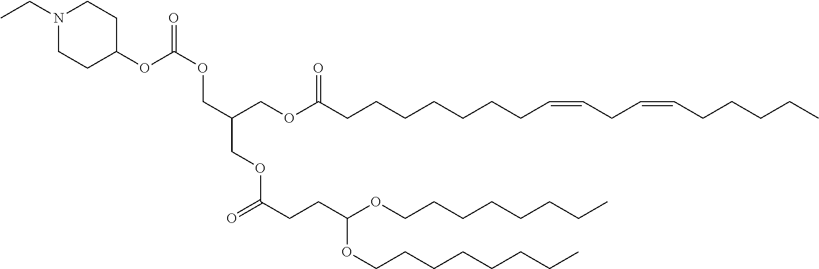 [SMILES: CCCCC/C=C\C/C=C\CCCCCCCC(=O)OCC(COC(=O)CCC(OCCCCCCCC)OCCCCCCCC)COC(=O)OC1CCN(CC)CC1] |
| 18 | 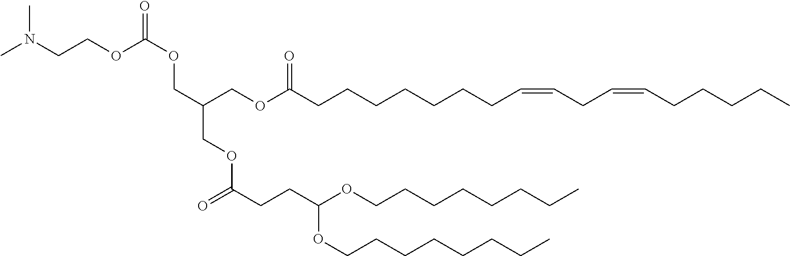 [SMILES: CCCCC/C=C\C/C=C\CCCCCCCC(=O)OCC(COC(=O)CCC(OCCCCCCCC)OCCCCCCCC)COC(=O)OCCN(C)C] |
| 19 | 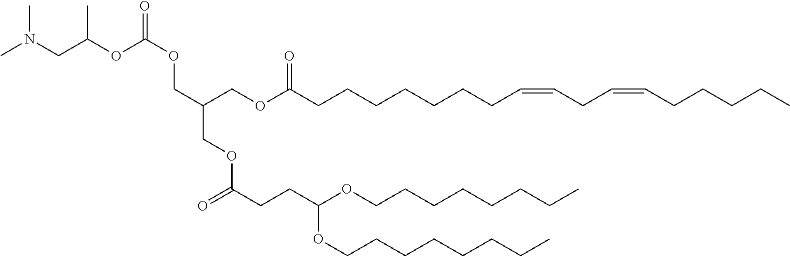 [SMILES: CCCCC/C=C\C/C=C\CCCCCCCC(=O)OCC(COC(=O)CCC(OCCCCCCCC)OCCCCCCCC)COC(=O)OC(C)CN(C)C] |
| 20 | 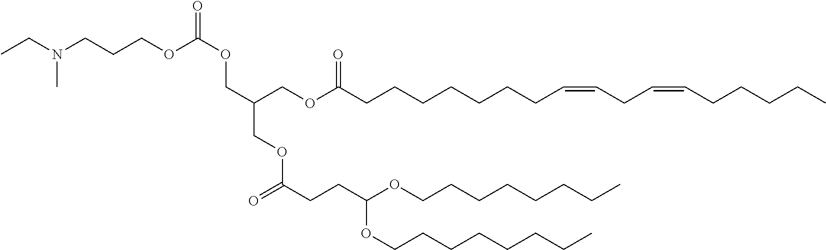 [SMILES: CCCCC/C=C\C/C=C\CCCCCCCC(=O)OCC(COC(=O)CCC(OCCCCCCCC)OCCCCCCCC)COC(=O)OCCCN(C)CC] |
| 21 | 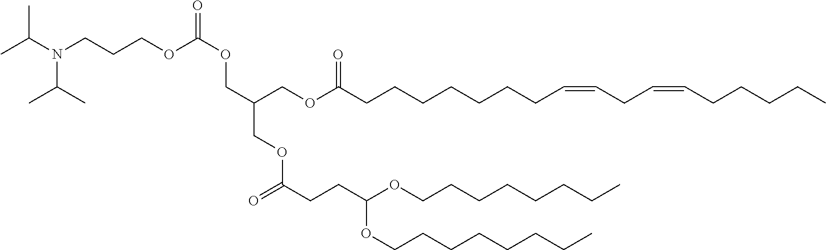 [SMILES: CCCCC/C=C\C/C=C\CCCCCCCC(=O)OCC(COC(=O)CCC(OCCCCCCCC)OCCCCCCCC)COC(=O)OCCCN(C(C)C)C(C)C] |
| 22 | 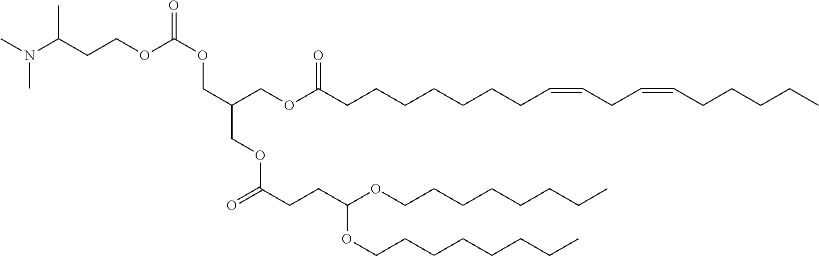 [SMILES: CCCCC/C=C\C/C=C\CCCCCCCC(=O)OCC(COC(=O)CCC(OCCCCCCCC)OCCCCCCCC)COC(=O)OCCC(C)N(C)C] |
| 23 | 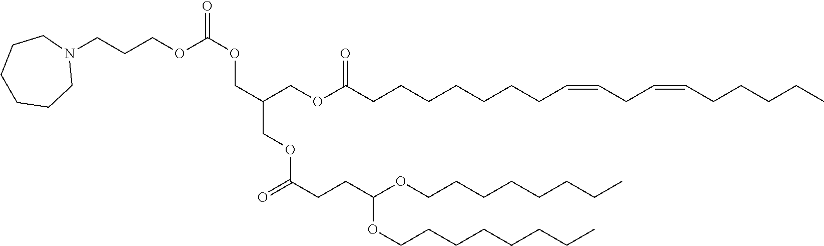 [SMILES: CCCCC/C=C\C/C=C\CCCCCCCC(=O)OCC(COC(=O)CCC(OCCCCCCCC)OCCCCCCCC)COC(=O)OCCCN1CCCCCC1] |
| 24 | 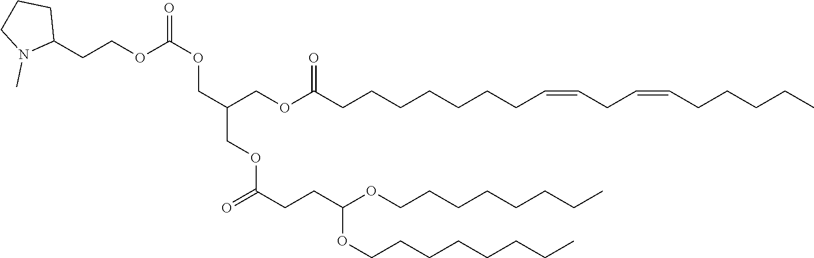 [SMILES: CCCCC/C=C\C/C=C\CCCCCCCC(=O)OCC(COC(=O)CCC(OCCCCCCCC)OCCCCCCCC)COC(=O)OCCC1CCCN1C] |
| 25 | 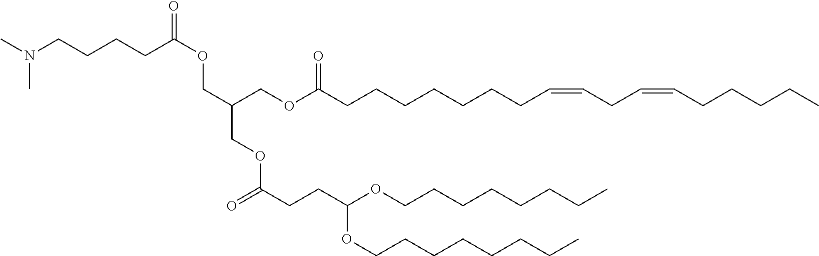 [SMILES: CCCCC/C=C\C/C=C\CCCCCCCC(=O)OCC(COC(=O)CCCCN(C)C)COC(=O)CCC(OCCCCCCCC)OCCCCCCCC] |
| 26 | 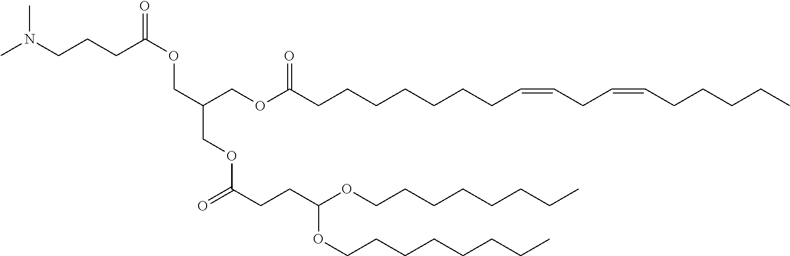 [SMILES: CCCCC/C=C\C/C=C\CCCCCCCC(=O)OCC(COC(=O)CCCN(C)C)COC(=O)CCC(OCCCCCCCC)OCCCCCCCC] |
| 27 | 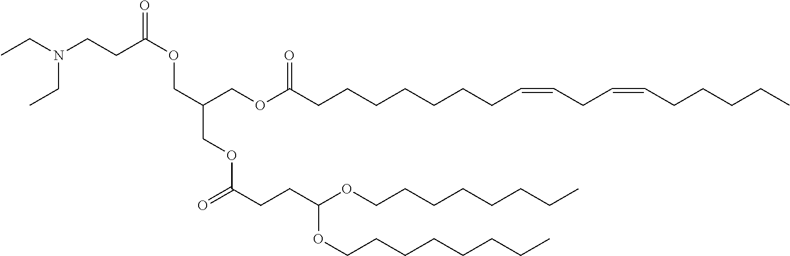 [SMILES: CCCCC/C=C\C/C=C\CCCCCCCC(=O)OCC(COC(=O)CCC(OCCCCCCCC)OCCCCCCCC)COC(=O)CCN(CC)CC] |
| 28 | 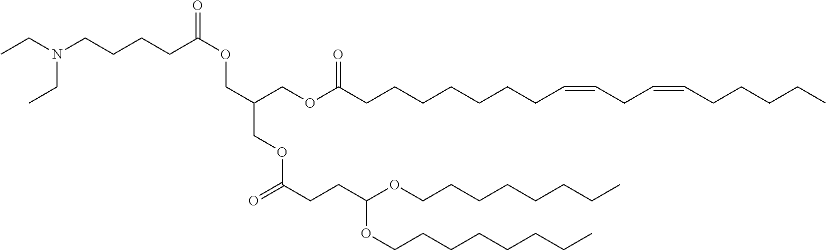 [SMILES: CCCCC/C=C\C/C=C\CCCCCCCC(=O)OCC(COC(=O)CCCCN(CC)CC)COC(=O)CCC(OCCCCCCCC)OCCCCCCCC] |
| 29 | 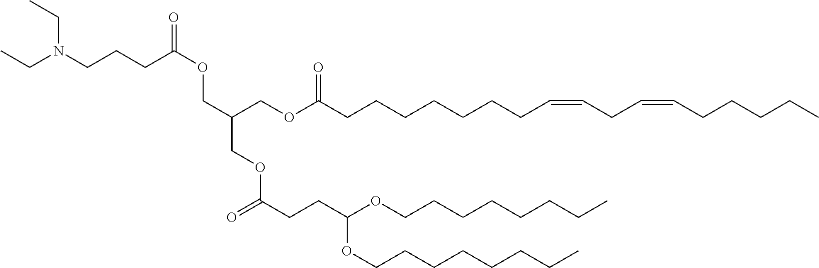 [SMILES: CCCCC/C=C\C/C=C\CCCCCCCC(=O)OCC(COC(=O)CCCN(CC)CC)COC(=O)CCC(OCCCCCCCC)OCCCCCCCC] |
| 30 | 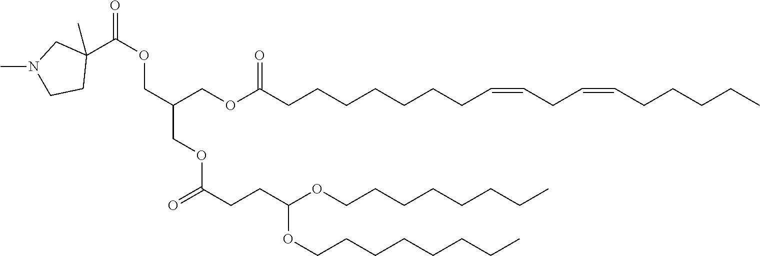 [SMILES: CCCCC/C=C\C/C=C\CCCCCCCC(=O)OCC(COC(=O)CCC(OCCCCCCCC)OCCCCCCCC)COC(=O)C1(C)CCN(C)C1] |
| 31 | 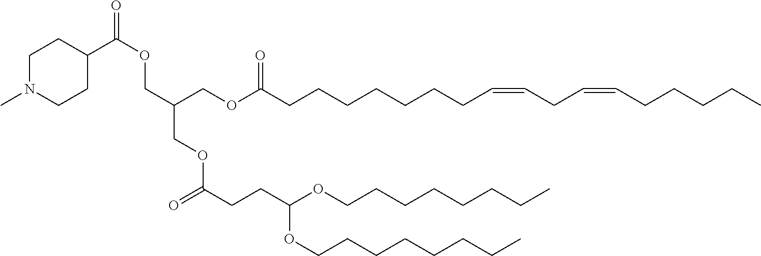 [SMILES: CCCCC/C=C\C/C=C\CCCCCCCC(=O)OCC(COC(=O)CCC(OCCCCCCCC)OCCCCCCCC)COC(=O)C1CCN(C)CC1] |
| 32 | 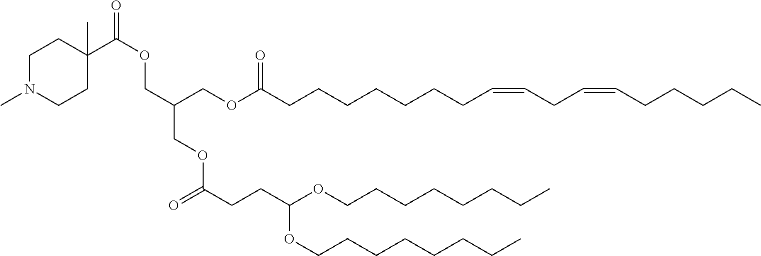 [SMILES: CCCCC/C=C\C/C=C\CCCCCCCC(=O)OCC(COC(=O)CCC(OCCCCCCCC)OCCCCCCCC)COC(=O)C1(C)CCN(C)CC1] |
| 33 | 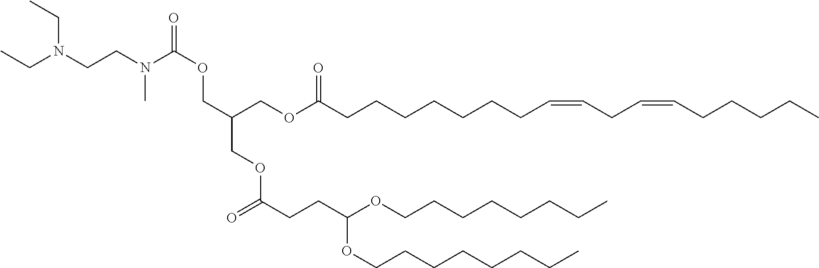 [SMILES: CCCCC/C=C\C/C=C\CCCCCCCC(=O)OCC(COC(=O)CCC(OCCCCCCCC)OCCCCCCCC)COC(=O)N(C)CCN(CC)CC] |
| 34 | 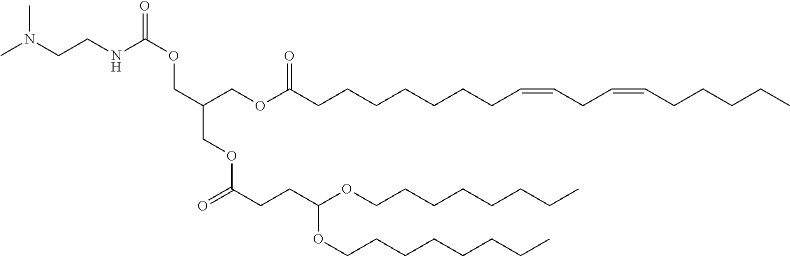 [SMILES: CCCCC/C=C\C/C=C\CCCCCCCC(=O)OCC(COC(=O)CCC(OCCCCCCCC)OCCCCCCCC)COC(=O)NCCN(C)C] |
| 35 | 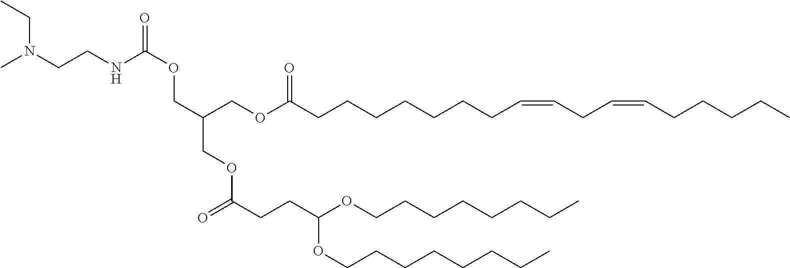 [SMILES: CCCCC/C=C\C/C=C\CCCCCCCC(=O)OCC(COC(=O)CCC(OCCCCCCCC)OCCCCCCCC)COC(=O)NCCN(C)CC] |
| 36 | 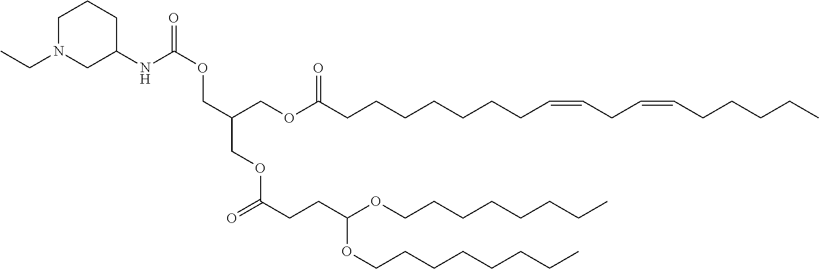 [SMILES: CCCCC/C=C\C/C=C\CCCCCCCC(=O)OCC(COC(=O)CCC(OCCCCCCCC)OCCCCCCCC)COC(=O)NC1CCCN(CC)C1] |
| 37 | 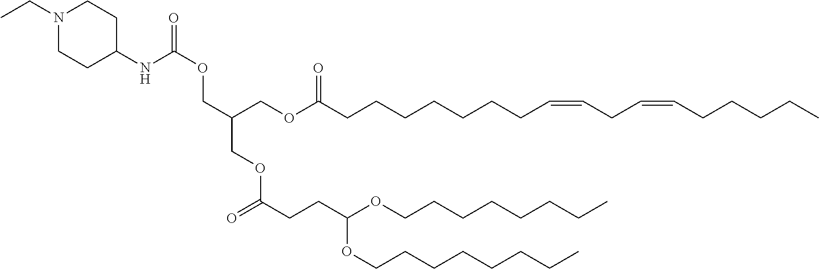 [SMILES: CCCCC/C=C\C/C=C\CCCCCCCC(=O)OCC(COC(=O)CCC(OCCCCCCCC)OCCCCCCCC)COC(=O)NC1CCN(CC)CC1] |
| 38 | 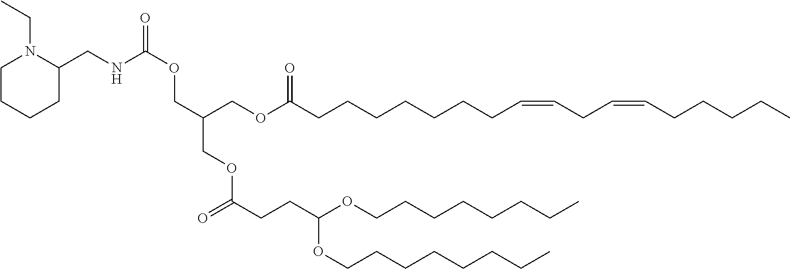 [SMILES: CCCCC/C=C\C/C=C\CCCCCCCC(=O)OCC(COC(=O)CCC(OCCCCCCCC)OCCCCCCCC)COC(=O)NCC1CCCCN1CC] |
| 39 | 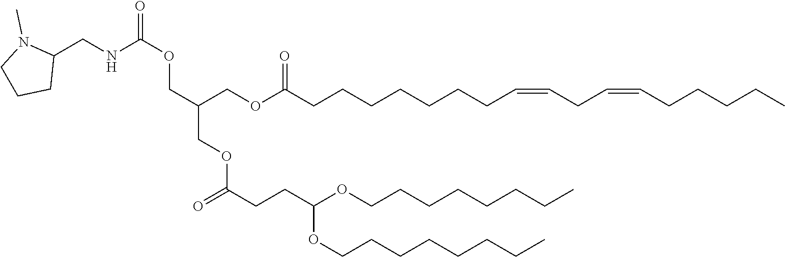 [SMILES: CCCCC/C=C\C/C=C\CCCCCCCC(=O)OCC(COC(=O)CCC(OCCCCCCCC)OCCCCCCCC)COC(=O)NCC1CCCN1C] |
| 40 | 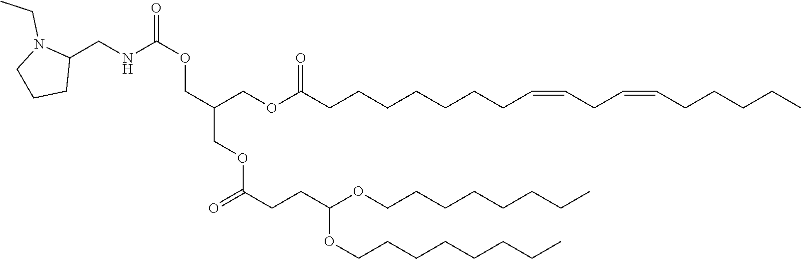 [SMILES: CCCCC/C=C\C/C=C\CCCCCCCC(=O)OCC(COC(=O)CCC(OCCCCCCCC)OCCCCCCCC)COC(=O)NCC1CCCN1CC] |
| 41 | 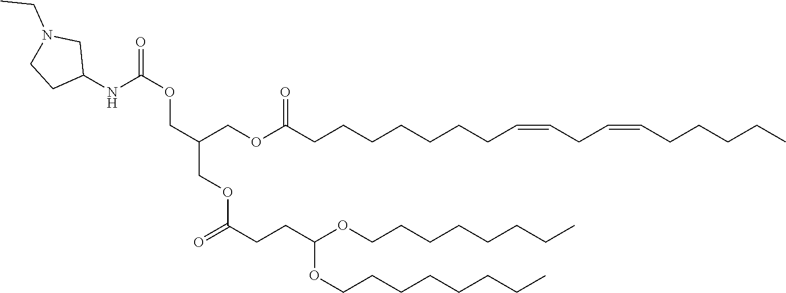 [SMILES: CCCCC/C=C\C/C=C\CCCCCCCC(=O)OCC(COC(=O)CCC(OCCCCCCCC)OCCCCCCCC)COC(=O)NC1CCN(CC)C1] |
| 42 | 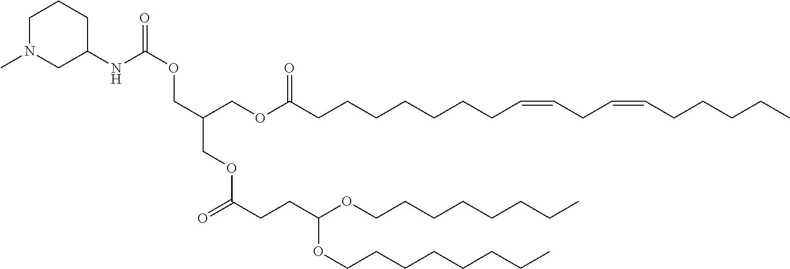 [SMILES: CCCCC/C=C\C/C=C\CCCCCCCC(=O)OCC(COC(=O)CCC(OCCCCCCCC)OCCCCCCCC)COC(=O)NC1CCCN(C)C1] |
| 43 | 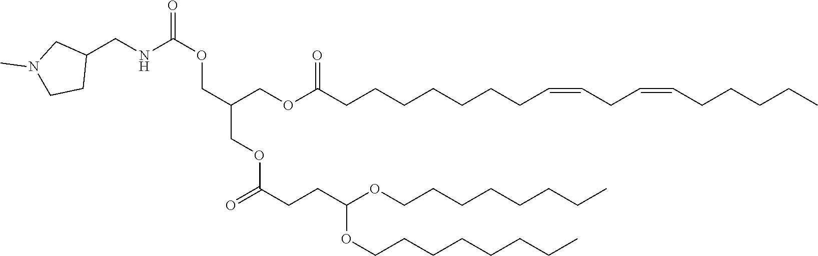 [SMILES: CCCCC/C=C\C/C=C\CCCCCCCC(=O)OCC(COC(=O)CCC(OCCCCCCCC)OCCCCCCCC)COC(=O)NCC1CCN(C)C1] |
| 44 | 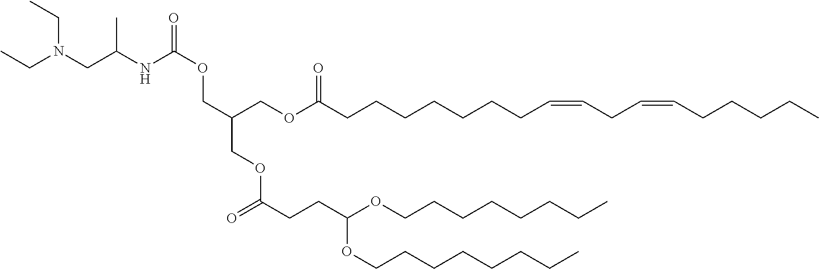 [SMILES: CCCCC/C=C\C/C=C\CCCCCCCC(=O)OCC(COC(=O)CCC(OCCCCCCCC)OCCCCCCCC)COC(=O)NC(C)CN(CC)CC] |
| 45 | 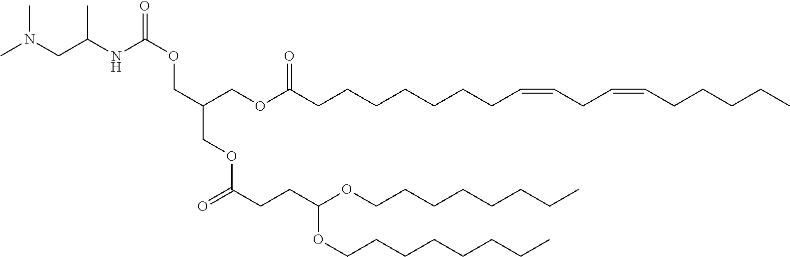 [SMILES: CCCCC/C=C\C/C=C\CCCCCCCC(=O)OCC(COC(=O)CCC(OCCCCCCCC)OCCCCCCCC)COC(=O)NC(C)CN(C)C] |
| 46 | 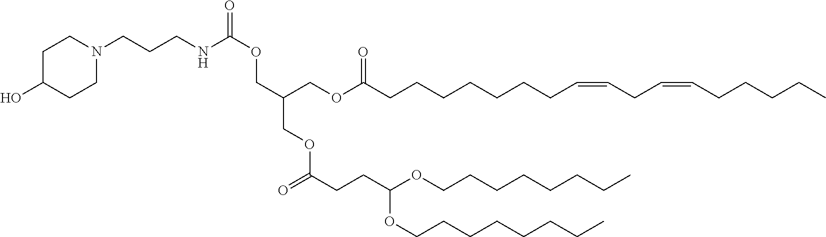 [SMILES: CCCCC/C=C\C/C=C\CCCCCCCC(=O)OCC(COC(=O)CCC(OCCCCCCCC)OCCCCCCCC)COC(=O)NCCCN1CCC(O)CC1] |
| 47 | 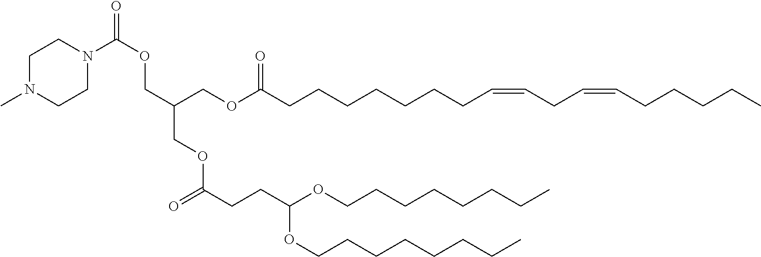 [SMILES: CCCCC/C=C\C/C=C\CCCCCCCC(=O)OCC(COC(=O)CCC(OCCCCCCCC)OCCCCCCCC)COC(=O)N1CCN(C)CC1] |
| 48 | 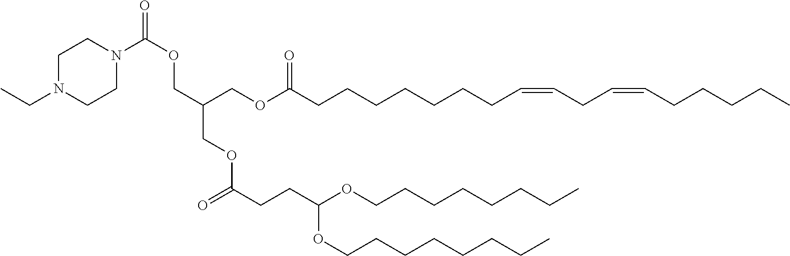 [SMILES: CCCCC/C=C\C/C=C\CCCCCCCC(=O)OCC(COC(=O)CCC(OCCCCCCCC)OCCCCCCCC)COC(=O)N1CCN(CC)CC1] |
| 49 | 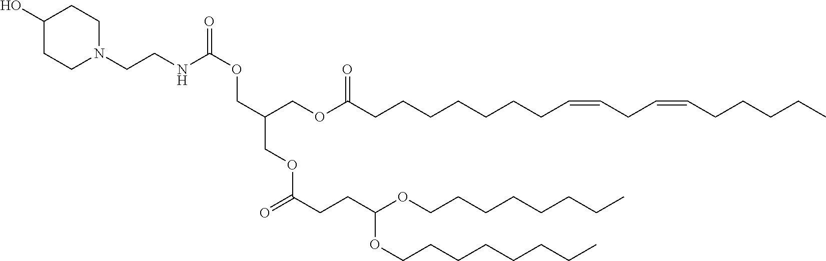 [SMILES: CCCCC/C=C\C/C=C\CCCCCCCC(=O)OCC(COC(=O)CCC(OCCCCCCCC)OCCCCCCCC)COC(=O)NCCN1CCC(O)CC1] |
| 50 | 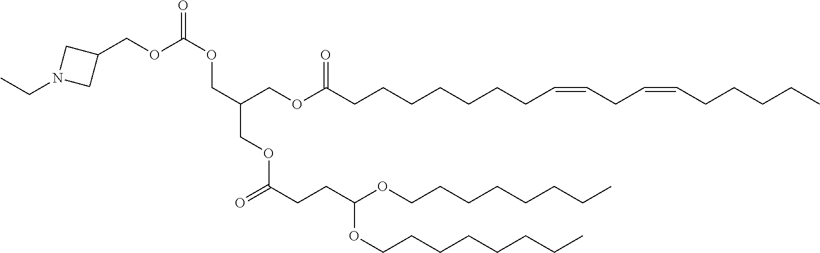 [SMILES: CCCCC/C=C\C/C=C\CCCCCCCC(=O)OCC(COC(=O)CCC(OCCCCCCCC)OCCCCCCCC)COC(=O)OCC1CN(CC)C1] |
| 51 | 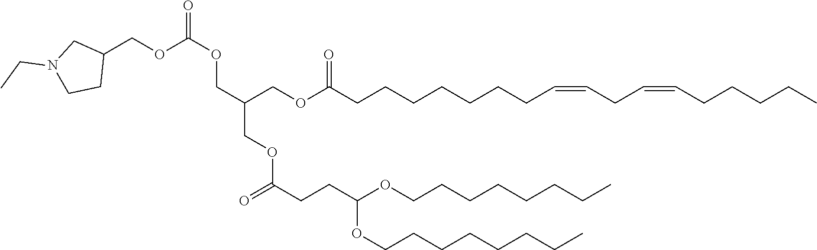 [SMILES: CCCCC/C=C\C/C=C\CCCCCCCC(=O)OCC(COC(=O)CCC(OCCCCCCCC)OCCCCCCCC)COC(=O)OCC1CCN(CC)C1] |
| 52 | 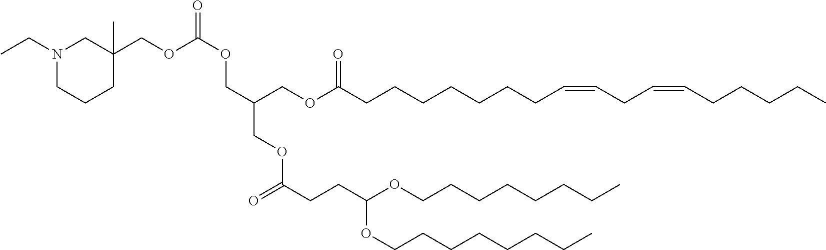 [SMILES: CCCCC/C=C\C/C=C\CCCCCCCC(=O)OCC(COC(=O)CCC(OCCCCCCCC)OCCCCCCCC)COC(=O)OCC1(C)CCCN(CC)C1] |
| 53 | 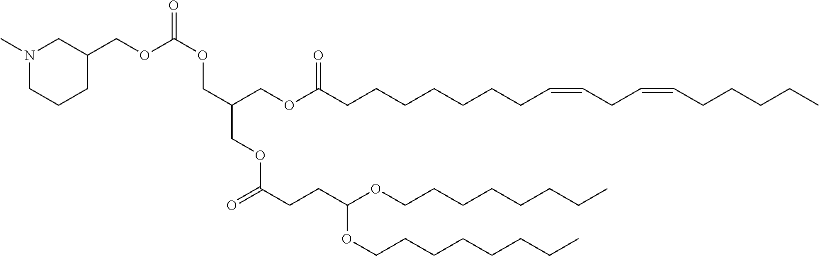 [SMILES: CCCCC/C=C\C/C=C\CCCCCCCC(=O)OCC(COC(=O)CCC(OCCCCCCCC)OCCCCCCCC)COC(=O)OCC1CCCN(C)C1] |
| 54 | 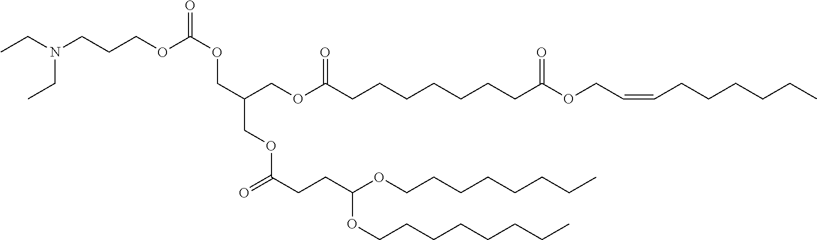 [SMILES: CCCCCC/C=C\COC(=O)CCCCCCCC(=O)OCC(COC(=O)CCC(OCCCCCCCC)OCCCCCCCC)COC(=O)OCCCN(CC)CC] |
| 55 | 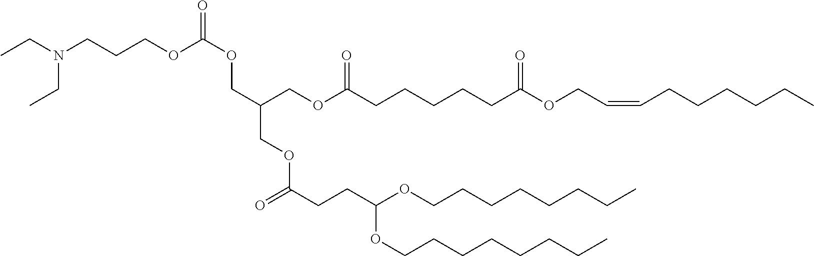 [SMILES: CCCCCC/C=C\COC(=O)CCCCCC(=O)OCC(COC(=O)CCC(OCCCCCCCC)OCCCCCCCC)COC(=O)OCCCN(CC)CC] |
| 56 | 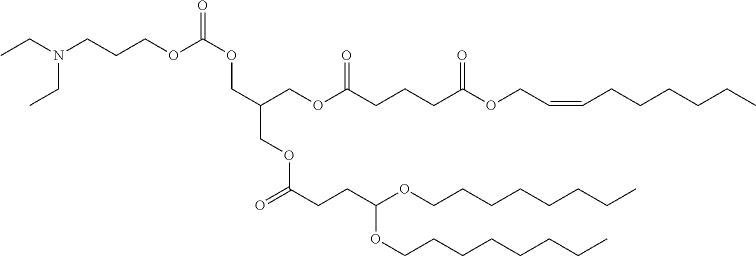 [SMILES: CCCCCC/C=C\COC(=O)CCCC(=O)OCC(COC(=O)CCC(OCCCCCCCC)OCCCCCCCC)COC(=O)OCCCN(CC)CC] |
| 57 | 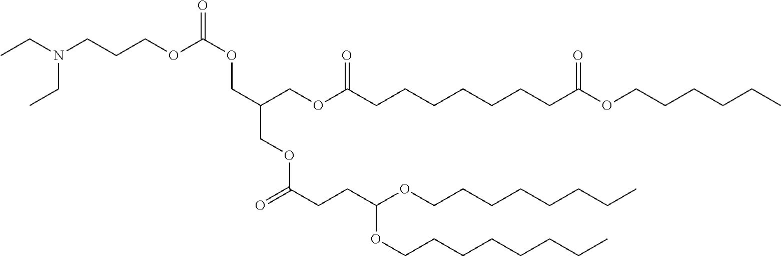 [SMILES: CCCCCCCCOC(CCC(=O)OCC(COC(=O)CCCCCCCC(=O)OCCCCCC)COC(=O)OCCCN(CC)CC)OCCCCCCCC] |
| 58 | 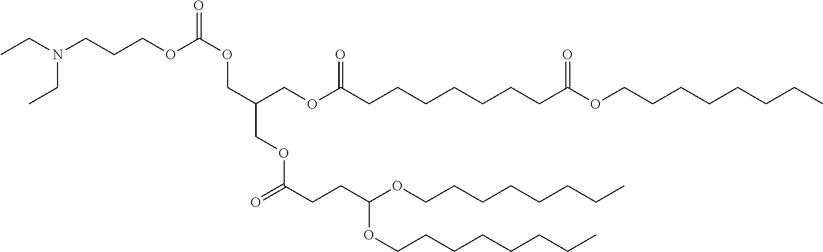 [SMILES: CCCCCCCCOC(=O)CCCCCCCC(=O)OCC(COC(=O)CCC(OCCCCCCCC)OCCCCCCCC)COC(=O)OCCCN(CC)CC] |
| 59 | 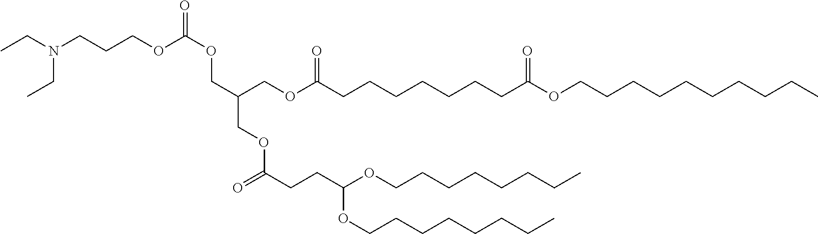 [SMILES: CCCCCCCCCCOC(=O)CCCCCCCC(=O)OCC(COC(=O)CCC(OCCCCCCCC)OCCCCCCCC)COC(=O)OCCCN(CC)CC] |
| 60 | 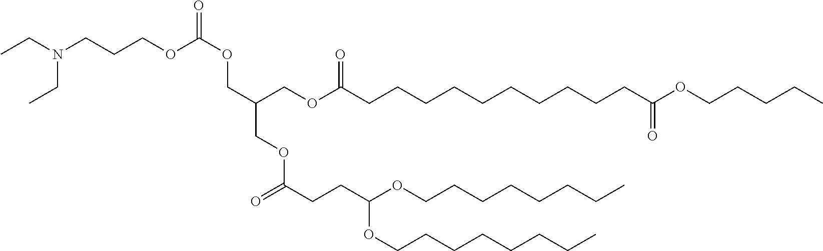 [SMILES: CCCCCCCCOC(CCC(=O)OCC(COC(=O)CCCCCCCCCCC(=O)OCCCCC)COC(=O)OCCCN(CC)CC)OCCCCCCCC] |
| 61 | 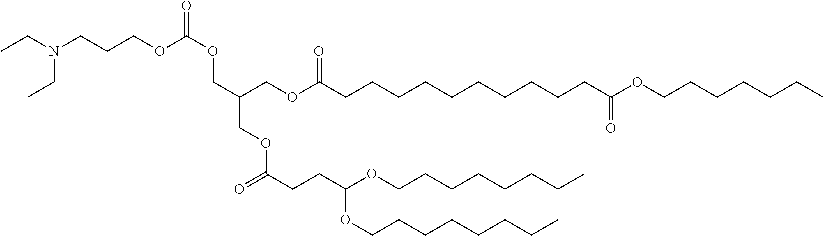 [SMILES: CCCCCCCCOC(CCC(=O)OCC(COC(=O)CCCCCCCCCCC(=O)OCCCCCCC)COC(=O)OCCCN(CC)CC)OCCCCCCCC] |
| 62 | 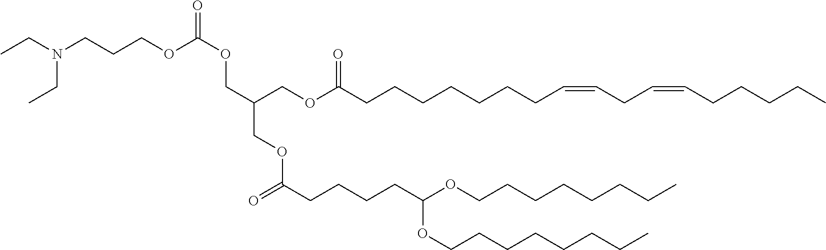 [SMILES: CCCCC/C=C\C/C=C\CCCCCCCC(=O)OCC(COC(=O)CCCCC(OCCCCCCCC)OCCCCCCCC)COC(=O)OCCCN(CC)CC] |
| 63 | 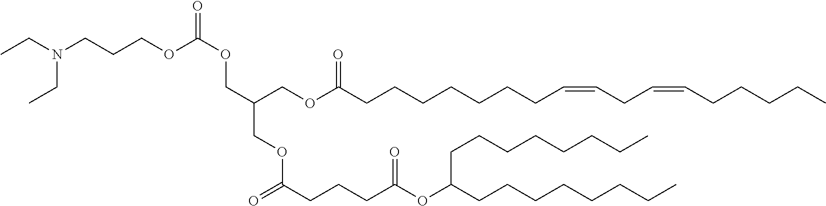 [SMILES: CCCCC/C=C\C/C=C\CCCCCCCC(=O)OCC(COC(=O)CCCC(=O)OC(CCCCCCCC)CCCCCCCC)COC(=O)OCCCN(CC)CC] |
| 64 | 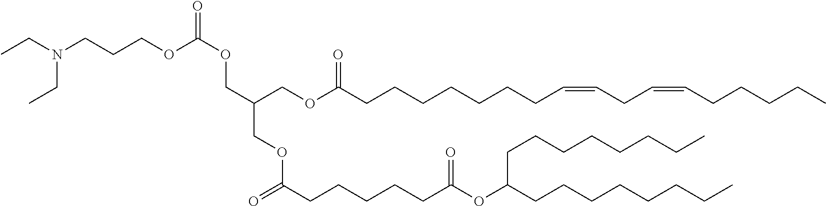 [SMILES: CCCCC/C=C\C/C=C\CCCCCCCC(=O)OCC(COC(=O)CCCCCC(=O)OC(CCCCCCCC)CCCCCCCC)COC(=O)OCCCN(CC)CC] |
| 65 | 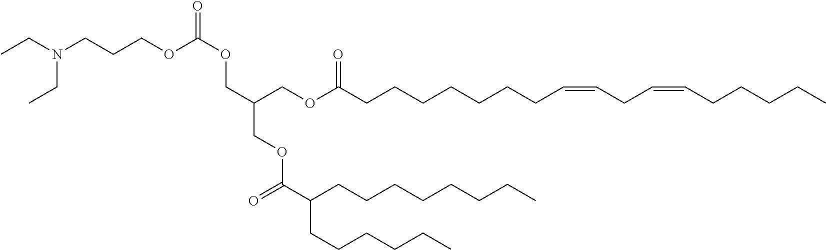 [SMILES: CCCCC/C=C\C/C=C\CCCCCCCC(=O)OCC(COC(=O)OCCCN(CC)CC)COC(=O)C(CCCCCC)CCCCCCCC] |
| 66 | 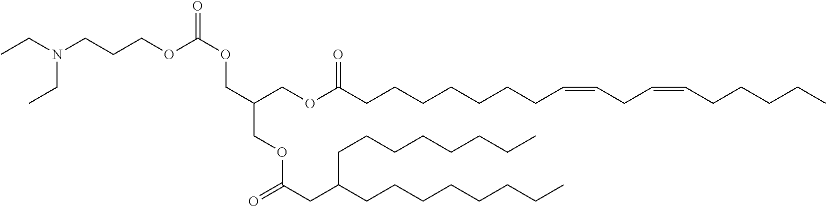 [SMILES: CCCCC/C=C\C/C=C\CCCCCCCC(=O)OCC(COC(=O)CC(CCCCCCCC)CCCCCCCC)COC(=O)OCCCN(CC)CC] |
| 67 | 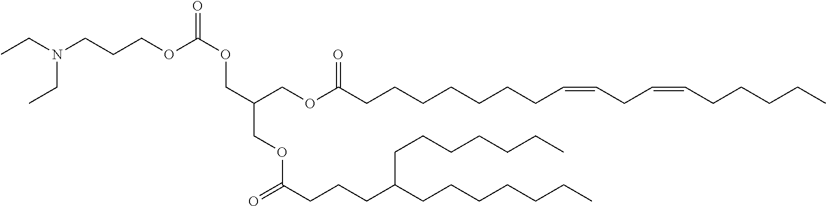 [SMILES: CCCCC/C=C\C/C=C\CCCCCCCC(=O)OCC(COC(=O)CCCC(CCCCCCC)CCCCCCC)COC(=O)OCCCN(CC)CC] |
| 68 | 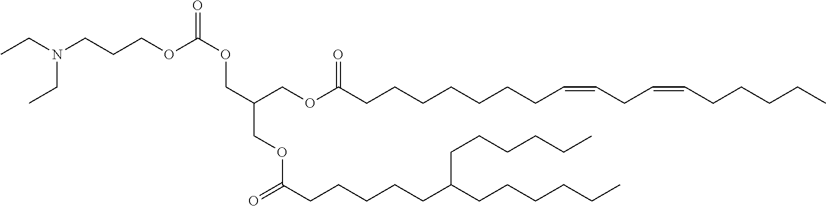 [SMILES: CCCCC/C=C\C/C=C\CCCCCCCC(=O)OCC(COC(=O)CCCCCC(CCCCCC)CCCCCC)COC(=O)OCCCN(CC)CC] |
| 69 | 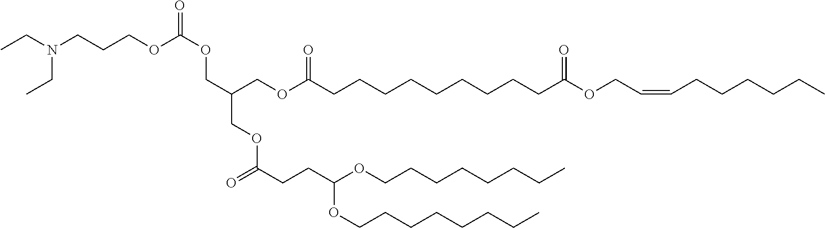 [SMILES: CCCCCC/C=C\COC(=O)CCCCCCCCCC(=O)OCC(COC(=O)CCC(OCCCCCCCC)OCCCCCCCC)COC(=O)OCCCN(CC)CC] |
| 70 | 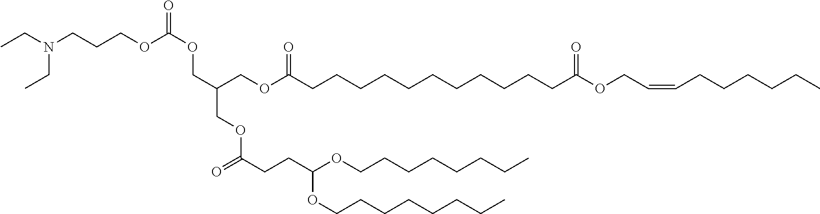 [SMILES: CCCCCC/C=C\COC(=O)CCCCCCCCCCCC(=O)OCC(COC(=O)CCC(OCCCCCCCC)OCCCCCCCC)COC(=O)OCCCN(CC)CC] |
| 71 | 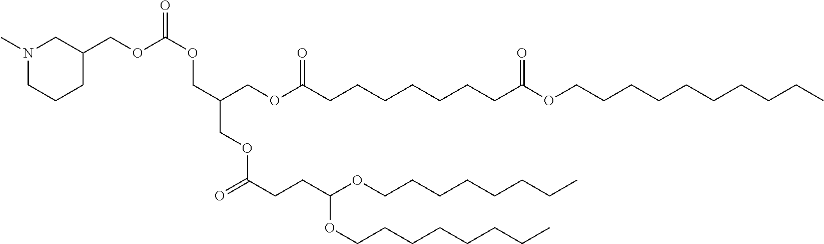 [SMILES: CCCCCCCCCCOC(=O)CCCCCCCC(=O)OCC(COC(=O)CCC(OCCCCCCCC)OCCCCCCCC)COC(=O)OCC1CCCN(C)C1] |
| 72 | 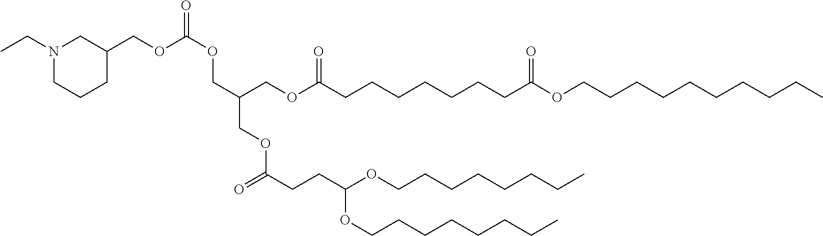 [SMILES: CCCCCCCCCCOC(=O)CCCCCCCC(=O)OCC(COC(=O)CCC(OCCCCCCCC)OCCCCCCCC)COC(=O)OCC1CCCN(CC)C1] |
| 73 | 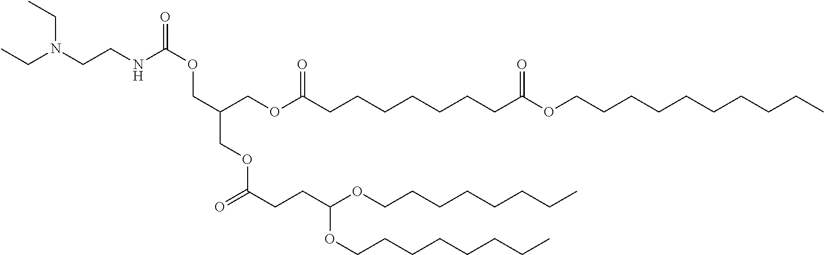 [SMILES: CCCCCCCCCCOC(=O)CCCCCCCC(=O)OCC(COC(=O)CCC(OCCCCCCCC)OCCCCCCCC)COC(=O)NCCN(CC)CC] |
| 74 | 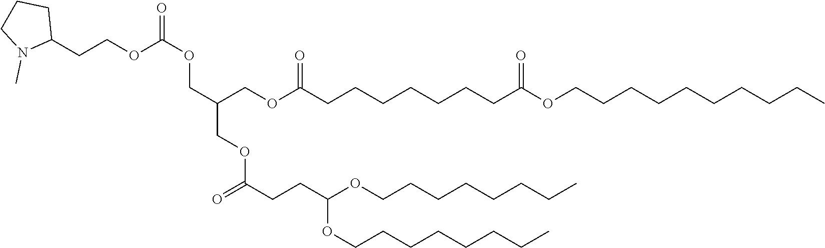 [SMILES: CCCCCCCCCCOC(=O)CCCCCCCC(=O)OCC(COC(=O)CCC(OCCCCCCCC)OCCCCCCCC)COC(=O)OCCC1CCCN1C] |
| 75 | 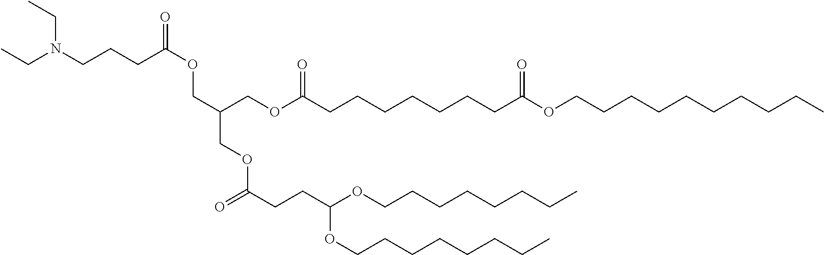 [SMILES: CCCCCCCCCCOC(=O)CCCCCCCC(=O)OCC(COC(=O)CCCN(CC)CC)COC(=O)CCC(OCCCCCCCC)OCCCCCCCC] |
| 76 | 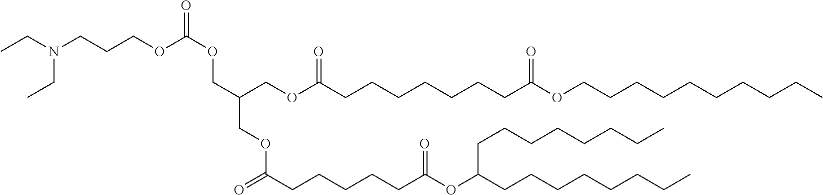 [SMILES: CCCCCCCCCCOC(=O)CCCCCCCC(=O)OCC(COC(=O)CCCCCC(=O)OC(CCCCCCCC)CCCCCCCC)COC(=O)OCCCN(CC)CC] |
| 77 | 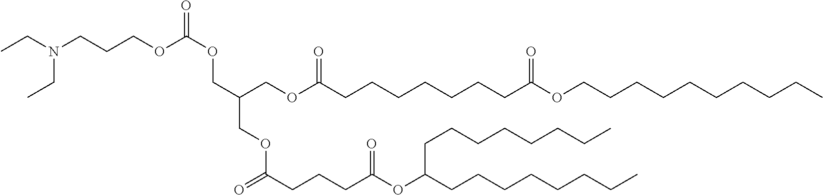 [SMILES: CCCCCCCCCCOC(=O)CCCCCCCC(=O)OCC(COC(=O)CCCC(=O)OC(CCCCCCCC)CCCCCCCC)COC(=O)OCCCN(CC)CC] |
| 78 | 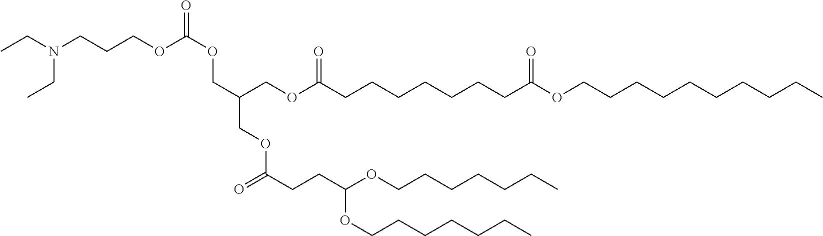 [SMILES: CCCCCCCCCCOC(=O)CCCCCCCC(=O)OCC(COC(=O)CCC(OCCCCCCC)OCCCCCCC)COC(=O)OCCCN(CC)CC] |
| 79 | 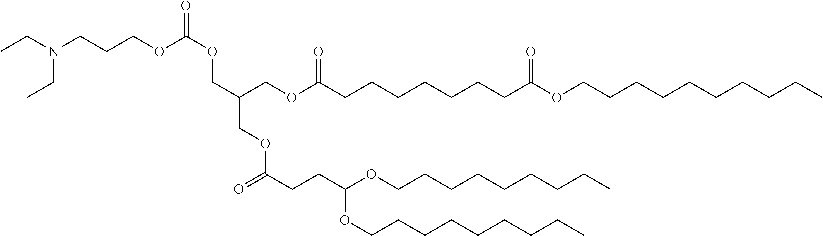 [SMILES: CCCCCCCCCCOC(=O)CCCCCCCC(=O)OCC(COC(=O)CCC(OCCCCCCCCC)OCCCCCCCCC)COC(=O)OCCCN(CC)CC] |
| 80 | 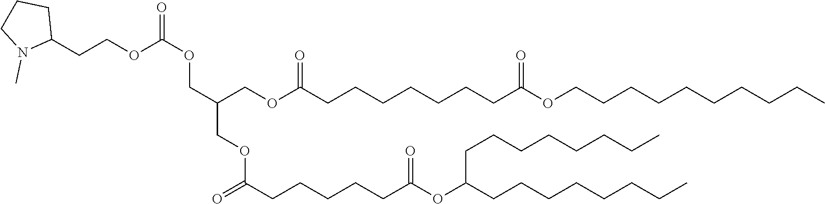 [SMILES: CCCCCCCCCCOC(=O)CCCCCCCC(=O)OCC(COC(=O)CCCCCC(=O)OC(CCCCCCCC)CCCCCCCC)COC(=O)OCCC1CCCN1C] |
| 81 | 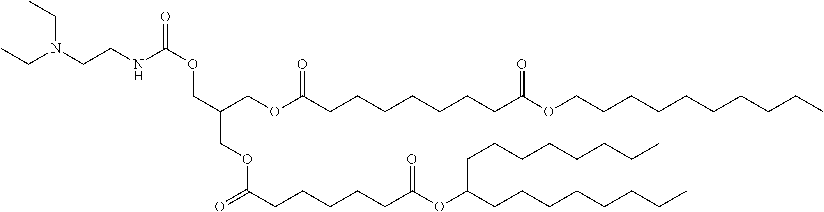 [SMILES: CCCCCCCCCCOC(=O)CCCCCCCC(=O)OCC(COC(=O)CCCCCC(=O)OC(CCCCCCCC)CCCCCCCC)COC(=O)NCCN(CC)CC] |
| 82 | 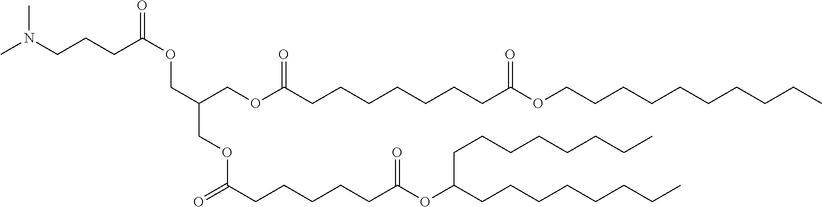 [SMILES: CCCCCCCCCCOC(=O)CCCCCCCC(=O)OCC(COC(=O)CCCCCC(=O)OC(CCCCCCCC)CCCCCCCC)COC(=O)CCCN(C)C] |
| 83 | 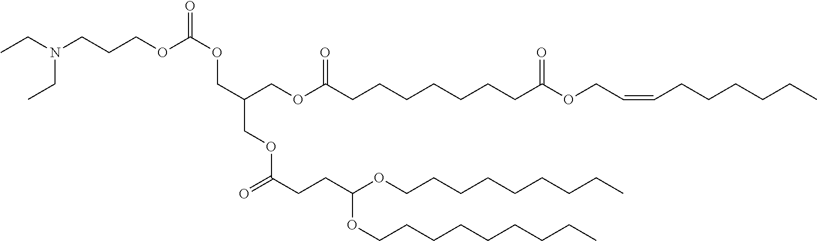 [SMILES: CCCCCC/C=C\COC(=O)CCCCCCCC(=O)OCC(COC(=O)CCC(OCCCCCCCCC)OCCCCCCCCC)COC(=O)OCCCN(CC)CC] |
| 84 | 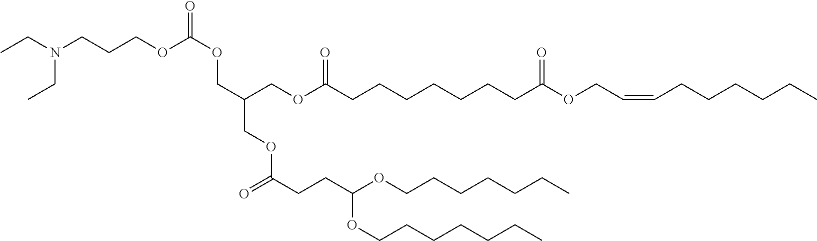 [SMILES: CCCCCC/C=C\COC(=O)CCCCCCCC(=O)OCC(COC(=O)CCC(OCCCCCCC)OCCCCCCC)COC(=O)OCCCN(CC)CC] |
| 85 | 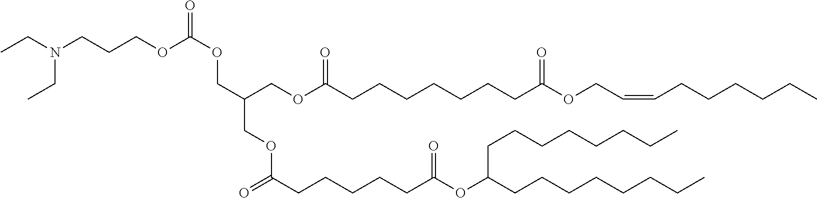 [SMILES: CCCCCC/C=C\COC(=O)CCCCCCCC(=O)OCC(COC(=O)CCCCCC(=O)OC(CCCCCCCC)CCCCCCCC)COC(=O)OCCCN(CC)CC] |
| 86 | 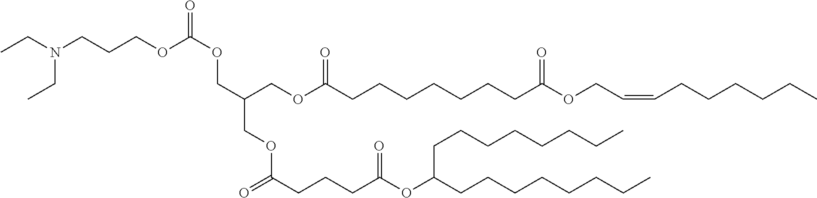 [SMILES: CCCCCC/C=C\COC(=O)CCCCCCCC(=O)OCC(COC(=O)CCCC(=O)OC(CCCCCCCC)CCCCCCCC)COC(=O)OCCCN(CC)CC] |
| 87 | 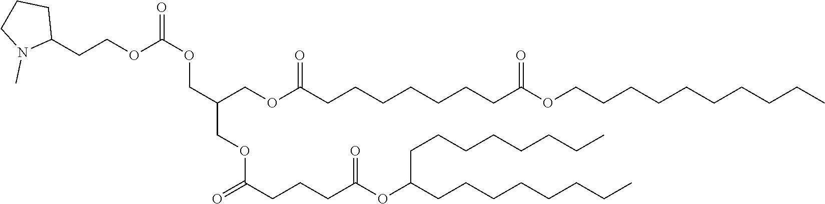 [SMILES: CCCCCCCCCCOC(=O)CCCCCCCC(=O)OCC(COC(=O)CCCC(=O)OC(CCCCCCCC)CCCCCCCC)COC(=O)OCCC1CCCN1C] |
| 88 | 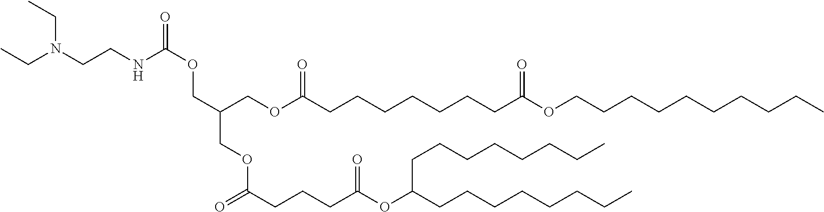 [SMILES: CCCCCCCCCCOC(=O)CCCCCCCC(=O)OCC(COC(=O)CCCC(=O)OC(CCCCCCCC)CCCCCCCC)COC(=O)NCCN(CC)CC] |
| 89 | 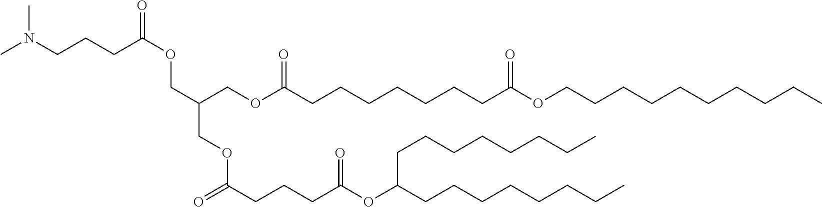 [SMILES: CCCCCCCCCCOC(=O)CCCCCCCC(=O)OCC(COC(=O)CCCC(=O)OC(CCCCCCCC)CCCCCCCC)COC(=O)CCCN(C)C] |
| 90 | 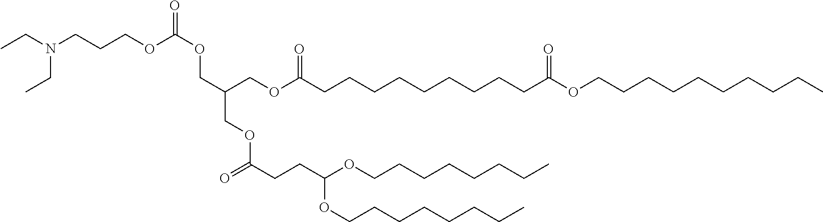 [SMILES: CCCCCCCCCCOC(=O)CCCCCCCCCC(=O)OCC(COC(=O)CCC(OCCCCCCCC)OCCCCCCCC)COC(=O)OCCCN(CC)CC] |
| 91 | 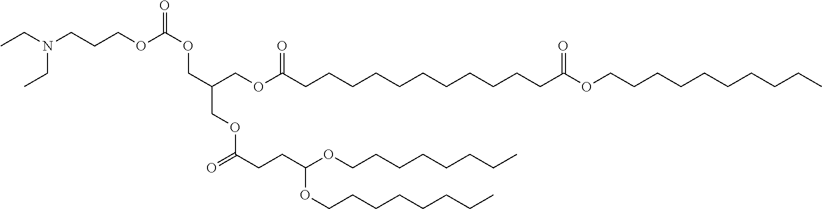 [SMILES: CCCCCCCCCCOC(=O)CCCCCCCCCCCC(=O)OCC(COC(=O)CCC(OCCCCCCCC)OCCCCCCCC)COC(=O)OCCCN(CC)CC] |
| 92 | 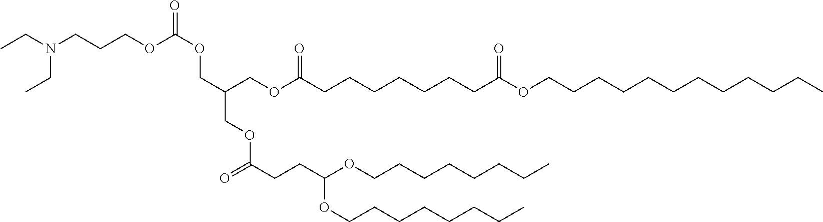 [SMILES: CCCCCCCCCCCCOC(=O)CCCCCCCC(=O)OCC(COC(=O)CCC(OCCCCCCCC)OCCCCCCCC)COC(=O)OCCCN(CC)CC] |
| 93 | 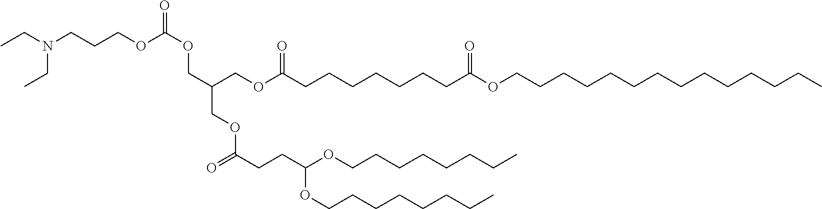 [SMILES: CCCCCCCCCCCCCCOC(=O)CCCCCCCC(=O)OCC(COC(=O)CCC(OCCCCCCCC)OCCCCCCCC)COC(=O)OCCCN(CC)CC] |
| 94 | 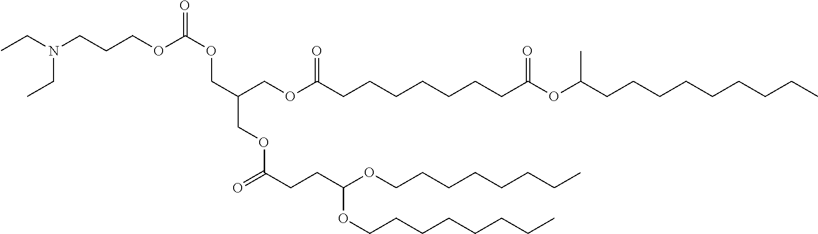 [SMILES: CCCCCCCCCC(C)OC(=O)CCCCCCCC(=O)OCC(COC(=O)CCC(OCCCCCCCC)OCCCCCCCC)COC(=O)OCCCN(CC)CC] |
| 95 | 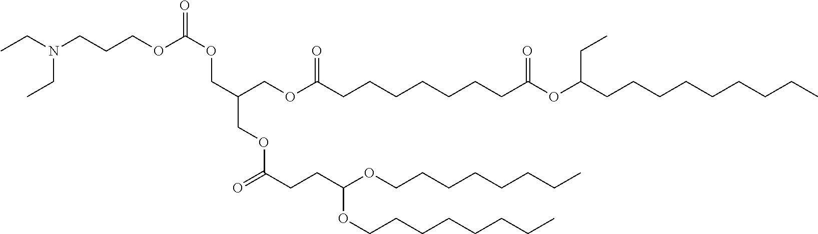 [SMILES: CCCCCCCCCC(CC)OC(=O)CCCCCCCC(=O)OCC(COC(=O)CCC(OCCCCCCCC)OCCCCCCCC)COC(=O)OCCCN(CC)CC] |
| 96 | 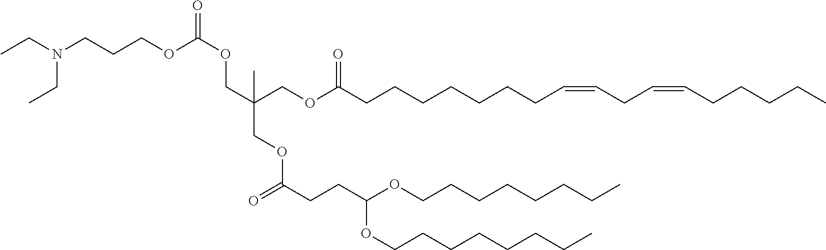 [SMILES: CCCCC/C=C\C/C=C\CCCCCCCC(=O)OCC(C)(COC(=O)CCC(OCCCCCCCC)OCCCCCCCC)COC(=O)OCCCN(CC)CC] |
| 97 | 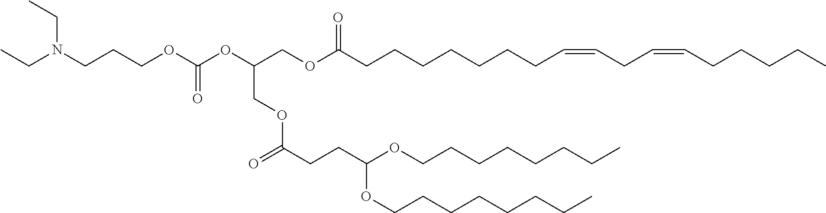 [SMILES: CCCCC/C=C\C/C=C\CCCCCCCC(=O)OCC(COC(=O)CCC(OCCCCCCCC)OCCCCCCCC)OC(=O)OCCCN(CC)CC] |
| 98 | 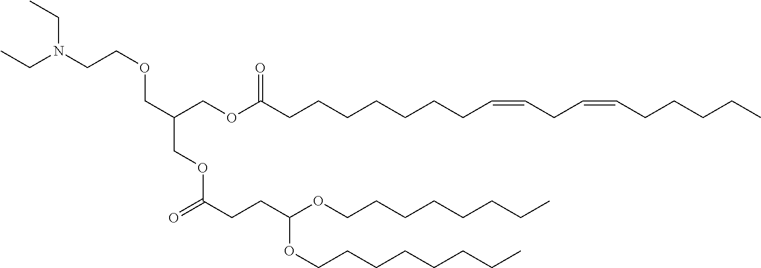 [SMILES: CCCCC/C=C\C/C=C\CCCCCCCC(=O)OCC(COCCN(CC)CC)COC(=O)CCC(OCCCCCCCC)OCCCCCCCC] |
| 99 | 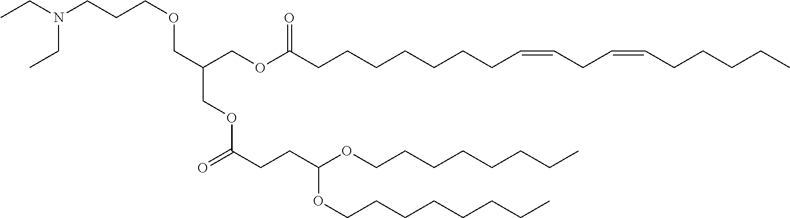 [SMILES: CCCCC/C=C\C/C=C\CCCCCCCC(=O)OCC(COCCCN(CC)CC)COC(=O)CCC(OCCCCCCCC)OCCCCCCCC] |
| 100 | 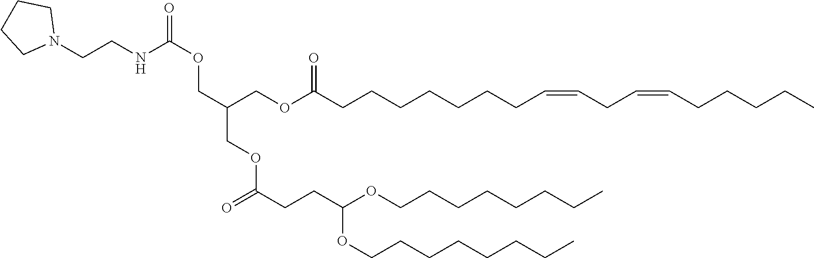 [SMILES: CCCCC/C=C\C/C=C\CCCCCCCC(=O)OCC(COC(=O)CCC(OCCCCCCCC)OCCCCCCCC)COC(=O)NCCN1CCCC1] |
| 101 | 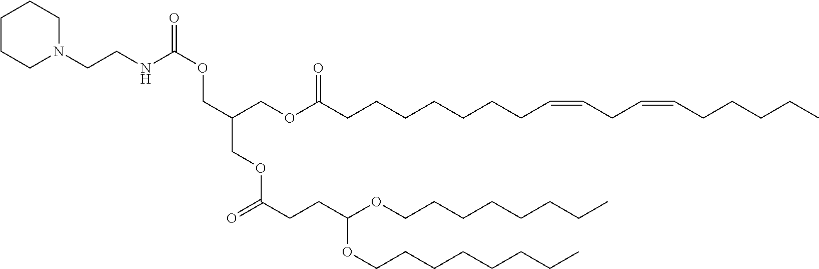 [SMILES: CCCCC/C=C\C/C=C\CCCCCCCC(=O)OCC(COC(=O)CCC(OCCCCCCCC)OCCCCCCCC)COC(=O)NCCN1CCCCC1] |
| 102 | 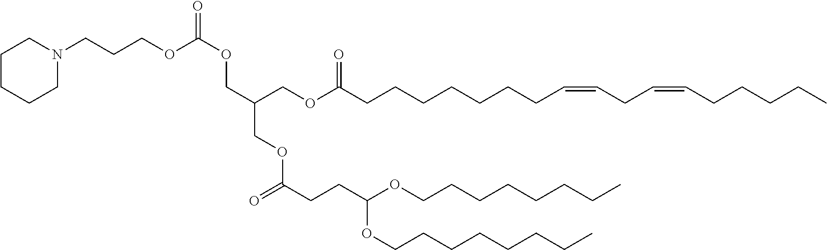 [SMILES: CCCCC/C=C\C/C=C\CCCCCCCC(=O)OCC(COC(=O)CCC(OCCCCCCCC)OCCCCCCCC)COC(=O)OCCCN1CCCCC1] |
| 103 | 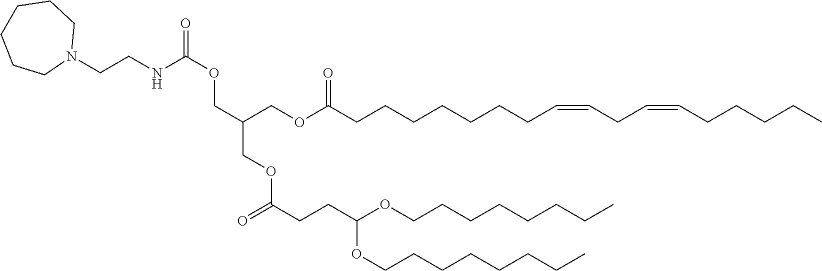 [SMILES: CCCCC/C=C\C/C=C\CCCCCCCC(=O)OCC(COC(=O)CCC(OCCCCCCCC)OCCCCCCCC)COC(=O)NCCN1CCCCCC1] |
| 107 | 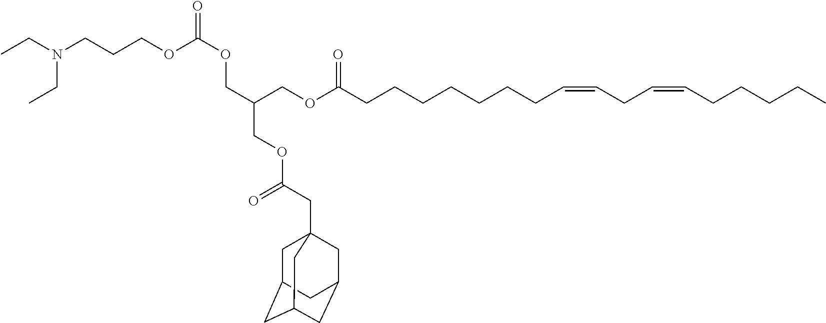 [SMILES: CCCCC/C=C\C/C=C\CCCCCCCC(=O)OCC(COC(=O)CC12CC3CC(CC(C3)C1)C2)COC(=O)OCCCN(CC)CC] |
| 113 | 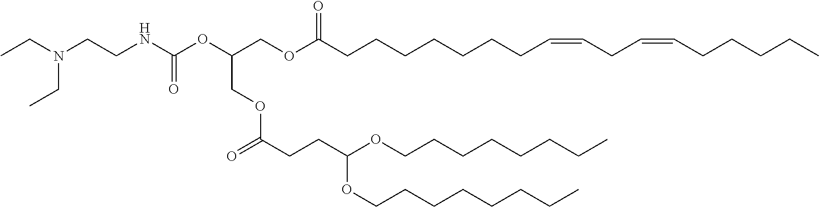 [SMILES: CCCCC/C=C\C/C=C\CCCCCCCC(=O)OCC(COC(=O)CCC(OCCCCCCCC)OCCCCCCCC)OC(=O)NCCN(CC)CC] |
| 114 | 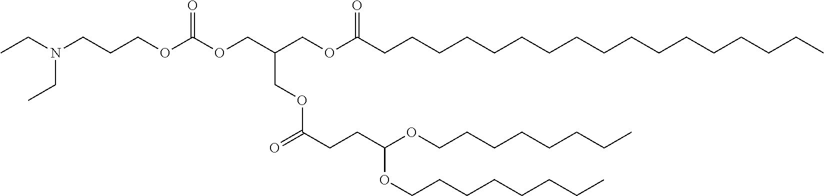 [SMILES: CCCCCCCCCCCCCCCCCC(=O)OCC(COC(=O)CCC(OCCCCCCCC)OCCCCCCCC)COC(=O)OCCCN(CC)CC] |
| 115 | 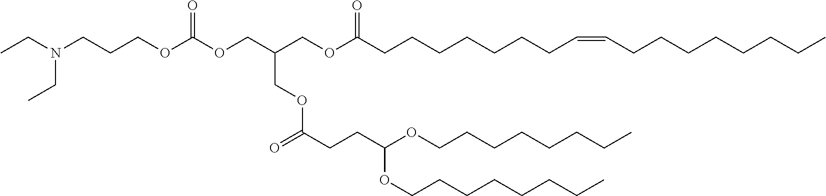 [SMILES: CCCCCCCC/C=C\CCCCCCCC(=O)OCC(COC(=O)CCC(OCCCCCCCC)OCCCCCCCC)COC(=O)OCCCN(CC)CC] |
| 116 | 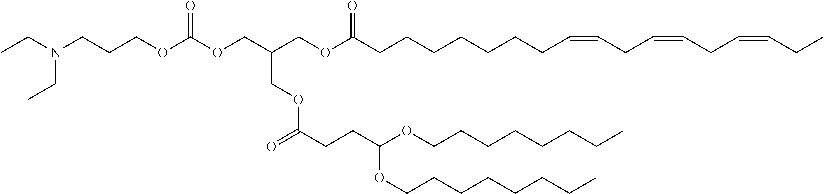 [SMILES: CC/C=C\C/C=C\C/C=C\CCCCCCCC(=O)OCC(COC(=O)CCC(OCCCCCCCC)OCCCCCCCC)COC(=O)OCCCN(CC)CC] |
| 117 | 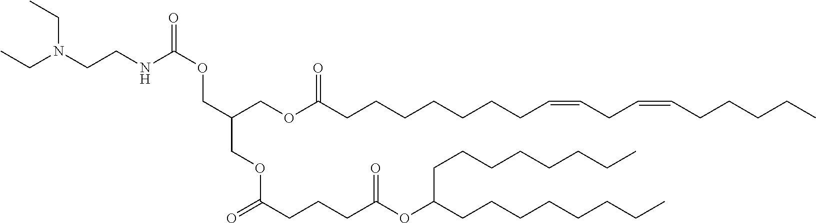 [SMILES: CCCCC/C=C\C/C=C\CCCCCCCC(=O)OCC(COC(=O)CCCC(=O)OC(CCCCCCCC)CCCCCCCC)COC(=O)NCCN(CC)CC] |
| 118 | 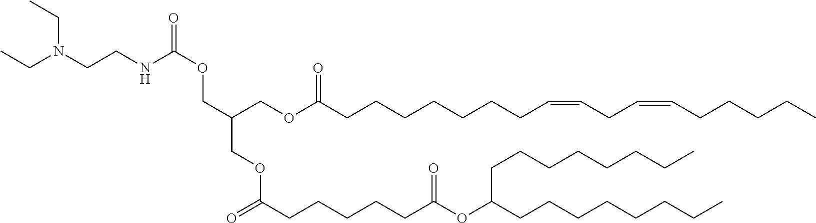 [SMILES: CCCCC/C=C\C/C=C\CCCCCCCC(=O)OCC(COC(=O)CCCCCC(=O)OC(CCCCCCCC)CCCCCCCC)COC(=O)NCCN(CC)CC] |

### Synthesis of Example

### Synthesis of Example 1

### Intermediate 1a: 3-hydroxy-2-(hydroxymethyl)propyl (9Z,12Z)-octadeca-9,12-dienoate

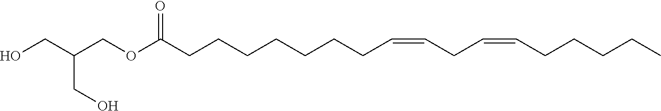 [SMILES: CCCCC/C=C\C/C=C\CCCCCCCC(=O)OCC(CO)CO]

To a solution of linoleic acid (13.2 g, 47.1 mmol), DMAP (1.15 g, 9.42 mmol), DIPEA (12.3 mL, 70.6 mmol), and 2-(hydroxymethyl)propane-1,3-diol (5 g, 47.1 mmol) in DCM (100 mL) was added EDC.HCl (13.5 g, 70.6 mmol) at rt. The reaction mixture was stirred at rt for 24 h, concentrated in vacuo, and directly purified using silica gel chromatography (120 g HC Si, 0-50% EtOAc in hexanes) to provide 6.7 g (18.1 mmol, 39% yield) of the desired product as an oil. ¹H NMR (400 MHz, CDCl₃) δ 5.44-5.25 (m, 4H), 4.25 (d, J=6.3 Hz, 2H), 3.76 (m, 4H), 2.77 (t, J=6.5 Hz, 2H), 2.43-2.27 (m, 4H), 2.11-1.96 (m, 5H), 1.62 (m, 2H), 1.43-1.22 (m, 14H), 0.89 (t, J=6.8 Hz, 3H) ppm.

### Intermediate 1b: 4,4-bis(octyloxy)butanenitrile

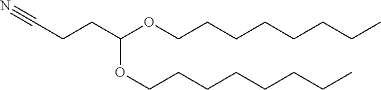 [SMILES: CCCCCCCCOC(CCC#N)OCCCCCCCC]

To a mixture of 4,4-diethoxybutanenitrile (9.4 g, 60 mmol, 1 equiv) and octan-1-ol (3 equiv) was added pyridiniump-toluenesulfonate (0.05 equiv) at rt. The reaction mixture was warmed to 105° C. and stirred for at least 18 h with the reaction vessel open to air and not fitted with a refluxing condenser. The reaction mixture was then cooled to rt and directly purified using silica gel chromatography (gradient of EtOAc in hexanes) to provide 10.1 g (31.0 mmol, 52% yield) of the desired product as a clear oil. ¹H NMR (400 MHz, CDCl₃) δ 4.55 (t, J=5.3 Hz, 1H), 3.60 (dt, J=9.2, 6.6 Hz, 2H), 3.43 (dt, J=9.2, 6.6 Hz, 2H), 2.42 (t, J=7.4 Hz, 2H), 1.94 (td, J=7.4, 5.3 Hz, 2H), 1.63-1.50 (m, 4H), 1.38-1.19 (m, 20H), 0.93-0.82 (m, 6H) ppm; MS: 348 m/z [M+Na].

### Intermediate 1c: 4,4-bis(octyloxy)butanoic acid

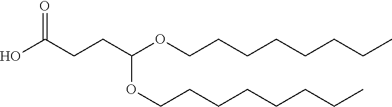 [SMILES: CCCCCCCCOC(CCC(=O)O)OCCCCCCCC]

To a solution of Intermediate 1b (8.42 g, 31 mmol, 1 equiv) in ethanol (1 M) was added aqueous potassium hydroxide (2.5 M, 2.5 equiv) at rt. Upon fitting the reaction vessel with a reflux condenser, the reaction mixture was warmed to 110° C. and stirred for 20-24 h. The reaction mixture was then cooled to room temperature, acidified with aqueous 1N HCl to pH 5, and extracted into hexanes. The combined organic extracts were washed with water and brine, dried over anhydrous sodium sulfate or magnesium sulfate, filtered, and concentrated in vacuo to afford 8.15 g (23.6 mmol, 76% yield) of the desired product as a clear oil, which was used crude without further purification. ¹H NMR (400 MHz, CDCl₃) δ 4.50 (t, J=5.5 Hz, 1H), 3.57 (dt, J=9.4, 6.7 Hz, 2H), 3.41 (dt, J=9.3, 6.7 Hz, 2H), 2.40 (t, J=7.4 Hz, 2H), 1.92 (td, J=7.4, 5.3 Hz, 2H), 1.56 (m, 4H), 1.37-1.21 (m, 20H), 0.92-0.83 (m, 6H) ppm; MS: 343 m/z [M−H].

### Intermediate 1d: 3-((4,4-bis(octyloxy)butanoyl)oxy)-2-(hydroxymethyl)propyl (9Z,12Z)-octadeca-9,12-dienoate

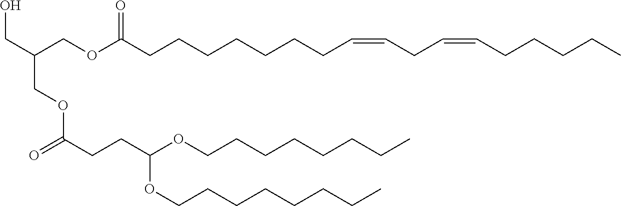 [SMILES: CCCCC/C=C\C/C=C\CCCCCCCC(=O)OCC(CO)COC(=O)CCC(OCCCCCCCC)OCCCCCCCC]

To a solution of Intermediate 1a (0.8-1.2 equiv) and Intermediate 1c (1.1 g, 3.2 mmol, 1 equiv in DCM (0.08-0.4 M) was added DMAP (0.1-0.2 equiv), DIPEA (1.4-3 equiv), and EDC.HCl (1.4-1.6 equiv) at rt. The reaction mixture was stirred at rt for at least 5 h, concentrated in vacuo, and directly purified using silica gel chromatography (a gradient of EtOAc in hexanes) to provide 1.08 g (1.55 mmol, 48% yield) of the desired product as a clear oil. ¹H NMR (400 MHz, CDCl₃) δ 5.36 (m, 4H), 4.49 (t, J=5.4 Hz, 1H), 4.17 (m, 4H), 3.66-3.53 (m, 4H), 3.40 (m, 2H), 2.77 (t, J=6.4 Hz, 2H), 2.41 (t, J=7.6 Hz, 2H), 2.32 (t, J=7.6 Hz, 2H), 2.19 (m, 2H), 2.05 (m, 4H), 1.93 (m, 2H), 1.58 (m, 7H), 1.31 (m, 32H), 0.88 (m, 9H) ppm.

### Example 1

### 3-((4,4-bis(octyloxy)butanoyl)oxy)-2-((((3-(diethylamino)propoxy)carbonyl)oxy)methyl)propyl (9Z,12Z)-octadeca-9,12-dienoate

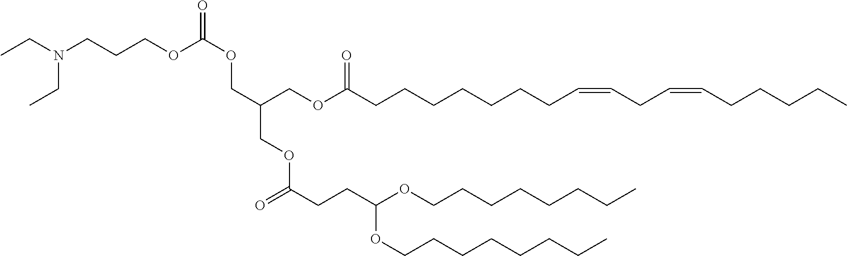 [SMILES: CCCCC/C=C\C/C=C\CCCCCCCC(=O)OCC(COC(=O)CCC(OCCCCCCCC)OCCCCCCCC)COC(=O)OCCCN(CC)CC]

To a solution of intermediate 1d (1.08 g, 1.55 mmol, 1 equiv) in acetonitrile (0.04-0.1 M) was added pyridine (2 equiv), DMAP (0.2 equiv), and 4-nitrophenyl chloroformate (1.5 equiv) sequentially at rt. Upon stirring for at least 2 h, 3-(diethylamino)propan-1-ol (3 equiv) was added and the resulting reaction mixture was stirred for an additional 2-24 h at rt. The reaction mixture was extracted into hexanes (20 mL) and washed with water. The resulting water layer was re-extracted with hexanes. The combined hexanes layers were dried over anhydrous MgSO₄ or Na₂SO₄, filtered, and concentrated in vacuo. The crude residue was purified using silica gel chromatography (a gradient of EtOAc in hexanes or methanol in DCM) to provide 711 mg (0.834 mmol, 54% yield) of the desired product as a clear oil. ¹H NMR (CDCl₃, 400 MHz) δ 5.35 (m, 4H), 4.48 (t, J=5.6 Hz, 1H), 4.17 (m, 8H), 3.56 (m, 2H), 3.40 (m, 2H), 2.77 (t, J=6.6 Hz, 2H), 2.55 (q, J=7.2 Hz, 6H), 2.40 (m, 3H), 2.30 (t, J=7.6 Hz, 2H), 2.05 (q, J=6.8 Hz, 4H), 1.92 (m, 2H), 1.84 (m, 2H), 1.57 (m, 6H), 1.30 (m, 34H), 1.03 (t, J=7.2 Hz, 6H), 0.88 (m, 9H) ppm; MS: 853 m/z [M+H].

### Synthesis of Examples 2-24

The following examples were synthesized from Intermediate 1d and an amino alcohol or diamine reagent using the method employed for Example 1.

### Example 2

### 3-((4,4-bis(octyloxy)butanoyl)oxy)-2-((((3-(dimethylamino)propoxy)carbonyl)oxy)methyl)propyl (9Z,12Z)-octadeca-9,12-dienoate

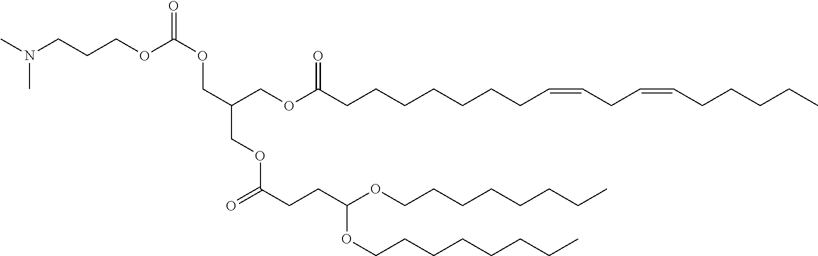 [SMILES: CCCCC/C=C\C/C=C\CCCCCCCC(=O)OCC(COC(=O)CCC(OCCCCCCCC)OCCCCCCCC)COC(=O)OCCCN(C)C]

42% yield; ¹H NMR (400 MHz, CDCl₃) δ 5.44-5.27 (m, 4H), 4.48 (t, J=5.5 Hz, 1H), 4.23-4.08 (m, 8H), 3.56 (dt, J=9.3, 6.7 Hz, 2H), 3.40 (dt, J=9.3, 6.7 Hz, 2H), 2.77 (t, J=6.5 Hz, 2H), 2.47-2.34 (m, 5H), 2.30 (t, J=7.6 Hz, 2H), 2.24 (s, 6H), 2.05 (q, J=6.9 Hz, 4H), 1.97-1.79 (m, 4H), 1.58 (m, 6H), 1.41-1.24 (m, 34H), 0.88 (m, 9H) ppm. MS: 825 m/z [M+H].

### Example 3

### 3-((4,4-bis(octyloxy)butanoyl)oxy)-2-((((3-(pyrrolidin-1-yl)propoxy)carbonyl)oxy)methyl)propyl (9Z,12Z)-octadeca-9,12-dienoate

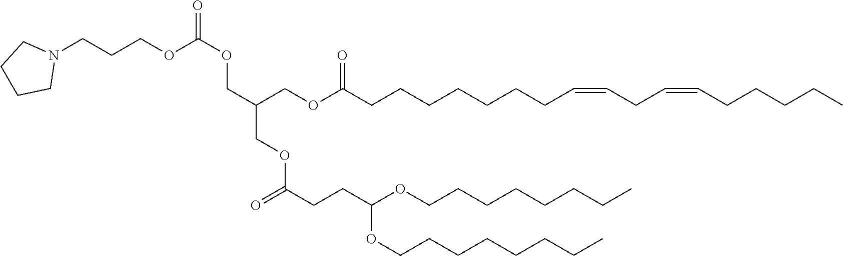 [SMILES: CCCCC/C=C\C/C=C\CCCCCCCC(=O)OCC(COC(=O)CCC(OCCCCCCCC)OCCCCCCCC)COC(=O)OCCCN1CCCC1]

38% yield; ¹H NMR (400 MHz, CDCl₃) δ 5.44-5.27 (m, 4H), 4.48 (t, J=5.5 Hz, 1H), 4.24-4.08 (m, 8H), 3.56 (dt, J=9.3, 6.7 Hz, 2H), 3.40 (dt, J=9.3, 6.7 Hz, 2H), 2.77 (t, J =6.5 Hz, 2H), 2.56 (m, 6H), 2.47-2.35 (m, 3H), 2.30 (t, J=7.6 Hz, 2H), 2.05 (q, J=6.8

Hz, 4H), 1.97-1.85 (m, 4H), 1.86-1.73 (m, 4H), 1.58 (m, 6H), 1.41-1.24 (m, 34H), 0.88 (m, 9H) ppm; MS: 851 m/z [M+H].

### Example 4

### 13-(((4,4-bis(octyloxy)butanoyl)oxy)methyl)-2,5-dimethyl-10-oxo-9,11-dioxa-2,5-diazatetradecan-14-yl (9Z,12Z)-octadeca-9,12-dienoate

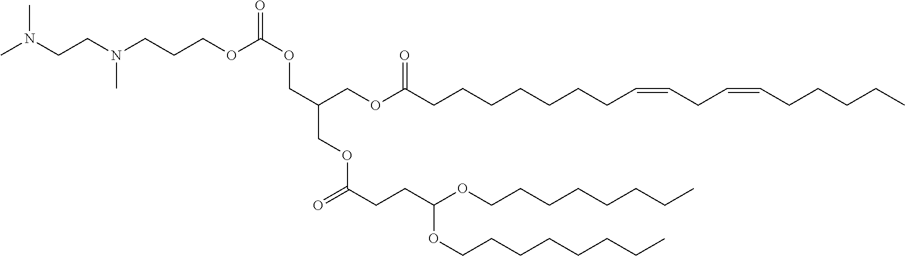 [SMILES: CCCCC/C=C\C/C=C\CCCCCCCC(=O)OCC(COC(=O)CCC(OCCCCCCCC)OCCCCCCCC)COC(=O)OCCCN(C)CCN(C)C]

25% yield; ¹H NMR (400 MHz, CDCl₃) δ 5.44-5.27 (m, 4H), 4.48 (t, J=5.5 Hz, 1H), 4.22-4.11 (m, 8H), 3.56 (dt, J=9.3, 6.7 Hz, 2H), 3.40 (dt, J=9.3, 6.7 Hz, 2H), 2.77 (t, J=6.5 Hz, 2H), 2.58-2.38 (m, 9H), 2.33-2.28 (m, 8H), 2.25 (s, 3H), 2.05 (q, J=6.8 Hz, 4H), 1.97-1.79 (m, 4H), 1.58 (m, 6H), 1.41-1.23 (m, 34H), 0.88 (m, 9H) ppm; MS: 882 m/z [M+H].

### Example 5

### 3-((4,4-bis(octyloxy)butanoyl)oxy)-2-(((((1-ethylpiperidin-3-yl)methoxy)carbonyl)oxy)methyl)propyl (9Z,12Z)-octadeca-9,12-dienoate

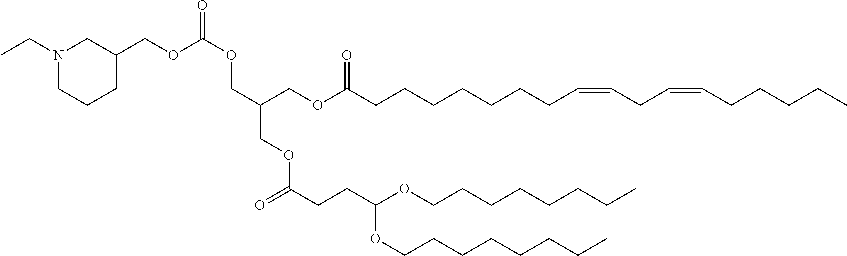 [SMILES: CCCCC/C=C\C/C=C\CCCCCCCC(=O)OCC(COC(=O)CCC(OCCCCCCCC)OCCCCCCCC)COC(=O)OCC1CCCN(CC)C1]

19% yield; ¹H NMR (400 MHz, CDCl₃) δ 5.44-5.27 (m, 4H), 4.48 (t, J=5.6 Hz, 1H), 4.13-3.95 (m, 8H), 3.56 (dt, J=9.3, 6.7 Hz, 2H), 3.40 (dt, J=9.3, 6.7 Hz, 2H), 2.96 (m, 2H), 2.77 (t, J=6.5 Hz, 2H), 2.54-2.35 (m, 5H), 2.30 (t, J=7.6 Hz, 2H), 2.05 (q, J=6.9 Hz, 5H), 2.02-1.54 (m, 15H), 1.42-1.24 (m, 31H), 1.16-1.00 (m, 5H), 0.88 (m, 9H) ppm; MS: 865 m/z [M+H].

### Example 6

### 3-((4,4-bis(octyloxy)butanoyl)oxy)-2-((((3-(dimethylamino)propyl)carbamoyl)oxy)methyl)propyl (9Z,12Z)-octadeca-9,12-dienoate

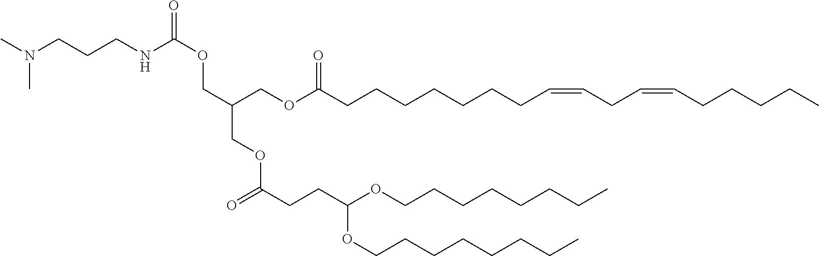 [SMILES: CCCCC/C=C\C/C=C\CCCCCCCC(=O)OCC(COC(=O)CCC(OCCCCCCCC)OCCCCCCCC)COC(=O)NCCCN(C)C]

50% yield; ¹H NMR (400 MHz, CDCl₃) δ 5.70 (br m, 1H), 5.44-5.27 (m, 4H), 4.48 (t, J=5.6 Hz, 1H), 4.13 (br m, 6H), 3.56 (dt, J=9.3, 6.7 Hz, 2H), 3.40 (dt, J=9.3, 6.7 Hz, 2H), 3.26 (q, J=6.2 Hz, 2H), 2.77 (t, J=6.5 Hz, 2H), 2.55 (t, J=6.8 Hz, 2H), 2.40 (m, 7H), 2.30 (t, J=7.6 Hz, 2H), 2.21-2.02 (m, 11H), 1.92 (m, 2H), 1.77 (m, 2H), 1.57 (m, 4H), 1.42-1.25 (m, 31H), 0.88 (m, 9H) ppm; MS: 824 m/z [M+H].

### Example 7

### 3-((4,4-bis(octyloxy)butanoyl)oxy)-2-((((3-(diethylamino)propyl)carbamoyl)oxy)methyl)propyl (9Z,12Z)-octadeca-9,12-dienoate

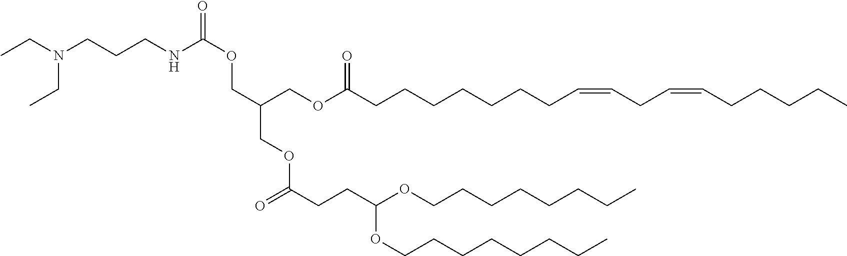 [SMILES: CCCCC/C=C\C/C=C\CCCCCCCC(=O)OCC(COC(=O)CCC(OCCCCCCCC)OCCCCCCCC)COC(=O)NCCCN(CC)CC]

37% yield; ¹H NMR (400 MHz, CDCl₃) δ 6.14 (s, 1H), 5.44-5.28 (m, 4H), 4.48 (t, J=5.6 Hz, 1H), 4.18-4.06 (m, 6H), 3.56 (dt, J=9.3, 6.7 Hz, 2H), 3.40 (dt, J=9.3, 6.7 Hz, 2H), 3.27 (q, J=5.8 Hz, 2H), 2.74 (m, 7H), 2.39 (m, 3H), 2.30 (t, J=7.6 Hz, 2H), 2.13-2.00 (m, 4H), 1.92 (td, J=7.6, 5.5 Hz, 2H), 1.78 (t, J=6.5 Hz, 2H), 1.57 (m, 6H), 1.40-1.22 (m, 35H), 1.14 (t, J=7.2 Hz, 6H), 0.88 (m, 9H) ppm; MS: 852 m/z [M+H].

### Example 8

### 3-((4,4-bis(octyloxy)butanoyl)oxy)-2-((((3-(pyrrolidin-1-yl)propyl)carbamoyl)oxy)methyl)propyl (9Z,12Z)-octadeca-9,12-dienoate

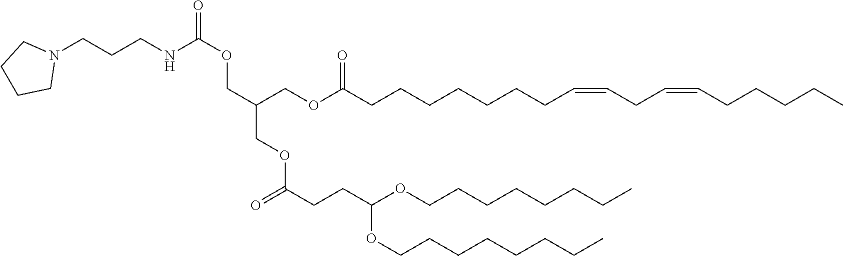 [SMILES: CCCCC/C=C\C/C=C\CCCCCCCC(=O)OCC(COC(=O)CCC(OCCCCCCCC)OCCCCCCCC)COC(=O)NCCCN1CCCC1]

44% yield; ¹H NMR (400 MHz, CDCl₃) δ 5.87 (br m, 1H), 5.43-5.27 (m, 4H), 4.48 (t, J=5.6 Hz, 1H), 4.11 (m, 6H), 3.56 (dt, J=9.2, 6.7 Hz, 2H), 3.40 (dt, J=9.3, 6.7 Hz, 2H), 3.29 (q, J=6.1 Hz, 2H), 2.95-2.68 (m, 8H), 2.44-2.26 (m, 5H), 2.05 (m, 4H), 1.98-1.81 (m, 8H), 1.58 (m, 7H), 1.41-1.18 (m, 33H), 0.88 (m, 9H) ppm; MS: 850 m/z [M+H].

### Example 9

### 3-((4,4-bis(octyloxy)butanoyl)oxy)-2-((((2-(diethylamino)ethoxy)carbonyl)oxy)methyl)propyl (9Z,12Z)-octadeca-9,12-dienoate

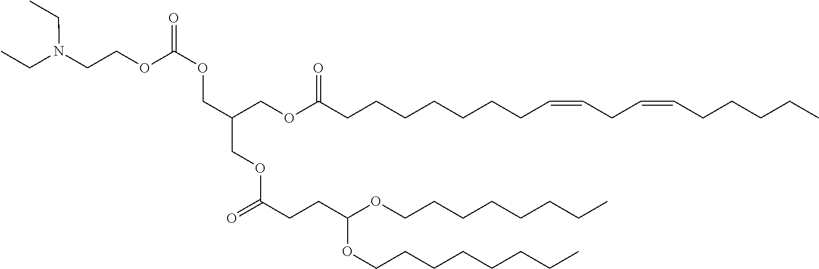 [SMILES: CCCCC/C=C\C/C=C\CCCCCCCC(=O)OCC(COC(=O)CCC(OCCCCCCCC)OCCCCCCCC)COC(=O)OCCN(CC)CC]

84% yield; ¹H NMR (400 MHz, CDCl₃) δ 5.44-5.27 (m, 4H), 4.48 (t, J=5.5 Hz, 1H), 4.28-4.06 (m, 8H), 3.56 (dt, J=9.3, 6.7 Hz, 2H), 3.40 (dt, J=9.3, 6.7 Hz, 2H), 2.79-2.60 (m, 8H), 2.46-2.35 (m, 3H), 2.30 (t, J=7.6 Hz, 2H), 2.05 (q, J=6.8 Hz, 4H), 1.92 (td, J=7.6, 5.4 Hz, 2H), 1.58 (m, 9H), 1.42-1.17 (m, 31H), 1.05 (br m, 6H), 0.88 (m, 9H) ppm; MS: 839 m/z [M+H].

### Example 10

### 3-((4,4-bis(octyloxy)butanoyl)oxy)-2-((((2-(pyrrolidin-1-yl)ethoxy)carbonyl)oxy)methyl)propyl (9Z,12Z)-octadeca-9,12-dienoate

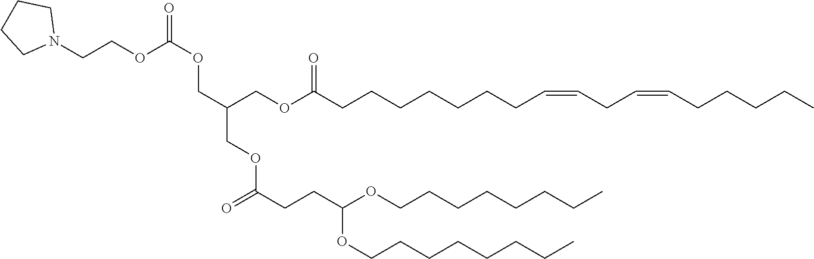 [SMILES: CCCCC/C=C\C/C=C\CCCCCCCC(=O)OCC(COC(=O)CCC(OCCCCCCCC)OCCCCCCCC)COC(=O)OCCN1CCCC1]

74% yield; ¹H NMR (400 MHz, CDCl₃) δ 5.44-5.27 (m, 4H), 4.48 (t, J=5.5 Hz, 1H), 4.27 (t, J=5.9 Hz, 2H), 4.23-4.08 (m, 6H), 3.56 (dt, J=9.3, 6.7 Hz, 2H), 3.40 (dt, J=9.3, 6.7 Hz, 2H), 2.77 (m, 4H), 2.59 (br s, 4H), 2.47-2.35 (m, 3H), 2.30 (t, J=7.6 Hz, 2H), 2.05 (q, J=6.8 Hz, 4H), 1.92 (td, J=7.6, 5.4 Hz, 2H), 1.86-1.74 (br m, 4H), 1.58 (m, 7H), 1.41-1.16 (m, 33H), 0.88 (m, 9H) ppm; MS: 837 m/z [M+H].

### Example 11

### 3-((4,4-bis(octyloxy)butanoyl)oxy)-2-((((4-(diethylamino)butoxy)carbonyl)oxy)methyl)propyl (9Z,12Z)-octadeca-9,12-dienoate

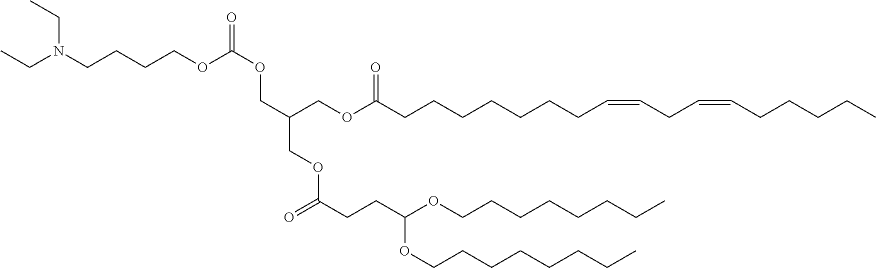 [SMILES: CCCCC/C=C\C/C=C\CCCCCCCC(=O)OCC(COC(=O)CCC(OCCCCCCCC)OCCCCCCCC)COC(=O)OCCCCN(CC)CC]

74% yield; ¹H NMR (400 MHz, CDCl₃) δ 5.44-5.27 (m, 4H), 4.48 (t, J=5.6 Hz, 1H), 4.22-4.08 (m, 8H), 3.56 (dt, J=9.2, 6.7 Hz, 2H), 3.40 (dt, J=9.3, 6.7 Hz, 2H), 2.77 (t, J=6.5 Hz, 2H), 2.65-2.35 (m, 8H), 2.30 (t, J=7.6 Hz, 2H), 2.05 (q, J=6.8 Hz, 4H), 1.92 (td, J=7.6, 5.4 Hz, 2H), 1.74-1.52 (m, 12H), 1.42-1.18 (m, 33H), 1.03 (t, J=7.2 Hz, 6H), 0.88 (m, 9H) ppm; MS: 867 m/z [M+H].

### Example 12

### 3-((4,4-bis(octyloxy)butanoyl)oxy)-2-((((2-(diethylamino)ethyl)carbamoyl)oxy)methyl)propyl (9Z,12Z)-octadeca-9,12-dienoate

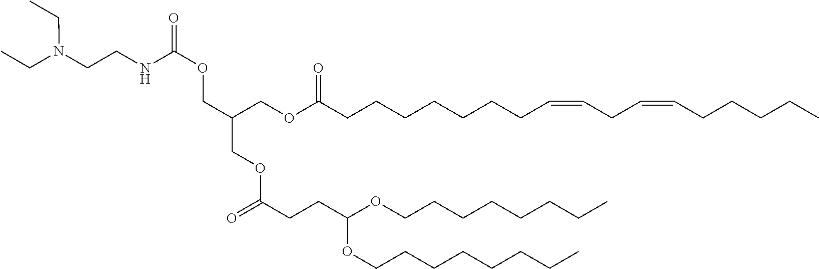 [SMILES: CCCCC/C=C\C/C=C\CCCCCCCC(=O)OCC(COC(=O)CCC(OCCCCCCCC)OCCCCCCCC)COC(=O)NCCN(CC)CC]

72% yield; ¹H NMR (400 MHz, CDCl₃) δ 5.43-5.27 (m, 4H), 4.48 (t, J=5.6 Hz, 1H), 4.12 (m, 6H), 3.56 (dt, J=9.3, 6.7 Hz, 2H), 3.40 (dt, J=9.3, 6.7 Hz, 2H), 3.25 (br m, 2H), 2.77 (t, J=6.5 Hz, 2H), 2.57 (s, 5H), 2.44-2.26 (m, 5H), 2.05 (q, J=6.8 Hz, 4H), 1.92 (td, J=7.6, 5.5 Hz, 2H), 1.58 (m, 9H), 1.41-1.18 (m, 32H), 1.05 (br m, 6H), 0.88 (m, 9H) ppm; MS: 838 m/z [M+H].

### Example 13

### 3-((4,4-bis(octyloxy)butanoyl)oxy)-2-((((4-(pyrrolidin-1-yl)butoxy)carbonyl)oxy)methyl)propyl (9Z,12Z)-octadeca-9,12-dienoate

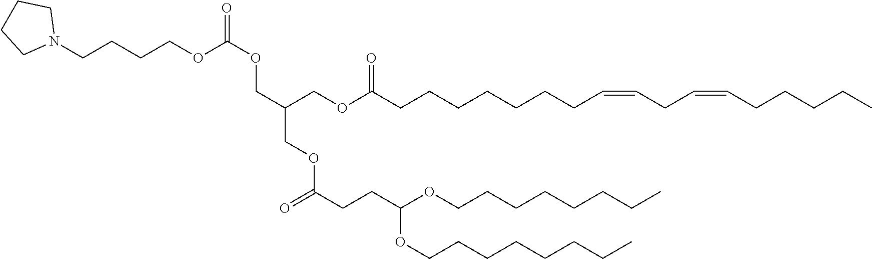 [SMILES: CCCCC/C=C\C/C=C\CCCCCCCC(=O)OCC(COC(=O)CCC(OCCCCCCCC)OCCCCCCCC)COC(=O)OCCCC(C)N1CCCC1]

58% yield; ¹H NMR (400 MHz, CDCl₃) δ 5.44-5.27 (m, 4H), 4.48 (t, J=5.5 Hz, 1H), 4.22-4.11 (m, 8H), 3.56 (dt, J=9.3, 6.7 Hz, 2H), 3.40 (dt, J=9.3, 6.7 Hz, 2H), 2.77 (t, J=6.5 Hz, 2H), 2.60-2.38 (m, 8H), 2.30 (t, J=7.6 Hz, 2H), 2.05 (q, J=6.8 Hz, 4H), 1.92 (td, J=7.6, 5.4 Hz, 2H), 1.86-1.77 (br m, 4H), 1.77-1.52 (m, 13H), 1.41-1.24 (m, 32H), 0.88 (m, 9H) ppm; MS: 865 m/z [M+H].

### Example 14

### 3-((4,4-bis(octyloxy)butanoyl)oxy)-2-(((((1-methylpyrrolidin-2-yl)methoxy)carbonyl)oxy)methyl)propyl (9Z,12Z)-octadeca-9,12-dienoate

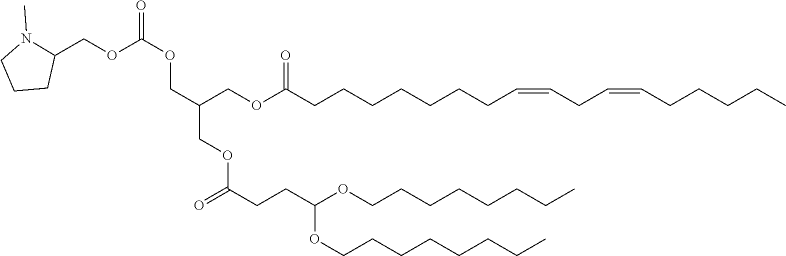 [SMILES: CCCCC/C=C\C/C=C\CCCCCCCC(=O)OCC(COC(=O)CCC(OCCCCCCCC)OCCCCCCCC)COC(=O)OCC1CCCN1C]

60% yield; ¹H NMR (400 MHz, CDCl₃) δ 5.44-5.27 (m, 4H), 4.48 (t, J=5.5 Hz, 1H), 4.21-4.05 (m, 8H), 3.56 (dt, J=9.3, 6.7 Hz, 2H), 3.40 (dt, J=9.3, 6.7 Hz, 2H), 3.11 (br m, 1H), 2.77 (t, J=6.5 Hz, 2H), 2.50-2.35 (m, 6H), 2.30 (t, J=7.6 Hz, 3H), 2.07-1.52 (m, 19H), 1.30 (m, 32H), 0.88 (m, 9H) ppm; MS: 837 m/z [M+H].

### Example 15

### 3-((4,4-bis(octyloxy)butanoyl)oxy)-2-((((3-(dipropylamino)propoxy)carbonyl)oxy)methyl)propyl (9Z,12Z)-octadeca-9,12-dienoate

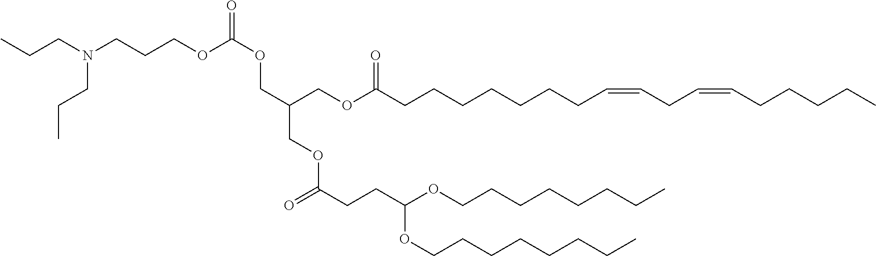 [SMILES: CCCCC/C=C\C/C=C\CCCCCCCC(=O)OCC(COC(=O)CCC(OCCCCCCCC)OCCCCCCCC)COC(=O)OCCCN(CCC)CCC]

79% yield; ¹H NMR (400 MHz, CDCl₃) δ 5.44-5.27 (m, 4H), 4.48 (t, J=5.5 Hz, 1H), 4.23-4.07 (m, 8H), 3.56 (dt, J=9.3, 6.7 Hz, 2H), 3.40 (dt, J=9.3, 6.7 Hz, 2H), 2.77 (t, J=6.5 Hz, 2H), 2.55-2.28 (m, 11H), 2.10-2.00 (m, 4H), 1.92 (td, J=7.6, 5.4 Hz, 2H), 1.81 (br m, 2H), 1.62-1.24 (m, 44H), 0.88 (m, 15H) ppm; MS: 881 m/z [M+H].

### Example 16

### 3-((4,4-bis(octyloxy)butanoyl)oxy)-2-((((2-phenyl-3-(pyrrolidin-1-yl)propoxy)carbonyl)oxy)methyl)propyl (9Z,12Z)-octadeca-9,12-dienoate

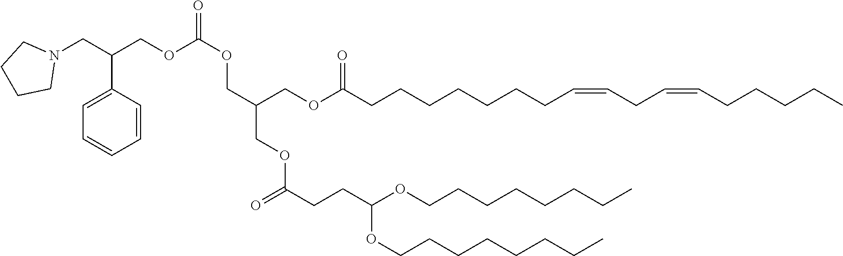 [SMILES: CCCCC/C=C\C/C=C\CCCCCCCC(=O)OCC(COC(=O)CCC(OCCCCCCCC)OCCCCCCCC)COC(=O)OCC(CN1CCCC1)c1ccccc1]

58% yield; ¹H NMR (400 MHz, CDCl₃) δ 7.36-7.17 (m, 5H), 5.44-5.27 (m, 4H), 4.53-4.43 (m, 2H), 4.33 (dd, J=10.7, 7.6 Hz, 1H), 4.11 (m, 6H), 3.55 (dt, J=9.3, 6.7 Hz, 2H), 3.39 (dt, J=9.3, 6.7 Hz, 2H), 3.24 (br m, 1H), 2.92 (br m, 1H), 2.77 (t, J=6.5 Hz, 2H), 2.61-2.33 (m, 7H), 2.28 (t, J=7.6 Hz, 2H), 2.05 (q, J=6.9 Hz, 4H), 1.91 (td, J=7.6, 5.4 Hz, 2H), 1.74 (br m, 4H), 1.66-1.51 (m, 8H), 1.41-1.24 (m, 33H), 0.88 (m, 9H) ppm; MS: 927 m/z [M+H].

### Example 17

### 3-((4,4-bis(octyloxy)butanoyl)oxy)-2-(((((1-ethylpiperidin-4-yl)oxy)carbonyl)oxy)methyl)propyl (9Z,12Z)-octadeca-9,12-dienoate

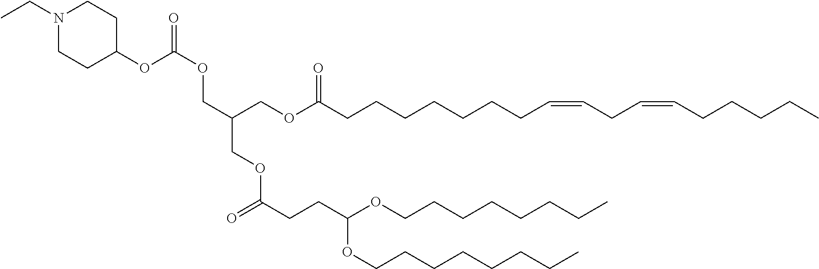 [SMILES: CCCCC/C=C\C/C=C\CCCCCCCC(=O)OCC(COC(=O)CCC(OCCCCCCCC)OCCCCCCCC)COC(=O)OC1CCN(CC)CC1]

40% yield; ¹H NMR (400 MHz, CDCl₃) δ 5.43-5.28 (m, 4H), 4.66 (br s, 1H), 4.48 (t, J=5.5 Hz, 1H), 4.22-4.11 (m, 6H), 3.56 (dt, J=9.3, 6.7 Hz, 2H), 3.40 (dt, J=9.3, 6.7 Hz, 2H), 2.77 (m, 4H), 2.50-2.24 (m, 8H), 2.05 (m, 6H), 1.92 (td, J=7.6, 5.4 Hz, 2H), 1.87-1.74 (m, 2H), 1.58 (m, 10H), 1.41-1.24 (m, 31H), 1.11 (t, J=7.2 Hz, 3H), 0.88 (m, 9H) ppm; MS: 851m/z [M+H].

### Example 18

### 3-((4,4-bis(octyloxy)butanoyl)oxy)-2-((((2-(dimethylamino)ethoxy)carbonyl)oxy)methyl)propyl (9Z,12Z)-octadeca-9,12-dienoate

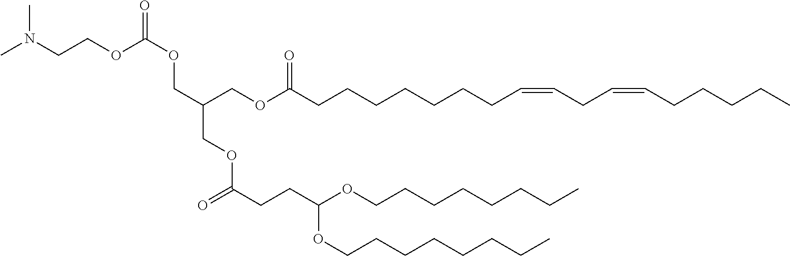 [SMILES: CCCCC/C=C\C/C=C\CCCCCCCC(=O)OCC(COC(=O)CCC(OCCCCCCCC)OCCCCCCCC)COC(=O)OCCN(C)C]

52% yield; ¹H NMR (400 MHz, CDCl₃) δ 5.44-5.27 (m, 4H), 4.48 (t, J=5.6 Hz, 1H), 4.29-4.18 (m, 4H), 4.18-4.08 (m, 4H), 3.56 (dt, J=9.3, 6.7 Hz, 2H), 3.40 (dt, J=9.3, 6.7 Hz, 2H), 2.77 (t, J=6.5 Hz, 2H), 2.64 (br t, J=5.8 Hz, 2H), 2.40 (m, 3H), 2.31 (m, 7H), 2.04 (dd, J=8.8, 5.0 Hz, 4H), 1.92 (td, J=7.6, 5.4 Hz, 2H), 1.56 (m, 10H), 1.30 (m, 31H), 0.88 (m, 9H) ppm; MS: 811 m/z [M+H].

### Example 19

### 3-((4,4-bis(octyloxy)butanoyl)oxy)-2-(((((1-(dimethylamino)propan-2-yl)oxy)carbonyl)oxy)methyl)propyl (9Z,12Z)-octadeca-9,12-dienoate

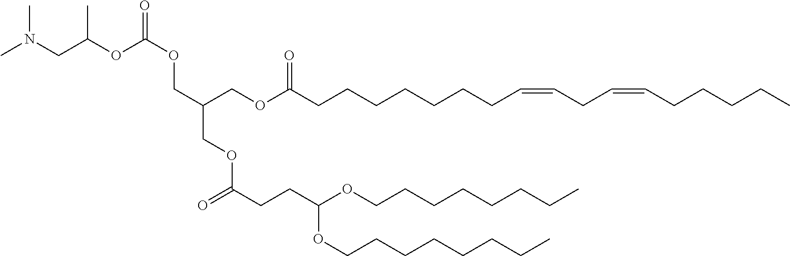 [SMILES: CCCCC/C=C\C/C=C\CCCCCCCC(=O)OCC(COC(=O)CCC(OCCCCCCCC)OCCCCCCCC)COC(=O)OC(C)CN(C)C]

63% yield; ¹H NMR (400 MHz, CDCl₃) δ 5.44-5.26 (m, 4H), 4.88 (m, 1H), 4.48 (t, J=5.6 Hz, 1H), 4.16 (m, 7H), 3.56 (dt, J=9.2, 6.7 Hz, 2H), 3.40 (dt, J=9.3, 6.7 Hz, 2H), 2.77 (t, J=6.5 Hz, 2H), 2.57 (dd, J=13.0, 7.7 Hz, 1H), 2.49-2.35 (m, 3H), 2.29 (m, 8H), 2.09-1.99 (m, 4H), 1.92 (td, J=7.6, 5.5 Hz, 2H), 1.69-1.53 (m, 10H), 1.30 (m, 33H), 0.88 (m, 9H) ppm; MS: 825 m/z [M+H].

### Example 20

### 3-((4,4-bis(octyloxy)butanoyl)oxy)-2-((((3-(ethyl(methyl)amino)propoxy)carbonyl)oxy)methyl)propyl (9Z,12Z)-octadeca-9,12-dienoate

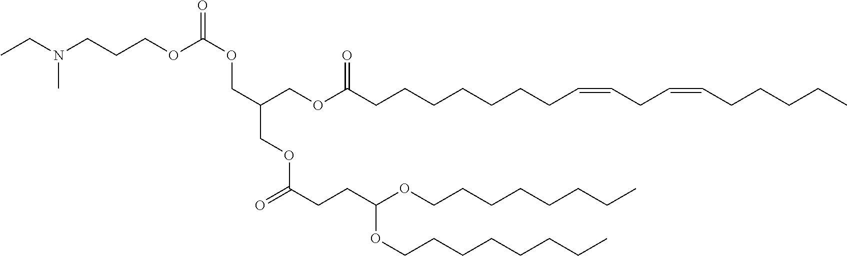 [SMILES: CCCCC/C=C\C/C=C\CCCCCCCC(=O)OCC(COC(=O)CCC(OCCCCCCCC)OCCCCCCCC)COC(=O)OCCCN(C)CC]

30% yield; ¹H NMR (400 MHz, CDCl₃) δ 5.44-5.27 (m, 4H), 4.48 (t, J=5.5 Hz, 1H), 4.24-4.09 (m, 8H), 3.56 (dt, J=9.3, 6.7 Hz, 2H), 3.40 (dt, J=9.3, 6.7 Hz, 2H), 2.77 (t, J=6.5 Hz, 2H), 2.57-2.38 (m, 6H), 2.32-2.20 (m, 5H), 2.05 (q, J=6.8 Hz, 4H), 1.95-1.82 (m, 4H), 1.58 (m, 7H), 1.42-1.25 (m, 34H), 1.09 (br m, 3H), 0.88 (m, 9H) ppm; MS: 839 m/z [M+H].

### Example 21

### 3-((4,4-bis(octyloxy)butanoyl)oxy)-2-((((3-(diisopropylamino)propoxy)carbonyl)oxy)methyl)propyl (9Z,12Z)-octadeca-9,12-dienoate

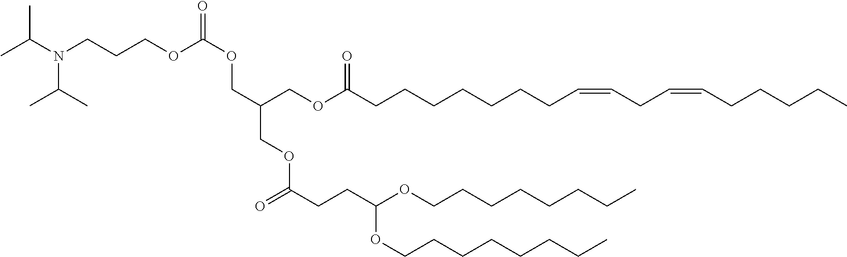 [SMILES: CCCCC/C=C\C/C=C\CCCCCCCC(=O)OCC(COC(=O)CCC(OCCCCCCCC)OCCCCCCCC)COC(=O)OCCCN(C(C)C)C(C)C]

31% yield; ¹H NMR (400 MHz, CDCl₃) δ 5.44-5.27 (m, 4H), 4.48 (t, J=5.5 Hz, 1H), 4.22-4.08 (m, 8H), 3.56 (dt, J=9.3, 6.7 Hz, 2H), 3.40 (dt, J=9.3, 6.7 Hz, 2H), 2.98 (m, J=6.7 Hz, 2H), 2.77 (t, J=6.4 Hz, 2H), 2.50 (t, J=6.9 Hz, 2H), 2.47-2.35 (m, 3H), 2.30 (t, J=7.6 Hz, 2H), 2.05 (q, J=6.8 Hz, 4H), 1.92 (td, J=7.6, 5.4 Hz, 2H), 1.74 (m, 2H), 1.63-1.51 (m, 10H), 1.42-1.24 (m, 36H), 0.98 (d, J=6.6 Hz, 9H), 0.88 (m, 9H) ppm; MS: 881 m/z [M+H].

### Example 22

### 3-((4,4-bis(octyloxy)butanoyl)oxy)-2-((((3-(dimethylamino)butoxy)carbonyl)oxy)methyl)propyl (9Z,12Z)-octadeca-9,12-dienoate

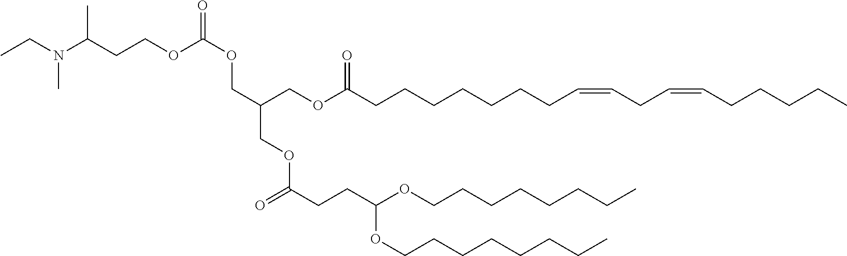 [SMILES: CCCCC/C=C\C/C=C\CCCCCCCC(=O)OCC(COC(=O)CCC(OCCCCCCCC)OCCCCCCCC)COC(=O)OCCC(C)N(C)CC]

35% yield; ¹H NMR (400 MHz, CDCl₃) δ 5.44-5.27 (m, 4H), 4.48 (t, J=5.5 Hz, 1H), 4.29-4.21 (m, 1H), 4.27-4.10 (m, 8H), 3.56 (dt, J=9.3, 6.7 Hz, 2H), 3.40 (dt, J=9.3, 6.7 Hz, 2H), 2.77 (t, J=6.4 Hz, 3H), 2.46-2.35 (m, 3H), 2.29 (m, 7H), 2.05 (q, J=6.8 Hz, 4H), 1.92 (td, J=7.6, 5.4 Hz, 3H), 1.60 (m, 8H), 1.42-1.25 (m, 33H), 1.02 (br s, 3H), 0.88 (m, 9H) ppm; MS: 839 m/z [M+H].

### Example 23

### 3-(((3-(azepan-1-yl)propoxy)carbonyl)oxy)-2-(((4,4-bis(octyloxy)butanoyl)oxy)methyl)propyl (9Z,12Z)-octadeca-9,12-dienoate

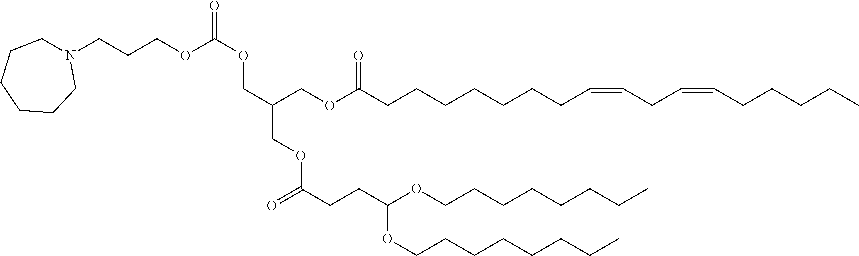 [SMILES: CCCCC/C=C\C/C=C\CCCCCCCC(=O)OCC(COC(=O)CCC(OCCCCCCCC)OCCCCCCCC)COC(=O)OCCCN1CCCCCC1]

59% yield; ¹H NMR (400 MHz, CDCl₃) δ 5.35 (m, 4H), 4.48 (t, J=5.5 Hz, 1H), 4.24-4.09 (m, 8H), 3.56 (dt, J=9.4, 6.7 Hz, 2H), 3.40 (dt, J=9.3, 6.7 Hz, 2H), 2.77 (t, J=6.5 Hz, 2H), 2.64 (br m, 6H), 2.47-2.35 (m, 3H), 2.30 (t, J=7.6 Hz, 2H), 2.04 (dd, J=7.9, 5.9 Hz, 4H), 1.95-1.81 (m, 4H), 1.70-1.52 (m, 14H), 1.41-1.23 (m, 34H), 0.88 (m, 9H) ppm; MS: 879 m/z [M+H].

### Example 24

### 3-((4,4-bis(octyloxy)butanoyl)oxy)-2-((((2-(1-methylpyrrolidin-2-yl)ethoxy)carbonyl)oxy)methyl)propyl (9Z,12Z)-octadeca-9,12-dienoate

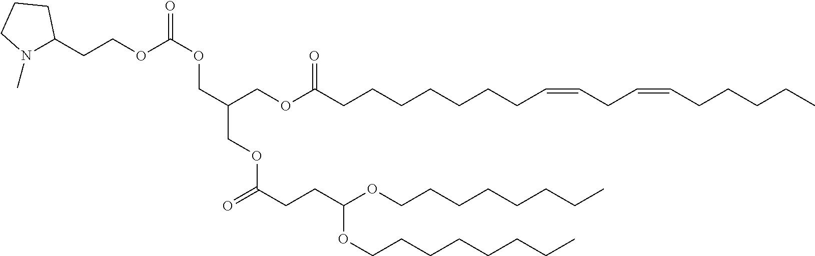 [SMILES: CCCCC/C=C\C/C=C\CCCCCCCC(=O)OCC(COC(=O)CCC(OCCCCCCCC)OCCCCCCCC)COC(=O)OCCC1CCCN1C]

56% yield; ¹H NMR (400 MHz, CDCl₃) δ 5.35 (m, 4H), 4.48 (t, J=5.5 Hz, 1H), 4.29-4.07 (m, 8H), 3.56 (dt, J=9.3, 6.7 Hz, 2H), 3.40 (dt, J=9.3, 6.7 Hz, 2H), 3.13 (br s, 1H), 2.77 (t, J=6.5 Hz, 2H), 2.45-1.50 (m, 28H) 1.29 (m, 34H), 0.88 (m, 9H) ppm; MS: 851 m/z [M+H].

### Synthesis of Example 25

### Example 25

### 3-((4,4-bis(octyloxy)butanoyl)oxy)-2-((((2-(1-methylpyrrolidin-2-yl)ethoxy)carbonyl)oxy)methyl)propyl (9Z,12Z)-octadeca-9,12-dienoate

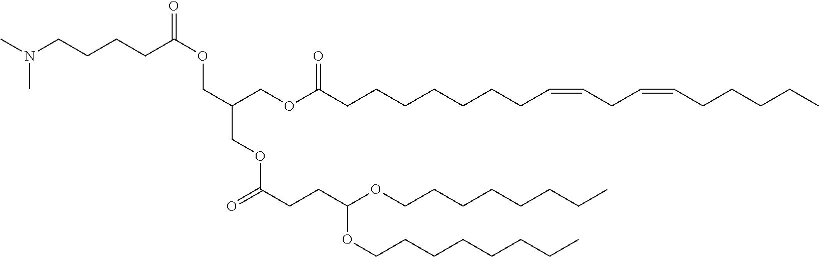 [SMILES: CCCCC/C=C\C/C=C\CCCCCCCC(=O)OCC(COC(=O)CCCCN(C)C)COC(=O)CCC(OCCCCCCCC)OCCCCCCCC]

To a solution of Intermediate 1d (300 mg, 0.43 mmol, 1 equiv), 5-(dimethylamino)pentanoic acid (1-3 equiv), DIPEA (1.4-3 equiv), and DMAP (0.1-0.2 equiv) in DCM (0.10-0.15 M) was added EDCHCl (1.4-1.5 equiv) at rt. Upon stirring for at least 2 h at rt, the reaction mixture was diluted with water and the organic layer was separated and concentrated in vacuo. The crude residue was purified using silica gel chromatography (gradient of EtOAc in hexanes or MeOH in DCM) to provide 159 mg (0.19 mmol, 44% yield) of the desired product. ¹H NMR (400 MHz, CDCl₃) δ 5.35 (m, 4H), 4.48 (t, J=5.5 Hz, 1H), 4.12 (m, 6H), 3.56 (dt, J=9.3, 6.7 Hz, 2H), 3.40 (dt, J=9.3, 6.7 Hz, 2H), 2.77 (t, J=6.5 Hz, 2H), 2.70 (br m, 2H), 2.56 (br s, 6H), 2.39 (m, 5H), 2.30 (t, J=7.6 Hz, 2H), 2.04 (m, 4H), 1.92 (td, J=7.6, 5.4 Hz, 2H), 1.81-1.52 (m, 10H), 1.41-1.24 (m, 34H), 0.88 (m, 9H) ppm; MS: 823.42 m/z [M+H].

### Synthesis of Examples 26-32

The following examples were synthesized from Intermediate 1d and a carboxylic acid reagent using the method employed for Example 25.

### Example 26

### 3-((4,4-bis(octyloxy)butanoyl)oxy)-2-(((4-(dimethylamino)butanoyl)oxy)methyl)propyl (9Z,12Z)-octadeca-9,12-dienoate

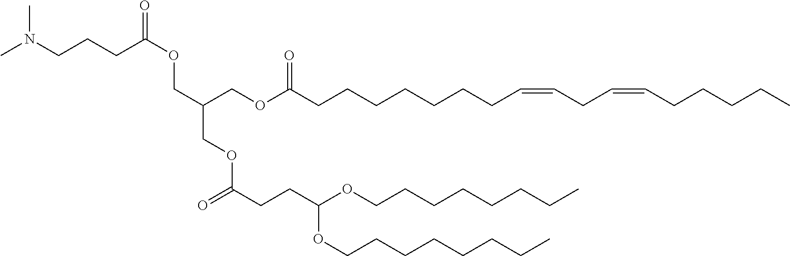 [SMILES: CCCCC/C=C\C/C=C\CCCCCCCC(=O)OCC(COC(=O)CCCN(C)C)COC(=O)CCC(OCCCCCCCC)OCCCCCCCC]

39% yield; ¹H NMR (400 MHz, CDCl₃) δ 5.44-5.27 (m, 4H), 4.48 (t, J=5.5 Hz, 1H), 4.13 (m, J=6.0, 2.2 Hz, 6H), 3.56 (dt, J=9.3, 6.7 Hz, 2H), 3.40 (dt, J=9.3, 6.7 Hz, 2H), 2.77 (t, J=6.5 Hz, 2H), 2.44-2.28 (m, 9H), 2.21 (s, 6H), 2.10-2.00 (m, 4H), 1.92 (td, J=7.6, 5.5 Hz, 2H), 1.78 (m, J=7.4 Hz, 2H), 1.58 (m, 6H), 1.42-1.22 (m, 34H), 0.88 (m, 9H) ppm; MS: 809.47 m/z [M+H].

### Example 27

### 3-((4,4-bis(octyloxy)butanoyl)oxy)-2-(((3-(diethylamino)propanoyl)oxy)methyl)propyl (9Z,12Z)-octadeca-9,12-dienoate

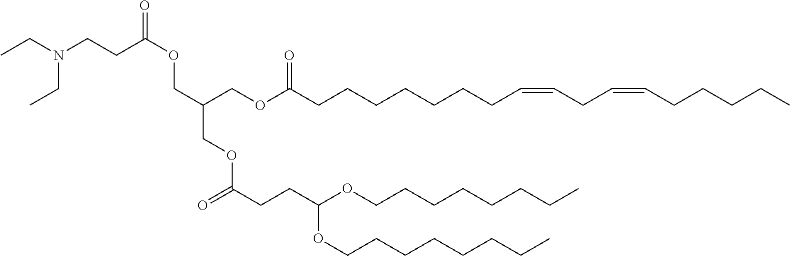 [SMILES: CCCCC/C=C\C/C=C\CCCCCCCC(=O)OCC(COC(=O)CCC(OCCCCCCCC)OCCCCCCCC)COC(=O)CCN(CC)CC]

78% yield; ¹H NMR (400 MHz, CDCl₃) δ 5.44-5.27 (m, 4H), 4.48 (t, J=5.5 Hz, 1H), 4.13 (m, 6H), 3.56 (dt, J=9.3, 6.7 Hz, 2H), 3.40 (dt, J=9.3, 6.7 Hz, 2H), 2.77 (m, 4H), 2.56-2.34 (m, 9H), 2.30 (t, J=7.6 Hz, 2H), 2.10-2.00 (m, 4H), 1.92 (td, J=7.6, 5.5 Hz, 2H), 1.58 (m, 6H), 1.42-1.22 (m, 34H), 1.01 (t, J=7.1 Hz, 6H), 0.88 (m, 9H) ppm; MS: 823.42 m/z [M+H].

### Example 28

### 3-((4,4-bis(octyloxy)butanoyl)oxy)-2-(((5-(diethylamino)pentanoyl)oxy)methyl)propyl (9Z,12Z)-octadeca-9,12-dienoate

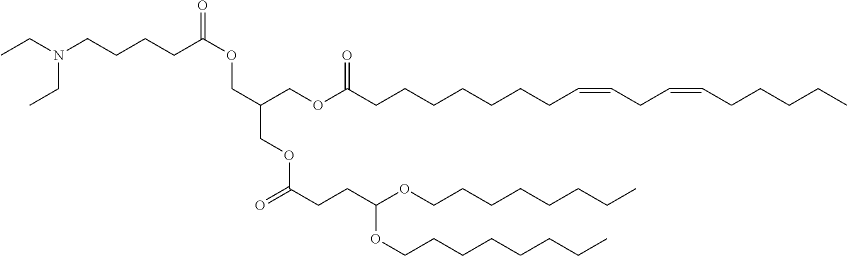 [SMILES: CCCCC/C=C\C/C=C\CCCCCCCC(=O)OCC(COC(=O)CCCCN(CC)CC)COC(=O)CCC(OCCCCCCCC)OCCCCCCCC]

77% yield; ¹H NMR (400 MHz, CDCl₃) δ 5.43-5.28 (m, 4H), 4.48 (t, J=5.6 Hz, 1H), 4.13 (m, 6H), 3.56 (dt, J=9.3, 6.7 Hz, 2H), 3.40 (dt, J=9.3, 6.7 Hz, 2H), 2.77 (t, J=6.7 Hz, 2H), 2.50 (q, J=7.2 Hz, 4H), 2.45-2.27 (m, 9H), 2.04 (dd, J=7.8, 6.2 Hz, 4H), 1.92 (td, J=7.6, 5.5 Hz, 2H), 1.64-1.44 (m, 11H), 1.31 (m, 33H), 1.01 (t, J=7.1 Hz, 6H), 0.88 (m, 9H) ppm. MS: 851.86 m/z [M+H].

### Example 29

### 3-((4,4-bis(octyloxy)butanoyl)oxy)-2-(((4-(diethylamino)butanoyl)oxy)methyl)propyl (9Z,12Z)-octadeca-9,12-dienoate

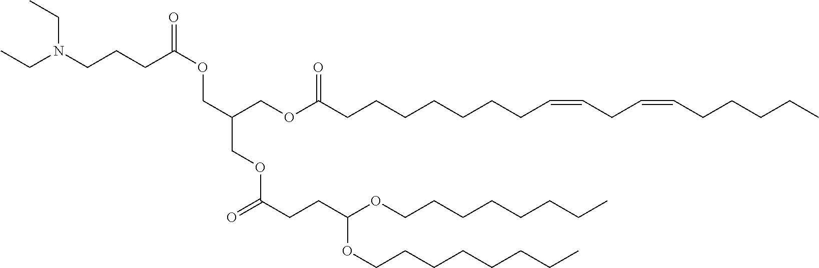 [SMILES: CCCCC/C=C\C/C=C\CCCCCCCC(=O)OCC(COC(=O)CCCN(CC)CC)COC(=O)CCC(OCCCCCCCC)OCCCCCCCC]

53% yield; ¹H NMR (500 MHz, CDCl₃) δ 5.43-5.28 (m, 4H), 4.48 (t, J=5.5 Hz, 1H), 4.13 (m, 6H), 3.56 (dt, J=9.3, 6.7 Hz, 2H), 3.40 (dt, J=9.3, 6.7 Hz, 2H), 2.77 (t, J=6.7 Hz, 2H), 2.50 (m, 5H), 2.45-2.29 (m, 10H), 2.05 (q, J=6.9 Hz, 4H), 1.92 (td, J=7.6, 5.5 Hz, 2H), 1.75 (m, 2H), 1.66-1.52 (m, 7H), 1.40-1.23 (m, 30H), 1.00 (m, 6H), 0.88 (m, 9H) ppm; MS: 837.01 m/z [M+H].

### Example 30

### 3-((4,4-bis(octyloxy)butanoyl)oxy)-2-((((9Z,12Z)-octadeca-9,12-dienoyl)oxy)methyl)propyl 1,3-dimethylpyrrolidine-3-carboxylate

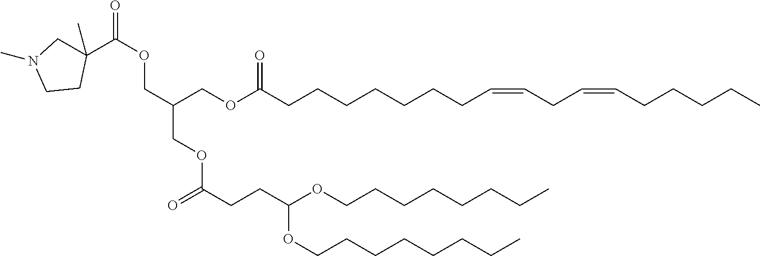 [SMILES: CCCCC/C=C\C/C=C\CCCCCCCC(=O)OCC(COC(=O)CCC(OCCCCCCCC)OCCCCCCCC)COC(=O)C1(C)CCN(C)C1]

45% yield; ¹H NMR (500 MHz, CDCl₃) δ 5.43-5.28 (m, 4H), 4.48 (t, J=5.5 Hz, 1H) 4.18-4.11 (m, 6H), 3.56 (dt, J=9.3, 6.7 Hz, 2H), 3.40 (dt, J=9.3, 6.7 Hz, 2H), 2.93 (d, J=9.4 Hz, 1H), 2.77 (t, J=6.7 Hz, 2H), 2.64-2.52 (m, 2H), 2.47-2.27 (m, 11H), 2.05 (q, J=7.0 Hz, 4H), 1.92 (td, J=7.6, 5.5 Hz, 2H), 1.71-1.52 (m, 9H), 1.40-1.24 (m, 34H), 0.88 (m, 9H) ppm; MS: 821.30 m/z [M+H].

### Example 31

### 3-((4,4-bis(octyloxy)butanoyl)oxy)-2-((((9Z,12Z)-octadeca-9,12-dienoyl)oxy)methyl)propyl 1-methylpiperidine-4-carboxylate

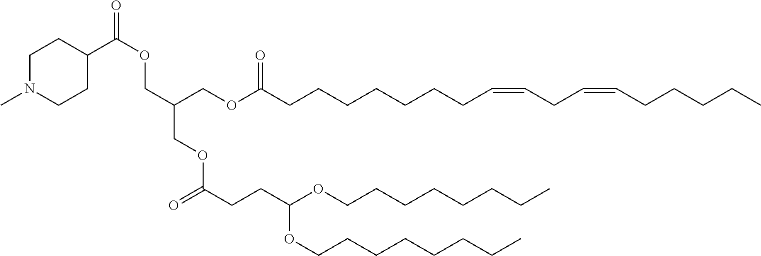 [SMILES: CCCCC/C=C\C/C=C\CCCCCCCC(=O)OCC(COC(=O)CCC(OCCCCCCCC)OCCCCCCCC)COC(=O)C1CCN(C)CC1]

87% yield; ¹H NMR (500 MHz, CDCl₃) δ 5.43-5.28 (m, 4H), 4.48 (t, J=5.5 Hz, 1H), 4.16-4.09 (m, 6H), 3.56 (dt, J=9.3, 6.7 Hz, 2H), 3.40 (dt, J=9.3, 6.7 Hz, 2H), 2.78 (m, 4H), 2.43-2.34 (m, 3H), 2.34-2.22 (m, 6H), 2.05 (q, J=6.9 Hz, 4H), 2.00-1.87 (m, 6H), 1.76 (m, 2H), 1.65-1.52 (m, 7H), 1.42-1.19 (m, 33H), 0.88 (m, 9H) ppm; MS: 821.80 m/z [M+H].

### Example 32

### 3-((4,4-bis(octyloxy)butanoyl)oxy)-2-((((9Z,12Z)-octadeca-9,12-dienoyl)oxy)methyl)propyl 1,4-dimethylpiperidine-4-carboxylate

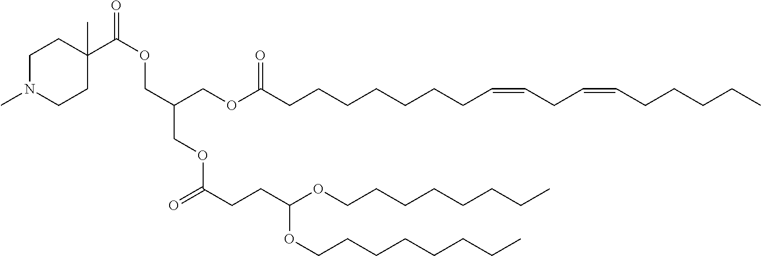 [SMILES: CCCCC/C=C\C/C=C\CCCCCCCC(=O)OCC(COC(=O)CCC(OCCCCCCCC)OCCCCCCCC)COC(=O)C1(C)CCN(C)CC1]

39% yield; ¹H NMR (500 MHz, CDCl₃) δ 5.43-5.28 (m, 4H), 4.48 (t, J=5.5 Hz, 1H), 4.17-4.09 (m, 6H), 3.56 (dt, J=9.3, 6.7 Hz, 2H), 3.40 (dt, J=9.3, 6.7 Hz, 2H), 2.77 (t, J=6.7 Hz, 2H), 2.58 (m, 2H), 2.41 (m, 3H), 2.31 (t, J=7.6 Hz, 2H), 2.24 (s, 3H), 2.15-2.01 (m, 8H), 1.92 (td, J=7.6, 5.3 Hz, 2H), 1.63-1.48 (m, 9H), 1.42-1.24 (m, 33H), 1.19 (s, 3H), 0.88 (m, 9H) ppm; MS: 835.79 m/z [M+H].

### Synthesis of Examples 33-36

The following examples were synthesized from Intermediate 1d and an amino alcohol or diamine reagent using the method employed for Example 1.

### Example 33

### 3-((4,4-bis(octyloxy)butanoyl)oxy)-2-((((2-(diethylamino)ethyl)(methyl)carbamoyl)oxy)methyl)propyl (9Z,12Z)-octadeca-9,12-dienoate

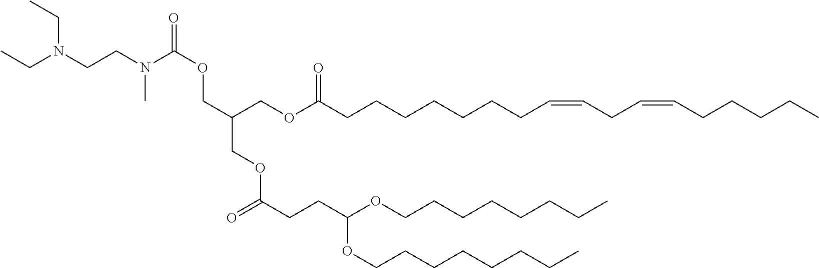 [SMILES: CCCCC/C=C\C/C=C\CCCCCCCC(=O)OCC(COC(=O)CCC(OCCCCCCCC)OCCCCCCCC)COC(=O)N(C)CCN(CC)CC]

62% yield; ¹H NMR (500 MHz, CDCl₃) δ 5.43-5.28 (m, 4H), 4.48 (t, J=5.6 Hz, 1H), 4.13 (m, 6H), 3.56 (dt, J=9.3, 6.7 Hz, 2H), 3.40 (dt, J=9.3, 6.7 Hz, 2H), 3.30 (m, 2H), 2.94 (s, 1.5H), 2.91 (s, 1.5H), 2.77 (t, J=6.7 Hz, 2H), 2.54 (m, 6H), 2.39 (m, 3H), 2.30 (t, J=7.6 Hz, 2H), 2.04 (m, 4H), 1.92 (td, J=7.6, 5.5 Hz, 2H), 1.64-1.54 (m, 7H), 1.39-1.22 (m, 33H), 1.01 (m, 6H), 0.88 (m, 9H) ppm; MS: 852.80 m/z [M+H].

### Example 34

### 3-((4,4-bis(octyloxy)butanoyl)oxy)-2-((((2-(dimethylamino)ethyl)carbamoyl)oxy)methyl)propyl (9Z,12Z)-octadeca-9,12-dienoate

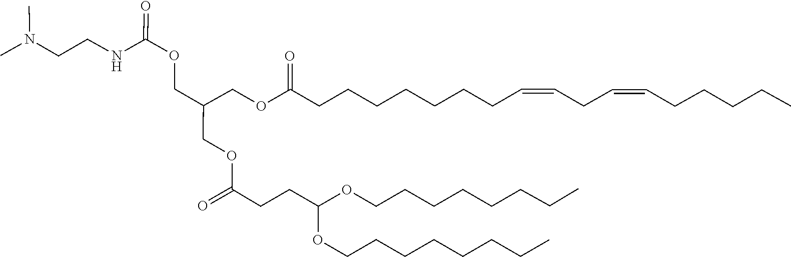 [SMILES: CCCCC/C=C\C/C=C\CCCCCCCC(=O)OCC(COC(=O)CCC(OCCCCCCCC)OCCCCCCCC)COC(=O)NCCN(C)C]

44% yield; ¹H NMR (500 MHz, CDCl₃) δ 5.43-5.28 (m, 4H), 5.20 (br t, J=5.1 Hz, 1H), 4.48 (t, J=5.5 Hz, 1H), 4.13 (m, 6H), 3.56 (dt, J=9.3, 6.7 Hz, 2H), 3.40 (dt, J=9.3, 6.7 Hz, 2H), 3.24 (m, 2H), 2.77 (t, J=6.7 Hz, 2H), 2.43-2.34 (m, 5H), 2.30 (t, J=7.6 Hz, 2H), 2.22 (s, 6H), 2.05 (q, J=6.9 Hz, 5H), 1.92 (td, J=7.6, 5.4 Hz, 2H), 1.63-1.52 (m, 7H), 1.40-1.24 (m, 32H), 0.88 (m, 9H) ppm; MS: 810.73 m/z [M+H].

### Example 35

### 3-((4,4-bis(octyloxy)butanoyl)oxy)-2-((((2-(ethyl(methyl)amino)ethyl)carbamoyl)oxy)methyl)propyl (9Z,12Z)-octadeca-9,12-dienoate

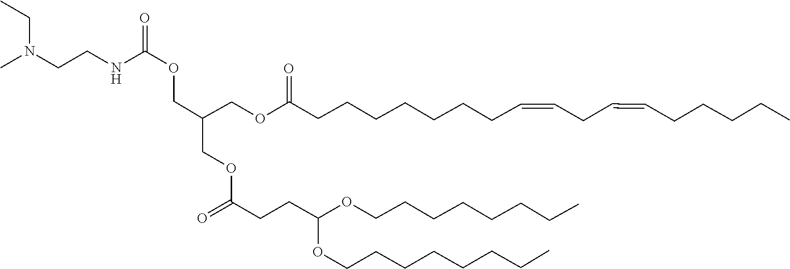 [SMILES: CCCCC/C=C\C/C=C\CCCCCCCC(=O)OCC(COC(=O)CCC(OCCCCCCCC)OCCCCCCCC)COC(=O)NCCN(C)CC]

53% yield; ¹H NMR (500 MHz, CDCl₃) δ 5.43-5.28 (m, 4H), 5.20 (t, J=5.1 Hz, 1H), 4.48 (t, J=5.6 Hz, 1H), 4.12 (m, 6H), 3.56 (dt, J=9.3, 6.7 Hz, 2H), 3.40 (dt, J=9.3, 6.7 Hz, 2H), 3.24 (m, 2H), 2.77 (t, J=6.7 Hz, 2H), 2.48-2.33 (m, 7H), 2.30 (t, J=7.6 Hz, 2H), 2.20 (s, 3H), 2.05 (q, J=6.9 Hz, 4H), 1.92 (td, J=7.7, 5.5 Hz, 2H), 1.62-1.52 (m, 7H), 1.35-1.23 (m, 33H), 1.04 (t, J=7.2 Hz, 3H), 0.88 (m, 9H) ppm; MS: 824.77 m/z [M+H].

### Example 36

### 3-((4,4-bis(octyloxy)butanoyl)oxy)-2-((((1-ethylpiperidin-3-yl)carbamoyl)oxy)methyl)propyl (9Z,12Z)-octadeca-9,12-dienoate

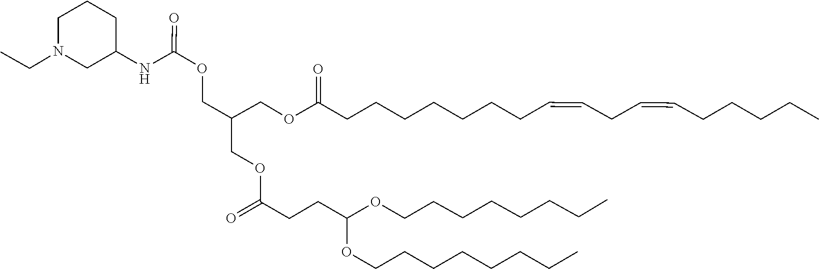 [SMILES: CCCCC/C=C\C/C=C\CCCCCCCC(=O)OCC(COC(=O)CCC(OCCCCCCCC)OCCCCCCCC)COC(=O)NC1CCCN(CC)C1]

46% yield; ¹H NMR (400 MHz, CDCl₃) δ 5.43-5.27 (m, 4H), 5.20 (br s, 1H), 4.48 (t, J=5.5 Hz, 1H), 4.12 (m, 6H), 3.78 (br s, 1H), 3.56 (dt, J=9.3, 6.7 Hz, 2H), 3.40 (dt, J=9.3, 6.7 Hz, 2H), 2.77 (t, J=6.4 Hz, 2H), 2.50-2.19 (m, 8H), 2.05 (q, J=6.8 Hz, 4H), 1.92 (td, J=7.6, 5.5 Hz, 2H), 1.73-1.52 (m, 14H), 1.41-1.23 (m, 33H), 1.04 (t, J=7.2 Hz, 3H), 0.88 (m, 9H) ppm; MS: 850.48 m/z [M+H].

### Synthesis of Example 37

### Example 37

### 3-((4,4-bis(octyloxy)butanoyl)oxy)-2-((((1-ethylpiperidin-4-yl)carbamoyl)oxy)methyl)propyl (9Z,12Z)-octadeca-9,12-dienoate

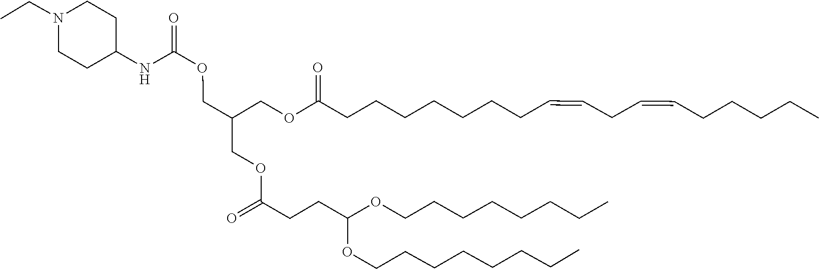 [SMILES: CCCCC/C=C\C/C=C\CCCCCCCC(=O)OCC(COC(=O)CCC(OCCCCCCCC)OCCCCCCCC)COC(=O)NC1CCN(CC)CC1]

To a solution of intermediate 1d (400 mg, 0.57 mmol) in acetonitrile (6 mL) was added pyridine (93 μL, 1.15 mmol), DMAP (14 mg, 0.08 mmol), and 4-nitrophenyl chloroformate (173 mg, 0.86 mmol) sequentially at rt. Upon stirring for 2 h, 1-ethylpiperidin-4-amine dihydrochloride (344 mg, 1.72 mmol) and DIPEA (800 μL, 4.60 mmol) was added and the resulting reaction mixture was stirred for an additional 4 h at rt. The reaction mixture was extracted into hexanes and washed with water. If needed, the resulting water layer was re-extracted with hexanes. The combined hexanes layers were dried over anhydrous MgSO_{4,} filtered, and concentrated in vacuo. The crude residue was purified using silica gel chromatography (gradient of EtOAc in hexanes or methanol in DCM) to provide 157 mg (0.18 mmol, 32% yield) of the desired product as a clear oil. ¹H NMR (400 MHz, CDCl₃) δ 5.43-5.27 (m, 4H), 4.59 (br d, J=8.1 Hz, 1H), 4.48 (t, J=5.6 Hz, 1H), 4.12 (m, 6H), 3.59-3.49 (m, 3H), 3.40 (dt, J=9.3, 6.7 Hz, 2H), 2.85 (br d, J=11.1 Hz, 2H), 2.77 (t, J=6.7 Hz, 2H), 2.45-2.35 (m, 5H), 2.30 (t, J=7.6 Hz, 2H), 2.05 (q, J=7.2 Hz, 6H), 1.99-1.87 (m, 4H), 1.65-1.21 (m, 43H), 1.07 (t, J=7.2 Hz, 3H), 0.88 (m, 9H) ppm; MS: 850.34 m/z [M+H].

### Synthesis of Examples 38-49

The following examples were synthesized from Intermediate 1d and a diamine reagent using the method employed for Example 1.

### Example 38

### 3-((4,4-bi s(octyloxy)butanoyl)oxy)-2-(((((1-ethylpiperidin-2-yl)methyl)carbamoyl)oxy)methyl)propyl (9Z,12Z)-octadeca-9,12-dienoate

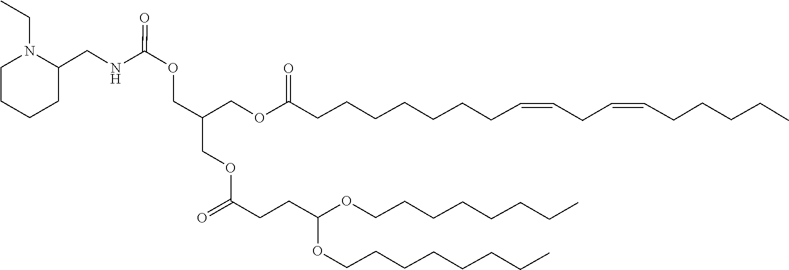 [SMILES: CCCCC/C=C\C/C=C\CCCCCCCC(=O)OCC(COC(=O)CCC(OCCCCCCCC)OCCCCCCCC)COC(=O)NCC1CCCCN1CC]

30% yield; ¹H NMR (400 MHz, CDCl₃) δ 5.41-5.27 (m, 4H), 5.14 (br s, 1H), 4.48 (t, J=5.6 Hz, 1H), 4.16-4.09 (m, 6H), 3.56 (dt, J=9.3, 6.7 Hz, 2H), 3.45-3.23 (m, 4H), 2.89 (m, 1H), 2.77 (m, 3H), 2.49-2.26 (m, 7H), 2.19 (m, 1H), 2.05 (q, J=6.8 Hz, 4H), 1.92 (td, J=7.5, 5.5 Hz, 2H), 1.73-1.25 (m, 46H), 1.02 (t, J=7.0 Hz, 3H), 0.93-0.84 (m, 9H) ppm; MS: 864.39 m/z [M+H].

### Example 39

### 3-((4,4-bis(octyloxy)butanoyl)oxy)-2-(((((1-methylpyrrolidin-2-yl)methyl)carbamoyl)oxy)methyl)propyl (9Z,12Z)-octadeca-9,12-dienoate

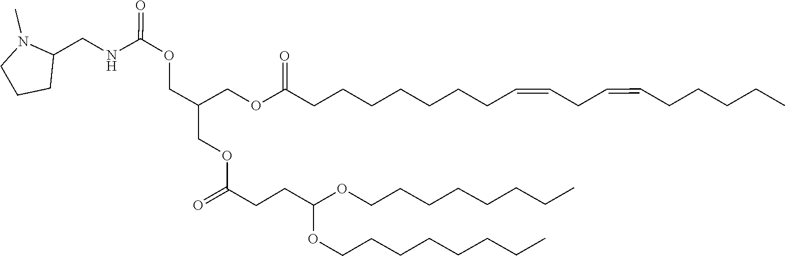 [SMILES: CCCCC/C=C\C/C=C\CCCCCCCC(=O)OCC(COC(=O)CCC(OCCCCCCCC)OCCCCCCCC)COC(=O)NCC1CCCN1C]

18% yield; ¹H NMR (400 MHz, CDCl₃) δ 5.43-5.27 (m, 4H), 5.13 (s, 1H), 4.48 (t, J=5.6 Hz, 1H), 4.13 (m, 6H), 3.56 (dt, J=9.3, 6.7 Hz, 2H), 3.40 (m, 3H), 3.20-2.98 (m, 2H), 2.77 (t, J=6.4 Hz, 2H), 2.41-2.17 (m, 10H), 2.05 (q, J=6.8 Hz, 4H), 1.98-1.78 (m, 3H), 1.72-1.52 (m, 11H), 1.41-1.24 (m, 32H), 0.88 (m, 9H) ppm; MS: 836.27 m/z [M+H].

### Example 40

### 3-((4,4-bis(octyloxy)butanoyl)oxy)-2-(((((1-ethylpyrrolidin-2-yl)methyl)carbamoyl)oxy)methyl)propyl (9Z,12Z)-octadeca-9,12-dienoate

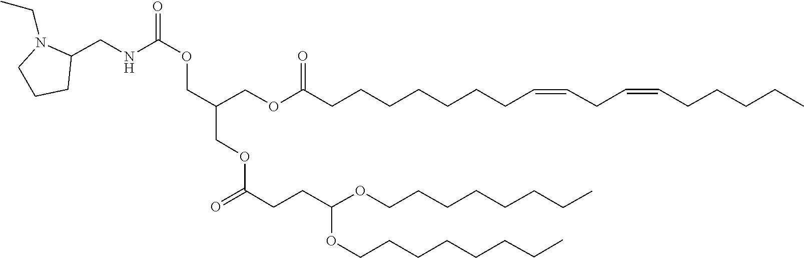 [SMILES: CCCCC/C=C\C/C=C\CCCCCCCC(=O)OCC(COC(=O)CCC(OCCCCCCCC)OCCCCCCCC)COC(=O)NCC1CCCN1CC]

13% yield; ¹H NMR (400 MHz, CDCl₃) δ 5.42-5.27 (m, 4H), 5.15 (br d, J=7.7 Hz, 1H), 4.48 (t, J=5.6 Hz, 1H), 4.13 (m, 6H), 3.56 (dt, J=9.3, 6.7 Hz, 2H), 3.43-3.31 (m, 3H), 3.19-3.05 (m, 2H), 2.78 (m, 3H), 2.50 (br m, 1H), 2.39 (m, 3H), 2.30 (t, J=7.6 Hz, 2H), 2.26-2.08 (m, 2H), 2.05 (q, J=6.8 Hz, 4H), 1.97-1.79 (m, 3H), 1.76-1.51 (m, 11H), 1.40-1.22 (m, 32H), 1.09 (t, J=7.2 Hz, 3H), 0.88 (m, 9H) ppm; MS: 850.20 m/z [M+H].

### Example 41

### 3-((4,4-bis(octyloxy)butanoyl)oxy)-2-((((1-ethylpyrrolidin-3-yl)carbamoyl)oxy)methyl)propyl (9Z,12Z)-octadeca-9,12-dienoate

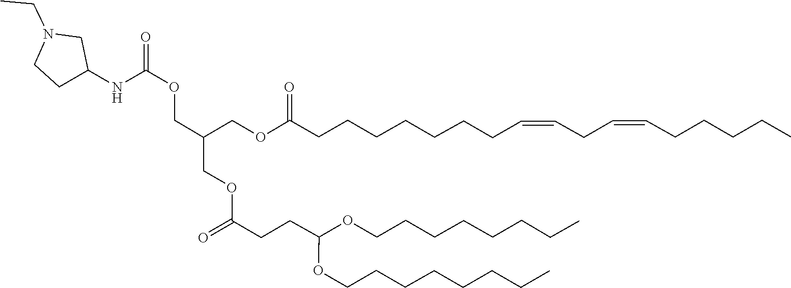 [SMILES: CCCCC/C=C\C/C=C\CCCCCCCC(=O)OCC(COC(=O)CCC(OCCCCCCCC)OCCCCCCCC)COC(=O)NC1CCN(CC)C1]

34% yield; ¹H NMR (400 MHz, CDCl₃) δ 5.44-5.27 (m, 4H), 5.01 (d, J=8.3 Hz, 1H), 4.48 (t, J=5.6 Hz, 1H), 4.20-4.02 (m, 7H), 3.56 (dt, J=9.3, 6.7 Hz, 2H), 3.40 (dt, J=9.3, 6.7 Hz, 2H), 2.86-2.73 (m, 3H), 2.62-2.19 (m, 11H), 2.05 (q, J=6.9 Hz, 4H), 1.92 (td, J=7.6, 5.5 Hz, 2H), 1.66-1.51 (m, 9H), 1.44-1.21 (m, 32H), 1.10 (t, J=7.2 Hz, 3H), 0.88 (m, 9H) ppm; MS: 836.33 m/z [M+H].

### Example 42

### 3-((4,4-bis(octyloxy)butanoyl)oxy)-2-((((1-methylpiperidin-3-yl)carbamoyl)oxy)methyl)propyl (9Z,12Z)-octadeca-9,12-dienoate

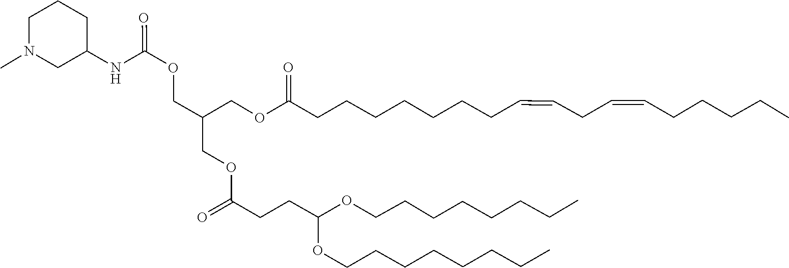 [SMILES: CCCCC/C=C\C/C=C\CCCCCCCC(=O)OCC(COC(=O)CCC(OCCCCCCCC)OCCCCCCCC)COC(=O)NC1CCCN(C)C1]

57% yield; ¹H NMR (400 MHz, CDCl₃) δ 5.43-5.27 (m, 4H), 5.18 (br m, 1H), 4.48 (t, J=5.6 Hz, 1H), 4.13 (m, 6H), 3.78 (br m, 1H), 3.56 (dt, J=9.2, 6.7 Hz, 2H), 3.40 (dt, J=9.2, 6.7 Hz, 2H), 2.77 (t, J=6.4 Hz, 2H), 2.47-2.13 (m, 11H), 2.05 (q, J=6.9 Hz, 4H), 1.92 (td, J=7.6, 5.5 Hz, 3H), 1.71-1.52 (m, 11H), 1.41-1.24 (m, 33H), 0.88 (m, 9H) ppm; MS: 836.51 m/z [M+H].

### Example 43

### 3-((4,4-bis(octyloxy)butanoyl)oxy)-2-(((((1-methylpyrrolidin-3-yl)methyl)carbamoyl)oxy)methyl)propyl (9Z,12Z)-octadeca-9,12-dienoate

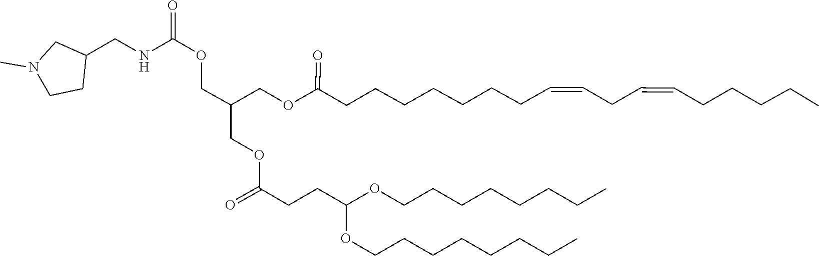 [SMILES: CCCCC/C=C\C/C=C\CCCCCCCC(=O)OCC(COC(=O)CCC(OCCCCCCCC)OCCCCCCCC)COC(=O)NCC1CCN(C)C1]

30% yield; ¹H NMR (400 MHz, CDCl₃) δ 5.44-5.30 (m, 4H), 5.19 (t, J=5.1 Hz, 1H), 4.51 (t, J=5.5 Hz, 1H), 4.18-4.11 (m, 6H), 3.58 (dt, J=9.2, 6.7 Hz, 2H), 3.42 (dt, J=9.3, 6.7 Hz, 2H), 3.19 (t, J=5.9 Hz, 2H), 2.84-2.79 (t, J=6.4 Hz, 2H), 2.64 (m, 1H), 2.56 (t, J=8.0 Hz, 1H), 2.44-2.31 (m, 11H), 2.10-1.92 (m, 7H), 1.71-1.46 (m, 9H), 1.44-1.19 (m, 33H), 0.91 (m, 9H) ppm; MS: 836.31 m/z [M+H].

### Example 44

### 3-((4,4-bis(octyloxy)butanoyl)oxy)-2-((((1-(diethylamino)propan-2-yl)carbamoyl)oxy)methyl)propyl (9Z,12Z)-octadeca-9,12-dienoate

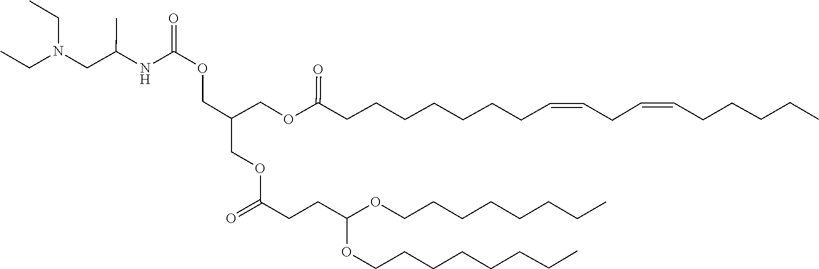 [SMILES: CCCCC/C=C\C/C=C\CCCCCCCC(=O)OCC(COC(=O)CCC(OCCCCCCCC)OCCCCCCCC)COC(=O)NC(C)CN(CC)CC]

78% yield; ¹H NMR (400 MHz, CDCl₃) δ 5.42-5.27 (m, 4H), 5.07 (d, J=5.6 Hz, 1H), 4.48 (t, J=5.6 Hz, 1H), 4.12 (m, 6H), 3.67-3.51 (m, 3H), 3.40 (dt, J=9.3, 6.7 Hz, 2H), 2.77 (t, J=6.4 Hz, 2H), 2.61-2.26 (m, 10H), 2.04 (dd, J=7.8, 5.9 Hz, 4H), 1.92 (td, J=7.6, 5.5 Hz, 2H), 1.68-1.50 (m, 7H), 1.41-1.24 (m, 34H), 1.17 (d, J=6.4 Hz, 3H), 0.99 (t, J=7.1 Hz, 6H), 0.88 (m, 9H) ppm; MS: 852.59 m/z [M+H].

### Example 45

### 3-((4,4-bis(octyloxy)butanoyl)oxy)-2-((((1-(dimethylamino)propan-2-yl)carbamoyl)oxy)methyl)propyl (9Z,12Z)-octadeca-9,12-dienoate

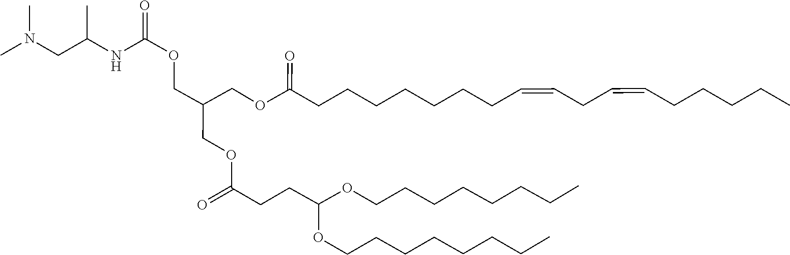 [SMILES: CCCCC/C=C\C/C=C\CCCCCCCC(=O)OCC(COC(=O)CCC(OCCCCCCCC)OCCCCCCCC)COC(=O)NC(C)CN(C)C]

68% yield; ¹H NMR (400 MHz, CDCl₃) δ 5.42-5.27 (m, 4H), 5.02 (br m, 1H), 4.48 (t, J=5.6 Hz, 1H), 4.18-4.07 (m, 6H), 3.66 (m, 1H), 3.56 (dt, J=9.3, 6.7 Hz, 2H), 3.40 (dt, J=9.3, 6.7 Hz, 2H), 2.77 (t, J=6.5 Hz, 2H), 2.42-2.27 (m, 6H), 2.22 (s, 6H), 2.14 (dd, J=12.4, 5.5 Hz, 1H), 2.05 (q, J=6.9 Hz, 4H), 1.92 (td, J=7.6, 5.5 Hz, 2H), 1.65-1.50 (m, 8H), 1.41-1.25 (m, 32H), 1.18 (d, J=6.4 Hz, 3H), 0.88 (m, 9H) ppm; MS: 824.28 m/z [M+H].

### Example 46

### 3-((4,4-bis(octyloxy)butanoyl)oxy)-2-((((3-(4-hydroxypiperidin-1-yl)propyl)carbamoyl)oxy)methyl)propyl (9Z,12Z)-octadeca-9,12-dienoate

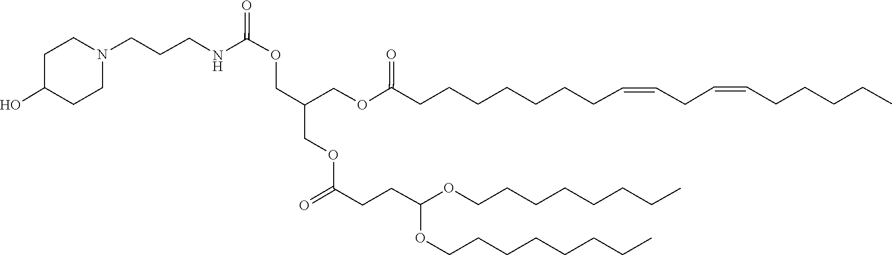 [SMILES: CCCCC/C=C\C/C=C\CCCCCCCC(=O)OCC(COC(=O)CCC(OCCCCCCCC)OCCCCCCCC)COC(=O)NCCCN1CCC(O)CC1]

26% yield; ¹H NMR (400 MHz, CDCl₃) δ 6.72 (br s, 1H), 5.42-5.27 (m, 4H), 4.48 (t, J=5.5 Hz, 1H), 4.23-4.05 (m, 6H), 3.62 (br s, 1H) 3.56 (dt, J=9.3, 6.7 Hz, 2H), 3.40 (dt, J=9.3, 6.7 Hz, 2H), 3.27 (q, J=5.7 Hz, 2H), 2.83 (d, J=11.2 Hz, 2H), 2.77 (m, 2H), 2.49-2.26 (m, 7H), 2.09-2.02 (m, 6H), 1.93 (m, 4H), 1.68-1.52 (m, 11H), 1.41-1.24 (m, 34H), 0.88 (m, 9H) ppm; MS: 880.46 m/z [M+H].

### Example 47

### 3-((4,4-bis(octyloxy)butanoyl)oxy)-2-((((9Z,12Z)-octadeca-9,12-dienoyl)oxy)methyl)propyl 4-methylpiperazine-1-carboxylate

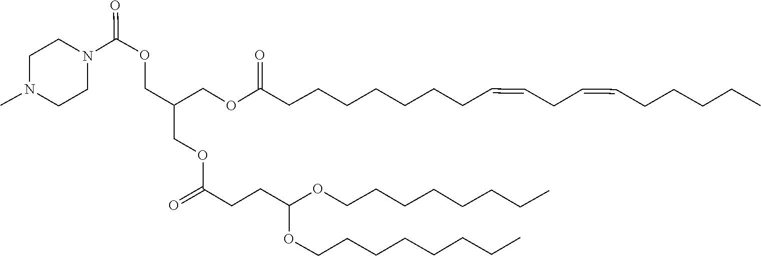 [SMILES: CCCCC/C=C\C/C=C\CCCCCCCC(=O)OCC(COC(=O)CCC(OCCCCCCCC)OCCCCCCCC)COC(=O)N1CCN(C)CC1]

61% yield; ¹H NMR (400 MHz, CDCl₃) δ 5.42-5.27 (m, 4H), 4.48 (t, J=5.5 Hz, 1H), 4.18-4.09 (m, 6H), 3.56 (dt, J=9.3, 6.7 Hz, 2H), 3.47 (br s, 4H), 3.40 (dt, J=9.3, 6.7 Hz, 2H), 2.81-2.73 (m, 2H), 2.45-2.26 (m, 11H), 2.05 (q, J=6.8 Hz, 4H), 1.92 (td, J=7.6, 5.5 Hz, 2H), 1.65-1.49 (m, 7H), 1.41-1.21 (m, 34H), 0.88 (m, 9H) ppm; MS: 822.25 m/z [M+H].

### Example 48

### 3-((4,4-bis(octyloxy)butanoyl)oxy)-2-((((9Z,12Z)-octadeca-9,12-dienoyl)oxy)methyl)propyl 4-ethylpiperazine-1-carboxylate

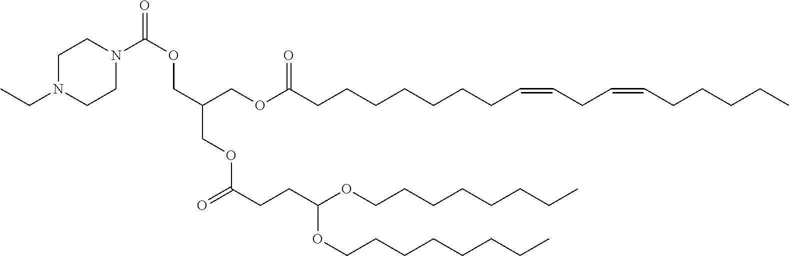 [SMILES: CCCCC/C=C\C/C=C\CCCCCCCC(=O)OCC(COC(=O)CCC(OCCCCCCCC)OCCCCCCCC)COC(=O)N1CCN(CC)CC1]

66% yield; ¹H NMR (400 MHz, CDCl₃) δ 5.44-5.27 (m, 4H), 4.48 (t, J=5.6 Hz, 1H), 4.19-4.09 (m, 6H), 3.56 (dt, J=9.3, 6.7 Hz, 2H), 3.48 (br s, 4H), 3.40 (dt, J=9.3, 6.7 Hz, 2H), 2.81-2.73 (m, 2H), 2.47-2.35 (m, 8H), 2.30 (t, J=7.6 Hz, 2H), 2.05 (q, J=6.9 Hz, 4H), 1.92 (td, J=7.6, 5.5 Hz, 2H), 1.65-1.52 (m, 7H), 1.41-1.24 (m, 34H), 1.09 (t, J=7.2 Hz, 3H), 0.88 (m, 9H) ppm; MS: 836.30 m/z [M+H].

### Example 49

### 3-((4,4-bis(octyloxy)butanoyl)oxy)-2-((((2-(4-hydroxypiperidin -1-yl)ethyl)carbamoyl)oxy)methyl)propyl (9Z,12Z)-octadeca-9,12-dienoate

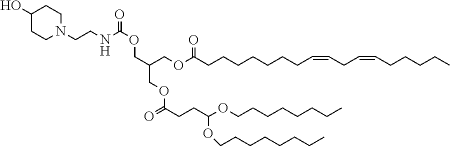 [SMILES: CCCCC/C=C\C/C=C\CCCCCCCC(=O)OCC(COC(=O)CCC(OCCCCCCCC)OCCCCCCCC)COC(=O)NCCN1CCC(O)CC1]

58% yield; ¹H NMR (400 MHz, CDCl₃) δ 5.43-5.27 (m, 3H), 5.18 (s, 1H), 4.48 (t, 1H), 4.17-4.09 (m, 6H), 3.76-3.65 (m, 1H), 3.56 (d, 2H), 3.40 (dt, 2H), 3.30-3.21 (m, 2H), 2.81-2.66 (m, 4H), 2.53-2.34 (m, 6H), 2.30 (t, 2H), 2.21-2.10 (m, 2H), 2.05 (q, 4H), 1.97-1.85 (m, 5H), 1.67-1.51 (m, 10H), 1.40-1.25 (m, 35H), 0.94-0.84 (m, 9H); MS: 866.41m/z [M+H].

### Synthesis of Example 50

### Example 50

### 3-((4,4-bis(octyloxy)butanoyl)oxy)-2-(((((1-ethylazetidin-3-yl)methoxy)carbonyl)oxy)methyl)propyl (9Z,12Z)-octadeca-9,12-dienoate

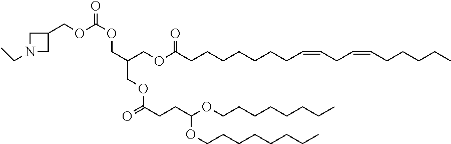 [SMILES: CCCCC/C=C\C/C=C\CCCCCCCC(=O)OCC(COC(=O)CCC(OCCCCCCCC)OCCCCCCCC)COC(=O)OCC1CN(CC)C1]

To a solution of intermediate 1d (300 mg, 0.43 mmol) in acetonitrile (6 mL) was added pyridine (69 uL, 86 mmol), DMAP (11 mg, 0.086 mmol), and 4-nitrophenyl chloroformate (134 mg, 0.65 mmol) sequentially at rt. Upon stirring for 2 h, (1-ethylazetidin-3-yl)methanol hydrochloride (194 mg, 1.29 mmol) and Et₃N (359 μL, 2.58 mmol) was added and the resulting reaction mixture was stirred for an additional 4 h at rt. The reaction mixture was extracted into hexanes and washed with water. If needed, the resulting water layer was re-extracted with hexanes. The combined hexanes layers were dried over anhydrous MgSO_{4,} filtered, and concentrated in vacuo. The crude residue was purified using silica gel chromatography (gradient of EtOAc in hexanes) to provide 181 mg (0.22 mmol, 50% yield) of the desired product as a clear oil. ¹H NMR (400 MHz, CDCl₃) δ 5.35 (m, 4H), 4.48 (t, J=5.5 Hz, 1H), 4.27-4.14 (m, 8H), 3.66-3.36 (m, 4H), 2.94 (br s, 1H), 2.78 (m, 3H), 2.42 (m, 5H), 2.30 (t, J=7.6 Hz, 2H), 2.05 (q, J=7.0 Hz, 4H), 1.92 (m, 2H), 1.62-1.52 (m, 8H), 1.31 (m, 35H), 0.95 (t, J=7.2 Hz, 3H), 0.89 (m, 9H) ppm; MS: 837.30 m/z [M+H].

### Synthesis of Examples 51-53

The following examples were synthesized from Intermediate 1d and an amino alcohol reagent using the method employed for Example 1.

### Example 51

### 3-((4,4-bis(octyloxy)butanoyl)oxy)-2-(((((1-ethylpyrrolidin-3-yl)methoxy)carbonyl)oxy)methyl)propyl (9Z,12Z)-octadeca-9,12-dienoate

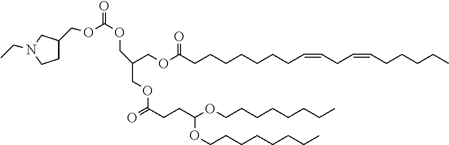 [SMILES: CCCCC/C=C\C/C=C\CCCCCCCC(=O)OCC(COC(=O)CCC(OCCCCCCCC)OCCCCCCCC)COC(=O)OCC1CCN(CC)C1]

57% yield; ¹H NMR (500 MHz, CDCl₃) δ 5.42-5.29 (m, 4H), 4.48 (t, J=5.5 Hz, 1H), 4.22-4.09 (m, 8H), 3.56 (dt, J=9.3, 6.7 Hz, 2H), 3.40 (dt, J=9.3, 6.6 Hz, 2H), 2.96-2.49 (m, 7H), 2.45-2.36 (m, 4H), 2.30 (t, J=7.6 Hz, 2H), 2.20-2.29 (m, 5H), 1.91 (td, J=7.6, 5.3 Hz, 2H), 1.75-1.52 (m, 8H), 1.40-1.16 (m, 37H), 0.88 (m, 9H) ppm; MS: 851.48 m/z [M+H].

### Example 52

### 3-((4,4-bis(octyloxy)butanoyl)oxy)-2-(((((1-ethyl-3-methylpiperidin-3-yl)methoxy)carbonyl)oxy)methyl)propyl (9Z,12Z)-octadeca-9,12-dienoate

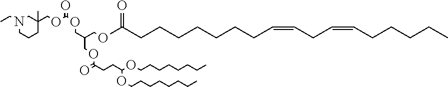 [SMILES: CCCCC/C=C\C/C=C\CCCCCCCC(=O)OCC(COC(=O)CCC(OCCCCCCCC)OCCCCCCCC)COC(=O)OCC1(C)CCCN(CC)C1]

66% yield; ¹H NMR (500 MHz, CDCl₃) 5.43-5.28 (m, 4H), 4.48 (t, J=5.5 Hz, 1H), 4.22-4.12 (m, 8H), 3.56 (dt, J=9.3, 6.7 Hz, 2H), 3.40 (dt, J=9.3, 6.7 Hz, 2H), 2.77 (t, J=6.7 Hz, 2H), 2.46-2.37 (m, 3H), 2.31 (t, J=7.6 Hz, 2H), 2.21 (br s, 6H), 2.05 (q, J=7.0 Hz, 4H), 1.92 (td, J=7.5, 5.4 Hz, 2H), 1.57 (m, 10H), 1.45-1.17 (m, 40H), 0.88 (m, 9H) ppm; MS: 865.58 m/z [M+H].

### Example 53

### 3-((4,4-bi s(octyl oxy)butanoyl)oxy)-2-(((((1-methylpip eri din-3-yl)methoxy)carbonyl)oxy)methyl)propyl (9Z,12Z)-octadeca-9,12-dienoate

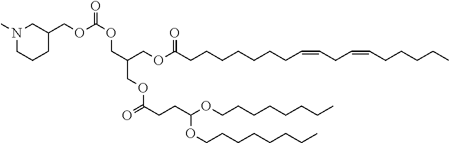 [SMILES: CCCCC/C=C\C/C=C\CCCCCCCC(=O)OCC(COC(=O)CCC(OCCCCCCCC)OCCCCCCCC)COC(=O)OCC1CCCN(C)C1]

42% yield; ¹H NMR (400 MHz, CDCl₃) δ 5.45-5.24 (m, 4H), 4.48 (t, J=5.5 Hz, 1H), 4.24-3.93 (m, 8H), 3.56 (dt, J=9.3, 6.7 Hz, 2H), 3.40 (dt, J=9.2, 6.7 Hz, 2H), 3.09 (br m, 2H), 2.77 (t, J=6.5 Hz, 1H), 2.70-2.16 (m, 7H), 2.05 (q, J=6.9 Hz, 4H), 1.92 (td, J=7.6, 5.5 Hz, 2H), 1.87-1.71 (m, 3H), 1.57 (m, 10H), 1.42-1.00 (m, 36H), 0.88 (m, 9H) ppm; MS: 850.77 m/z [M+H].

### Synthesis of Example 54

### Intermediate 54a: (Z)-9-(non-2-en-1-yloxy)-9-oxononanoic acid

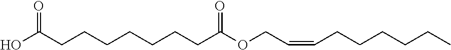 [SMILES: CCCCCC/C=C\COC(=O)CCCCCCCC(=O)O]

To a solution of nonanedioic acid (25 g, 132 mmol, 1 equiv) in THF (0.4-0.5 M) was added oxalyl chloride (1.1-1.5 equiv) at 15-25° C. Upon stirring for at least 10 min, DMF (0.01-0.1 equiv) was added, followed by at least 10 min of additional stirring. Then, (Z)-non-2-en-1-ol (24.3 g, 171 mmol, 1.3 equiv) was added dropwise and the resulting mixture was stirred at least 30 min at 15-25° C. The reaction mixture was concentrated and directly purified using silica gel chromatography to provide 17.3 g (55.4 mmol, 42% yield) of the desired product as an oil. ¹H NMR (400 MHz, CDCl₃) δ 5.65 (m, 1H), 5.53 (m, 1H), 4.62 (d, J=6.8 Hz, 2H), 2.33 (m, 4H), 2.10 (m, 2H), 1.63 (m, 4H), 1.28 (m, 14H), 0.88 (t, J=6.8 Hz, 3H) ppm; MS: 335.27 m/z [M+Na].

### Intermediate 54b: 3-hydroxy-2-(hydroxymethyl)propyl 4,4-bis(octyloxy)butanoate

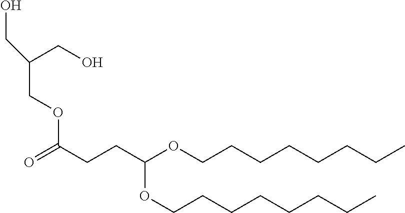 [SMILES: CCCCCCCCOC(CCC(=O)OCC(CO)CO)OCCCCCCCC]

To a solution of Intermediate 1c (16.0 g, 46.4 mmol), DMAP (1.1 g, 9.28 mmol), DIPEA (24.1 mL, 139 mmol), and 2-(hydroxymethyl)propane-1,3-diol (6.39 g, 60.3 mmol) in DCM (231 mL) was added EDCHCl (10.6 g, 55.6 mmol) at rt. The reaction mixture was stirred at rt for 18 h, diluted with water, and the organic layer was concentrated in vacuo. The crude residue was purified using silica gel chromatography (0-100% EtOAc in hexanes) to provide 6.7 g (15.4 mmol, 33% yield) of the desired product. ¹H NMR (400 MHz, CDCl₃) δ 4.48 (t, J=5.5 Hz, 1H), 4.24 (d, J=6.3 Hz, 2H), 3.75 (m, 4H), 3.55 (dt, J=9.3, 6.7 Hz, 2H), 3.39 (dt, J=9.3, 6.7 Hz, 2H), 2.54 (br s, 2H), 2.41 (t, J=7.5 Hz, 2H), 2.02 (m, 1H), 1.93 (td, J=7.5, 5.4 Hz, 2H), 1.59-1.48 (m, 4H), 1.37-1.20 (m, 20H), 0.87 (t, J=7.0 Hz, 6H) ppm.

### Intermediate 54c: (Z)-1-(3-((4,4-bis(octyloxy)butanoyl)oxy)-2-(hydroxymethyl)propyl) 9-(non-2-en-1-yl) nonanedioate

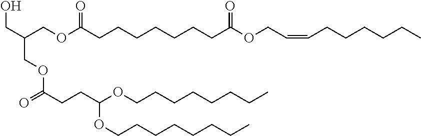 [SMILES: CCCCCC/C=C\COC(=O)CCCCCCCC(=O)OCC(CO)COC(=O)CCC(OCCCCCCCC)OCCCCCCCC]

To a solution of Intermediate 54a (1-1.2 equiv), DMAP (0.15 equiv), DIPEA (1.6-3.0 equiv), and Intermediate 54b (900 mg, 2.08 mmol, 1 equiv) in DCM (0.2 M) was added EDCI.HCl (1.6 equiv) at rt. The reaction mixture was stirred at rt for at least 2 h, concentrated in vacuo, and directly purified using silica gel chromatography (gradient of EtOAc in hexanes) to provide 503 mg (0.69 mmol, 33% yield) of the desired product as a clear oil. ¹H NMR (400 MHz, CDCl₃) δ 5.64 (m, 1H), 5.51 (m, 1H), 4.61 (d, J=6.7 Hz, 2H), 4.48 (t, J=5.5 Hz, 1H), 4.17 (m, 4H), 3.65-3.51 (m, 4H), 3.39 (dt, J=9.4, 6.7 Hz, 2H), 2.40 (t, J=7.5 Hz, 2H), 2.29 (m, 5H), 2.19 (m, 1H), 2.09 (q, J=7.2 Hz, 2H), 1.93 (td, J=7.5, 5.4 Hz, 2H), 1.67-1.50 (m, 8H), 1.40-1.19 (m, 32H), 0.87 (t, J=6.7 Hz, 9H) ppm; MS: 749.69 m/z [M+Na].

### Example 54

### (Z)-1-(3-((4,4-bis(octyloxy)butanoyl)oxy)-2-((((3-(diethylamino)propoxy)carbonyl)oxy)methyl)propyl) 9-(non-2-en-1-yl) nonanedioate

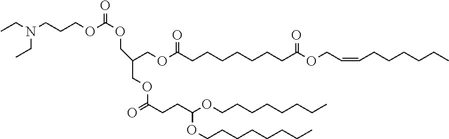 [SMILES: CCCCCC/C=C\COC(=O)CCCCCCCC(=O)OCC(COC(=O)CCC(OCCCCCCCC)OCCCCCCCC)COC(=O)OCCCN(CC)CC]

Example 54 was synthesized in 48% yield from Intermediate 54c and 3-(diethylamino)propan-1-ol using the method employed for Example 1. ¹H NMR (400 MHz, CDCl₃) δ 5.68-5.58 (m, 1H), 5.55-5.46 (m, 1H), 4.61 (dd, J=6.8, 1.2 Hz, 2H), 4.47 (t, J=5.5 Hz, 1H), 4.21-4.06 (m, 8H), 3.55 (dt, J=9.3, 6.7 Hz, 2H), 3.39 (dt, J=9.3, 6.7 Hz, 2H), 2.50 (m, 6H), 2.45-2.35 (m, 3H), 2.29 (t, J=7.6 Hz, 4H), 2.09 (m, 2H), 1.91 (m, 2H), 1.80 (m, 2H), 1.67-1.49 (m, 8H), 1.40-1.21 (m, 34H), 1.00 (t, J=7.1 Hz, 6H), 0.87 (m, 9H) ppm; MS: 885.65 m/z [M+H].

### Synthesis of Example 55

### Intermediate 55a: (Z)-7-(non-2-en-1-yloxy)-7-oxoheptanoic acid

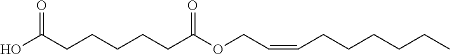 [SMILES: CCCCCC/C=C\COC(=O)CCCCCC(=O)O]

Intermediate 54a was synthesized from heptanedioic acid and (Z)-non-2-en-1-ol using the method employed for Intermediate 54a. ¹H NMR (400 MHz, CDCl₃) δ 5.58 (m, 1H), 5.44 (m, 1H), 4.55 (d, J=6.8 Hz, 2H), 2.26 (m, 4H), 2.03 (m, 2H), 1.59 (m, 4H), 1.30 (m, 10H), 0.81 (t, J=6.8 Hz, 3H) ppm.

### Intermediate 55b: (Z)-1-(3-((4,4-bis(octyloxy)butanoyl)oxy)-2-(hydroxymethyl)propyl) 7-(non-2-en-1-yl) heptanedioate

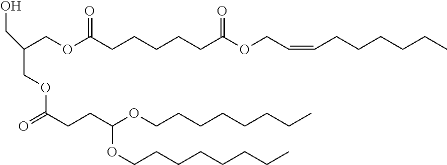 [SMILES: CCCCCC/C=C\COC(=O)CCCCCC(=O)OCC(CO)COC(=O)CCC(OCCCCCCCC)OCCCCCCCC]

Intermediate 55b was synthesized in 30% yield from Intermediate 55a and Intermediate 54b using the method employed for Intermediate 54c. ¹H NMR (400 MHz, CDCl₃) δ 5.64 (m, 1H), 5.51 (m, 1H), 4.62 (dd, J=6.8, 1.2 Hz, 2H), 4.48 (t, J=5.5 Hz, 1H), 4.22-4.07 (m, 4H), 3.67-3.51 (m, 4H), 3.40 (dt, J=9.3, 6.7 Hz, 2H), 2.41 (t, J=7.5 Hz, 2H), 2.32 (td, J=7.5, 5.9 Hz, 4H), 2.19 (m, 1H), 2.09 (m, 2H), 1.93 (td, J=7.5, 5.4 Hz, 2H), 1.68-1.50 (m, 8H), 1.39-1.23 (m, 30H), 0.88 (m, 9H) ppm; MS: 721.67 m/z [M+Na].

### Example 55

### (Z)-1-(3-((4,4-bis(octyloxy)butanoyl)oxy)-2-((((3-(diethylamino)propoxy)carbonyl)oxy)methyl)propyl) 7-(non-2-en-1-yl) heptanedioate

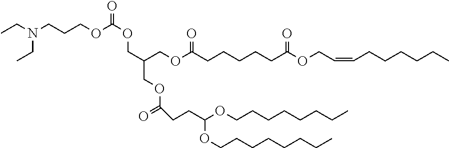 [SMILES: CCCCCC/C=C\COC(=O)CCCCCC(=O)OCC(COC(=O)CCC(OCCCCCCCC)OCCCCCCCC)COC(=O)OCCCN(CC)CC]

Example 55 was synthesized in 46% yield from Intermediate 55b and 3-(diethylamino)propan-1-ol using the method employed for Example 1. ¹H NMR (400 MHz, CDCl₃) δ ¹H NMR (400 MHz, CDCl₃) δ 5.69-5.59 (m, 1H), 5.55-5.47 (m, 1H), 4.62 (dd, J=6.9, 1.3 Hz, 2H), 4.48 (t, J=5.6 Hz, 1H), 4.20 (m, 4H), 4.13 (m, 4H), 3.56 (dt, J=9.3, 6.7 Hz, 2H), 3.40 (dt, J=9.3, 6.7 Hz, 2H), 2.94-2.57 (br m, 6H), 2.47-2.36 (m, 3H), 2.31 (td, J=7.5, 2.0 Hz, 4H), 2.19-1.89 (m, 6H) 1.70-1.49 (m, 9H), 1.41 —1.08 (m, 35H), 0.88 (m, 9H); MS: 857.57 m/z [M+H].

### Synthesis of Example 56

### Intermediate 56a: (Z)-5-(non-2-en-1-yloxy)-5-oxopentanoic acid

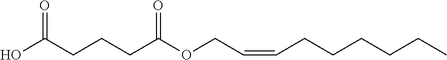 [SMILES: CCCCCC/C=C\COC(=O)CCCC(=O)O]

Intermediate 56a was synthesized from pentanedioic acid and (Z)-non-2-en-1-ol using the method employed for Intermediate 54a. ¹H NMR (400 MHz, CDCl₃) δ 5.66 (m, 1H), 5.52 (m, 1H), 4.64 (d, J=6.8 Hz, 2H), 2.43 (m, 4H), 2.15 (m, 2H), 1.97 (m, 2H), 1.28 (m, 8H), 0.89 (t, J=6.8 Hz, 3H) ppm.

### Intermediate 56b: (Z)-3-((4,4-bis(octyloxy)butanoyl)oxy)-2-(hydroxymethyl)propyl non-2-en-1-yl glutarate

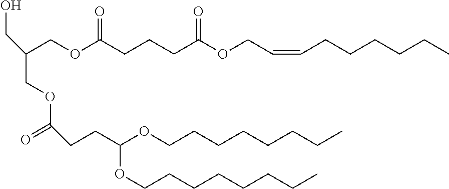 [SMILES: CCCCCC/C=C\COC(=O)CCCC(=O)OCC(CO)COC(=O)CCC(OCCCCCCCC)OCCCCCCCC]

Intermediate 56b was synthesized 32% yield from Intermediate 56a and Intermediate 54b using the method employed for Intermediate 54c. ¹H NMR (400 MHz, CDCl₃) δ 5.65 (m, 1H), 5.51 (m, 1H), 4.63 (dd, J=6.9, 1.3 Hz, 2H), 4.49 (t, J=5.5 Hz, 1H), 4.18 (m, 4H), 3.64 (m, 2H), 3.56 (dt, J=9.3, 6.7 Hz, 2H), 3.40 (dt, J=9.3, 6.7 Hz, 2H), 2.40 (m, 6H), 2.19 (m, 1H), 2.20 (m, 2H), 1.94 (m, 4H), 1.56 (m, 4H), 1.39-1.19 (m, 28H), 0.88 (m, 9H) ppm; MS: 693.64 [M+Na].

### Example 56

### (Z)-3-((4,4-bis(octyloxy)butanoyl)oxy)-2-((((3-(diethylamino)propoxy)carbonyl)oxy)methyl)propyl non-2-en-1-yl glutarate

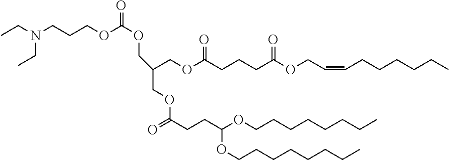 [SMILES: CCCCCC/C=C\COC(=O)CCCC(=O)OCC(COC(=O)CCC(OCCCCCCCC)OCCCCCCCC)COC(=O)OCCCN(CC)CC]

Example 56 was synthesized in 66% yield from Intermediate 56b and 3-(diethylamino)propan-1-ol using the method employed for Example 1. ¹H NMR (400 MHz, CDCl₃) δ 5.74-5.58 (m, 1H), 5.58-5.45 (m, 1H), 4.63 (dd, J=6.8, 1.3 Hz, 2H), 4.48 (t, J=5.5 Hz, 1H), 4.25-4.17 (m, 4H), 4.14 (dd, J=6.2, 3.1 Hz, 4H), 3.56 (dt, J=9.3, 6.7 Hz, 2H), 3.40 (dt, J=9.3, 6.7 Hz, 2H), 2.71 (br m, 6H), 2.48-2.35 (m, 7H), 2.09 (qd, J=7.3, 1.5 Hz, 3H), 2.04-1.87 (m, 5H), 1.66-1.52 (m, 5H), 1.42-1.30 (m, 30H), 1.15 (br m, 3H), 0.88 (m, 9H); MS: 829.67 m/z [M+H].

### Synthesis of Example 57

### Intermediate 57a: 9-(hexyloxy)-9-oxononanoic acid

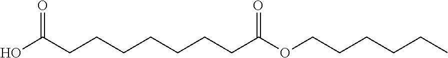 [SMILES: CCCCCCOC(=O)CCCCCCCC(=O)O]

Intermediate 57a was synthesized from nonanedioic acid and hexan-1-ol using the method employed for Intermediate 54a. ¹H NMR (400 MHz, CDCl₃) δ 4.05 (t, J=6.8 Hz, 2H), 2.36-2.26 (m, 4H), 1.60 (m, 6H), 1.32 (m, 12H), 0.88 (t, J=6.8 Hz, 3H) ppm.

### Intermediate 57b: 1-(3-((4,4-bis(octyloxy)butanoyl)oxy)-2-(hydroxymethyl)propyl) 9-hexyl nonanedioate

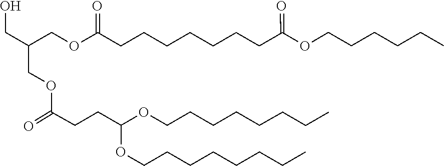 [SMILES: CCCCCCCCOC(CCC(=O)OCC(CO)COC(=O)CCCCCCCC(=O)OCCCCCC)OCCCCCCCC]

Intermediate 57b was synthesized in 30% yield from Intermediate 57a and Intermediate 54b using the method employed for Intermediate 54c. ¹H NMR (400 MHz, CDCl₃) δ 4.48 (t, J=5.5 Hz, 1H), 4.21-4.12 (m, 4H), 4.04 (t, J=6.7 Hz, 2H), 3.61 (t, J=5.9 Hz, 2H), 3.55 (dt, J=9.3, 6.7 Hz, 2H), 3.39 (dt, J=9.3, 6.7 Hz, 2H), 2.40 (t, J=7.5 Hz, 2H), 2.29 (m, 5H), 2.19 (m, 1H), 1.92 (td, J=7.5, 5.4 Hz, 2H), 1.66-1.49 (m, 9H), 1.38-1.24 (m, 32H), 0.87 (m, 9H) ppm; MS: 709.64 m/z [M+Na].

### Example 57

### 1-(3-((4,4-bis(octyloxy)butanoyl)oxy)-2-((((3-(diethylamino)propoxy)carbonyl)oxy)methyl)propyl) 9-hexyl nonanedioate

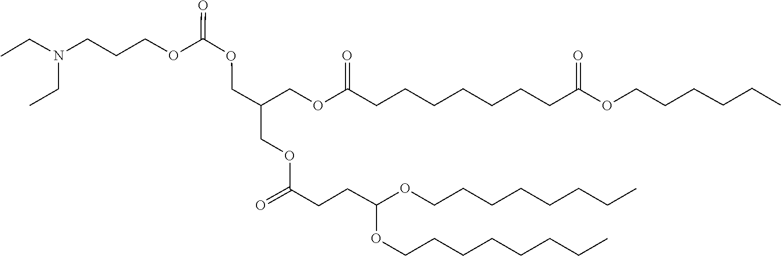 [SMILES: CCCCCCCCOC(CCC(=O)OCC(COC(=O)CCCCCCCC(=O)OCCCCCC)COC(=O)OCCCN(CC)CC)OCCCCCCCC]

Example 57 was synthesized in 65% yield from Intermediate 57b and 3-(diethylamino)propan-1-ol using the method employed for Example 1. ¹H NMR (400 MHz, CDCl₃) δ 4.47 (t, J=5.5 Hz, 1H), 4.23-4.08 (m, 8H), 4.05 (t, J=6.8 Hz, 2H), 3.55 (dt, J=9.3, 6.7 Hz, 2H), 3.39 (dt, J=9.3, 6.7 Hz, 2H), 2.50 (m, 6H), 2.40 (m, 3H), 2.29 (td, J=7.5, 5.9 Hz, 4H), 1.91 (m, 2H), 1.80 (m, 2H), 1.66-1.49 (m, 10H), 1.39-1.21 (m, 32H), 1.00 (t, J=7.1 Hz, 6H), 0.88 (m, 9H) ppm; MS: 845.63 m/z [M+H].

### Synthesis of Example 58

### Intermediate 58a: 9-(octyloxy)-9-oxononanoic acid

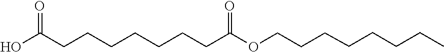 [SMILES: CCCCCCCCOC(=O)CCCCCCCC(=O)O]

Intermediate 58a was synthesized from nonanedioic acid and octan-1-ol using the method employed for Intermediate 54a. ¹H NMR (400 MHz, MeOD) δ 4.05 (t, J=6.6 Hz, 2H), 2.28 (m, 4H), 1.61 (m, 6H), 1.32 (m, 16H), 0.90 (t, J=6.6 Hz, 3H) ppm.

### Intermediate 58b: 1-(3-((4,4-bis(octyloxy)butanoyl)oxy)-2-(hydroxymethyl)propyl) 9-octyl nonanedioate

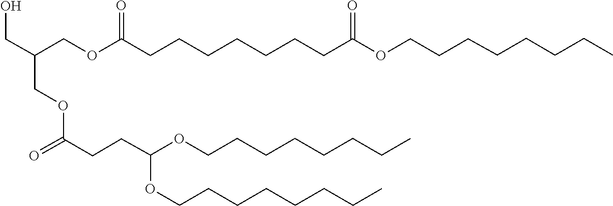 [SMILES: CCCCCCCCOC(=O)CCCCCCCC(=O)OCC(CO)COC(=O)CCC(OCCCCCCCC)OCCCCCCCC]

Intermediate 58b was synthesized in 26% yield from Intermediate 58a and Intermediate 54b using the method employed for Intermediate 54c. ¹H NMR (400 MHz, CDCl₃) δ 4.48 (t, J=5.5 Hz, 1H), 4.22-4.13 (m, 4H), 4.05 (t, J=6.7 Hz, 2H), 3.62 (t, J=5.9 Hz, 2H), 3.56 (dt, J=9.3, 6.7 Hz, 2H), 3.39 (dt, J=9.4, 6.7 Hz, 2H), 2.40 (t, J=7.5 Hz, 2H), 2.34-2.24 (m, 5H), 2.19 (m, 1H), 1.93 (td, J=7.5, 5.4 Hz, 2H), 1.66-1.49 (m, 10H), 1.39-1.19 (m, 36H), 0.87 (m, 9H) ppm.

### Example 58

### 1-(3-((4,4-bis(octyloxy)butanoyl)oxy)-2-((((3-(diethylamino)propoxy)carbonyl)oxy)methyl)propyl) 9-octyl nonanedioate

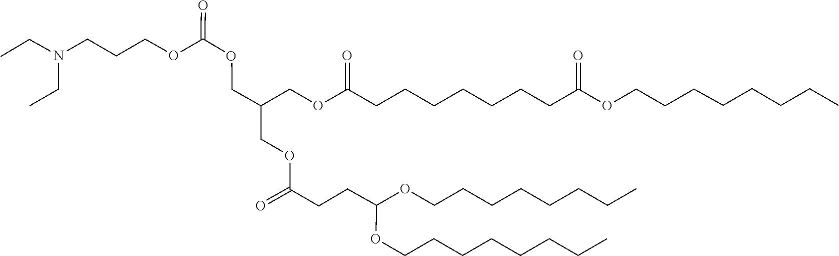 [SMILES: CCCCCCCCOC(=O)CCCCCCCC(=O)OCC(COC(=O)CCC(OCCCCCCCC)OCCCCCCCC)COC(=O)OCCCN(CC)CC]

Example 58 was synthesized from Intermediate 58b and 3-(diethylamino)propan-1-ol using the method employed for Example 1. ¹H NMR (400 MHz, CDCl₃) δ 4.47 (t, J=5.5 Hz, 1H), 4.22-4.08 (m, 8H), 4.05 (t, J=6.8 Hz, 2H), 3.55 (dt, J=9.3, 6.7 Hz, 2H), 3.39 (dt, J=9.3, 6.7 Hz, 2H), 2.50 (m, 6H), 2.40 (m, 3H), 2.29 (td, J=7.6, 6.2 Hz, 4H), 1.91 (td, J=7.6, 5.5 Hz, 2H), 1.80 (m, 2H), 1.66-1.51 (m, 10H), 1.38-1.21 (m, 36H), 1.00 (t, J=7.1 Hz, 6H), 0.88 (m, 9H) ppm; MS: 873.67 m/z [M+H].

### Synthesis of Example 59

### Intermediate 59a: 9-(decyloxy)-9-oxononanoic acid

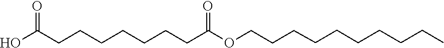 [SMILES: CCCCCCCCCCOC(=O)CCCCCCCC(=O)O]

Intermediate 59a was synthesized in 39% yield from nonanedioic acid and decan-1-ol using the method employed for Intermediate 54a. ¹H NMR (400 MHz, MeOD) δ 4.08 (t, J=6.6 Hz, 2H), 2.31 (m, 4H), 1.63 (m, 6H), 1.33 (m, 20H), 0.92 (t, J=6.8 Hz, 3H) ppm.

### Intermediate 59b: 1-(3-((4,4-bis(octyloxy)butanoyl)oxy)-2-(hydroxymethyl)propyl) 9-decyl nonanedioate

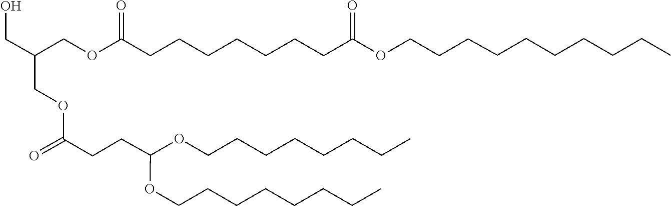 [SMILES: CCCCCCCCCCOC(=O)CCCCCCCC(=O)OCC(CO)COC(=O)CCC(OCCCCCCCC)OCCCCCCCC]

Intermediate 59b was synthesized in 43% yield from Intermediate 59a and Intermediate 54b using the method employed for Intermediate 54c. ¹H NMR (400 MHz, CDCl₃) δ 4.48 (t, J=5.5 Hz, 1H), 4.22-4.12 (m, 4H), 4.04 (t, J=6.8 Hz, 2H), 3.61 (t, J=5.9 Hz, 2H), 3.56 (dt, J=9.3, 6.7 Hz, 2H), 3.39 (dt, J=9.3, 6.7 Hz, 2H), 2.40 (t, J=7.5 Hz, 2H), 2.34-2.24 (m, 5H), 2.19 (m, 1H), 1.93 (td, J=7.5, 5.4 Hz, 2H), 1.66-1.50 (m, 10H), 1.37-1.21 (m, 40H), 0.87 (m, 9H) ppm; MS: 765.68 m/z [M+Na].

### Example 59

### 1-(3-((4,4-bis(octyloxy)butanoyl)oxy)-2-((((3-(diethylamino)propoxy)carbonyl)oxy)methyl)propyl) 9-decyl nonanedioate

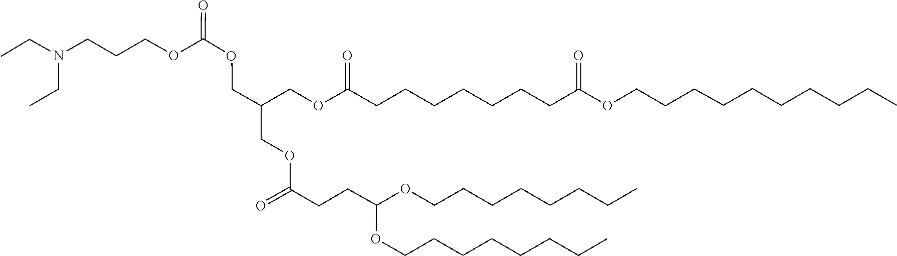 [SMILES: CCCCCCCCCCOC(=O)CCCCCCCC(=O)OCC(COC(=O)CCC(OCCCCCCCC)OCCCCCCCC)COC(=O)OCCCN(CC)CC]

Example 59 was synthesized in 65% yield from Intermediate 59b and 3-(diethylamino)propan-1-ol using the method employed for Example 1. ¹H NMR (400 MHz, CDCl₃) δ 4.47 (t, J=5.5 Hz, 1H), 4.21-4.09 (m, 8H), 4.04 (t, J=6.7 Hz, 2H), 3.55 (dt, J=9.3, 6.7 Hz, 2H), 3.39 (dt, J=9.3, 6.7 Hz, 2H), 2.50 (m, 6H), 2.39 (m, 3H), 2.28 (m, 4H), 1.91 (td, J=7.6, 5.5 Hz, 2H), 1.80 (m, 2H), 1.66-1.49 (m, 10H), 1.37-1.21 (m, 40H), 1.00 (t, J=7.1 Hz, 6H), 0.88 (m, 9H) ppm; MS: 901.76 m/z [M+H].

### Synthesis of Example 60

### Intermediate 60a: 12-oxo-12-(pentyloxy)dodecanoic acid

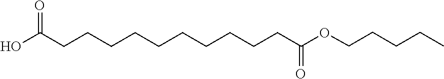 [SMILES: CCCCCOC(=O)CCCCCCCCCCC(=O)O]

Intermediate 60a was synthesized from dodecanedioic acid and pentan-1-ol using the method employed for Intermediate 54a. ¹H NMR (400 MHz, CDCl₃) δ 4.07 (t, J=6.8 Hz, 2H), 2.30 (m, 4H), 1.63 (m, 6H), 1.33 (m, 16H), 0.93 (t, J=6.8 Hz, 3H) ppm.

### Intermediate 60b: 1-(3-((4,4-bis(octyloxy)butanoyl)oxy)-2-(hydroxymethyl)propyl) 12-pentyl dodecanedioate

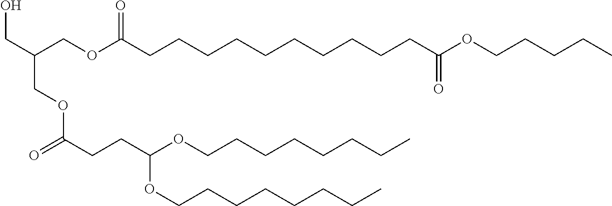 [SMILES: CCCCCCCCOC(CCC(=O)OCC(CO)COC(=O)CCCCCCCCCCC(=O)OCCCCC)OCCCCCCCC]

Intermediate 60b was synthesized in 21% yield from Intermediate 60a and Intermediate 54b using the method employed for Intermediate 54c. ¹H NMR (400 MHz, CDCl₃) δ 4.48 (t, J=5.5 Hz, 1H), 4.22-4.11 (m, 4H), 4.05 (t, J=6.7 Hz, 2H), 3.61 (t, J=5.9 Hz, 2H), 3.56 (dt, J=9.3, 6.7 Hz, 2H), 3.39 (dt, J=9.3, 6.7 Hz, 2H), 2.40 (t, J=7.5 Hz, 2H), 2.34-2.24 (m, 5H), 2.19 (m, 1H), 1.93 (td, J=7.5, 5.4 Hz, 2H), 1.65-1.50 (m, 10H), 1.29 (m, 36H), 0.89 (m, 9H) ppm; MS: 737.68 m/z [M+Na].

### Example 60

### 1-(3-((4,4-bis(octyloxy)butanoyl)oxy)-2-((((3-(diethylamino)propoxy)carbonyl)oxy)methyl)propyl) 12-pentyl dodecanedioate

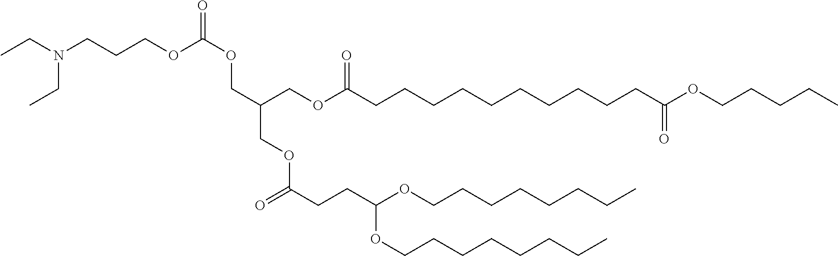 [SMILES: CCCCCCCCOC(CCC(=O)OCC(COC(=O)CCCCCCCCCCC(=O)OCCCCC)COC(=O)OCCCN(CC)CC)OCCCCCCCC]

Example 60 was synthesized in 65% yield from Intermediate 60b and 3-(diethylamino)propan-1-ol using the method employed for Example 1. ¹H NMR (400 MHz, CDCl₃) δ 4.47 (t, J=5.5 Hz, 1H), 4.24-4.09 (m, 8H), 4.05 (t, J=6.7 Hz, 2H), 3.55 (dt, J=9.2, 6.7 Hz, 2H), 3.39 (dt, J=9.3, 6.7 Hz, 2H), 2.50 (m, 6H), 2.40 (m, 3H), 2.29 (td, J=7.6, 5.8 Hz, 4H), 1.91 (td, J=7.6, 5.5 Hz, 2H), 1.80 (dq, J=8.7, 6.7 Hz, 2H), 1.68-1.49 (m, 10H), 1.38-1.24 (m, 36H), 1.00 (t, J=7.1 Hz, 6H), 0.89 (m, 9H) ppm; MS: 873.67 m/z [M+H].

### Synthesis of Example 61

### Intermediate 61a: 12-(heptyloxy)-12-oxododecanoic acid

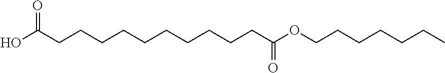 [SMILES: CCCCCCCOC(=O)CCCCCCCCCCC(=O)O]

Intermediate 61a was synthesized from dodecanedioic acid and heptan-1-ol using the method employed for Intermediate 54a. ¹H NMR (400 MHz, CDCl₃) δ 4.05 (t, J=6.8 Hz, 2H), 2.31 (m, 4H), 1.61 (m, 6H), 1.30 (m, 20H), 0.88 (t, J=6.8 Hz, 3H) ppm.

### Intermediate 61b: 1-(3-((4,4-bis(octyloxy)butanoyl)oxy)-2-(hydroxymethyl)propyl) 12-heptyl dodecanedioate

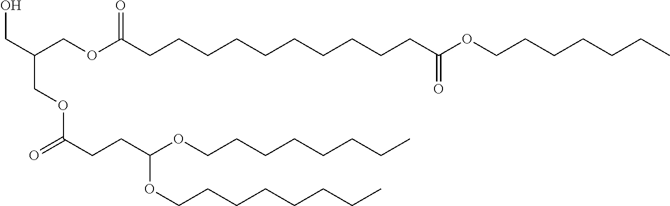 [SMILES: CCCCCCCCOC(CCC(=O)OCC(CO)COC(=O)CCCCCCCCCCC(=O)OCCCCCCC)OCCCCCCCC]

Intermediate 61b was synthesized in 33% yield from Intermediate 61a and Intermediate 54b using the method employed for Intermediate 54c. ¹H NMR (400 MHz, CDCl₃) δ 4.48 (t, J=5.5 Hz, 1H), 4.22-4.12 (m, 4H), 4.05 (t, J=6.7 Hz, 2H), 3.64-3.59 (m, 2H), 3.56 (dt, J=9.3, 6.7 Hz, 2H), 3.39 (dt, J=9.3, 6.7 Hz, 2H), 2.40 (t, J=7.5 Hz, 2H), 2.35-2.23 (m, 5H), 2.19 (m, 1H), 1.93 (td, J=7.5, 5.5 Hz, 2H), 1.66-1.50 (m, 10H), 1.37-1.23 (m, 40H), 0.87 (m, 9H) ppm; MS: 765.68 m/z [M+Na].

### Example 61

### 1-(3-((4,4-bi s(octyloxy)butanoyl)oxy)-2-((((3-(diethylamino)propoxy)carbonyl)oxy)methyl)propyl) 12-heptyl dodecanedioate

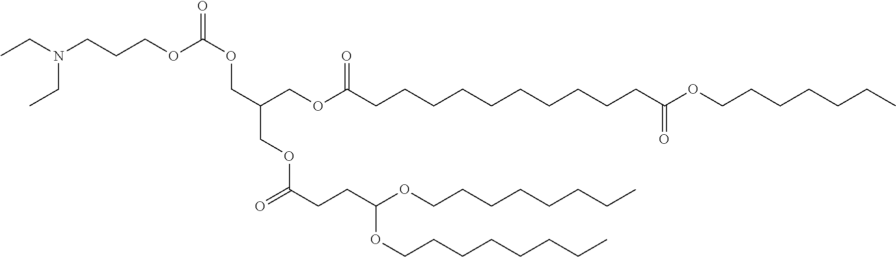 [SMILES: CCCCCCCCOC(CCC(=O)OCC(COC(=O)CCCCCCCCCCC(=O)OCCCCCCC)COC(=O)OCCCN(CC)CC)OCCCCCCCC]

Example 61 was synthesized in 69% yield from Intermediate 61b and 3-(diethylamino)propan-1-ol using the method employed for Example 1. ¹H NMR (400 MHz, CDCl₃) δ 4.47 (t, J=5.5 Hz, 1H), 4.22-4.09 (m, 8H), 4.05 (t, J=6.7 Hz, 2H), 3.55 (dt, J=9.3, 6.7 Hz, 2H), 3.39 (dt, J=9.3, 6.6 Hz, 2H), 2.50 (m, 6H), 2.40 (m, 3H), 2.29 (td, J=7.6, 6.3 Hz, 4H), 1.91 (td, J=7.6, 5.5 Hz, 2H), 1.80 (m, 2H), 1.66-1.49 (m, 10H), 1.37-1.24 (m, 40H), 1.00 (t, J=7.1 Hz, 6H), 0.88 (m, 9H) ppm; MS: 901.72 m/z [M+H].

### Synthesis of Example 62

### Intermediate 62a: methyl 6-hydroxyhexanoate

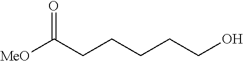

A mixture of oxepan-2-one (100 g, 876 mmol) in HCl in MeOH (1000 mL) was stirred at 70° C. for 12 h. The reaction mixture as adjusted to pH 8 by addition of aq. NaHCO₃, and then it was extracted into EtOAc (3×1000 mL). The combined organic layers were washed with brine (50 mL), dried over anhydrous Na₂SO₄, filtered, and concentrated in vacuo to provide 100 g (685 mmol, 78% yield) of the crude product as colorless oil, which did not require further purification. ¹H NMR (400 MHz, CDCl₃) δ 3.65 (m, 5H), 2.33 (t, J=7.6 Hz, 2H), 1.69-1.57 (m, 4H), 1.41 (m, 3H) ppm.

### Intermediate 62b: methyl 6-oxohexanoate

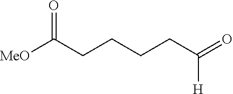

To a solution of Intermediate 62a (93 g, 636 mmol) in DCM (1200 mL) was added Et₃N (266 mL, 1.91 mol) at 0° C. Then, pyridinesulfur trioxide (203 g, 1.27 mol) in DMSO (497 mL) was added drop wise at 0° C. The resulting reaction mixture was stirred at 15° C. for 2 h, diluted with water (1000 mL), and extracted into DCM (2×1000 mL). The combined organic layers were washed with brine (1000 mL), dried over anhydrous Na₂SO₄, filtered, and concentrated in vacuo. The crude residue was purified using silica gel chromatography (0-5% EtOAc in petroleum ether) to provide 63 g (437 mmol, 69% yield) of the desired product as a colorless oil. ¹H NMR (400 MHz, CDCl₃) δ 9.77 (m, 1H), 3.67 (s, 3H), 2.47 (m, 2H), 2.33 (m, 2H), 1.67 (m, 4H) ppm.

### Intermediate 62c: methyl 6,6-dimethoxyhexanoate

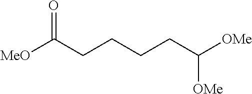 [SMILES: COC(=O)CCCCC(OC)OC]

To a solution of Intermediate 62b (60 g, 416 mmol) in MeOH (300 mL) was added H₂SO₄ (2.22 mL, 4.08 g, 41.6 mmol). The reaction mixture was stirred at 80° C. for 12 h. The reaction mixture was concentrated under reduced pressure to give a residue, diluted with sat. NaHCO₃ to pH 7, and extracted into EtOAc (3×500 mL). The combined organic layers were washed with brine (500 mL), dried over anhydrous Na₂SO₄, filtered, and concentrated in vacuo. The crude residue was purified using silica gel chromatography (0-5% EtOAc in petroleum ether) to provide 30 g (158 mmol, 38% yield) of the desired product as a colorless oil. ¹H NMR (400 MHz, CDCl₃) δ 4.36 (t, J=5.6 Hz, 1H), 3.67 (s, 3H), 3.32 (s, 6H), 2.32 (t, J=7.6 Hz, 2H), 1.67-1.60 (m, 4H), 1.35 (m, 2H) ppm.

### Intermediate 62d: methyl 6,6-bis(octyloxy)hexanoate

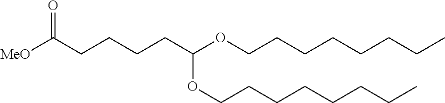 [SMILES: CCCCCCCCOC(CCCCC(=O)OC)OCCCCCCCC]

To a solution of Intermediate 62c (30 g, 158 mmol) in octan-1-ol (80 mL) was added KHSO₄ (10.7 g, 78.9 mmol). The reaction mixture was stirred at 80° C. for 12 h, diluted with petroleum ether (150 mL, and directly purified using silica gel chromatography (petroleum ether) to provide 35 g (90.5 mmol, 57% yield) of the desired product as a colorless oil. ¹H NMR (400 MHz, CDCl₃) δ 4.46 (t, J=5.6 Hz, 1H), 3.67 (s, 3H), 3.57 (m, 2H), 3.40 (m, 2H), 2.32 (t, J=7.6 Hz, 2H), 1.67-1.55 (m, 8H), 1.36-1.28 (m, 22H), 0.80 (t, J=6.8 Hz, 6H) ppm.

### Intermediate 62e: 6,6-bis(octyloxy)hexanoic acid

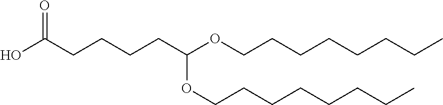 [SMILES: CCCCCCCCOC(CCCCC(=O)O)OCCCCCCCC]

To a solution of Intermediate 62d (35 g, 90.5 mmol) in MeOH (150 mL) was added a solution of NaOH (10.9 g, 272 mmol) in H₂O (50 mL). Upon stirring at 15° C. for 5 h, the reaction mixture was concentrated under reduced pressure, diluted with water (150 mL), and extracted into petroleum ether (200 mL). The organic layer was washed with brine (200 mL), dried over anhydrous Na₂SO₄, filtered, and concentrated in vacuo. The crude residue was purified using silica gel chromatography (0-5% EtOAc in petroleum ether) to provide 9.5 g (25.5 mmol, 28% yield) of the desired product as a yellow oil. ¹H NMR (400 MHz, CDCl₃) δ 4.47 (t, J=5.6 Hz, 1H), 3.56 (m, 2H), 3.40 (m, 2H), 2.37 (t, J=7.6 Hz, 2H), 1.69-1.55 (m, 8H), 1.33 (m, 22H), 0.89 (t, J=6.8 Hz, 6H) ppm; MS: 371.3 [M−H].

### Intermediate 62f: 3-((6,6-bis(octyloxy)hexanoyl)oxy)-2-(hydroxymethyl)propyl (9Z,12Z)-octadeca-9,12-dienoate

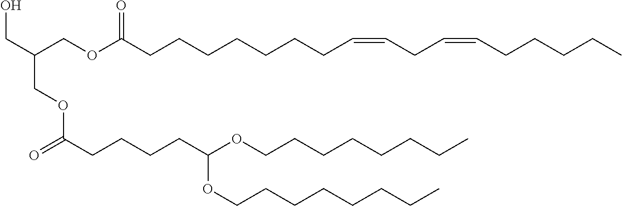 [SMILES: CCCCC/C=C\C/C=C\CCCCCCCC(=O)OCC(CO)COC(=O)CCCCC(OCCCCCCCC)OCCCCCCCC]

Intermediate 62f was synthesized in 34% yield from Intermediate 1a and Intermediate 62e using the method employed for Intermediate 1d. ¹H NMR (400 MHz, CDCl₃) δ 5.45-5.29 (m, 4H), 4.47 (t, J=5.7 Hz, 1H), 4.26-4.11 (m, 4H), 3.63 (t, J=5.9 Hz, 2H), 3.57 (dt, J=9.3, 6.7 Hz, 2H), 3.41 (dt, J=9.3, 6.7 Hz, 2H), 2.78 (m, 2H), 2.35 (td, J=7.6, 6.4 Hz, 4H), 2.28 (t, J=6.3 Hz, 1H), 2.21 (m, 1H), 2.07 (q, J=6.8 Hz, 4H), 1.72-1.53 (m, 9H), 1.45-1.23 (m, 38H), 0.90 (m, 9H) ppm; MS: 745.74 [M+Na].

### Example 62

### 3-((6,6-bis(octyloxy)hexanoyl)oxy)-2-((((3-(diethylamino)propoxy)carbonyl)oxy)methyl)propyl (9Z,12Z)-octadeca-9,12-dienoate

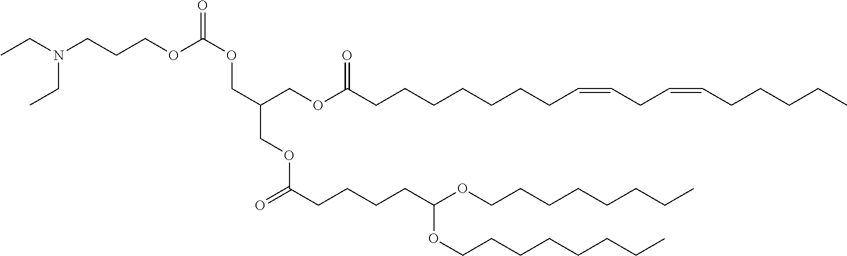 [SMILES: CCCCC/C=C\C/C=C\CCCCCCCC(=O)OCC(COC(=O)CCCCC(OCCCCCCCC)OCCCCCCCC)COC(=O)OCCCN(CC)CC]

Example 62 was synthesized in 47% yield from Intermediate 62f and 3-(diethylamino)propan-1-ol using the method employed for Example 1. ¹H NMR (400 MHz, CDCl₃) δ 5.43-5.27 (m, 4H), 4.44 (t, J=5.7 Hz, 1H), 4.22-4.07 (m, 8H), 3.55 (dt, J=9.3, 6.7 Hz, 2H), 3.39 (dt, J=9.3, 6.7 Hz, 2H), 2.77 (t, J=6.4 Hz, 2H), 2.51 (q, J=6.9 Hz, 6H), 2.41 (m, 1H), 2.31 (td, J=7.6, 5.4 Hz, 4H), 2.04 (q, J=6.8 Hz, 4H), 1.81 (m, 2H), 1.60 (m, 10H), 1.44-1.20 (m, 36H), 1.00 (t, J=7.1 Hz, 6H), 0.88 (m, 9H) ppm; MS: 881.76 m/z [M+H].

### Synthesis of Example 63

### Intermediate 63a: heptadecan-9-ol

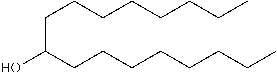 [SMILES: CCCCCCCCC(O)CCCCCCCC]

To a solution of nonanal (40 g, 281.22 mmol) in THF (400 mL) was added bromo (octyl) magnesium (1 M, 309.34 mL) at −40° C. The mixture was stirred at 16° C. for 2 h. The residue was poured into sat. NH₄Cl (500 mL) and extracted into ethyl acetate (3×700 mL). The combined organic layers were washed with brine (800 mL), dried over anhydrous Na₂SO₄, filtered, and concentrated in vacuo. The crude residue was purified using silica gel chromatography (0-1.5% EtOAc in petroleum ether) to provide 32 g (124.8 mmol, 44% yield) of the desired product as a white solid. ¹H NMR (400 MHz, CDCl₃) δ 3.52 (m, 1H), 1.42-1.21 (m, 28H), 0.81 (t, J=6.6 Hz, 6H) ppm.

### Intermediate 63b: 5-(heptadecan-9-yloxy)-5-oxopentanoic acid

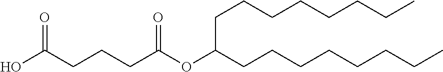 [SMILES: CCCCCCCCC(CCCCCCCC)OC(=O)CCCC(=O)O]

Intermediate 63b was synthesized from pentanedioic acid and Intermediate 63a using the method employed for Intermediate 54a. ¹H NMR (400 MHz, MeOD) δ 4.90 (m, 1H), 2.36 (m, 4H), 1.91 (m, 2H), 1.54 (m, 4H), 1.29 (m, 24H) 0.90 (t, J=6.6 Hz, 6H) ppm.

### Intermediate 63c: heptadecan-9-yl (3-hydroxy-2-((((9Z,12Z)-octadeca-9,12-dienoyl)oxy)methyl)propyl) glutarate

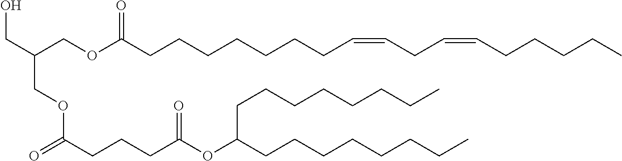 [SMILES: CCCCC/C=C\C/C=C\CCCCCCCC(=O)OCC(CO)COC(=O)CCCC(=O)OC(CCCCCCCC)CCCCCCCC]

Intermediate 63c was synthesized in 41% yield from Intermediate 1a and Intermediate 63b using the method employed for Intermediate 1d. ¹H NMR (400 MHz, CDCl₃) δ 5.43-5.26 (m, 4H), 4.86 (m, 1H), 4.22-4.08 (m, 4H), 3.61 (t, J=5.9 Hz, 2H), 2.77 (m, 2H), 2.43-62.28 (m, 6H), 2.26-2.16 (m, 2H), 2.04 (q, J=6.8 Hz, 4H), 1.95 (m, 2H), 1.65-1.58 (m, 2H), 1.50 (m, 4H), 1.40-1.18 (m, 37H), 0.88 (m, 9H) ppm; MS: 721.84 m/z [M+H].

### Example 63

### 3-(((3-(diethylamino)propoxy)carbonyl)oxy)-2-((((9Z,12Z)-octadeca-9,12-dienoyl)oxy)methyl)propyl heptadecan-9-yl glutarate

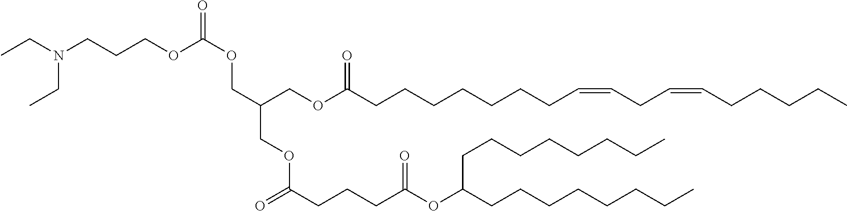 [SMILES: CCCCC/C=C\C/C=C\CCCCCCCC(=O)OCC(COC(=O)CCCC(=O)OC(CCCCCCCC)CCCCCCCC)COC(=O)OCCCN(CC)CC]

Example 63 was synthesized in 59% yield from Intermediate 63c and 3-(diethylamino)propan-1-ol using the method employed for Example 1. ¹H NMR (500 MHz, CDCl₃) δ 5.42-5.28 (m, 4H), 4.86 (m, 1H), 4.21-4.08 (m, 8H), 2.76 (t, J=6.7 Hz, 2H), 2.50 (q, J=7.0 Hz, 6H), 2.46-2.27 (m, 7H), 2.04 (d, J=6.2 Hz, 4H), 1.94 (m, J=7.5 Hz, 2H), 1.80 (m, J=6.8 Hz, 2H), 1.60 (t, J=7.3 Hz, 2H), 1.50 (q, J=6.2 Hz, 4H), 1.35 (t, J=7.1 Hz, 4H), 1.33-1.22 (m, 34H), 1.00 (t, J=7.1 Hz, 6H), 0.88 (m, 9H) ppm; MS: 879.78 m/z [M+H].

### Synthesis of Example 64

### Intermediate 64a: 7-(heptadecan-9-yloxy)-7-oxoheptanoic acid

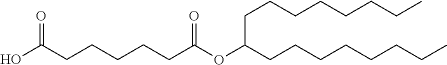 [SMILES: CCCCCCCCC(CCCCCCCC)OC(=O)CCCCCC(=O)O]

Intermediate 64 was synthesized from heptanedioic acid and Intermediate 63a using the method employed for Intermediate 54a. ¹H NMR (400 MHz, MeOD) δ 4.90 (m, 1H), 2.30 (m, 4H), 1.62 (m, 4H), 1.53 (m, 4H), 1.29 (m, 26H), 0.90 (t, J=6.8 Hz, 6H) ppm.

### Intermediate 64b: 1-(heptadecan-9-yl) 7-(3-hydroxy-2-((((9Z,12Z)-octadeca-9,12-dienoyl)oxy)methyl)propyl) heptanedioate

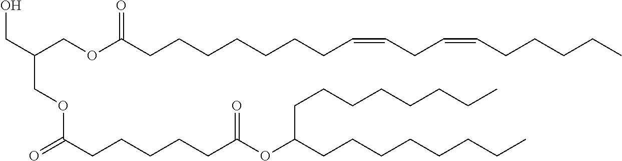 [SMILES: CCCCC/C=C\C/C=C\CCCCCCCC(=O)OCC(CO)COC(=O)CCCCCC(=O)OC(CCCCCCCC)CCCCCCCC]

Intermediate 64b was synthesized in 43% yield from Intermediate 1a and Intermediate 64a using the method employed for Intermediate 1d. ¹H NMR (400 MHz, CDCl₃) δ 5.42-5.27 (m, 4H), 4.85 (m, 1H), 4.22-4.12 (m, 4H), 3.61 (t, J=5.9 Hz, 2H), 2.76 (dd, J=7.2, 5.9 Hz, 2H), 2.36-2.24 (m, 6H), 2.19 (m, 1H), 2.04 (t, J=3.5 Hz, 4H), 1.69-1.58 (m, 6H), 1.49 (t, J=6.2 Hz, 4H), 1.41-1.19 (m, 40H), 0.88 (m, 9H) ppm; MS: 749.83 m/z [M+H].

### Example 64

### 1-(3-(((3-(diethylamino)propoxy)carbonyl)oxy)-2-((((9Z,12Z)-octadeca-9,12-dienoyl)oxy)methyl)propyl) 7-(heptadecan-9-yl) heptanedioate

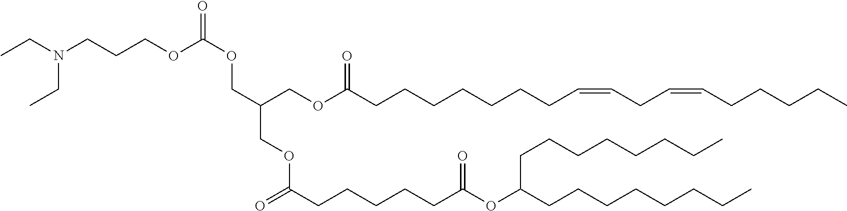 [SMILES: CCCCC/C=C\C/C=C\CCCCCCCC(=O)OCC(COC(=O)CCCCCC(=O)OC(CCCCCCCC)CCCCCCCC)COC(=O)OCCCN(CC)CC]

Example 64 was synthesized in 63% yield from Intermediate 64b and 3-(diethylamino)propan-1-ol using the method employed for Example 1. ¹H NMR (400 MHz, CDCl₃) δ 5.42-5.26 (m, 4H), 4.85 (m, 1H), 4.22-4.07 (m, 8H), 2.76 (m, 2H), 2.50 (m, 6H), 2.41 (m, 1H), 2.35-2.24 (m, 7H), 2.04 (q, J=6.2 Hz, 4H), 1.80 (m, 2H), 1.63 (m, 7H), 1.49 (q, J=6.1 Hz, 4H), 1.38-1.24 (m, 38H), 1.00 (t, J=7.1 Hz, 6H), 0.88 (m, 9H) ppm; MS: 907.61 m/z [M+H].

### Synthesis of Example 65

### Intermediate 65a: 3-((2-hexyldecanoyl)oxy)-2-(hydroxymethyl)propyl (9Z,12Z)-octadeca-9,12-dienoate

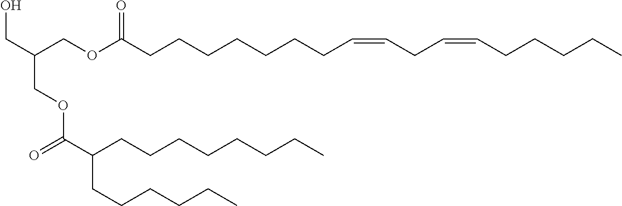 [SMILES: CCCCC/C=C\C/C=C\CCCCCCCC(=O)OCC(CO)COC(=O)C(CCCCCC)CCCCCCCC]

Intermediate 65a was synthesized in 40% yield from Intermediate 1a and 2-hexyldecanoic acid using the method employed for Intermediate 1d. ¹H NMR (400 MHz, CDCl₃) δ 5.45-5.25 (m, 4H), 4.25-4.10 (m, 4H), 3.61 (t, J=6.0 Hz, 2H), 2.77 (t, J=6.4 Hz, 2H), 2.39-2.29 (m, 3H), 2.27-2.16 (m, 2H), 2.04 (q, J=6.8 Hz, 4H), 1.60 (m, 4H), 1.50-1.16 (m, 36H), 0.88 (m, 9H) ppm; MS: 607.77 m/z [M+H].

### Example 65

### 3-(((3-(diethylamino)propoxy)carbonyl)oxy)-2-(((2-hexyldecanoyl)oxy)methyl)propyl (9Z,12Z)-octadeca-9,12-dienoate

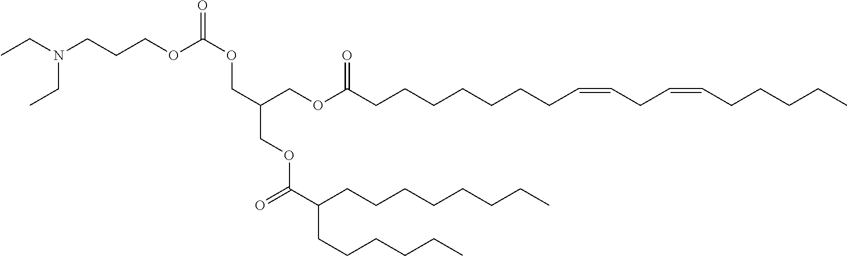 [SMILES: CCCCC/C=C\C/C=C\CCCCCCCC(=O)OCC(COC(=O)OCCCN(CC)CC)COC(=O)C(CCCCCC)CCCCCCCC]

Example 65 was synthesized in 63% yield from Intermediate 65a and 3-(diethylamino)propan-1-ol using the method employed for Example 1. ¹H NMR (400 MHz, CDCl₃) δ 5.43-5.27 (m, 4H), 4.22-4.07 (m, 8H), 2.77 (t, J=6.5 Hz, 2H), 2.50 (q, J=7.1 Hz, 6H), 2.42 (m, 1H), 2.36-2.26 (m, 3H), 2.09-2.00 (m, 4H), 1.80 (m, 2H), 1.63-1.51 (m, 4H), 1.43 (m, 2H), 1.39-1.19 (m, 34H), 1.00 (t, J=7.1 Hz, 6H), 0.87 (m, 9H) ppm; MS: 765.68 m/z [M+H].

### Synthesis of Example 66

### Intermediate 66a: heptadecan-9-one

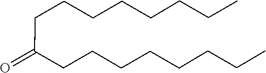 [SMILES: CCCCCCCCC(=O)CCCCCCCC]

To a solution of Intermediate 63a (32.8 g, 127 mmol) in DCM (300 mL) was added Dess-Martin periodinane (81.4 g, 192 mmol). The reaction mixture was stirred at 15° C. for 5 h, concentrated in vacuo, and directly purified using silica gel chromatography (petroleum ether) to provide 20 g (70.5 mmol, 56% yield) of the desired product as a white solid. ¹H NMR (400 MHz, CDCl₃) δ 2.39 (t, J=7.4 Hz, 4H), 1.56 (m, 4H), 1.30 (m, 20H), 0.88 (t, J=6.8 Hz, 6H) ppm.

### Intermediate 66b: methyl 3-octylundec-2-enoate

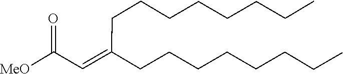 [SMILES: CCCCCCCCC(=CC(=O)OC)CCCCCCCC]

To a solution of methyl 2-dimethoxyphosphorylacetate (2 equiv) in DMF (75 mL) was added NaH (2 equiv) at 15° C. Upon stirring for 1 h, Intermediate 66a (7.5 g, 29.48 mmol, 1 equiv) was added and the reaction mixture was stirred for an additional 1 h. Then, the reaction mixture was warmed to 90-110° C. and stirred for 10-48 h. The reaction mixture was poured into water and extracted into ethyl acetate. The combined organic layers were washed with brine, dried over anhydrous Na₂SO₄, filtered, and concentrated in vacuo. The crude residue was purified using silica gel chromatography to provide 12 g (38.65 mmol, 66% yield) of the desired product as a yellow oil.

### Intermediate 66c: methyl 3-octvlundecanoate

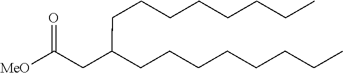 [SMILES: CCCCCCCCC(CCCCCCCC)CC(=O)OC]

To a solution of Intermediate 66b (7.5 g, 24 mmol) in EtOH (75 mL) was added Pd/C (1 g) under N₂. The suspension was degassed under vacuum and purged with H₂ three times. The mixture was stirred under H₂ (15 psi) at 15° C. for 12 h. The reaction mixture was filtered and washed with MeOH (1 L). The filtrate was concentrated in vacuo and directly purified using silica gel chromatography (petroleum ether) to provide 10 g (32.00 mmol, 66% yield) of the desired product as a colorless oil. ¹H NMR (400 MHz, CDCl₃) δ 3.59 (s, 3H), 2.16 (d, J=7.2 Hz, 2H), 1.77 (br m, 1H), 1.19 (m, 28H), 0.81 (t, J=6.8 Hz, 6H) ppm.

### Intermediate 66d: 3-octylundecanoic acid

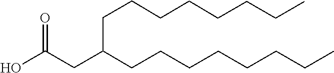 [SMILES: CCCCCCCCC(CCCCCCCC)CC(=O)O]

To a solution of Intermediate 66c (10 g, 32.0 mmol) in EtOH (50 mL) and H₂O (50 mL) was added LiOH.H₂O (2.69 g, 64.0) and NaOH (2.56 g, 64.0 mmol). The reaction mixture was warmed to 60° C. and stirred for 12 h. The reaction mixture was concentrated to remove EtOH and then poured into water (30 mL). The resulting mixture was acidified with 1M HCl (aq.) to pH 6 and then extracted into ethyl acetate (3×60 mL). The combined organic layers were washed with brine (2×40 mL), dried over anhydrous Na₂SO₄, filtered, and concentrated in vacuo. The crude residue was purified using silica gel chromatography (0-50% EtOAc in petroleum ether) to provide 3.6 g (12.4 mmol, 38% yield) of the desired product as a colorless oil. ¹H NMR (400 MHz, CDCl₃) δ 2.20 (br m, 2H), 1.82 (br m, 1H), 1.31 (m, 28H), 0.89 (t, J=6.8 Hz, 6H) ppm.

### Intermediate 66e: 3-hydroxy-2(((3-octylundecanoyl)oxy)methyl)propyl (9Z,12Z)-octadeca-9,12-dienoate

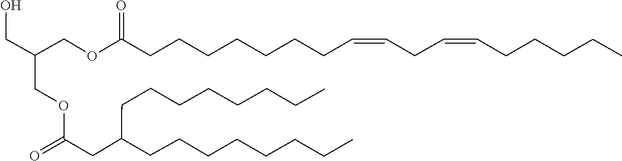 [SMILES: CCCCC/C=C\C/C=C\CCCCCCCC(=O)OCC(CO)COC(=O)CC(CCCCCCCC)CCCCCCCC]

Intermediate 66e was synthesized in 39% yield from Intermediate 1a and Intermediate 66d using the method employed for Intermediate 1d. ¹H NMR (400 MHz, CDCl₃) δ 5.45-5.26 (m, 4H), 4.24-4.10 (m, 4H), 3.61 (t, J=6.0 Hz, 2H), 2.77 (t, J=6.4 Hz, 2H), 2.32 (t, J=7.6 Hz, 2H), 2.28-2.14 (m, 4H), 2.05 (q, J=6.8 Hz, 4H), 1.83 (m, 1H), 1.61 (m, 2H), 1.28 (m, 42H), 0.88 (m, 9H) ppm; MS: 649.67 m/z [M+H].

### Example 66

### 3-(((3-(diethylamino)propoxy)carbonyl)oxy)-2-(((3-octylundecanoyl)oxy)methyl)propyl (9Z,12Z)-octadeca-9,12-dienoate

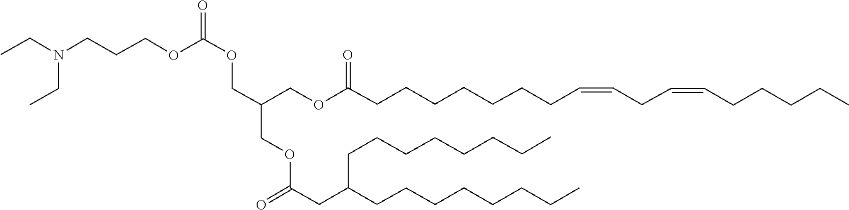 [SMILES: CCCCC/C=C\C/C=C\CCCCCCCC(=O)OCC(COC(=O)CC(CCCCCCCC)CCCCCCCC)COC(=O)OCCCN(CC)CC]

Example 66 was synthesized in 53% yield from Intermediate 66e and 3-(diethylamino)propan-1-ol using the method employed for Example 1. ¹H NMR (500 MHz, CDCl₃) δ 5.43-5.28 (m, 4H), 4.19-4.13 (m, 8H), 2.77 (t, J=6.7 Hz, 2H), 2.50 (q, J=7.0 Hz, 6H), 2.42 (m, 1H), 2.30 (t, J=7.6 Hz, 2H), 2.24 (d, J=6.8 Hz, 2H), 2.05 (m, 4H), 1.80 (m, 3H), 1.61 (m, 2H), 1.42-1.15 (m, 42H), 1.00 (t, J=7.1 Hz, 6H), 0.88 (m, 9H) ppm; MS: 807.58 m/z [M+H].

### Synthesis of Example 67

### Intermediate 67a: methyl 3-heptyldec-2-enoate

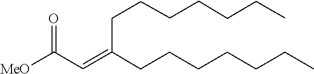 [SMILES: CCCCCCCC(=CC(=O)OC)CCCCCCC]

To a solution of NaH (14.13 g, 353.36 mmol, 2 equiv) in THF (0.4 M) was slowly added methyl 2-dimethoxyphosphorylacetate (64.35 g, 353.36 mmol, 2 equiv) at 0° C. Upon stirring for 1 h, pentadecan-8-one (40 g, 176.68 mmol) was slowly added and the reaction mixture was warmed to 15° C. After additional stirring for 1 h, the reaction mixture was heated to 70° C. and stirred for 48 h. The reaction mixture was cooled to 0° C., diluted with water, and extracted into petroleum ether. The combined organic layers were washed with brine, dried over anhydrous sodium sulfate or magnesium sulfate, filtered and concentrated in vacuo. The crude residue was purified by using silica gel chromatography to provide 20 g (56.65 mmol, 32% yield) of the desired product as a colorless oil.

### Intermediate 67b: 3-heptyldec-2-en-1-ol

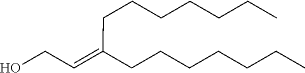 [SMILES: CCCCCCCC(=CCO)CCCCCCC]

To a solution of Intermediate 67a (21.5 g, 76.1 mmol) in THF (200 mL) was added DIBAL (1 M, 228.4 mL) at 0° C. The mixture was stirred at 0° C. for 30 min and then at 15° C. for 12 h. The reaction mixture was diluted with water (20 mL) at 0° C., followed by an additional 200 mL before being extracted into EtOAc (2×200 mL). The combined organic layers were washed with brine (200 mL), dried over anhydrous Na₂SO₄, filtered, and concentrated in vacuo. The crude residue was purified using silica gel chromatography (0-2% EtOAc in petroleum ether) to provide 17 g (40.1 mmol, 53% yield) of the desired product as a colorless oil. ¹H NMR (400 MHz, CDCl₃) δ 5.39 (t, J=7.0 Hz, 1H), 4.16 (d, J=7.2 Hz, 2H), 3.65 (t, J=6.6 Hz, 1H), 2.03 (m, 4H), 1.35-1.28 (m, 20H), 0.89 (t, J=6.8 Hz, 6H) ppm.

### Intermediate 67c: 3-heptyldec-2-enal

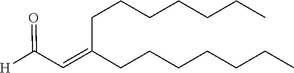 [SMILES: CCCCCCCC(=CC=O)CCCCCCC]

To a stirred suspension of IBX (1.5-3.5 M) in DMSO (1.5-3.5 M) was added Intermediate 67b (17 g, 66.8 mmol, 1 equiv) in THF (0.5-1 M) at 30° C. Upon stirring for at least 2 h at 30° C., the reaction mixture was diluted with petroleum ether, washed with water and brine, dried over anhydrous Na₂SO₄, filtered, and concentrated in vacuo. The crude residue was purified using silica gel chromatography to provide 12 g (47.5 mmol, 71% yield) of the desired product as a colorless oil. ¹H NMR (400 MHz, CDCl₃) δ 9.99 (d, J=8.4 Hz, 1H), 5.86 (d, J=8.4 Hz, 1H), 2.55 (m, 2H), 2.21 (m, 2H) 1.52 (m, 4H), 1.30 (m, 16H), 0.89 (t, J=6.8 Hz, 6H) ppm.

### Intermediate 67d: methyl (E)-5-heptyldodeca-2,4-dienoate

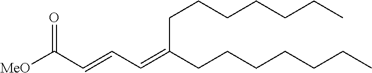 [SMILES: CCCCCCCC(=C/C=C/C(=O)OC)CCCCCCC]

Intermediate 67d was synthesized in 44% yield from Intermediate 67c and methyl 2-dimethoxyphosphorylacetate using the method employed for 67a. ¹H NMR (400 MHz, CDCl₃) δ 7.60 (dd, J=15.0 Hz, 11.8 Hz, 1H), 5.97 (d, J=11.6 Hz, 1H), 5.79 (d, J=15.2 Hz, 1H), 3.75 (s, 3H), 2.27 (t, J=7.6 Hz, 2H), 2.13 (t, J=7.6 Hz, 2H) 1.48-1.29 (m, 20H), 0.89 (t, J=6.8 Hz, 6H) ppm.

### Intermediate 67e: methyl 5-heptyldodecanoate

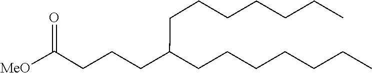 [SMILES: CCCCCCCC(CCCCCCC)CCCC(=O)OC]

To a solution of Intermediate 67d (8 g, 20.75 mmol) in MeOH (100 mL) was added Pd/C (10 g, 339.59 umol, 33.96 10% purity). The suspension was degassed under vacuum and purged with H₂ several times. The mixture was stirred under H₂ (15 psi) at 15° C. for 12 h. The reaction mixture was filtered and concentrated in vacuo to provide 6 g (15.36 mmol, 74% yield) of the desired product as a colorless oil, which did not require further purification. ¹H NMR (400 MHz, CDCl₃) δ 3.68 (s, 3H), 2.29 (t, J=7.6 Hz, 2H), 1.59 (m, 2H), 1.28 (m, 27H), 0.88 (t, J=6.8 Hz, 6H) ppm.

### Intermediate 67f: 5-heptyldodecanoic acid

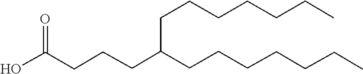 [SMILES: CCCCCCCC(CCCCCCC)CCCC(=O)O]

To a solution of Intermediate 67e (6 g, 15.36 mmol) in THF (120 mL) was added a solution of NaOH (3.07 g, 76.79 mmol) in water (60 mL). The reaction mixture was stirred at 60° C. for 5 h, diluted with water (20 mL), neutralized to pH 4 with 1 M HCl, and extracted into EtOAc (3×20 mL). The combined organic layers were washed with brine (20 mL), dried over Na₂SO₄, filtered, and concentrated in vacuo. The crude residue was purified using silica gel chromatography (0-2% EtOAc in petroleum ether) to provide 3.51 g (11.68 mmol, 76.03% yield) of the desired product as a colorless oil. ¹H NMR (400 MHz, MeOD) δ 2.27 (t, J=7.4 Hz, 2H), 1.59 (m, 2H), 1.29 (m, 27H), 0.91 (t, J=6.8 Hz, 6H) ppm; MS: 297.2 m/z [M−H].

### Intermediate 67g: 3-((5-heptyldodecanoyl)oxy)-2-(hydroxymethyl)propyl (9Z,12Z)-octadeca-9,12-dienoate

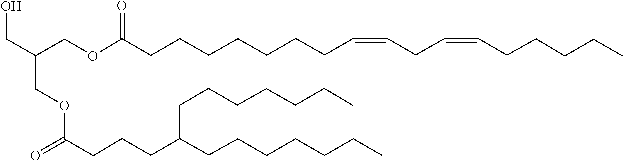 [SMILES: CCCCC/C=C\C/C=C\CCCCCCCC(=O)OCC(CO)COC(=O)CCCC(CCCCCCC)CCCCCCC]

Intermediate 67g was synthesized in 42% yield from Intermediate 1a and Intermediate 67f using the method employed for Intermediate 1d. ¹H NMR (400 MHz, CDCl₃) δ 5.44-5.26 (m, 4H), 4.17 (m, 4H), 3.61 (t, J=5.9 Hz, 2H), 2.76 (m, 2H), 2.31 (q, J=7.3 Hz, 4H), 2.26-2.13 (m, 2H), 2.04 (q, J=6.8 Hz, 4H), 1.59 (m, 5H), 1.41-1.16 (m, 39H), 0.88 (m, 9H) ppm. MS: 649.67 m/z [M+H].

### Example 67

### 3-(((3-(di ethylamino)propoxy)carbonyl)oxy)-2-(((5-heptyldodecanoyl)oxy)methyl)propyl (9Z,12Z)-octadeca-9,12-dienoate

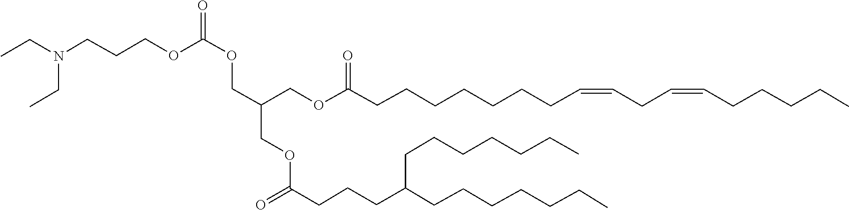 [SMILES: CCCCC/C=C\C/C=C\CCCCCCCC(=O)OCC(COC(=O)CCCC(CCCCCCC)CCCCCCC)COC(=O)OCCCN(CC)CC]

Example 67 was synthesized in 51% yield from Intermediate 67g and 3-(diethylamino)propan-1-ol using the method employed for Example 1. ¹H NMR (500 MHz, CDCl₃) δ 5.42-5.28 (m, 4H), 4.22-4.08 (m, 8H), 2.77 (t, J=6.7 Hz, 2H), 2.50 (q, J=7.0 Hz, 6H), 2.42 (m, 1H), 2.29 (m, 4H), 2.04 (m, 4H), 1.80 (m, 2H), 1.59 (m, 4H), 1.40-1.20 (m, 42H), 1.00 (t, J=7.1 Hz, 6H), 0.88 (m, 9H) ppm; MS: 807.53 m/z [M+H].

### Synthesis of Example 68

### Intermediate 68a: methyl 3-hexylnon-2-enoate

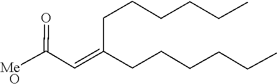 [SMILES: CCCCCCC(=CC(=O)OC)CCCCCC]

Intermediate 68a was synthesized in 55% yield from tridecan-7-one and methyl 2-dimethoxyphosphorylacetate using the method employed for 66b. ¹H NMR (400 MHz, CDCl₃) δ 5.63 (s, 1H), 3.68 (s, 3H), 2.60 (t, J=7.8 Hz, 2H), 2.14 (t, J=7.2 Hz, 2H), 1.45-1.27 (m, 16H), 0.89 (t, J=6.4 Hz, 6H) ppm.

### Intermediate 68b: 3-heptyldec-2-en-1-ol

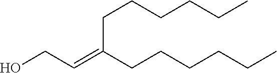 [SMILES: CCCCCCC(=CCO)CCCCCC]

Intermediate 68b was synthesized in 49% yield from Intermediate 68a using the method employed for Intermediate 67b. ¹H NMR (400 MHz, CDCl₃) δ 5.38 (t, J=7.0 Hz, 1H), 4.14 (d, J=7.2 Hz, 2H), 2.01 (m, 4H), 1.39-1.27 (m, 16H), 0.88 (t, J=6.6 Hz, 6H) ppm.

### Intermediate 68c: 3-heptyldec-2-enal

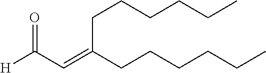 [SMILES: CCCCCCC(=CC=O)CCCCCC]

Intermediate 68c was synthesized in 73% yield from Intermediate 68b using the method employed for Intermediate 67c. ¹H NMR (400 MHz, CDCl₃) δ 9.99 (d, J=8.4 Hz, 1H), 5.85 (d, J=7.6 Hz, 1H), 2.55 (t, J=7.8 Hz, 2H), 2.21 (t, J=7.2 Hz, 2H) 1.49 (m, 4H), 1.31 (m, 12H), 0.89 (t, J=6.8 Hz, 6H) ppm.

### Intermediate 68d: (E)-7-hexyltrideca-4,6-dienoic acid

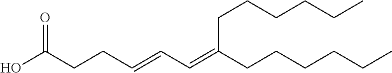 [SMILES: CCCCCCC(=C/C=C/CCC(=O)O)CCCCCC]

To a solution of Intermediate 68c (9 g, 40.1 mmol) in HMPA (8 mL) and THF (104 mL) was added NaHMDS (1 M, 160.4 mL) at 0° C. 3-carboxypropyl(triphenyl)phosphonium (22.42 g, 64.18 mmol) in THF (28 mL) was added to the reaction mixture, which was further stirred at 15° C. for 12 h. The reaction mixture was poured into water (200 mL), acidified with 2N HCl (aq.), and extracted into EtOAc (4×150 mL). The combined organic layers were washed with brine (200 mL), dried over anhydrous Na₂SO₄, filtered, and concentrated in vacuo. The crude residue was purified using silica gel chromatography (1-100% EtOAc in petroleum ether) to provide 8 g (19.0 mmol, 24% yield) of the desired product as a yellow oil. ¹H NMR (400 MHz, CDCl₃) δ 6.25 (m, 1H), 6.05 (d, J=7.6 Hz, 1H), 5.30 (m, 1H), 2.59-2.45 (m, 4H), 2.13-2.07 (m, 4H), 1.40-1.29 (m, 16H), 0.89 (t, J=6.6 Hz, 6H) ppm; MS: 295.2 [M+H].

### Intermediate 68e: 7-hexyltridecanoic acid

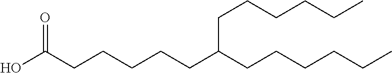 [SMILES: CCCCCCC(CCCCCC)CCCCCC(=O)O]

To a solution of Intermediate 68d (4 g, 13.6 mmol) in MeOH (50 mL) was added Pd/C (0.4 g, 13.58 mmol) under N₂. The suspension was degassed under vacuum and purged with H₂ several times. The reaction mixture was stirred under H₂ (15 psi) at 35° C. for 12 h, filtered and washed with MeOH (300 mL), and concentrated in vacuo. The crude residue was purified using silica gel chromatography (petroleum ether) to provide 5.3 g (16.0 mmol, 59% yield) of the desired product as a yellow oil. ¹H NMR (400 MHz, CDCl₃) δ 2.35 (t, J=7.4 Hz, 2H), 1.65 (m, 2H), 1.25 (m, 27H), 0.89 (t, J=6.6 Hz, 6H) ppm; MS: 297.2 m/z [M−H].

### Intermediate 68f: 3-((7-hexyltridecanoyl)oxy)-2-(hydroxymethyl)propyl (9Z,12Z)-octadeca-9,12-dienoate

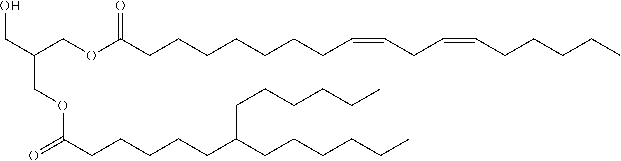 [SMILES: CCCCC/C=C\C/C=C\CCCCCCCC(=O)OCC(CO)COC(=O)CCCCCC(CCCCCC)CCCCCC]

Intermediate 68f was synthesized in 60% yield from Intermediate 1a and Intermediate 68e using the method employed for Intermediate 1d. ¹H NMR (400 MHz, CDCl₃) δ 5.45-5.27 (m, 4H), 4.17 (m, 4H), 3.61 (t, J=5.2 Hz, 2H), 2.77 (t, J=6.4 Hz, 2H), 2.32 (t, J=7.6 Hz, 4H), 2.25-2.14 (m, 2H), 2.04 (q, J=6.8 Hz, 4H), 1.68-1.55 (m, 5H), 1.40-1.15 (m, 39H), 0.88 (m, 9H) ppm; MS: 649.72 m/z [M+H].

### Example 68

### 3-(((3-(diethylamino)propoxy)carbonyl)oxy)-2-(((7-hexyltridecanoyl)oxy)methyl)propyl (9Z,12Z)-octadeca-9,12-dienoate

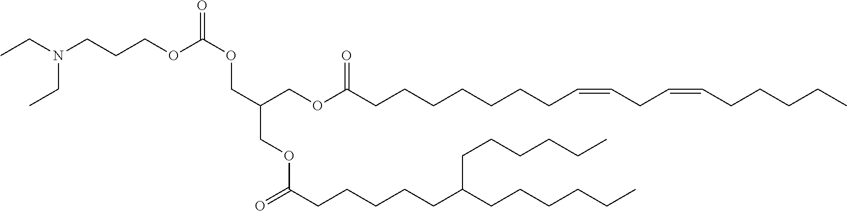 [SMILES: CCCCC/C=C\C/C=C\CCCCCCCC(=O)OCC(COC(=O)CCCCCC(CCCCCC)CCCCCC)COC(=O)OCCCN(CC)CC]

Example 68 was synthesized in 43% yield from Intermediate 68f and 3-(diethylamino)propan-1-ol using the method employed for Example 1. ¹H NMR (400 MHz, CDCl₃) δ 5.43-5.27 (m, 4H), 4.22-4.07 (m, 8H), 2.77 (t, J=6.5 Hz, 2H), 2.55-2.46 (m, 6H), 2.46-2.37 (m, 1H), 2.30 (t, J=7.6 Hz, 4H), 2.04 (m, 4H), 1.86-1.75 (m, 2H), 1.61 (t, J=7.3 Hz, 4H), 1.42-1.11 (m, 41H), 1.00 (t, J=7.1 Hz, 6H), 0.88 (m, 9H) ppm; MS: 807.72 m/z [M+H].

### Synthesis of Example 69

### Intermediate 69a: (Z)-11-(non-2-en-1-yloxy)-11-oxoundecanoic acid

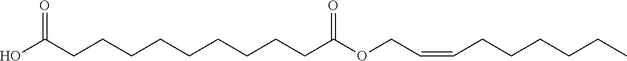 [SMILES: CCCCCC/C=C\COC(=O)CCCCCCCCCC(=O)O]

Intermediate 69a was synthesized in 36% yield from undecanedioic acid and (Z)-non-2-en-1-ol using the method employed for Intermediate 54a. ¹H NMR (400 MHz, CDCl₃) δ 5.65 (m, 1H), 5.52 (m, 1H), 4.61 (dd, J=6.9, 1.2 Hz, 2H), 2.32 (m, 4H), 2.09 (m, 2H), 1.61 (m, 4H), 1.41-1.20 (m, 18H), 0.88 (t, J=6.8 Hz, 3H) ppm.

### Intermediate 69b: (Z)-1-(3-((4,4-bis(octyloxy)butanoyl)oxy)-2-(hydroxymethyl)propyl) 11-(non-2-en-1-yl) undecanedioate

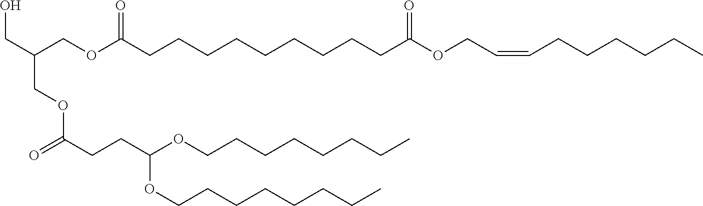 [SMILES: CCCCCC/C=C\COC(=O)CCCCCCCCCC(=O)OCC(CO)COC(=O)CCC(OCCCCCCCC)OCCCCCCCC]

Intermediate 69b was synthesized in 35% yield from Intermediate 69a and Intermediate 54b using the method employed for Intermediate 54c. ¹H NMR (400 MHz, CDCl₃) δ 5.64 (m, 1H), 5.52 (m, 1H), 4.62 (d, J=6.7 Hz, 2H), 4.49 (t, J=5.5 Hz, 1H), 4.24-4.10 (m, 4H), 3.67-3.52 (m, 4H), 3.40 (dt, J=9.3, 6.7 Hz, 2H), 2.41 (t, J=7.5 Hz, 2H), 2.30 (q, J=7.1 Hz, 4H), 2.21 (m, 2H), 2.14-2.05 (m, 2H), 1.93 (td, J=7.5, 5.4 Hz, 2H), 1.57 (m, 9H), 1.40-1.21 (m, 36H), 0.88 (m, 9H) ppm; MS: 777.78 m/z [M+Na].

### Example 69

### (Z)-1-(3-((4,4-bi s(octyloxy)butanoyl)oxy)-2-((((3-(diethylamino)propoxy)carbonyl)oxy)methyl)propyl) 11-(non-2-en-1-yl) undecanedioate

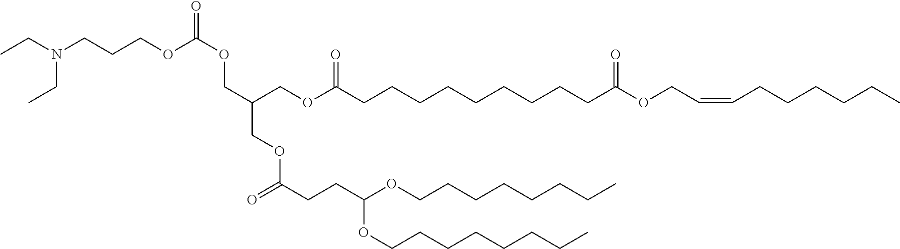 [SMILES: CCCCCC/C=C\COC(=O)CCCCCCCCCC(=O)OCC(COC(=O)CCC(OCCCCCCCC)OCCCCCCCC)COC(=O)OCCCN(CC)CC]

Example 69 was synthesized in 19% yield from Intermediate 69b and 3-(diethylamino)propan-1-ol using the method employed for Example 1. ¹H NMR (400 MHz, CDCl₃) δ 5.62 (m, 1H), 5.53 (m, 1H), 4.61 (d, J=7.0, 1.3 Hz, 2H), 4.48 (t, J=5.5 Hz, 1H), 4.22-4.09 (m, 8H), 3.55 (m, 2H), 3.39 (m, 2H), 2.56 (q, J=7.2 Hz, 6H), 2.47-2.35 (m, 3H), 2.29 (t, J=7.6 Hz, 4H), 2.09 (m, 2H), 1.97-1.78 (m, 4H), 1.65-1.49 (m, 8H), 1.40-1.24 (m, 35H), 1.03 (t, J=7.2 Hz, 6H), 0.88 (m, 9H) ppm; MS: 913.37 m/z [M+H].

### Synthesis of Example 70

### Intermediate 70a: ((Z)-13-(non-2-en-1-yloxy)-13-oxotridecanoic acid

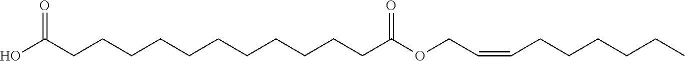 [SMILES: CCCCCC/C=C\COC(=O)CCCCCCCCCCCC(=O)O]

Intermediate 70a was synthesized in 40% yield from tridecanedioic acid and (Z)-non-2-en-1-ol using the method employed for Intermediate 54a. ¹H NMR (400 MHz, CDCl₃) δ 5.62 (m, 1H), 5.50 (m, 1H), 4.59 (dd, J=6.8, 1.2 Hz, 2H), 2.29 (m, 4H), 2.07 (m, 2H), 1.59 (m, 4H), 1.39-1.18 (m, 22H), 0.85 (t, J=6.8 Hz, 3H) ppm.

### Intermediate 70b: (Z)-1-(3-((4,4-bis(octyloxy)butanoyl)oxy)-2-(hydroxymethyl)propyl) 13-(non-2-en-1-yl) tridecanedioate

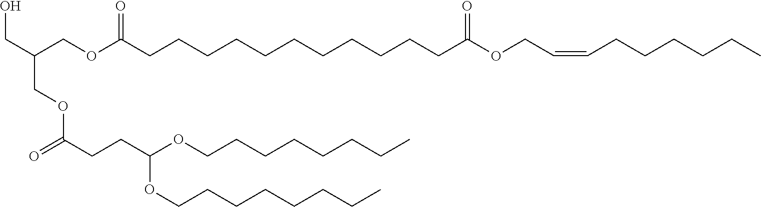 [SMILES: CCCCCC/C=C\COC(=O)CCCCCCCCCCCC(=O)OCC(CO)COC(=O)CCC(OCCCCCCCC)OCCCCCCCC]

Intermediate 70b was synthesized in 34% yield from Intermediate 70a and Intermediate 54b using the method employed for Intermediate 54c. ¹H NMR (500 MHz, CDCl₃) δ 5.64 (m, 1H), 5.52 (m, 1H), 4.62 (d, J=6.8 Hz, 2H), 4.49 (t, J=5.5 Hz, 1H), 4.17 (m, 4H), 3.62 (t, J=5.7 Hz, 2H), 3.56 (dt, J=9.3, 6.7 Hz, 2H), 3.40 (dt, J=9.3, 6.7 Hz, 2H), 2.41 (t, J=7.5 Hz, 2H), 2.31 (q, J=7.9 Hz, 4H), 2.25-2.15 (m, 2H), 2.09 (q, J=7.3 Hz, 2H), 1.93 (td, J=7.5, 5.4 Hz, 2H), 1.58 (m, 8H), 1.39-1.21 (m, 41H), 0.88 (m, 9H) ppm; MS: 805.63 m/z [M+Na].

### Example 70

### (Z)-1-(3-((4,4-bi s(octyloxy)butanoyl)oxy)-2-((((3-(diethylamino)propoxy)carbonyl)oxy)methyl)propyl) 13-(non-2-en-1-yl) tridecanedioate

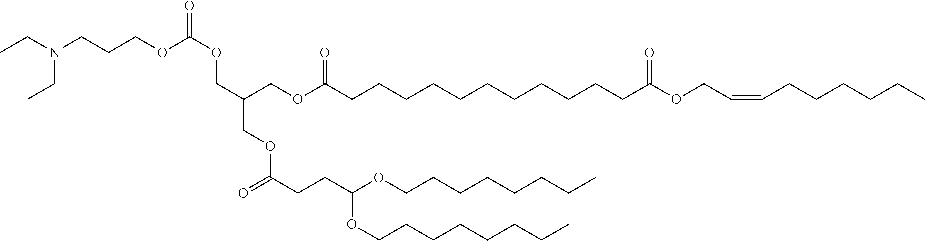 [SMILES: CCCCCC/C=C\COC(=O)CCCCCCCCCCCC(=O)OCC(COC(=O)CCC(OCCCCCCCC)OCCCCCCCC)COC(=O)OCCCN(CC)CC]

Example 70 was synthesized in 15% yield from Intermediate 70b and 3-(diethylamino)propan-1-ol using the method employed for Example 1. ¹H NMR (400 MHz, CDCl₃) δ 5.64 (m, 1H), 5.51 (m, 1H), 4.61 (d, J=6.9, 1.2 Hz, 2H), 4.48 (t, J=5.6 Hz, 1H), 4.22-4.09 (m, 8H), 3.55 (m, 2H), 3.40 (m, 2H), 2.51 (q, J=7.0 Hz, 6H), 2.46-2.35 (m, 3H), 2.34-2.26 (m, 4H), 2.15-2.04 (m, 2H), 1.97-1.87 (m, 2H), 1.86-1.75 (m, 4H), 1.67-1.49 (m, 8H), 1.40-1.23 (m, 40H), 1.01 (t, J=7.2 Hz, 6H), 0.88 (m, 9H) ppm; MS: 942.04 m/z [M+H].

### Synthesis of Example 71-74

The following examples were synthesized from Intermediate 59b and an amino alcohol or diamine reagent using the method employed for Example 1.

### Example 71

### 1-(3-((4,4-bis(octyloxy)butanoyl)oxy)-2-(((((1-methylpiperidin-3-yl)methoxy)carbonyl)oxy)methyl)propyl) 9-decyl nonanedioate

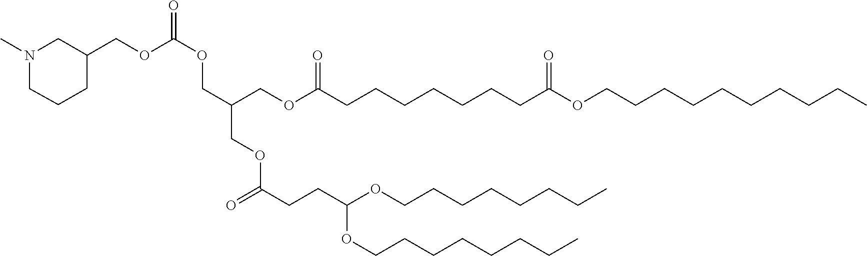 [SMILES: CCCCCCCCCCOC(=O)CCCCCCCC(=O)OCC(COC(=O)CCC(OCCCCCCCC)OCCCCCCCC)COC(=O)OCC1CCCN(C)C1]

19% yield; ¹H NMR (400 MHz, CDCl₃) δ 4.48 (t, J=5.5 Hz, 1H), 4.19 (d, J=6.0 Hz, 2H), 4.14 (dt, J=6.0, 1.4 Hz, 4H), 4.05 (t, J=6.7 Hz, 3H), 3.96 (dd, J=10.6, 7.3 Hz, 1H), 3.56 (dt, J=9.3, 6.7 Hz, 2H), 3.40 (dt, J=9.3, 6.7 Hz, 2H), 2.78 (m, 2H), 2.47-2.36 (m, 3H), 2.33-2.23 (m, 7H), 2.07-1.86 (m, 4H), 1.79-1.50 (m, 16H), 1.29 (m, 38H), 1.00 (m, 1H), 0.88 (m, 9H) ppm; MS: 899.87 m/z [M+H].

### Example 72

### 1-(3-((4,4-bis(octyloxy)butanoyl)oxy)-2-(((((1-ethylpiperidin-3-yl)methoxy)carbonyl)oxy)methyl)propyl) 9-decyl nonanedioate

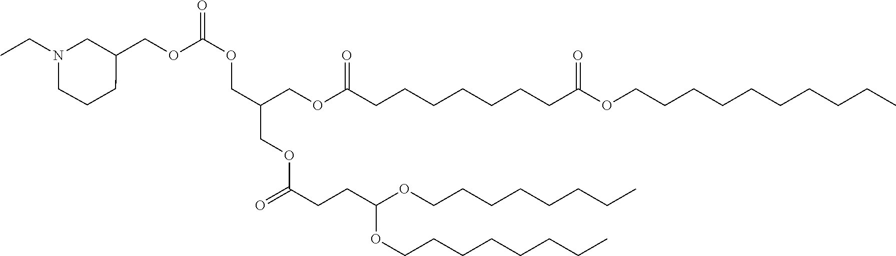 [SMILES: CCCCCCCCCCOC(=O)CCCCCCCC(=O)OCC(COC(=O)CCC(OCCCCCCCC)OCCCCCCCC)COC(=O)OCC1CCCN(CC)C1]

19% yield; ¹H NMR (400 MHz, CDCl₃) δ 4.48 (t, J=5.5 Hz, 1H), 4.19 (d, J=6.0 Hz, 2H), 4.14 (dt, J=6.0, 1.4 Hz, 4H), 4.05 (td, J=6.9, 6.4, 3.7 Hz, 3H), 3.96 (dd, J=10.7, 7.2 Hz, 1H), 3.56 (dt, J=9.3, 6.7 Hz, 2H), 3.40 (dt, J=9.3, 6.7 Hz, 2H), 2.87 m, 2H), 2.48-2.36 (m, 5H), 2.34-2.24 (m, 4H), 2.05-1.85 (m, 3H), 1.79-1.50 (m, 16H), 1.39-1.20 (m, 40H), 1.12-0.97 (m, 3H), 0.87 (m, 9H) ppm; MS: 913.46 m/z [M+H].

### Example 73

### 1-(3-((4,4-bis(octyloxy)butanoyl)oxy)-2-((((2-(diethylamino)ethyl)carbamoyl)oxy)methyl)propyl) 9-decyl nonanedioate

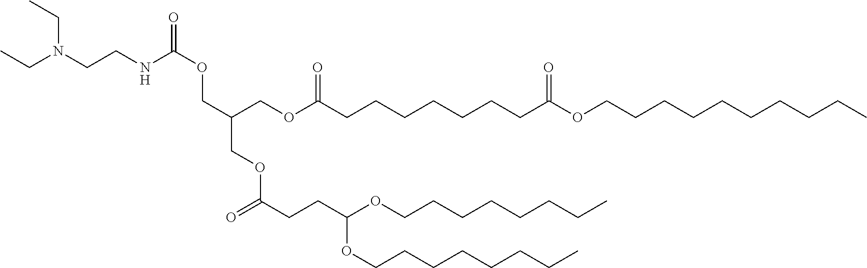 [SMILES: CCCCCCCCCCOC(=O)CCCCCCCC(=O)OCC(COC(=O)CCC(OCCCCCCCC)OCCCCCCCC)COC(=O)NCCN(CC)CC]

14% yield; ¹H NMR (400 MHz, CDCl₃) δ 5.22 (br s, 1H), 4.48 (t, J=5.6 Hz, 1H), 4.20-4.08 (m, 6H), 4.05 (t, J=6.8 Hz, 2H), 3.56 (dt, J=9.3, 6.7 Hz, 2H), 3.40 (dt, J=9.3, 6.7 Hz, 2H), 3.21 (br m, 2H), 2.52 (q, J=7.1, 6.2 Hz, 6H), 2.40 (t, J=7.6 Hz, 2H), 2.29 (td, J=7.6, 5.9 Hz, 4H), 1.92 (td, J=7.6, 5.5 Hz, 2H), 1.76-1.49 (m, 13H), 1.39-1.20 (m, 40H), 1.00 (t, J=7.1 Hz, 6H), 0.88 (m, 9H) ppm; MS: 886.69 m/z [M+H].

### Example 74

### 1-(3-((4,4-bis(octyloxy)butanoyl)oxy)-2-((((2-(1-methylpyrrolidin-2-yl)ethoxy)carbonyl)oxy)methyl)propyl) 9-decyl nonanedioate

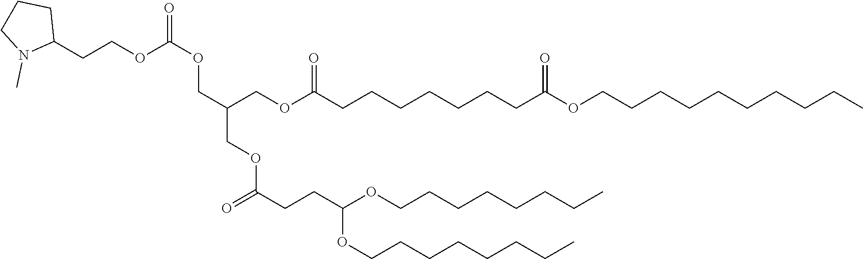 [SMILES: CCCCCCCCCCOC(=O)CCCCCCCC(=O)OCC(COC(=O)CCC(OCCCCCCCC)OCCCCCCCC)COC(=O)OCCC1CCCN1C]

26% yield; ¹H NMR (400 MHz, CDCl₃) δ 4.48 (t, J=5.6 Hz, 1H), 4.28-4.09 (m, 8H), 4.05 (t, J=6.8 Hz, 2H), 3.56 (dt, J=9.3, 6.7 Hz, 2H), 3.40 (dt, J=9.3, 6.7 Hz, 2H), 3.05 (m, 1H), 2.46-2.36 (m, 3H), 2.34-2.24 (m, 7H), 2.21-1.87 (m, 4H), 1.84-1.43 (m, 12H), 1.40-1.18 (m, 40H), 0.88 (m, 9H) ppm; MS: 899.38 m/z [M+H].

### Synthesis of Example 75

### Example 75

### 1-(3-((4,4-bis(octyloxy)butanoyl)oxy)-2-(((4-(diethylamino)butanoyl)oxy)methyl)propyl) 9-decyl nonanedioate

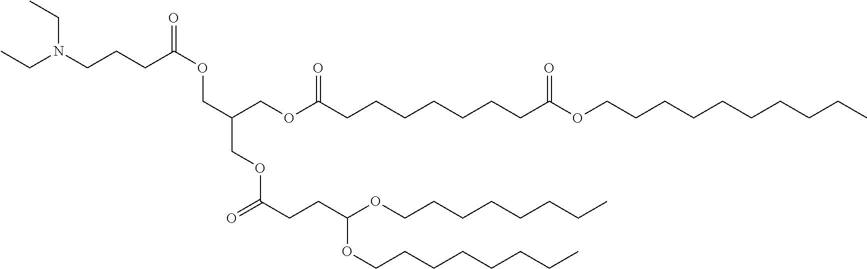 [SMILES: CCCCCCCCCCOC(=O)CCCCCCCC(=O)OCC(COC(=O)CCCN(CC)CC)COC(=O)CCC(OCCCCCCCC)OCCCCCCCC]

Example 75 was synthesized in 61% yield from Intermediate 59b and 4-(diethylamino)butanoic acid using the method employed for Example 25. ¹H NMR (400 MHz, CDCl₃) δ 4.48 (t, J=5.5 Hz, 1H), 4.12 (dd, J=6.1, 1.6 Hz, 6H), 4.05 (t, J=6.8 Hz, 2H), 3.56 (dt, J=9.3, 6.7 Hz, 2H), 3.40 (dt, J=9.3, 6.7 Hz, 2H), 2.50 (q, J=7.1 Hz, 4H), 2.45-2.24 (m, 10H), 1.92 (m, 2H), 1.75 (m, 2H), 1.65-1.50 (m, 12H), 1.38-1.20 (m, 40H), 1.00 (t, J=7.1 Hz, 6H), 0.88 (m, 8H) ppm; MS: 885.43 m/z [M+H].

### Synthesis of Example 76

### Intermediate 76a: 1-decyl 9-(3-hydroxy-2-(hydroxymethyl)propyl) nonanedioate

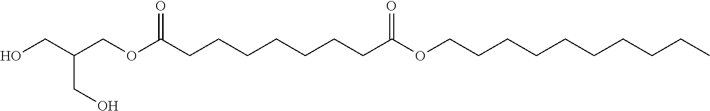 [SMILES: CCCCCCCCCCOC(=O)CCCCCCCC(=O)OCC(CO)CO]

To a solution of Intermediate 59a (15 g, 45.6 mmol, 1 equiv), (2,2-dimethyl-1,3-dioxan-5-yl)methanol (1-1.2 equiv), DMAP (0.2 equiv), and DIPEA (1.5-3 equiv) in DCM (0.2 M) was added EDC.HCl (1.5 equiv) at rt. The reaction mixture was stirred for at least 16 h, diluted with water, washed sequentially with 1M HCl and 5% sodium bicarbonate, dried over magnesium sulfate, filtered, and concentrated in vacuo. The crude acetonide-protected intermediate was resuspended in MeOH and Dowex® 50W X8 resin was added. The resulting mixture was stirred at rt for at least 12 h, filtered and washed with MeOH, and concentrated in vacuo. The crude residue was purified using silica gel chromatography (gradient of EtOAc in hexanes) to provide 12.2 g (29.3 mmol, 65% yield) of the desired product as a white solid. ¹H NMR (400 MHz, CDCl₃) δ 4.24 (d, J=6.3 Hz, 2H), 4.04 (t, J=6.7 Hz, 2H), 3.76 (m, 4H), 2.52 (br s, 2H), 2.30 (dt, J=16.1, 7.5 Hz, 4H), 2.03 (m, 1H), 1.61 (m, 6H), 1.38-1.20 (m, 20H), 0.87 (t, J=6.8 Hz, 3H) ppm; MS: 439.56 m/z [M+Na].

### Intermediate 76b: 1-decyl 9-(3-((7-(heptadecan-9-yloxy)-7-oxoheptanoyl)oxy)-2-(hydroxymethyl)propyl) nonanedioate

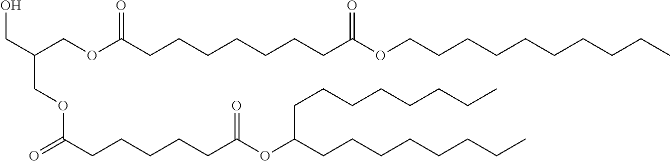 [SMILES: CCCCCCCCCCOC(=O)CCCCCCCC(=O)OCC(CO)COC(=O)CCCCCC(=O)OC(CCCCCCCC)CCCCCCCC]

Intermediate 76b was synthesized in 37% yield from Intermediate 76a and Intermediate 64a using the method employed for Intermediate 1d. ¹H NMR (400 MHz, CDCl₃) δ 4.85 (m, 1H), 4.21-4.12 (m, 4H), 4.04 (t, J=6.8 Hz, 2H), 3.61 (d, J=5.6 Hz, 2H), 2.37-2.24 (m, 8H), 2.18 (m, 1H), 1.63 (m, 11H), 1.49 (m, 7H), 1.39-1.19 (m, 42H), 0.87 (m, 9H) ppm; MS: 797.96 m/z [M+H].

### Example 76

### 1-decyl 9-(3-(((3-(diethylamino)propoxy)carbonyl)oxy)-2-(((7-(heptadecan-9-yloxy)-7-oxoheptanoyl)oxy)methyl)propyl) nonanedioate

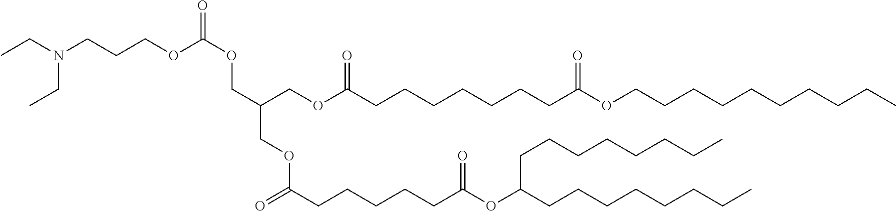 [SMILES: CCCCCCCCCCOC(=O)CCCCCCCC(=O)OCC(COC(=O)CCCCCC(=O)OC(CCCCCCCC)CCCCCCCC)COC(=O)OCCCN(CC)CC]

Example 76 was synthesized in 22% yield from Intermediate 76b and 3-(diethylamino)propan-1-ol using the method employed for Example 1. ¹H NMR (400 MHz, CDCl₃) δ 4.85 (m, 1H), 4.22-4.10 (m, 8H), 4.05 (t, J=6.8 Hz, 2H), 2.55-2.50 (m, 6H), 2.41 (m, 1H), 2.35-2.24 (m, 8H), 1.81 (m, 2H), 1.70-1.55 (m, 12H), 1.50 (m, 4H), 1.41-1.23 (m, 44H), 1.00 (t, J=7.1 Hz, 6H), 0.88 (m, 9H) ppm; MS: 956.17 m/z [M+H].

### Synthesis of Example 77

### Intermediate 77a: 1-decyl 9-(3-((5-(heptadecan-9-yloxy)-5-oxopentanoyl)oxy)-2-(hydroxymethyl)propyl) nonanedioate

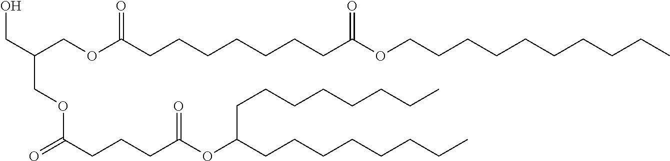 [SMILES: CCCCCCCCCCOC(=O)CCCCCCCC(=O)OCC(CO)COC(=O)CCCC(=O)OC(CCCCCCCC)CCCCCCCC]

Intermediate 77a was synthesized in 26% yield from Intermediate 76a and Intermediate 63b using the method employed for Intermediate 1d. ¹H NMR (400 MHz, CDCl₃) δ 4.86 (m, 1H), 4.23-4.13 (m, 4H), 4.05 (t, J=6.8 Hz, 2H), 3.62 (t, J=5.5 Hz, 2H), 2.33 (m, 8H), 2.19 (m, 1H), 1.95 (m, 2H), 1.61 (m, 8H), 1.50 (m, 4H), 1.38-1.19 (m, 42H), 0.87 (m, 9H) ppm; MS: 769.91 m/z [M+H].

### Example 77

### 1-decyl 9-(3-(((3-(diethylamino)propoxy)carbonyl)oxy)-2-(((5-(heptadecan-9-yloxy)-5-oxopentanoyl)oxy)methyl)propyl) nonanedioate

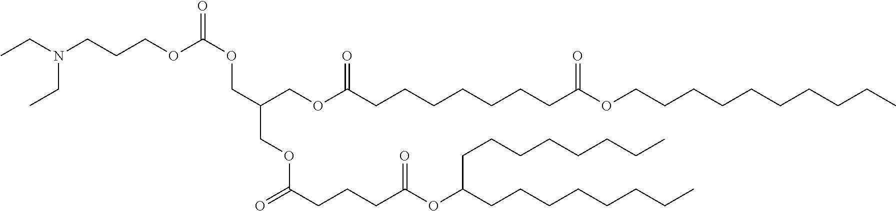 [SMILES: CCCCCCCCCCOC(=O)CCCCCCCC(=O)OCC(COC(=O)CCCC(=O)OC(CCCCCCCC)CCCCCCCC)COC(=O)OCCCN(CC)CC]

Example 77 was synthesized from Intermediate 77a and 3-(diethylamino)propan-1-ol using the method employed for Example 1. ¹H NMR (400 MHz, CDCl₃) δ 4.87 (m, 1H), 4.16 (m, 8H), 4.05 (t, J=6.8 Hz, 2H), 2.51 (m, 6H), 2.46-2.24 (m, 9H), 1.94 (m, 2H), 1.87-1.76 (m, 2H), 1.69-1.57 (m, 6H), 1.49 (m, 4H), 1.40-1.17 (m, 44H), 1.00 (t, J=7.1 Hz, 6H), 0.89 (m, 9H) ppm; MS: 928.07 m/z [M+H].

### Synthesis of Example 78

### Intermediate 78a: 4,4-bis(heptyloxy)butanenitrile

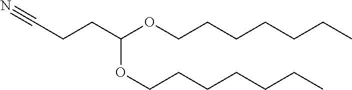 [SMILES: CCCCCCCOC(CCC#N)OCCCCCCC]

Intermediate 78a was synthesized in 99% yield from 4,4-diethoxybutanenitrile and heptan-1-ol using the method employed for Intermediate 1b. ¹H NMR (400 MHz, CDCl₃) δ 4.55 (t, J=5.3 Hz, 1H), 3.60 (dt, J=9.3, 6.6 Hz, 2H), 3.43 (dt, J=9.3, 6.6 Hz, 2H), 2.42 (t, J=7.4 Hz, 2H), 1.94 (td, J=7.4, 5.3 Hz, 2H), 1.57 (m, 4H), 1.40-1.23 (m, 16H), 0.88 (m, 6H) ppm.

### Intermediate 78b: 4,4-bis(heptyloxy)butanoic acid

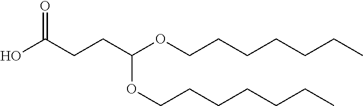 [SMILES: CCCCCCCOC(CCC(=O)O)OCCCCCCC]

Intermediate 78b was synthesized in 92% yield from Intermediate 78a using the method employed for Intermediate 1c. ¹H NMR (400 MHz, CDCl₃) δ 8.85 (br s, 1H), 4.46 (t, J=5.6 Hz, 1H), 3.52 (dt, J=9.4, 6.8 Hz, 2H), 3.39 (dt, J=9.3, 6.8 Hz, 2H), 2.26 (t, J=7.6 Hz, 2H), 1.85 (q, J=7.0 Hz, 2H), 1.53 (m, 4H), 1.29 (m, 16H), 0.94-0.80 (m, 6H) ppm; MS: 315 m/z [M−H].

### Intermediate 78c: 1-(3-((4,4-bis(heptyloxy)butanoyl)oxy)-2-(hydroxymethyl)propyl) 9-decyl nonanedioate

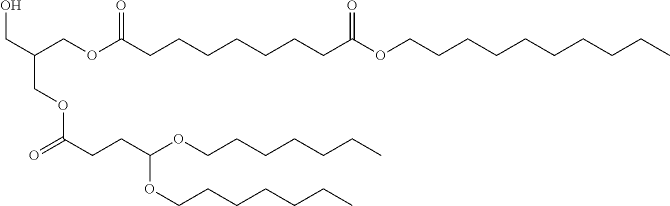 [SMILES: CCCCCCCCCCOC(=O)CCCCCCCC(=O)OCC(CO)COC(=O)CCC(OCCCCCCC)OCCCCCCC]

Intermediate 78c was synthesized in 46% yield from Intermediate 76a and Intermediate 78b using the method employed for Intermediate 1d. ¹H NMR (400 MHz, CDCl₃) δ 4.48 (t, J=5.5 Hz, 1H), 4.23-4.11 (m, 4H), 4.04 (t, J=6.7 Hz, 2H), 3.65-3.50 (m, 4H), 3.39 (dt, J=9.3, 6.7 Hz, 2H), 2.40 (t, J=7.5 Hz, 2H), 2.29 (m, 5H), 2.19 (m, 1H), 1.93 (td, J=7.5, 5.4 Hz, 2H), 1.68-1.49 (m, 11H), 1.38-1.19 (m, 34H), 0.88 (m, 9H) ppm; MS: 737.82 m/z [M+Na].

### Example 78

### 1-(3-((4,4-bis(heptyloxy)butanoyl)oxy)-2-((((3-(diethylamino)propoxy)carbonyl)oxy)methyl)propyl) 9-decyl nonanedioate

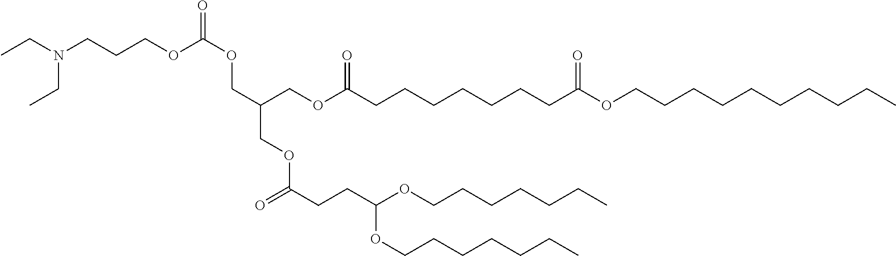 [SMILES: CCCCCCCCCCOC(=O)CCCCCCCC(=O)OCC(COC(=O)CCC(OCCCCCCC)OCCCCCCC)COC(=O)OCCCN(CC)CC]

Example 78 was synthesized in 35% yield from Intermediate 78c and 3-(diethylamino)propan-1-ol using the method employed for Example 1. ¹H NMR (400 MHz, CDCl₃) δ 4.48 (t, J=5.6 Hz, 1H), 4.22-4.09 (m, 8H), 4.05 (t, J=6.8 Hz, 2H), 3.56 (m, 2H), 3.40 (m, 2H), 2.51 (m, 6H), 2.46-2.35 (m, 3H), 2.29 (m, 4H), 1.92 (m, 2H), 1.81 (m, 2H), 1.66-1.49 (m, 12H), 1.39-1.23 (m, 34H), 1.00 (t, J=7.1 Hz, 6H), 0.88 (m, 9H) ppm; MS: 873.52 m/z [M+H].

### Synthesis of Example 79

### Intermediate 79a: 4,4-bis(nonyloxy)butanenitrile

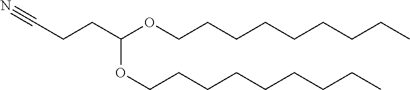 [SMILES: CCCCCCCCCOC(CCC#N)OCCCCCCCCC]

Intermediate 79a was synthesized in 16% yield from 4,4-diethoxybutanenitrile and nonan-1-ol using the method employed for Intermediate 1b. ¹H NMR (400 MHz, CDCl₃) δ 4.55 (t, J=5.3 Hz, 1H), 3.60 (dt, J=9.2, 6.6 Hz, 2H), 3.43 (dt, J=9.3, 6.6 Hz, 2H), 2.42 (t, J=7.4 Hz, 2H), 1.94 (td, J=7.4, 5.3 Hz, 2H), 1.57 (m, 4H), 1.38-1.24 (m, 24H), 0.88 (t, J=6.7 Hz, 6H) ppm.

### Intermediate 79b: 4,4-bis(nonyloxy)butanoic acid

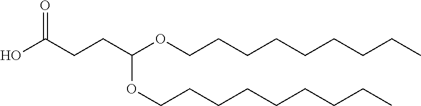 [SMILES: CCCCCCCCCOC(CCC(=O)O)OCCCCCCCCC]

Intermediate 79b was synthesized in 100% yield from Intermediate 79a using the method employed for Intermediate 1c. ¹H NMR (400 MHz, CDCl₃) δ 5.32 (br s, 1H), 4.44 (t, J=5.6 Hz, 1H), 3.49 (dt, J=9.3, 6.9 Hz, 2H), 3.38 (dt, J=9.4, 6.9 Hz, 2H), 2.10 (t, J=7.6 Hz, 2H), 1.78 (q, J=7.0 Hz, 2H), 1.53 (m, 4H), 1.27 (m, 24H), 0.88 (t, J=6.6 Hz, 6H) ppm; MS: 371 m/z [M−H].

### Intermediate 79c: 1-(3-((4,4-bis(nonyloxy)butanoyl)oxy)-2-(hydroxymethyl)propyl) 9-decyl nonanedioate

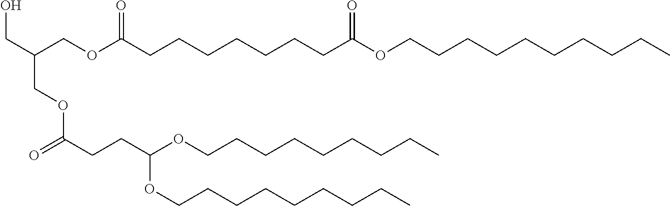 [SMILES: CCCCCCCCCCOC(=O)CCCCCCCC(=O)OCC(CO)COC(=O)CCC(OCCCCCCCCC)OCCCCCCCCC]

Intermediate 79c was synthesized in 43% yield from Intermediate 76a and Intermediate 79b using the method employed for Intermediate 1d. ¹H NMR (400 MHz, CDCl₃) δ 4.48 (t, J=5.5 Hz, 1H), 4.23-4.12 (m, 4H), 4.05 (t, J=6.8 Hz, 2H), 3.67-3.51 (m, 4H), 3.40 (dt, J=9.3, 6.7 Hz, 2H), 2.40 (t, J=7.5 Hz, 2H), 2.30 (dt, J=11.9, 7.6 Hz, 5H), 2.19 (m, 1H), 1.93 (td, J=7.6, 5.4 Hz, 2H), 1.66-1.48 (m, 11H), 1.37-1.20 (m, 42H), 0.88 (m, 9H) ppm; MS: 793.91 m/z [M+Na].

### Example 79

### 1-(3-((4,4-bis(nonyloxy)butanoyl)oxy)-2-((((3-(diethylamino)propoxy)carbonyl)oxy)methyl)propyl) 9-decyl nonanedioate

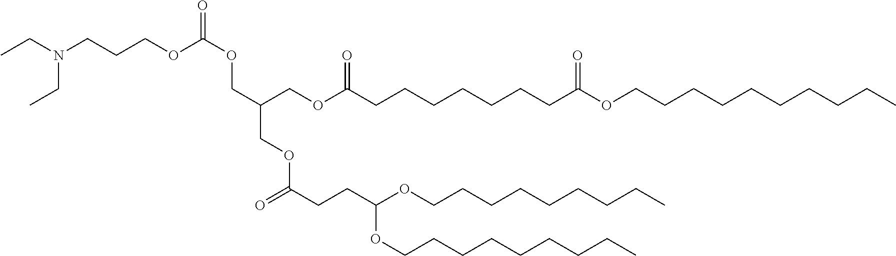 [SMILES: CCCCCCCCCCOC(=O)CCCCCCCC(=O)OCC(COC(=O)CCC(OCCCCCCCCC)OCCCCCCCCC)COC(=O)OCCCN(CC)CC]

Example 79 was synthesized in 35% yield from Intermediate 79c and 3-(diethylamino)propan-1-ol using the method employed for Example 1. ¹H NMR (400 MHz, CDCl₃) δ 4.48 (t, J=5.5 Hz, 1H), 4.22-4.10 (m, 8H), 4.05 (t, J=6.8 Hz, 2H), 3.55 (m, 2H), 3.39 (m, 2H), 2.50 (m, 6H), 2.45-2.35 (m, 3H), 2.29 (m, 4H), 1.92 (m, 2H), 1.86-4 1.74 (m, 3H), 1.66-1.49 (m, 10H), 1.35-1.23 (m, 43H), 1.00 (t, J=7.1 Hz, 6H), 0.87 (m, 9H) ppm; MS: 929.60 m/z [M+H].

### Synthesis of Examples 80-81

The following examples were synthesized from Intermediate 76b and amino alcohol or diamine reagent using the method employed for Example 1.

### Example 80

### 1-decyl 9-(3-((7-(heptadecan-9-yloxy)-7-oxoheptanoyl)oxy)-2-((((2-(1-methylpyrrolidin-2-yl)ethoxy)carbonyl)oxy)methyl)propyl) nonanedioate

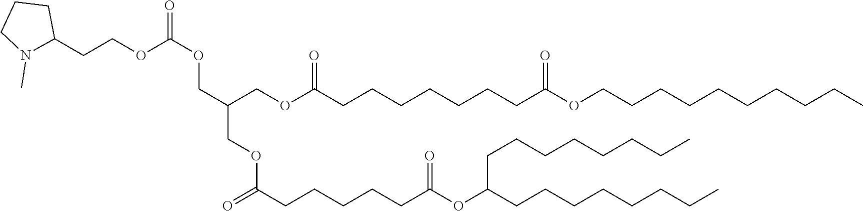 [SMILES: CCCCCCCCCCOC(=O)CCCCCCCC(=O)OCC(COC(=O)CCCCCC(=O)OC(CCCCCCCC)CCCCCCCC)COC(=O)OCCC1CCCN1C]

48% yield; ¹H NMR (400 MHz, CDCl₃) δ 4.85 (m, 1H), 4.26-4.09 (m, 8H), 4.04 (t, J=6.7 Hz, 2H), 3.05 (m, 1H), 2.41 (m, 1H), 2.34-2.26 (m, 11H), 2.19-1.89 (m, 4H), 1.83-1.43 (m, 18H), 1.40-1.17 (m, 46H), 0.87 (m, 9H) ppm; MS: 954.23 m/z [M+H].

### Example 81

### 1-decyl 9-(3-(((2-(diethylamino)ethyl)carbamoyl)oxy)-2-(((7-(heptadecan-9-yloxy)-7-oxoheptanoyl)oxy)methyl)propyl) nonanedioate

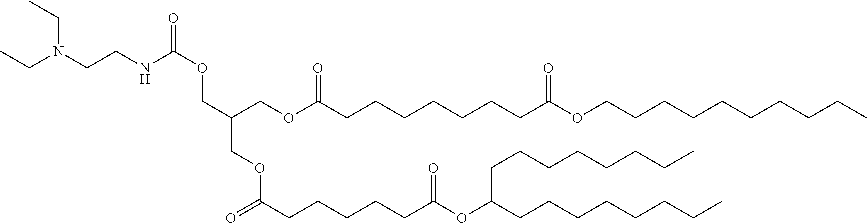 [SMILES: CCCCCCCCCCOC(=O)CCCCCCCC(=O)OCC(COC(=O)CCCCCC(=O)OC(CCCCCCCC)CCCCCCCC)COC(=O)NCCN(CC)CC]

43% yield; ¹H NMR (400 MHz, CDCl₃) δ 5.21 (br t, J=5.2 Hz, 1H), 4.85 (m, 1H), 4.12 (d, J=6.0 Hz, 6H), 4.05 (t, J=6.8 Hz, 2H), 3.21 (br q, J=5.8 Hz, 2H), 2.51 (q, J=7.1, 6.3 Hz, 6H), 2.43-2.23 (m, 8H), 1.62 (m, 11H), 1.50 (q, J=6.1 Hz, 4H), 1.40-1.20 (m, 46H), 0.99 (t, J=7.1 Hz, 6H), 0.87 (m, 9H) ppm; MS: 940.46 m/z [M+H].

### Synthesis of Example 82

### Example 82

### 1-decyl 9-(3-((4-(dimethylamino)butanoyl)oxy)-2-(((7-(heptadecan-9-yloxy)-7-oxoheptanoyl)oxy)methyl)propyl) nonanedioate

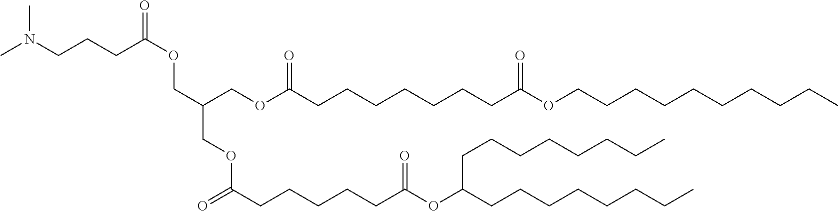 [SMILES: CCCCCCCCCCOC(=O)CCCCCCCC(=O)OCC(COC(=O)CCCCCC(=O)OC(CCCCCCCC)CCCCCCCC)COC(=O)CCCN(C)C]

Example 82 was synthesized in 55% yield from Intermediate 76b and 4-(dimethylamino)butanoic acid using the method employed for Example 25. ¹H NMR (400 MHz, CDCl₃) δ 4.85 (m, 1H), 4.12 (dd, J=6.0, 2.3 Hz, 6H), 4.04 (t, J=6.8 Hz, 2H), 2.42-2.23 (m, 13H), 2.20 (s, 6H), 1.77 (m, 2H), 1.62 (m, 11H), 1.49 (q, J=6.0 Hz, 4H), 1.40-1.19 (m, 45H), 0.87 (m, 9H) ppm; MS: 911.44 m/z [M+H].

### Synthesis of Example 83

### Intermediate 83a: (Z)-1-(3-hydroxy-2-(hydroxymethyl)propyl) 9-(non-2-en-1-yl) nonanedioate

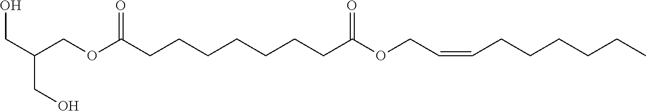 [SMILES: CCCCCC/C=C\COC(=O)CCCCCCCC(=O)OCC(CO)CO]

Intermediate 83a was synthesized in 84% yield from Intermediate 54a and 2-(hydroxymethyl)propane-1,3-diol using the method employed for 76a. ¹H NMR (400 MHz, CDCl₃) δ 5.65 (m, 1H), 5.51 (m, 1H), 4.61 (dd, J=6.9, 1.2 Hz, 2H), 4.24 (d, J=6.3 Hz, 2H), 3.76 (m, 4H), 2.52 (br s, 2H), 2.31 (m, 4H), 2.12-1.98 (m, 3H), 1.61 (m, 4H), 1.40-1.21 (m, 14H), 0.87 (t, J=6.8 Hz, 3H) ppm.

### Intermediate 83b: (Z)-1-(3-((4,4-bis(nonyloxy)butanoyl)oxy)-2-(hydroxymethyl)propyl) 9-(non-2-en-1-yl) nonanedioate

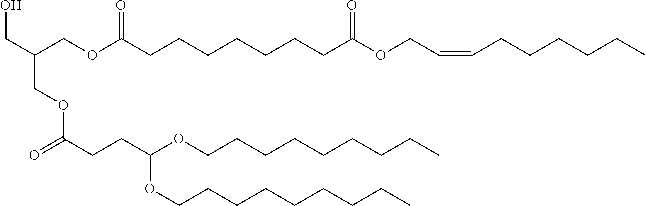 [SMILES: CCCCCC/C=C\COC(=O)CCCCCCCC(=O)OCC(CO)COC(=O)CCC(OCCCCCCCCC)OCCCCCCCCC]

Intermediate 83b was synthesized in 33% yield from Intermediate 83a and Intermediate 79b using the method employed for Intermediate 1d. ¹H NMR (400 MHz, CDCl₃) δ 5.64 (m, 1H), 5.51 (m, 1H), 4.61 (dd, J=6.9, 1.2 Hz, 2H), 4.48 (t, J=5.5 Hz, 1H), 4.17 (m, 4H), 3.62 (br m, 2H), 3.56 (dt, J=9.3, 6.7 Hz, 2H), 3.40 (dt, J=9.3, 6.7 Hz, 2H), 2.40 (t, J=7.5 Hz, 2H), 2.30 (m, 5H), 2.19 (m, 1H), 2.09 (m, 2H), 1.93 (td, J=7.5, 5.4 Hz, 2H), 1.66-1.50 (m, 10H), 1.39-1.21 (m, 36H), 0.0.87 (m, 9H) ppm; MS: 777.87 m/z [M+Na].

### Example 83

### (Z)-1-(3-((4,4-bi s(nonyloxy)butanoyl)oxy)-2-((((3-(di ethylamino)propoxy)carbonyl)oxy)methyl)propyl) 9-(non-2-en-1-yl) nonanedioate

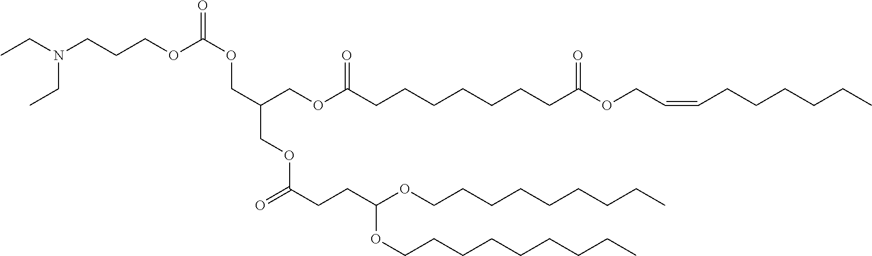 [SMILES: CCCCCC/C=C\COC(=O)CCCCCCCC(=O)OCC(COC(=O)CCC(OCCCCCCCCC)OCCCCCCCCC)COC(=O)OCCCN(CC)CC]

Example 83 was synthesized in 60% yield from Intermediate 83b and 3-(diethylamino)propan-1-ol using the method employed for Example 1. ¹H NMR (400 MHz, CDCl₃) δ 5.62 (m, 1H), 5.51 (m, 1H), 4.61 (dd, J=6.8, 1.2 Hz, 2H), 4.48 (t, J=5.5 Hz, 1H), 4.21-4.09 (m, 8H), 3.55 (dt, J=9.3, 6.7 Hz, 2H), 3.39 (dt, J=9.3, 6.7 Hz, 2H), 2.51 (m, 6H), 2.45-2.35 (m, 3H), 2.29 (t, J=7.6 Hz, 4H), 2.09 (m, 2H), 1.92 (td, J=7.6, 5.5 Hz, 2H), 1.81 (m, 2H), 1.67-1.48 (m, 8H), 1.42-1.20 (m, 38H), 1.00 (t, J=7.1 Hz, 6H), 0.88 (m, 9H) ppm; MS: 913.71 m/z [M+H].

### Synthesis of Example 84

### Intermediate 84a: (Z)-1-(3-((4,4-bis(heptyloxy)butanoyl)oxy)-2-(hydroxymethyl)propyl) 9-(non-2-en-1-yl) nonanedioate

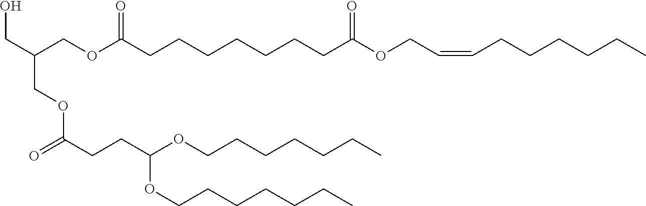 [SMILES: CCCCCC/C=C\COC(=O)CCCCCCCC(=O)OCC(CO)COC(=O)CCC(OCCCCCCC)OCCCCCCC]

Intermediate 84a was synthesized in 38% yield from Intermediate 83a and Intermediate 78b using the method employed for Intermediate 1d. ¹H NMR (400 MHz, CDCl₃) δ 5.64 (m, 1H), 5.51 (m, 1H), 4.61 (dd, J=6.9, 1.2 Hz, 2H), 4.48 (t, J=5.5 Hz, 1H), 4.23-4.12 (m, 4H), 3.62 (br t, J=5.2 Hz, 2H), 3.56 (dt, J=9.3, 6.7 Hz, 2H), 3.40 (dt, J=9.3, 6.7 Hz, 2H), 2.40 (t, J=7.5 Hz, 2H), 2.30 (td, J=7.5, 5.8 Hz, 5H), 2.19 (m, 1H), 2.09 (m, 2H), 1.93 (td, J=7.5, 5.4 Hz, 2H), 1.66-1.50 (m, 8H), 1.39-1.25 (m, 30H), 0.87 (m, 9H) ppm; MS: 721.65 [M+Na].

### Example 84

### (Z)-1-(3-((4,4-bi s(heptyloxy)butanoyl)oxy)-2-((((3-(di ethylamino)propoxy)carbonyl)oxy)methyl)propyl) 9-(non-2-en-1-yl) nonanedioate

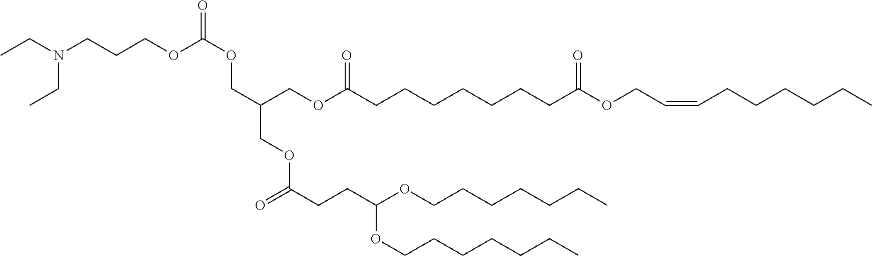 [SMILES: CCCCCC/C=C\COC(=O)CCCCCCCC(=O)OCC(COC(=O)CCC(OCCCCCCC)OCCCCCCC)COC(=O)OCCCN(CC)CC]

Example 84 was synthesized from Intermediate 84a and 3-(diethylamino)propan-1-ol using the method employed for Example 1. ¹H NMR (400 MHz, CDCl₃) δ 5.64 (m, 1H), 5.51 (m, 1H), 4.61 (dd, J=6.8, 1.2 Hz, 2H), 4.48 (t, J=5.5 Hz, 1H), 4.22-4.10 (m, 8H), 3.56 (dt, J=9.3, 6.7 Hz, 2H), 3.39 (dt, J=9.3, 6.7 Hz, 2H), 2.50 (m, 6H), 2.46-2.35 (m, 3H), 2.30 (t, J=7.5 Hz, 4H), 2.09 (m, 2H), 1.92 (m, 2H), 1.80 (dq, J=8.2, 6.6 Hz, 2H), 1.58 (m, 8H), 1.40-1.21 (m, 30H), 1.00 (t, J=7.1 Hz, 6H), 0.89 (m, 9H) ppm; MS: 857.54 m/z [M+H].

### Synthesis of Example 85

### Intermediate 85a: (Z)-1-(3-((7-(heptadecan-9-yloxy)-7-oxoheptanoyl)oxy)-2-(hydroxymethyl)propyl) 9-(non-2-en-1-yl) nonanedioate

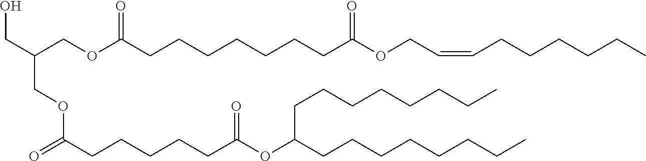 [SMILES: CCCCCC/C=C\COC(=O)CCCCCCCC(=O)OCC(CO)COC(=O)CCCCCC(=O)OC(CCCCCCCC)CCCCCCCC]

Intermediate 85a was synthesized in 37% yield from Intermediate 83a and Intermediate 64a using the method employed for Intermediate 1d. ¹H NMR (400 MHz, CDCl₃) δ 5.64 (m, 1H), 5.51 (m, 1H), 4.85 (m, 1H), 4.61 (dd, J=6.9, 1.2 Hz, 2H), 4.22-4.12 (m, 4H), 3.61 (m, 2H), 2.30 (m, 9H), 2.19 (m, 1H), 2.09 (m, 2H), 1.64 (m, 8H), 1.49 (m, 4H), 1.41-1.18 (m, 40H), 0.87 (m, 9H) ppm; MS: 781.73 m/z [M+H].

### Example 85

### (Z)-1-(3-(((3-(diethylamino)propoxy)carbonyl)oxy)-2-(((7-(heptadecan-9-yloxy)-7-oxoheptanoyl)oxy)methyl)propyl) 9-(non-2-en-1-yl) nonanedioate

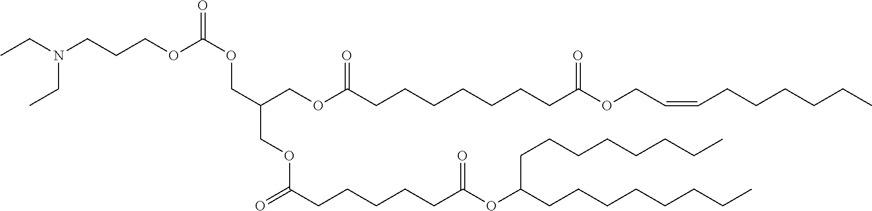 [SMILES: CCCCCC/C=C\COC(=O)CCCCCCCC(=O)OCC(COC(=O)CCCCCC(=O)OC(CCCCCCCC)CCCCCCCC)COC(=O)OCCCN(CC)CC]

Example 85 was synthesized in 48% yield from Intermediate 85a and 3-(diethylamino)propan-1-ol using the method employed for Example 1. ¹H NMR (400 MHz, CDCl₃) δ 5.64 (m, 1H), 5.51 (m, 1H), 4.85 (m, 1H), 4.61 (dd, J=6.8, 1.2 Hz, 2H), 4.24-4.09 (m, 8H), 2.51 (m, 6H), 2.41 (m, 1H), 2.35-2.21 (m, 8H), 2.09 (m, 2H), 1.80 (m, 2H), 1.69-1.55 (m, 10H), 1.50 (q, J=6.2 Hz, 4H), 1.40-1.18 (m, 38H), 1.00 (t, J=7.1 Hz, 6H), 0.88 (m, 9H) ppm; MS: 939.19 m/z [M+H].

### Synthesis of Example 86-88

### Intermediate 86a: (Z)-1-(3-((5-(heptadecan-9-yloxy)-5-oxopentanoyl)oxy)-2-(hydroxymethyl)propyl) 9-(non-2-en-1-yl) nonanedioate

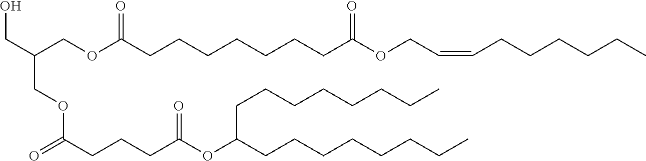 [SMILES: CCCCCC/C=C\COC(=O)CCCCCCCC(=O)OCC(CO)COC(=O)CCCC(=O)OC(CCCCCCCC)CCCCCCCC]

Intermediate 86a was synthesized in 39% yield from Intermediate 83a and Intermediate 63b using the method employed for Intermediate 1d. ¹H NMR (400 MHz, CDCl₃) δ 5.64 (m, 1H), 5.51 (m, 1H), 4.86 (m, 1H), 4.61 (dd, J=7.0, 1.2 Hz, 2H), 4.23-4.12 (m, 4H), 3.62 (t, J=4.4 Hz, 2H), 2.44-2.24 (m, 9H), 2.19 (m, 1H), 2.09 (m, 2H), 1.95 (m, 2H), 1.61 (m, 5H), 1.50 (m, 4H), 1.38-1.20 (m, 37H), 0.87 (m, 9H) ppm; MS: 753.74 m/z [M+H].

The following examples were synthesized from Intermediate 86a and an amino alcohol or diamine reagent using the method employed for Example 1.

### Example 86

### (Z)-1-(3-(((3-(diethylamino)propoxy)carbonyl)oxy)-2-(((5-(heptadecan-9-yloxy)-5-oxopentanoyl)oxy)methyl)propyl) 9-(non-2-en-1-yl) nonanedioate

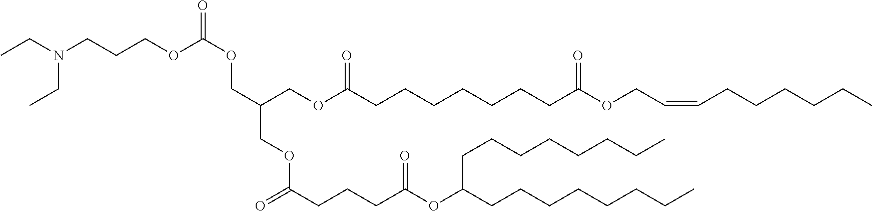 [SMILES: CCCCCC/C=C\COC(=O)CCCCCCCC(=O)OCC(COC(=O)CCCC(=O)OC(CCCCCCCC)CCCCCCCC)COC(=O)OCCCN(CC)CC]

48% yield; ¹H NMR (400 MHz, CDCl₃) δ 5.65 (m, 1H), 5.51 (m, 1H), 4.86 (m, 1H), 4.61 (dd, J=6.9, 1.3 Hz, 2H), 4.22-4.09 (m, 8H), 2.50 (q, J=7.1 Hz, 6H), 2.46-2.25 (m, 9H), 2.09 (m, 2H), 1.94 (m, 2H), 1.86-1.74 (m, 3H), 1.61 (m, 4H), 1.50 (m, 4H), 1.40-1.23 (m, 37H), 1.00 (t, J=7.1 Hz, 6H), 0.87 (m, 9H) ppm; MS: 912.23 m/z [M+H].

### Example 87

### 1-decyl 9-(3-((5-(heptadecan-9-yloxy)-5-oxopentanoyl)oxy)-2-((((2-(1-methylpyrrolidin-2-yl)ethoxy)carbonyl)oxy)methyl)propyl) nonanedioate

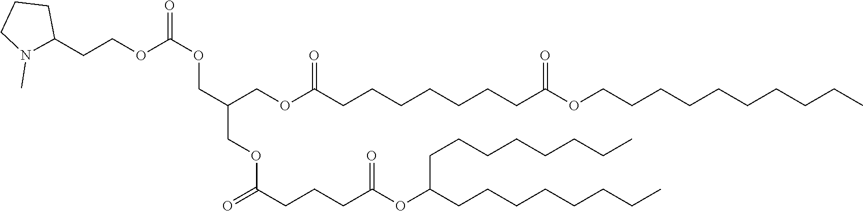 [SMILES: CCCCCCCCCCOC(=O)CCCCCCCC(=O)OCC(COC(=O)CCCC(=O)OC(CCCCCCCC)CCCCCCCC)COC(=O)OCCC1CCCN1C]

50% yield; ¹H NMR (400 MHz, CDCl₃) δ 4.86 (m, 1H), 4.27-4.08 (m, 8H), 4.04 (t, J=6.7 Hz, 2H), 3.05 (m, 1H), 2.48-2.24 (m, 12H), 2.20-1.88 (m, 6H), 1.82-1.41 (m, 15H), 1.37-1.21 (m, 43H), 0.87 (m, 9H) ppm; MS: 925.43 m/z [M+H].

### Example 88

### 1-decyl 9-(3-(((2-(diethylamino)ethyl)carbamoyl)oxy)-2-(((5-(heptadecan-9-yloxy)-5-oxopentanoyl)oxy)methyl)propyl) nonanedioate

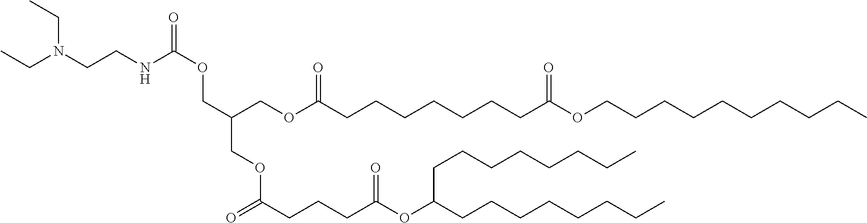 [SMILES: CCCCCCCCCCOC(=O)CCCCCCCC(=O)OCC(COC(=O)CCCC(=O)OC(CCCCCCCC)CCCCCCCC)COC(=O)NCCN(CC)CC]

49% yield; ¹H NMR (500 MHz, CDCl₃) δ 5.23 (br t, J=5.3 Hz, 1H), 4.86 (m, 1H), 4.12 (m, 6H), 4.04 (t, J=6.8 Hz, 2H), 3.20 (br q, J=5.9 Hz, 2H), 2.51 (q, J=7.0 Hz, 6H), 2.41-2.25 (m, 9H), 1.93 (m, 2H), 1.61 (m, 6H), 1.50 (m, 4H), 1.33-1.23 (m, 44H), 0.99 (t, J=7.1 Hz, 6H), 0.87 (m, 9H) ppm; MS: 912.43 m/z [M+H].

### Synthesis of Example 89

### Example 89

### 1-decyl 9-(3-((4-(dimethylamino)butanoyl)oxy)-2-(((5-(heptadecan-9-yloxy)-5-oxopentanoyl)oxy)methyl)propyl) nonanedioate

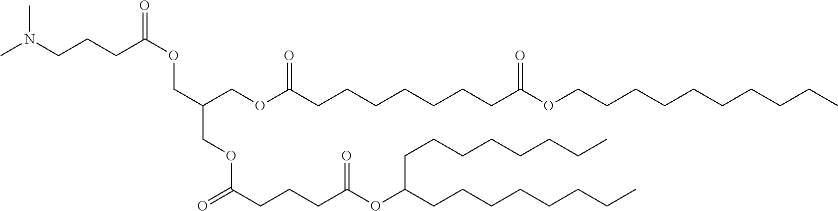 [SMILES: CCCCCCCCCCOC(=O)CCCCCCCC(=O)OCC(COC(=O)CCCC(=O)OC(CCCCCCCC)CCCCCCCC)COC(=O)CCCN(C)C]

Example 89 was synthesized in 43% yield from Intermediate 86a and 4-(dimethylamino)butanoic acid using the method employed for Example 25. ¹H NMR (500 MHz, CDCl₃) δ 4.91-4.82 (m, 1H), 4.12 (m, 6H), 4.05 (t, J=6.8 Hz, 2H), 2.42-2.23 (m, 13H), 2.20 (s, 6H), 1.94 (m, 2H), 1.75 (m, 2H), 1.61 (m, 6H), 1.49 (m, 4H), 1.37-1.16 (m, 44H), 0.87 (m, 9H) ppm; MS: 883.85 m/z [M+H].

### Synthesis of Example 90

### Intermediate 90a: 11-chloro-11-oxoundecanoic acid

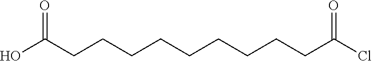 [SMILES: O=C(O)CCCCCCCCCC(=O)Cl]

To a solution of undecanedioic acid (15 g, 69.36 mmol, 1 equiv) in THF (0.6 M) was added oxalyl chloride (1.1-1.3 equiv). After addition, the mixture was stirred and DMF (0.01-0.05 equiv) was added dropwise. The resulting mixture was stirred at 25° C. for 2 h and then concentrated to give 16 g (47.0 mmol, 99% yield) of the desired crude product as yellow oil, which did not require further purification.

### Intermediate 90b: 11-(decyloxy)-11-oxoundecanoic acid

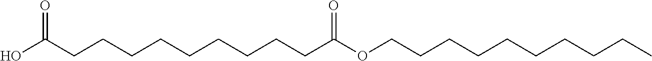 [SMILES: CCCCCCCCCCOC(=O)CCCCCCCCCC(=O)O]

To a solution of Intermediate 90a (16.28 g, 69.36 mmol, 1 equiv) in THF (0.45 M) was added decan-1-ol (1.1 equiv). The mixture was stirred at 25° C. for 2 h and then concentrated in vacuo. The crude residue was purified by column chromatography (gradient of EtOAc in petroleum ether) and, as needed, recrystallized in petroleum ether to provide 5.5 g (15.5 mmol, 22% yield) of the desired product as a white solid. ¹H NMR (400 MHz, CDCl₃) δ 4.06 (t, J=6.8 Hz, 2H), 2.37-2.28 (m, 4H), 1.62 (m, 6H), 1.28 (m, 24H), 0.89 (t, J=6.8 Hz, 3H) ppm; MS: 355.2 m/z [M−H].

### Intermediate 90c: 1-(3-((4,4-bis(octyloxy)butanoyl)oxy)-2-(hydroxymethyl)propyl) 11-decyl undecanedioate

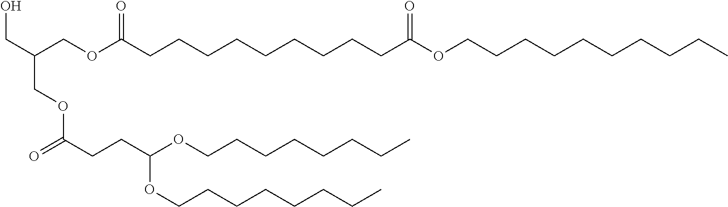 [SMILES: CCCCCCCCCCOC(=O)CCCCCCCCCC(=O)OCC(CO)COC(=O)CCC(OCCCCCCCC)OCCCCCCCC]

Intermediate 90c was synthesized in 39% yield from Intermediate 90b and Intermediate 54b using the method employed for Intermediate 54c. ¹H NMR (400 MHz, CDCl₃) δ 4.48 (t, J=5.5 Hz, 1H), 4.18 (m, 4H), 4.05 (t, J=6.8 Hz, 2H), 3.64-3.51 (m, 4H), 3.40 (dt, J=9.3, 6.7 Hz, 2H), 2.41 (t, J=7.5 Hz, 2H), 2.34-2.23 (m, 5H), 2.19 (m, 1H), 1.93 (td, J=7.6, 5.5 Hz, 2H), 1.65-1.50 (m, 10H), 1.37-1.21 (m, 44H), 0.88 (m, 9H) ppm; MS: 793.73 m/z [M+Na].

### Example 90

### 1-(3-((4,4-bis(octyloxy)butanoyl)oxy)-2-((((3-(diethylamino)propoxy)carbonyl)oxy)methyl)propyl) 11-decyl undecanedioate

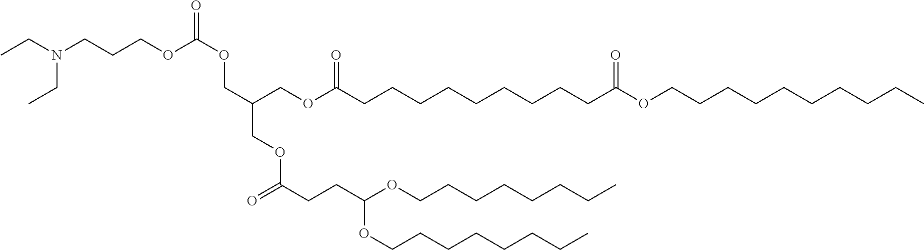 [SMILES: CCCCCCCCCCOC(=O)CCCCCCCCCC(=O)OCC(COC(=O)CCC(OCCCCCCCC)OCCCCCCCC)COC(=O)OCCCN(CC)CC]

Example 90 was synthesized in 59% yield from Intermediate 90c and 3-(diethylamino)propan-1-ol using the method employed for Example 1. ¹H NMR (500 MHz, CDCl₃) δ 4.47 (t, J=5.6 Hz, 1H), 4.21-4.08 (m, 8H), 4.05 (t, J=6.7 Hz, 2H), 3.55 (dt, J=9.3, 6.7 Hz, 2H), 3.39 (dt, J=9.3, 6.7 Hz, 2H), 2.50 (q, J=7.0 Hz, 6H), 2.46-2.36 (m, 3H), 2.29 (q, J=7.7 Hz, 4H), 1.92 (m, 2H), 1.80 (m, 2H), 1.65-1.51 (m, 10H), 1.36-1.24 (m, 44H), 1.00 (t, J=7.1 Hz, 6H), 0.87 (m, 9H) ppm. MS: 929.93 m/z [M+H].

### Synthesis of Example 91

### Intermediate 91a: 13-chloro-13-oxotridecanoic acid

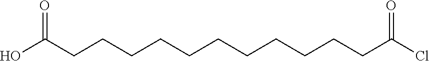 [SMILES: O=C(O)CCCCCCCCCCCC(=O)Cl]

Intermediate 91a was synthesized in quantitative yield from tridecanodioic acid using the method employed for 90a.

### Intermediate 91b: 13-(decyloxy)-13-oxotridecanoic acid

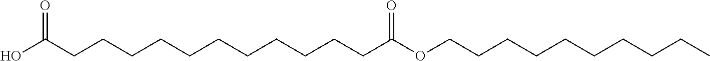 [SMILES: CCCCCCCCCCOC(=O)CCCCCCCCCCCC(=O)O]

Intermediate 91b was synthesized in 17% yield from Intermediate 91a and decan-1-ol using the method employed for Intermediate 90b. ¹H NMR (400 MHz, CDCl₃) δ 4.06 (t, J=6.8 Hz, 2H), 2.37-2.27 (m, 4H), 1.64 (m, 6H), 1.27 (m, 28H), 0.89 (t, J=6.8 Hz, 3H) ppm; MS: 383.3 m/z [M−H].

### Intermediate 91c: 1-(3-((4,4-bis(octyloxy)butanoyl)oxy)-2-(hydroxymethyl)propyl) 13-decyl tridecanedioate

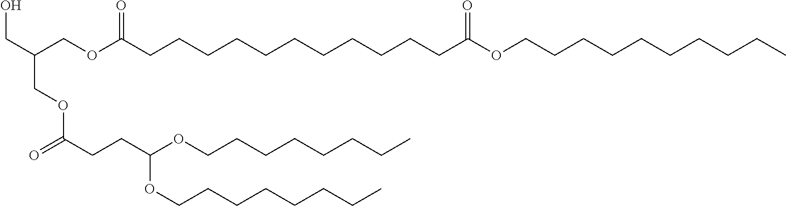 [SMILES: CCCCCCCCCCOC(=O)CCCCCCCCCCCC(=O)OCC(CO)COC(=O)CCC(OCCCCCCCC)OCCCCCCCC]

Intermediate 91c was synthesized in 40% yield from Intermediate 91b and Intermediate 54b using the method employed for Intermediate 54c. ¹H NMR (400 MHz, CDCl₃) δ 4.48 (t, J=5.5 Hz, 1H), 4.23-4.12 (m, 4H), 4.05 (t, J=6.7 Hz, 2H), 3.65-3.51 (m, 4H), 3.40 (dt, J=9.3, 6.7 Hz, 2H), 2.41 (t, J=7.5 Hz, 2H), 2.34-2.22 (m, 5H), 2.19 (m, 1H), 1.93 (td, J=7.5, 5.4 Hz, 2H), 1.58 (m, 10H), 1.38-1.20 (m, 48H), 0.87 (m, 9H) ppm; MS: 821.77 m/z [M+Na].

### Example 91

### 1-(3-((4,4-bi s(octyloxy)butanoyl)oxy)-2-((((3-(di ethylamino)propoxy)carbonyl)oxy)methyl)propyl) 13-decyl tridecanedioate

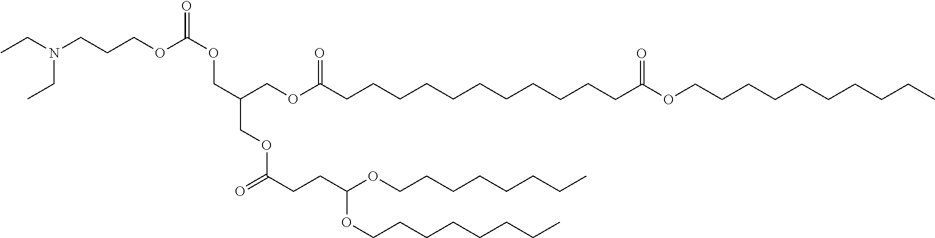 [SMILES: CCCCCCCCCCOC(=O)CCCCCCCCCCCC(=O)OCC(COC(=O)CCC(OCCCCCCCC)OCCCCCCCC)COC(=O)OCCCN(CC)CC]

Example 91 was synthesized in 56% yield from Intermediate 91c and 3-(diethylamino)propan-1-ol using the method employed for Example 1. ¹H NMR (500 MHz, CDCl₃) δ 4.48 (t, J=5.6 Hz, 1H), 4.21-4.08 (m, 8H), 4.05 (t, J=6.7 Hz, 2H), 3.56 (dt, J=9.3, 6.7 Hz, 2H), 3.39 (dt, J=9.3, 6.7 Hz, 2H), 2.50 (q, J=7.1 Hz, 6H), 2.46-2.36 (m, 3H), 2.29 (q, J=7.8 Hz, 4H), 1.92 (m, 2H), 1.81 (m, 2H), 1.65-1.52 (m, 10H), 1.36-1.22 (m, 48H), 1.00 (t, J=7.1 Hz, 6H), 0.88 (m, 9H) ppm; MS: 958.01 m/z [M+H].

### Synthesis of Example 92

### Intermediate 92a: 9-chloro-9-oxononanoic acid

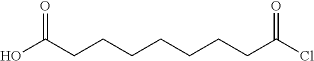 [SMILES: O=C(O)CCCCCCCC(=O)Cl]

Intermediate 92a was synthesized in quantitative yield from nonanedioc acid using the method employed for 90a.

### Intermediate 92b: 9-(dodecyloxy)-9-oxononanoic acid

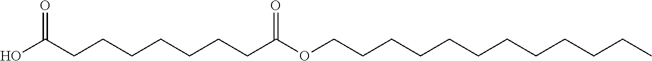 [SMILES: CCCCCCCCCCCCOC(=O)CCCCCCCC(=O)O]

Intermediate 92b was synthesized in 31% yield from Intermediate 92a and dodecan-1-ol using the method employed for Intermediate 90b. ¹H NMR (400 MHz, CDCl₃) δ 4.06 (t, J=6.8 Hz, 2H), 2.37-2.28 (m, 4H), 1.63 (m, 6H), 1.37-1.27 (m, 24H), 0.89 (t, J=6.8 Hz, 3H) ppm; MS: 355.2 m/z [M−H].

### Intermediate 92c: 1-(3-((4,4-bis(octyloxy)butanoyl)oxy)-2-(hydroxymethyl)propyl) 9-dodecyl nonanedioate

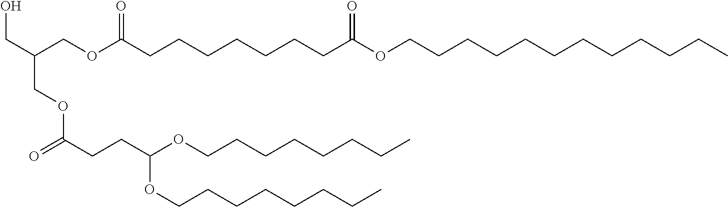 [SMILES: CCCCCCCCCCCCOC(=O)CCCCCCCC(=O)OCC(CO)COC(=O)CCC(OCCCCCCCC)OCCCCCCCC]

Intermediate 92c was synthesized in 43% yield from Intermediate 92b and Intermediate 54b using the method employed for Intermediate 54c. ¹H NMR (400 MHz, CDCl₃) δ 4.48 (t, J=5.5 Hz, 1H), 4.23-4.12 (m, 4H), 4.05 (t, J=6.8 Hz, 2H), 3.65-3.51 (m, 4H), 3.40 (dt, J=9.3, 6.7 Hz, 2H), 2.41 (t, J=7.5 Hz, 2H), 2.30 (dt, J=12.0, 7.6 Hz, 5H), 2.19 (m, 1H), 1.93 (td, J=7.5, 5.5 Hz, 2H), 1.66-1.48 (m, 11H), 1.37-1.22 (m, 43H), 0.87 (m, 9H) ppm; MS: 793.77 m/z [M+Na].

### Example 92

### 1-(3-((4,4-bis(octyloxy)butanoyl)oxy)-2-((((3-(diethylamino)propoxy)carbonyl)oxy)methyl)propyl) 9-dodecyl nonanedioate

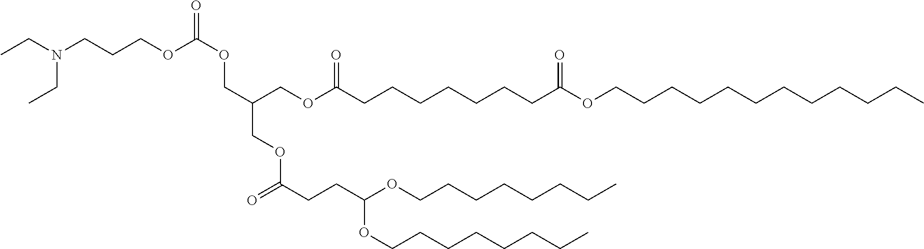 [SMILES: CCCCCCCCCCCCOC(=O)CCCCCCCC(=O)OCC(COC(=O)CCC(OCCCCCCCC)OCCCCCCCC)COC(=O)OCCCN(CC)CC]

Example 92 was synthesized in 53% yield from Intermediate 92c and 3-(diethylamino)propan-1-ol using the method employed for Example 1. ¹H NMR (400 MHz, CDCl₃) δ 4.48 (t, J=5.5 Hz, 1H), 4.22-4.09 (m, 8H), 4.05 (t, J=6.8 Hz, 2H), 3.56 (dt, J=9.3, 6.7 Hz, 2H), 3.39 (dt, J=9.3, 6.7 Hz, 2H), 2.50 (m, 6H), 2.45-2.35 (m, 3H), 2.34-2.24 (m, 4H), 1.92 (m, 2H), 1.80 (m, 2H), 1.71-1.51 (m, 10H), 1.37-1.21 (m, 44H), 1.00 (t, J=7.1 Hz, 6H), 0.87 (m, 9H) ppm; MS: 929.53 m/z [M+H].

### Synthesis of Example 93

### Intermediate 93a: 9-oxo-9-(tetradecyloxy)nonanoic acid

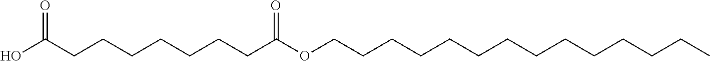 [SMILES: CCCCCCCCCCCCCCOC(=O)CCCCCCCC(=O)O]

Intermediate 93a was synthesized in 11% yield from Intermediate 92a and tetradecan-1-ol using the method employed for Intermediate 90b. ¹H NMR (400 MHz, CDCl₃) δ 4.06 (t, J=6.8 Hz, 2H), 2.37-2.28 (m, 4H), 1.62 (m, 6H), 1.33-1.26 (m, 30H), 0.88 (t, J=6.8 Hz, 3H) ppm; MS: 383.3 m/z [M−H].

### Intermediate 93b: 1-(3-((4,4-bis(octyloxy)butanoyl)oxy)-2-(hydroxymethyl)propyl) 9-tetradecyl nonanedioate

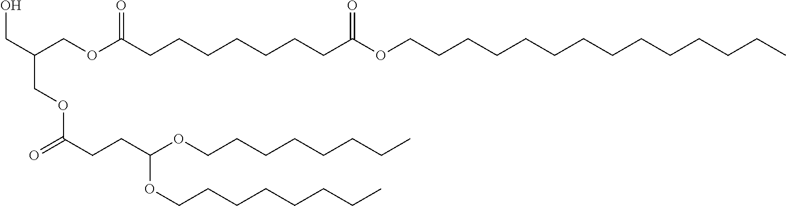 [SMILES: CCCCCCCCCCCCCCOC(=O)CCCCCCCC(=O)OCC(CO)COC(=O)CCC(OCCCCCCCC)OCCCCCCCC]

Intermediate 93b was synthesized in 45% yield from Intermediate 93a and Intermediate 54b using the method employed for Intermediate 54c. ¹H NMR (400 MHz, CDCl₃) δ 4.48 (t, J=5.5 Hz, 1H), 4.23-4.12 (m, 4H), 4.05 (t, J=6.8 Hz, 2H), 3.65-3.50 (m, 4H), 3.40 (dt, J=9.3, 6.7 Hz, 2H), 2.41 (t, J=7.5 Hz, 2H), 2.35-2.24 (m, 5H), 2.19 (m, 1H), 1.93 (td, J=7.5, 5.5 Hz, 2H), 1.66-1.49 (m, 11H), 1.38-1.21 (m, 46H), 0.87 (m, 9H) ppm; MS: 822.00 m/z [M+Na].

### Example 93

### 1-(3-((4,4-bis(octyloxy)butanoyl)oxy)-2-((((3-(diethylamino)propoxy)carbonyl)oxy)methyl)propyl) 9-tetradecyl nonanedioate

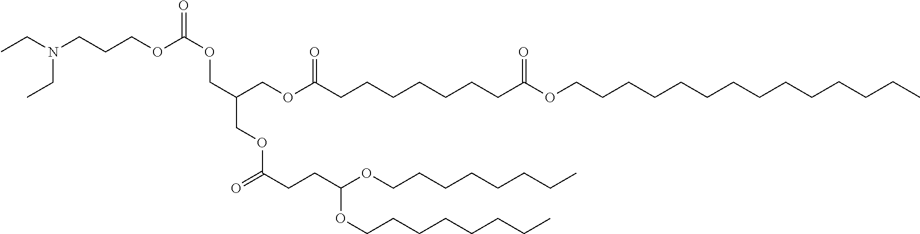 [SMILES: CCCCCCCCCCCCCCOC(=O)CCCCCCCC(=O)OCC(COC(=O)CCC(OCCCCCCCC)OCCCCCCCC)COC(=O)OCCCN(CC)CC]

Example 93 was synthesized in 47% yield from Intermediate 93b and 3-(diethylamino)propan-1-ol using the method employed for Example 1. ¹H NMR (400 MHz, CDCl₃) δ 4.48 (t, J=5.5 Hz, 1H), 4.22-4.08 (m, 8H), 4.05 (t, J=6.8 Hz, 2H), 3.56 (dt, J=9.3, 6.7 Hz, 2H), 3.39 (dt, J=9.3, 6.7 Hz, 2H), 2.50 (q, J=7.1 Hz, 6H), 2.45-2.35 (m, 3H), 2.29 (m, 4H), 1.92 (m, 2H), 1.81 (m, 2H), 1.67-1.49 (m, 12H), 1.37-1.21 (m, 47H), 1.00 (t, J=7.1 Hz, 5H), 0.88 (m, 9H) ppm; MS: 943.03 m/z [M+H].

### Synthesis of Example 94

### Intermediate 94a: 9-oxo-9-(undecan-2-yloxy)nonanoic acid

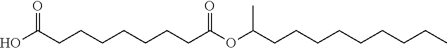 [SMILES: CCCCCCCCCC(C)OC(=O)CCCCCCCC(=O)O]

Intermediate 94a was synthesized in 30% yield from Intermediate 92a and undecan-2-ol using the method employed for Intermediate 90b. ¹H NMR (400 MHz, CDCl₃) δ 4.90 (m, 1H), 2.35 (t, J=7.6 Hz, 2H), 2.27 (t, J=7.4 Hz, 2H), 1.66-1.30 (m, 26H), 1.20 (d, J=6.0 Hz, 3H), 0.89 (t, J=7.0 Hz, 3H) ppm; MS: 341.2 m/z [M−H].

### Intermediate 94b: 1-(3-((4,4-bis(octyloxy)butanoyl)oxy)-2-(hydroxymethyl)propyl) 9-(undecan-2-yl) nonanedioate

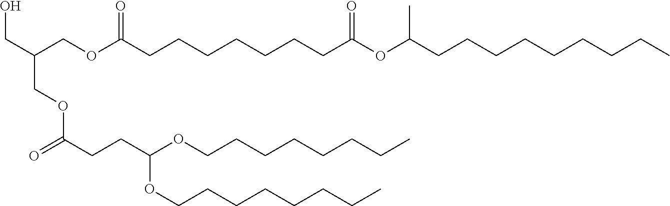 [SMILES: CCCCCCCCCC(C)OC(=O)CCCCCCCC(=O)OCC(CO)COC(=O)CCC(OCCCCCCCC)OCCCCCCCC]

Intermediate 94b was synthesized in 39% yield from Intermediate 94a and Intermediate 54b using the method employed for Intermediate 54c. ¹H NMR (400 MHz, CDCl₃) δ 4.88 (m, 1H), 4.48 (t, J=5.5 Hz, 1H), 4.23-4.11 (m, 4H), 3.66-3.52 (m, 4H), 3.40 (dt, J=9.3, 6.7 Hz, 2H), 2.41 (t, J=7.5 Hz, 2H), 2.35-2.23 (m, 5H), 2.19 (m, 1H), 1.93 (td, J=7.5, 5.4 Hz, 2H), 1.66-1.22 (m, 50H), 1.19 (d, J=6.2 Hz, 3H), 0.87(m, 9H) ppm; MS: 779.78 m/z [M+Na].

### Example 94

### 1-(344,4-bis(octyloxy)butanoyl)oxy)-2-((((3-(diethylamino)propoxy)carbonyl)oxy)methyl)propyl) 9-(undecan-2-yl) nonanedioate

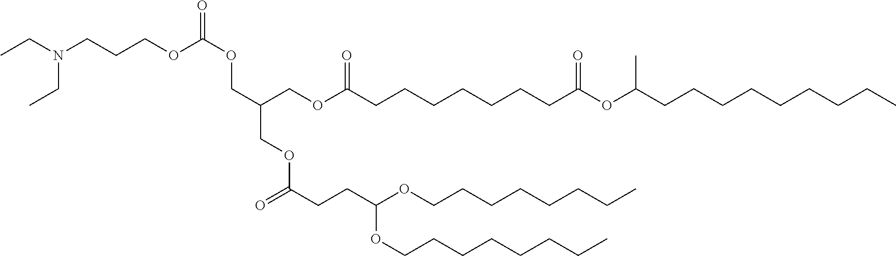 [SMILES: CCCCCCCCCC(C)OC(=O)CCCCCCCC(=O)OCC(COC(=O)CCC(OCCCCCCCC)OCCCCCCCC)COC(=O)OCCCN(CC)CC]

Example 94 was synthesized in 63% yield from Intermediate 94b and 3-(diethylamino)propan-1-ol using the method employed for Example 1. ¹H NMR (400 MHz, CDCl₃) δ 4.89 (m, 1H), 4.48 (t, J=5.5 Hz, 1H), 4.22-4.07 (m, 8H), 3.56 (dt, J=9.3, 6.7 Hz, 2H), 3.39 (dt, J=9.3, 6.7 Hz, 2H), 2.55-2.45 (m, 6H), 2.45-2.35 (m, 3H), 2.34-2.22 (m, 4H), 1.92 (m, 2H), 1.81 (m, 2H), 1.64-1.26 (m, 50H), 1.19 (d, J=6.3 Hz, 3H), 1.00 (t, J=7.1 Hz, 6H), 0.88 (m, 9H) ppm; MS: 915.44 m/z [M+H].

### Synthesis of Example 95

### Intermediate 95a: dodecan-3-ol

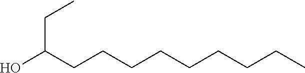

To a solution of decanal (12.8 mL, 64 mmol) in THF (100 mL) was added bromo(ethyl)magnesium (3 M, 19.20 mL) dropwise at -78° C. The reaction mixture was stirred at 25° C. for 2 h, diluted with water (200 mL), and extracted into EtOAc (2×200 mL). The combined organic layers were dried over anhydrous Na₂SO₄, filtered, and concentrated in vacuo. The crude residue was purified using silica gel chromatography (2-2.5% EtOAc in petroleum ether) to provide 6.9 g (37 mmol, 64% yield) of the desired product as a colorless oil. ¹H NMR (400 MHz, CDCl₃) δ 3.53 (m, 1H), 1.49-1.27 (m, 18H), 0.95 (t, J=7.4 Hz, 3H), 0.89 (t, J=6.8 Hz, 3H) ppm.

### Intermediate 95b: 9-(dodecan-3-yloxy)-9-oxononanoic acid

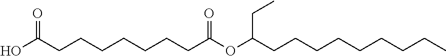 [SMILES: CCCCCCCCCC(CC)OC(=O)CCCCCCCC(=O)O]

Intermediate 95b was synthesized in 38% yield from Intermediate 92a and dodecan-3-ol using the method employed for Intermediate 90b. ¹H NMR (400 MHz, CDCl₃) δ 4.82 (m, 1H), 2.37-2.27 (m, 4H), 1.63 (m, 8H), 1.34-1.26 (m, 20H), 0.88 (m, 6H) ppm; MS: 355.2 m/z [M−H].

### Intermediate 95c: 1-(3-((4,4-bis(octyloxy)butanoyl)oxy)-2-(hydroxymethyl)propyl) 9-(dodecan-3-yl) nonanedioate

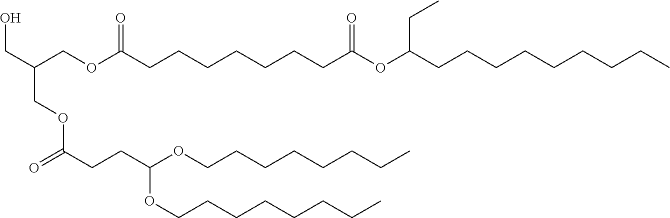 [SMILES: CCCCCCCCCC(CC)OC(=O)CCCCCCCC(=O)OCC(CO)COC(=O)CCC(OCCCCCCCC)OCCCCCCCC]

Intermediate 95c was synthesized in 42% yield from Intermediate 95b and Intermediate 54b using the method employed for Intermediate 54c. ¹H NMR (400 MHz, CDCl₃) δ 4.80 (m, 1H), 4.48 (t, J=5.5 Hz, 1H), 4.23-4.10 (m, 4H), 3.66-3.50 (m, 4H), 3.39 (dt, J=9.3, 6.7 Hz, 2H), 2.40 (t, J=7.5 Hz, 2H), 2.36-2.24 (m, 5H), 2.19 (m, 1H), 1.93 (td, J=7.6, 5.4 Hz, 2H), 1.67-1.46 (m, 11H), 1.38-1.18 (m, 40H), 0.87 (m, 12H) ppm; MS: 793.77 m/z [M+Na].

### Example 95

### 1-(3-((4,4-bi s(octyloxy)butanoyl)oxy)-2-((((3-(di ethylamino)propoxy)carbonyl)oxy)methyl)propyl) 9-(dodecan-3-yl) nonanedioate

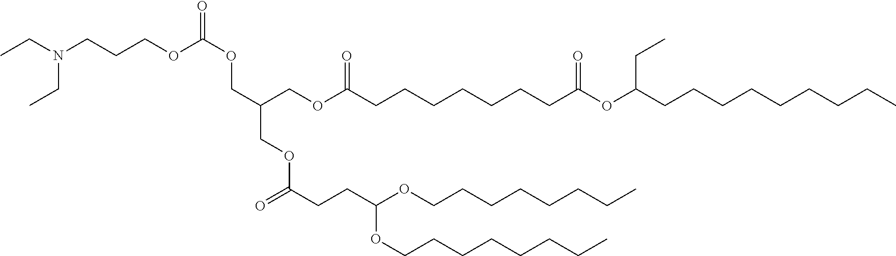 [SMILES: CCCCCCCCCC(CC)OC(=O)CCCCCCCC(=O)OCC(COC(=O)CCC(OCCCCCCCC)OCCCCCCCC)COC(=O)OCCCN(CC)CC]

Example 95 was synthesized in 63% yield from Intermediate 95c and 3-(diethylamino)propan-1-ol using the method employed for Example 1. ¹H NMR (400 MHz, CDCl₃) δ 4.82 (m, 1H), 4.48 (t, J=5.5 Hz, 1H), 4.22-4.07 (m, 8H), 3.56 (dt, J=9.3, 6.7 Hz, 2H), 3.40 (dt, J=9.3, 6.7 Hz, 2H), 2.50 (q, J=6.9 Hz, 6H), 2.45-2.35 (m, 3H), 2.29 (q, J=7.5 Hz, 4H), 1.92 (m, 2H), 1.81 (m, 2H), 1.68-1.47 (m, 11H), 1.37-1.23 (m, 41H), 1.00 (t, J=7.1 Hz, 6H), 0.87 (m, 12H) ppm; MS: 929.03 m/z [M+H].

### Synthesis of Example 96

### Intermediate 96a: (2,2,5-trimethyl-1,3-dioxan-5-yl)methyl (9Z,12Z)-octadeca-9,12-dienoate

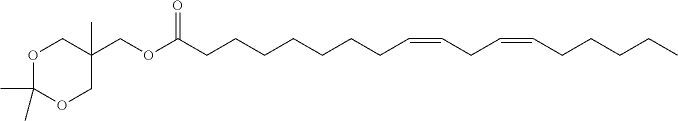 [SMILES: CCCCC/C=C\C/C=C\CCCCCCCC(=O)OCC1(C)COC(C)(C)OC1]

To a solution (2,2,5-trimethyl-1,3-dioxan-5-yl)methanol (2 g, 12.4 mmol), linoleic acid (4.23 mL, 13.6 mmol), DMAP (302 mg, 2.48 mmol), and DIPEA (4.32 mL, 24.8 mmol) in DCM (50 mL) was added EDCHCl (3.56 g, 18.6 mmol) at rt. The reaction mixture was stirred at rt for 48 h and then diluted with water (25 mL). The organic layer was collected, washed with water (25 mL), dried over anhydrous sodium sulfate, and concentrated in vacuo. The crude residue was purified using silica gel chromatography (0-100% EtOAc in hexanes) to provide 2.16 g (5.11 mmol, 41% yield) of the desired product as a clear oil. MS: 867.22 m/z [M+Na].

### Intermediate 96b: 3-hydroxy-2-(hydroxymethyl)-2-methylpropyl (9Z,12Z)-octadeca-9,12-dienoate

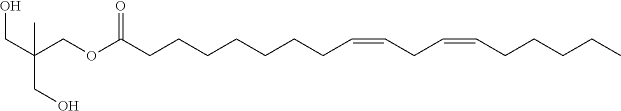 [SMILES: CCCCC/C=C\C/C=C\CCCCCCCC(=O)OCC(C)(CO)CO]

Intermediate 96a (2.16 g, 5.11 mmol) was dissolved in methanol (50 mL) and Dowex 50×8 resin was added. The resulting reaction mixture was stirred for 16 h at rt, filtered, and concentrated in vacuo. The crude residue was purified using silica gel chromatography (0-60% EtOAc in hexanes) to provide 1.43 g (3.73 mmol, 73% yield) of the desired product as a clear oil. ¹H NMR (400 MHz, CDCl₃) δ 5.44-5.27 (m, 4H), 4.20 (s, 2H), 3.58 (dd, J=11.3, 4.2 Hz, 2H), 3.51 (dd, J=11.3, 4.9 Hz, 2H), 2.81-2.74 (m, 2H), 2.71 (m, 2H), 2.36 (t, J=7.5 Hz, 2H), 2.05 (dt, J=8.5, 6.7 Hz, 4H), 1.69-1.56 (m, 2H), 1.40-1.25 (m, 14H), 0.89 (m, 3H), 0.84 (s, 3H) ppm.

### Intermediate 96c: 3-((4,4-bis(octyloxy)butanoyl)oxy)-2-(hydroxymethyl)-2-methylpropyl (9Z,12Z)-octadeca-9,12-dienoate

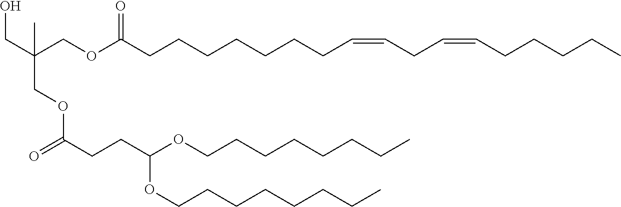 [SMILES: CCCCC/C=C\C/C=C\CCCCCCCC(=O)OCC(C)(CO)COC(=O)CCC(OCCCCCCCC)OCCCCCCCC]

Intermediate 96c was synthesized in 67% yield from Intermediate 96b and Intermediate 1c using the method employed for Intermediate 1d. ¹H NMR (400 MHz, CDCl₃) δ 5.44-5.27 (m, 4H), 4.49 (t, J=5.5 Hz, 1H), 4.02 (d, J=2.7 Hz, 4H), 3.56 (dt, J=9.3, 6.7 Hz, 2H), 3.44-3.36 (m, 4H), 2.80-2.74 (m, 2H), 2.42 (t, J=7.5 Hz, 3H), 2.33 (t, J=7.6 Hz, 2H), 2.05 (q, J=6.8 Hz, 4H), 1.94 (td, J=7.5, 5.4 Hz, 2H), 1.69-1.50 (m, 7H), 1.40-1.20 (m, 34H), 0.95 (s, 3H), 0.88 (m, 9H) ppm.

### Example 96

### 3-((4,4-bis(octyloxy)butanoyl)oxy)-2-((((3-(diethylamino)propoxy)carbonyl)oxy)methyl)-2-methylpropyl (9Z,12Z)-octadeca-9,12-dienoate

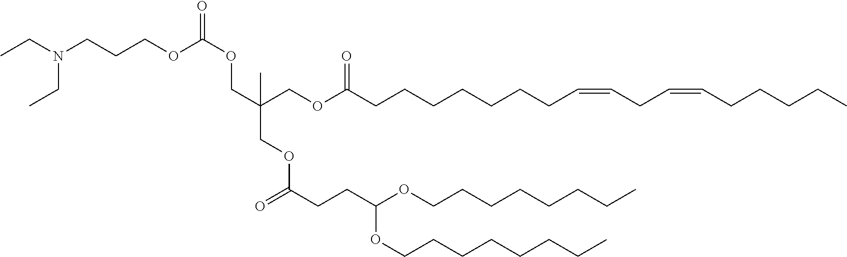 [SMILES: CCCCC/C=C\C/C=C\CCCCCCCC(=O)OCC(C)(COC(=O)CCC(OCCCCCCCC)OCCCCCCCC)COC(=O)OCCCN(CC)CC]

Example 96 was synthesized in 32% yield from Intermediate 96c and 3-(diethylamino)propan-1-ol using the method employed for Example 1. ¹H NMR (400 MHz, CDCl₃) δ 5.44-5.27 (m, 4H), 4.48 (t, J=5.5 Hz, 1H), 4.18 (t, J=6.6 Hz, 2H), 4.06 (s, 2H), 4.01 (m, 4H), 3.56 (dt, J=9.3, 6.7 Hz, 2H), 3.40 (dt, J=9.3, 6.7 Hz, 2H), 2.77 (t, J=6.4 Hz, 2H), 2.51 (q, J=7.1 Hz, 6H), 2.40 (t, J=7.6 Hz, 2H), 2.31 (t, J=7.6 Hz, 2H), 2.05 (q, J=6.8 Hz, 5H), 1.92 (td, J=7.6, 5.5 Hz, 2H), 1.81 (m, 2H), 1.67-1.49 (m, 12H), 1.42-1.19 (m, 38H), 1.05-0.96 (m, 9H), 0.88 (m, 9H) ppm; MS: 867.22 m/z [M+H].

### Synthesis of Example 97

### Intermediate 97a: 2,3-dihydroxypropyl (9Z,12Z)-octadeca-9,12-dienoate

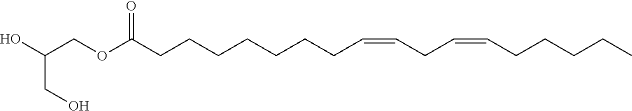 [SMILES: CCCCC/C=C\C/C=C\CCCCCCCC(=O)OCC(O)CO]

To a solution of glycerol (1.59 g, 17.3 mmol) in DCM (172 mL) was added linoleic acid (35.6 mmol), DMAP (423 mg, 3.47 mmol), and DIPEA (7.23 mL, 41.6 mmol) at rt. EDCHCl (8.05 g, 41.6 mmol) was added in three portions over 15 min, followed by addition of DMF (1 mL). The resulting reaction mixture was stirred for 16 h, washed with water, 1M HCl and 5% NaHCO₃, dried over anhydrous sodium sulfate, and concentrated in vacuo. The crude residue was purified using silica gel chromatography (gradient of EtOAc in hexanes) to provide 2.10 g (5.9 mmol, 34% yield) of the desired product. ¹H NMR (400 MHz, CDCl₃) δ 5.44-5.27 (m, 4H), 4.24-4.06 (m, 2H), 3.92 (m, 1H), 3.69 (m, 1H), 3.59 (dt, J=11.0, 5.1 Hz, 1H), 2.76 (m, 2H), 2.67 (d, J=4.9 Hz, 1H), 2.34 (t, J=7.6 Hz, 2H), 2.25 (m, 1H), 2.04 (m, 4H), 1.63 (m, 2H), 1.41-1.22 (m, 14H), 0.89 (t, J=6.8 Hz, 3H) ppm.

### Intermediate 97b: 3-((4,4-bis(octyloxy)butanoyl)oxy)-2-hydroxypropyl (9Z,12Z)-octadeca-9,12-dienoate

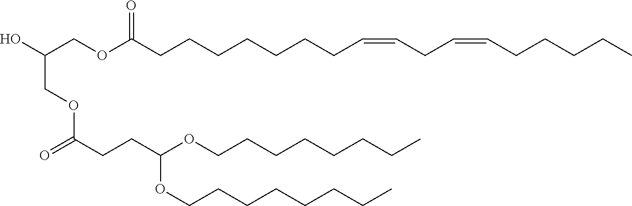 [SMILES: CCCCC/C=C\C/C=C\CCCCCCCC(=O)OCC(O)COC(=O)CCC(OCCCCCCCC)OCCCCCCCC]

Intermediate 97b was synthesized in 11% yield from Intermediate 97a and Intermediate 1c using the method employed for Intermediate 1d. ¹H NMR (400 MHz, CDCl₃) δ 5.35 (m, 4H), 4.50 (t, J=5.5 Hz, 1H), 4.23-4.05 (m, 5H), 3.56 (dt, J=9.3, 6.7 Hz, 2H), 3.40 (dt, J=9.3, 6.7 Hz, 2H), 2.77 (m, 2H), 2.55 (d, J=4.6 Hz, 1H), 2.44 (t, J=7.4 Hz, 2H), 2.35 (t, J=7.6 Hz, 2H), 2.05 (q, J=6.8 Hz, 4H), 1.95 (td, J=7.4, 5.5 Hz, 2H), 1.70-1.51 (m, 8H), 1.39-1.21 (m, 34H), 0.88 (m, 9H) ppm.

### Example 97

### 3-((4,4-bis(octyloxy)butanoyl)oxy)-2-(((3-(diethylamino)propoxy)carbonyl)oxy)propyl (9Z,12Z)-octadeca-9,12-dienoate

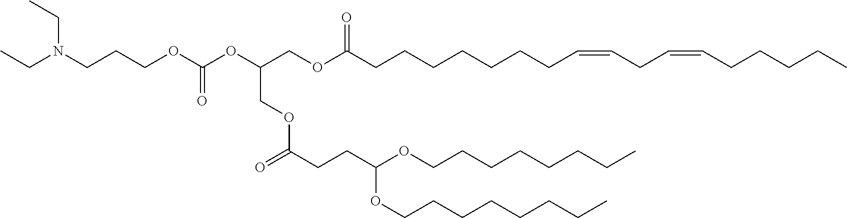 [SMILES: CCCCC/C=C\C/C=C\CCCCCCCC(=O)OCC(COC(=O)CCC(OCCCCCCCC)OCCCCCCCC)OC(=O)OCCCN(CC)CC]

To a solution of Intermediate 96b (170 mg, 0.25 mmol) in acetonitrile (5 mL) was added pyridine (40 μL, 0.5 mmol) and 4-nitrophenyl chloroformate (70 mg, 0.35 mmol) at rt. Upon stirring for 4 h, 3-diethylamino-1-propanol (111 μL, 0.75 mmol) was added and the resulting reaction mixture was stirred an additional 2 h. The reaction mixture was extracted into hexanes (10 mL), washed with water, dried over anhydrous sodium sulfate and concentrated in vacuo. The crude residue was purified using silica gel chromatography (0-100% EtOAc in hexanes) to provide 52 mg (0.062 mmol, 25% yield) of the desired product as a clear oil. ¹H NMR (400 MHz, CDCl₃) δ 5.36 (m, 4H), 5.08 (m, 1H), 4.48 (t, J=5.6 Hz, 1H), 4.34 (m, 2H), 4.25-4.13 (m, 4H), 3.56 (dt, J=9.3, 6.7 Hz, 2H), 3.40 (dt, J=9.3, 6.7 Hz, 2H), 2.77 (t, J=6.6 Hz, 2H), 2.56-2.46 (m, 6H), 2.41 (t, J=7.6 Hz, 2H), 2.36-2.28 (m, 2H), 2.05 (q, J=6.9 Hz, 4H), 1.92 (td, J=7.6, 5.5 Hz, 2H), 1.82 (m, 2H), 1.67-1.52 (10H), 1.40-1.23 (m, 34H), 1.01 (t, J=7.1 Hz, 6H), 0.88 (m, 9H) ppm; MS: 840.03 m/z [M+H].

### Synthesis of Example 98

### Intermediate 98a: 2-((2,2-dim ethyl-1,3-di oxan-5-yl)methoxy)-N,N-di ethyl ethan-1-amine

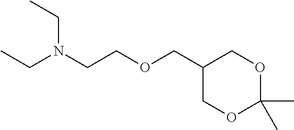 [SMILES: CCN(CC)CCOCC1COC(C)(C)OC1]

To a mixture of 95% sodium hydride (2.5 equiv) in anhydrous DCM (1.7-4.3 M) at 0° C. was added a solution of (2,2-dimethyl-1,3-dioxan-5-yl)methanol (1 equiv) in anhydrous DCM (0.45-1.1 M). The reaction mixture was stirred at 0° C. for 15 min, followed by addition of (2-bromoethyl)diethylamine hydrobromide (1.23 g, 4.78 mmol, 1.4 equiv). The reaction was warmed to rt and stirred for 2 h, before recooling to 0° C., diluting with water, and extracting into EtOAc. The organic layer was collected, dried over anhydrous magnesium sulfate, and concentrated in vacuo to afford 370 mg (1.50 mmol, 44% yield) of the desired product as a clear oil, which was not further purified. ¹H NMR (500 MHz, CDCl₃) δ 3.95 (m, 2H), 3.75 (m, 2H), 3.51 (t, J=6.2 Hz, 2H), 3.47 (d, J=6.7 Hz, 2H), 2.64 (t, J=6.2 Hz, 2H), 2.57 (q, J=7.1 Hz, 4H), 1.98 (m, 1H), 1.42 (s, 3H), 1.40 (s, 3H) 1.02 (t, J=7.1 Hz, 6H) ppm; MS: 246.40 [M+H].

### Intermediate 98b: 3-(2-(diethylamino)ethoxy)-2-(hydroxymethyl)propyl (9Z,12Z)-octadeca-9,12-dienoate

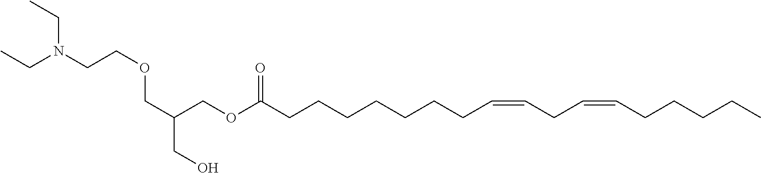 [SMILES: CCCCC/C=C\C/C=C\CCCCCCCC(=O)OCC(CO)COCCN(CC)CC]

4 N HCl in 1,4-dioxane (10 mL, 40 equiv) was added neat to Intermediate 98a (1 equiv). The resulting reaction mixture was stirred at rt for 2 h and then concentrated in vacuo to provide the acetonide-deprotected intermediate. The crude residue, linoleic acid (0.9-1.5 equiv), DIPEA (2-3.2 equiv), and DMAP (0.15-0.26 equiv) were dissolved in DCM (0.2-0.3 M). EDC-HCl (1.2-1.9 equiv) was added to the solution and reaction mixture was stirred for 16 h at rt. The reaction mixture was concentrated in vacuo and then purified using silica gel chromatography (0-100% EtOAc in hexanes, followed by 0-10% MeOH in DCM) to provide 550 mg (78% yield for two steps) of the desired product as a white gum. MS: 469.08 [M+H].

### Example 98

### 3-((4,4-bis(octyloxy)butanoyl)oxy)-2-((2-(diethylamino)ethoxy)methyl)propyl (9Z,12Z)-octadeca-9,12-dienoate

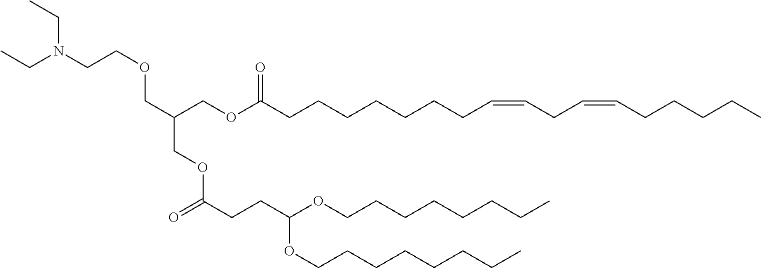 [SMILES: CCCCC/C=C\C/C=C\CCCCCCCC(=O)OCC(COCCN(CC)CC)COC(=O)CCC(OCCCCCCCC)OCCCCCCCC]

To a solution of Intermediate 98b (1 equiv), DIPEA (1.5 equiv), DMAP (0.2 equiv), and Intermediate 1c (1 equiv) in DCM (0.1-0.5 M) was added EDC-HCl (1.5 equiv) at rt. The reaction mixture was stirred for 16 h at rt, concentrated in vacuo, and purified using silica gel chromatography (0-10% MeOH in DCM with 0.1% ammonium hydroxide). Product fractions were pooled, concentrated in vacuo, and azeotroped with 50 mL of toluene (three times) to remove residual ammonium hydroxide and to provide 174 mg (0.22 mmol, 19% yield) of the desired product as a pale yellow oil. ¹H NMR (400 MHz, CDCl₃) δ 5.43-5.28 (m, 4H), 4.48 (t, J=5.6 Hz, 1H), 4.18-4.07 (m, 4H), 3.56 (dt, J=9.3, 6.7 Hz, 2H), 3.52-3.32 (m, 6H), 2.77 (t, J=6.4 Hz, 2H), 2.63 (t, J=6.2 Hz, 2H), 2.55 (q, J=7.2 Hz, 4H), 2.39 (t, J=7.6 Hz, 2H), 2.29 (m, 3H), 2.05 (q, J=6.8 Hz, 4H), 1.92 (td, J=7.6, 5.5 Hz, 2H), 1.57 (m, 7H), 1.31 (m, 33H), 1.02 (t, J=7.1 Hz, 6H), 0.93-0.84 (m, 9H) ppm; MS: 795.56 M+H (ESI+).

### Synthesis of Example 99

### Intermediate 99a: 3-((2,2-dimethyl-1,3-dioxan-5-yl)methoxy)-N,N-diethylpropan-1-amine

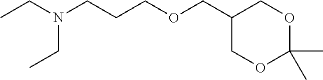 [SMILES: CCN(CC)CCCOCC1COC(C)(C)OC1]

Intermediate 99a was synthesized in 77% yield from (3-bromopropyl)diethylamine hydrobromide and (2,2-dimethyl-1,3-dioxan-5-yl)methanol using the method employed for Intermediate 98a. MS: 260.58 [M+H].

### Intermediate 99b: 3-(3-(diethylamino)propoxy)-2-(hydroxymethyl)propyl (9Z,12Z)-octadeca-9,12-dienoate

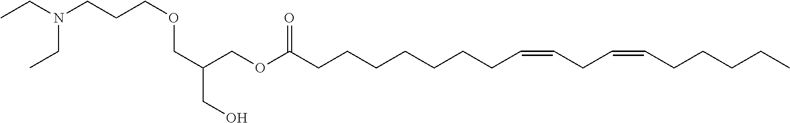 [SMILES: CCCCC/C=C\C/C=C\CCCCCCCC(=O)OCC(CO)COCCCN(CC)CC]

Intermediate 99b was synthesized in 50% yield (two steps) from Intermediate 99a and linoleic acid using the method employed for Intermediate 98b. MS: 483.12 [M+H].

### Example 99

### 3-((4,4-bis(octyloxy)butanoyl)oxy)-2-((3-(diethylamino)propoxy)methyl)propyl (9Z,12Z)-octadeca-9,12-dienoate

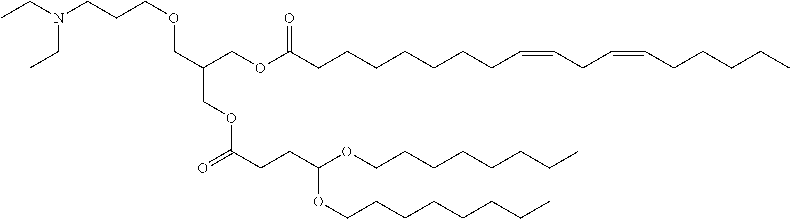 [SMILES: CCCCC/C=C\C/C=C\CCCCCCCC(=O)OCC(COCCCN(CC)CC)COC(=O)CCC(OCCCCCCCC)OCCCCCCCC]

Example 99 was synthesized in 20% yield from Intermediate 99b and Intermediate 1c using the method employed for Example 98. ¹H NMR (400 MHz, CDCl₃) δ 5.42-5.27 (m, 4H), 4.48 (t, J=5.6 Hz, 1H), 4.13 (m, 4H), 3.56 (dt, J=9.3, 6.7 Hz, 2H), 3.46-3.34 (m, 6H), 2.77 (t, J=6.4 Hz, 2H), 2.56-2.43 (m, 6H), 2.39 (t, J=7.6 Hz, 2H), 2.29 (m, 3H), 2.05 (q, J=6.8 Hz, 4H), 1.93 (m, 2H), 1.64 (m, 9H), 1.36-1.26 (m, 33H), 1.01 (t, J=7.1 Hz, 6H), 0.88 (m, 9H) ppm; MS: 809.74 [M+H].

### Synthesis of Example 100

### Intermediate 100a: 3-((4,4-bis(octyloxy)butanoyl)oxy)-2-((((4-nitrophenoxy)carbonyl)oxy)methyl)propyl (9Z,12Z)-octadeca-9,12-dienoate

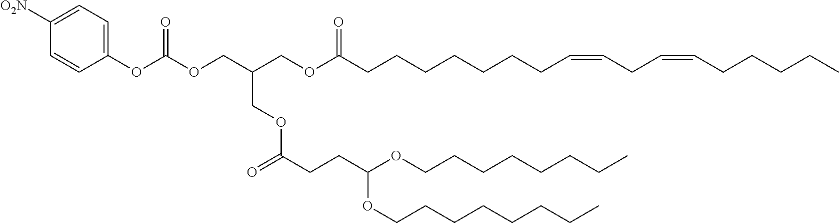 [SMILES: CCCCC/C=C\C/C=C\CCCCCCCC(=O)OCC(COC(=O)CCC(OCCCCCCCC)OCCCCCCCC)COC(=O)Oc1ccc([N+](=O)[O-])cc1]

To a solution of Intermediate 1d (3 g, 1.0 equiv.) and (4-nitrophenyl)carbonochloridate (1.74 g, 2.0 equiv.) in DCM (30 mL) was added pyridine (1.05 mL, 3.0 equiv.) at 0° C. The mixture was stirred at 20° C. for 2 h under N₂ atmosphere. The reaction mixture was concentrated under reduced pressure to remove DCM. The residue was diluted with H₂O and extracted with 2× with EtOAc. The combined organic layers were concentrated under reduced pressure to give a residue. The residue was purified by column chromatography to afford product as a colorless oil (2.5 g, 67%).

Example 100

### 3-((4,4-bis(octyloxy)butanoyl)oxy)-2-((((2-(pyrrolidin-1-yl)ethyl)carbamoyl)oxy)methyl)propyl (9Z,12Z)-octadeca-9,12-dienoate

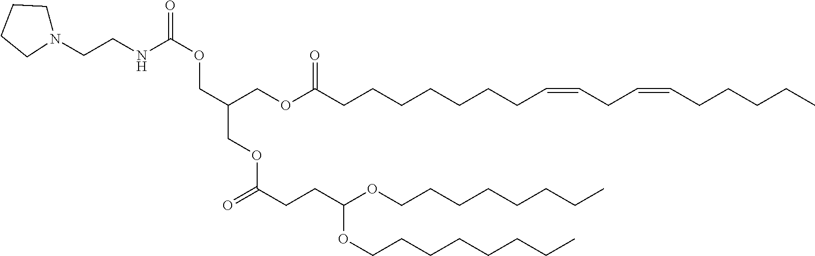 [SMILES: CCCCC/C=C\C/C=C\CCCCCCCC(=O)OCC(COC(=O)CCC(OCCCCCCCC)OCCCCCCCC)COC(=O)NCCN1CCCC1]

To a solution of Intermediate 100a (500 mg, 1.0 equiv.) in MeCN (9 mL) was added 2-pyrrolidin-1-ylethanamine (133 mg, 2.0 equiv.), pyridine (94 uL, 2.0 equiv.) and DMAP (71 mg, 1.0 equiv.). The mixture was stirred at 20° C. for 5 h under N₂ atmosphere. The mixture was concentrated under reduced pressure to remove solvent. The residue was diluted with EtOAc and washed 5× with 1 N NaHCO₃ and 3× with H₂O. The organic layer was dried over Na₂SO₄, filtered and the filtrate was concentrated under reduced pressure to give a residue. The residue was purified by column chromatography to afford product as a colorless oil (150 mg, 31%). ¹H NMR (400 MHz, CDCl₃) δ 5.36-5.22 (m, 4H), 5.20 (d, J=15.9 Hz, 1H), 4.42 (t, J=5.6 Hz, 1H), 4.05 (t, J=5.4 Hz, 6H), 3.49 (dt, J=9.4, 6.7 Hz, 2H), 3.38-3.30 (m, 2H), 3.21 (q, J=5.8 Hz, 2H), 2.70 (t, J=6.4 Hz, 2H), 2.51 (t, J=6.1 Hz, 2H), 2.43 (d, J=6.2 Hz, 4H), 2.33 (dd, J=9.7, 5.5 Hz, 3H), 2.23 (t, J=7.6 Hz, 3H), 1.98 (q, J=6.9 Hz, 4H), 1.85 (td, J=7.6, 5.5 Hz, 2H), 1.70 (q, J=3.3 Hz, 4H), 1.57-1.44 (m, 12H), 1.23 (ddt, J=18.4, 10.5, 6.3 Hz, 37H), 0.84-0.78 (m, 9H). MS: 836.3 m/z [M+H].

### Synthesis of Examples 101-103

The following examples were synthesized from Intermediate 100a and an amino alcohol or diamine reagent using the method employed for Example 100.

### Example 101

### Example 101

### 3-((4,4-bis(octyloxy)butanoyl)oxy)-2-((((2-(piperidin-1-yl)ethyl)carbamoyl)oxy)methyl)propyl (9Z,12Z)-octadeca-9,12-dienoate

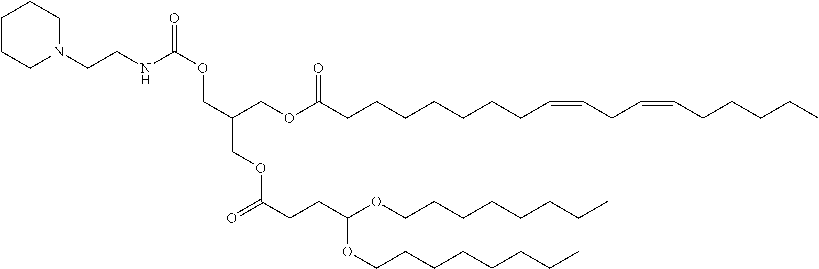 [SMILES: CCCCC/C=C\C/C=C\CCCCCCCC(=O)OCC(COC(=O)CCC(OCCCCCCCC)OCCCCCCCC)COC(=O)NCCN1CCCCC1]

32% yield; ¹H NMR (400 MHz, CDCl₃) δ 5.36-5.17 (m, 4H), 4.41 (t, J=5.5 Hz, 1H), 4.18-4.02 (m, 8H), 3.49 (dt, J=9.3, 6.7 Hz, 2H), 3.33 (dt, J=9.3, 6.6 Hz, 2H), 2.68 (dt, J=19.3, 6.5 Hz, 4H), 2.50 (q, J=7.2 Hz, 4H), 2.40-2.28 (m, 3H), 2.23 (t, J=7.6 Hz, 2H), 1.98 (q, J=6.9 Hz, 4H), 1.85 (td, J=7.6, 5.5 Hz, 2H), 1.61-1.44 (m, 12H), 1.32-1.14 (m, 35H), 0.96 (t, J=7.1 Hz, 6H), 0.82 (td, J=6.9, 4.0 Hz, 9H). MS: 850.3 m/z [M+H].

### Example 102

### Example 102

### 3-((4,4-bis(octyloxy)butanoyl)oxy)-2-((((3-(piperidin-1-yl)propoxy)carbonyl)oxy)methyl)propyl (9Z,12Z)-octadeca-9,12-dienoate

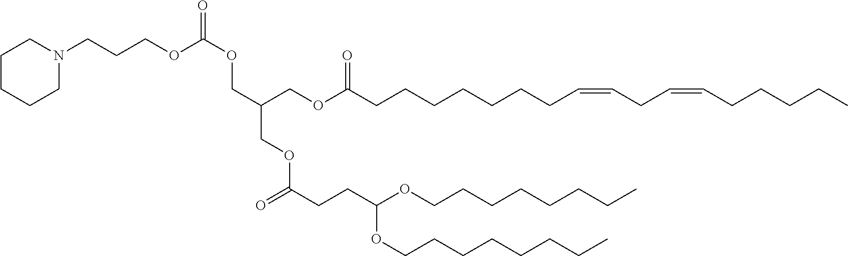 [SMILES: CCCCC/C=C\C/C=C\CCCCCCCC(=O)OCC(COC(=O)CCC(OCCCCCCCC)OCCCCCCCC)COC(=O)OCCCN1CCCCC1]

91% yield; ¹H NMR (400 MHz, CDCl₃) δ 5.36-5.21 (m, 4H), 4.41 (t, J=5.5 Hz, 1H), 4.16-4.00 (m, 8H), 3.49 (dt, J=9.3, 6.7 Hz, 2H), 3.33 (dt, J=9.3, 6.7 Hz, 2H), 2.70 (t, J=6.5 Hz, 2H), 2.40-2.19 (m, 11H), 1.98 (q, J=6.8 Hz, 4H), 1.89-1.74 (m, 4H), 1.60-1.43 (m, 14H), 1.41-1.08 (m, 36H), 0.82 (td, J=6.9, 4.0 Hz, 9H). MS: 865.3 m/z [M+H].

### Example 103

### Example 103

### 3-(((2-(azepan-1-yl)ethyl)carbamoyl)oxy)-2-(((4,4-bis(octyloxy)butanoyl)oxy)methyl)propyl (9Z,12Z)-octadeca-9,12-dienoate

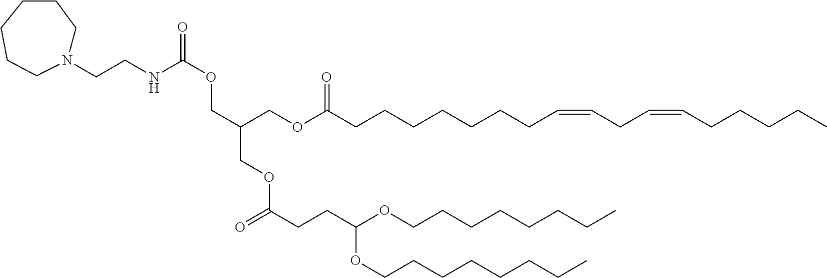 [SMILES: CCCCC/C=C\C/C=C\CCCCCCCC(=O)OCC(COC(=O)CCC(OCCCCCCCC)OCCCCCCCC)COC(=O)NCCN1CCCCCC1]

47% yield; ¹H NMR (400 MHz, CDCl₃) δ 5.67 (s, 1H), 5.42-5.29 (m, 4H), 4.48 (t, J=5.6 Hz, 1H), 4.12 (qt, J=6.2, 4.1, 3.7 Hz, 7H), 3.56 (dt, J=9.3, 6.7 Hz, 2H), 3.44-3.38 (m, 2H), 3.27 (q, J=5.6 Hz, 2H), 2.71 (ddt, J=30.8, 24.9, 12.7 Hz, 8H), 2.35 (dt, J=37.4, 7.7 Hz, 6H), 2.27-1.99 (m, 13H), 1.92 (td, J=7.6, 5.5 Hz, 2H), 1.60 (ddt, J=24.3, 21.1, 6.7 Hz, 14H), 1.40-1.19 (m, 35H), 0.88 (td, J=6.8, 4.0 Hz, 9H). MS: 864.3 m/z [M+H].

### Synthesis of Example 107

### Intermediate 107a: 3-(2-((3r,5r,7r)-adamantan-1-yl)acetoxy)-2-(hydroxymethyl)propyl (9Z,12Z)-octadeca-9,12-dienoate

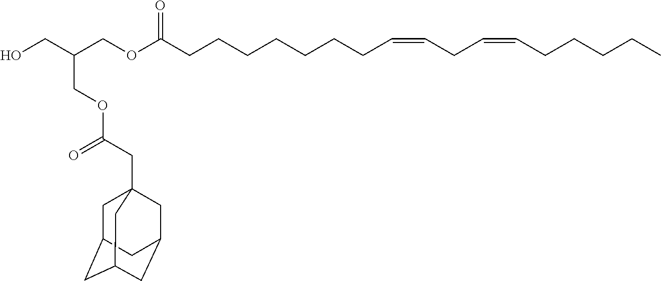 [SMILES: CCCCC/C=C\C/C=C\CCCCCCCC(=O)OCC(CO)COC(=O)CC12CC3CC(CC(C3)C1)C2]

To a solution of Intermediate 1a (4 g, 1.0 equiv.) in DCM (40 mL) was added EDCI (2.5 g, 1.2 equiv.), DMAP (132 mg, 0.1 equiv.) and DIPEA (3.78 mL, 2.0 equiv-(1-adamantyl)acetic acid (2.11 g, 1.0 equiv.) was added to the above mixture at 0° C. The mixture was stirred at 20° C. for 2 h under N2 atmosphere. The reaction mixture was concentrated under reduced pressure to remove DCM. The residue was diluted with H₂O and extracted 2× with EtOAc. The combined organic layers were concentrated under reduced pressure to give a residue. The residue was purified by column chromatography to afford product as a colorless oil (4 g, 68%). ¹H NMR (400 MHz, CDCl₃) δ 5.37-5.20 (m, 4H), 4.19-4.01 (m, 4H), 3.56 (d, J=5.6 Hz, 2H), 2.70 (t, J=6.5 Hz, 2H), 2.25 (t, J=7.5 Hz, 2H), 2.13 (dt, J=11.6, 5.8 Hz, 2H), 2.04-1.95 (m, 6H), 1.90 (q, J=3.2 Hz, 3H), 1.64 (dt, J=12.3, 3.0 Hz, 3H), 1.55 (dd, J=15.4, 2.5 Hz, 11H), 1.33-1.15 (m, 15H), 0.87-0.77 (m, 3H).

### Intermediate 107b: 3-(2-((3r,5r,7r)-adamantan-1-yl)acetoxy)-2-((((4-nitrophenoxy)carbonyl)oxy)methyl)propyl (9Z,12Z)-octadeca-9,12-dienoate

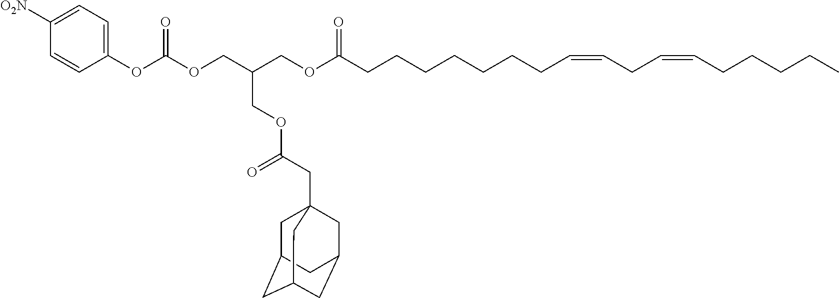 [SMILES: CCCCC/C=C\C/C=C\CCCCCCCC(=O)OCC(COC(=O)CC12CC3CC(CC(C3)C1)C2)COC(=O)Oc1ccc([N+](=O)[O-])cc1]

To a solution of Intermediate 107a (3 g, 1.0 equiv.), (4-nitrophenyl) carbonochloridate (3.3 g, 3.0 equiv.) in DCM (30 mL) was added pyridine (1.3 mL, 3.0 equiv.) at 0° C. The mixture was stirred at 20° C. for 2-5 h under N2 atmosphere. The reaction mixture was concentrated under reduced pressure to remove solvent. The residue was diluted with hexanes, filtered and the filtrate was concentrated under reduced pressure to give a residue. The residue was purified by silica gel chromatography to afford product as a colorless oil (3 g, 76% yield). ¹H NMR (400 MHz, CDCl₃) δ 8.31 (dd, J=9.3, 3.1 Hz, 2H), 7.41 (dd, J=9.3, 3.0 Hz, 2H), 5.37 (dtp, J=11.1, 7.1, 3.9 Hz, 4H), 4.39 (dd, J=6.0, 3.0 Hz, 2H), 4.23 (dt, J=9.5, 4.1 Hz, 4H), 2.80 (d, J=6.5 Hz, 2H), 2.54 (ddt, J=11.6, 8.5, 4.3 Hz, 1H), 2.35 (td, J=7.6, 3.0 Hz, 2H), 2.17-1.95 (m, 9H), 1.78-1.58 (m, 15H), 1.42-1.26 (m, 15H), 0.91 (td, J=6.8, 3.0 Hz, 3H).

### Example 107

### 3-(2-((3 r,5 r, 7r)-adamantan-1-yl)acetoxy)-2-((((3-(diethylamino)propoxy)carbonyl)oxy)methyl)propyl (9Z,12Z)-octadeca-9,12-dienoate

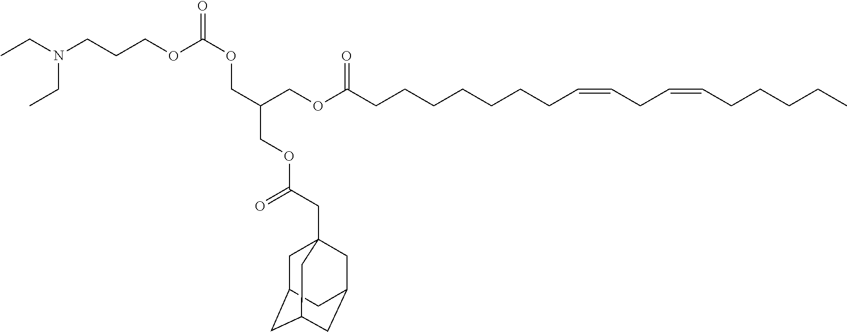 [SMILES: CCCCC/C=C\C/C=C\CCCCCCCC(=O)OCC(COC(=O)CC12CC3CC(CC(C3)C1)C2)COC(=O)OCCCN(CC)CC]

sTo a solution of Intermediate 107b (1 g, 1.0 equiv.) in MeCN (10 mL) was added 3-(diethylamino)propan-1-ol (555 mg, 3.0 equiv.), pyridine (342 uL, 2.0 equiv.) and DMAP (17 mg, 0.1 equiv.). The mixture was stirred at 20° C. for 12 h under N2 atmosphere. The reaction mixture was concentrated under reduced pressure to remove solvent. The residue was diluted with H₂O and and extracted 3× with EtOAc. The organic layer was dried over Na₂SO₄, filtered and the filtrate was concentrated under reduced pressure to give a residue. The residue was purified by column chromatography to afford product as a pale yellow oil (432 mg, 44%). ¹H NMR (400 MHz, CDCl₃) δ 5.28 (tt, J=11.2, 5.5 Hz, 4H), 4.21-3.97 (m, 8H), 2.70 (t, J=6.5 Hz, 2H), 2.40 (dq, J=29.5, 6.5, 6.0 Hz, 7H), 2.24 (t, J=7.6 Hz, 2H), 1.98 (dd, J=14.6, 7.7 Hz, 6H), 1.94-1.85 (m, 3H), 1.74 (p, J=6.8 Hz, 2H), 1.68-1.46 (m, 16H), 1.24 (d, J=6.6 Hz, 14H), 0.94 (t, J=7.1 Hz, 6H), 0.82 (t, J=6.7 Hz, 3H). MS: 703.3 m/z [M+H].

### Synthesis of Example 113

### Intermediate 113a: 1-((tert-butyldimethylsilyl)oxy)-3-hydroxypropan-2-one

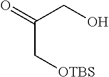 [SMILES: CC(C)(C)[Si](C)(C)OCC(=O)CO]

To a mixture of 1,3-dihydroxypropan-2-one (20 g, 1.0 equiv.) in THF (150 mL) was added imidazole (15.12 g, 1.0 equiv.). Then a mixture of TB SCl (1.0 equiv.) in THF (150 mL) was added dropwise to the above mixture at 0° C. The mixture was stirred at 15° C. for 2 h under N2. Upon completion, the reaction mixture was poured into water and extracted 3× with EtOAc. The combined organic phase was washed 2× with brine, dried with anhydrous Na₂SO₄, filtered and concentrated in vacuum. The reaction mixture was purified by column chromatography to afford product as a colorless oil (5.5 g, 12%). ¹H NMR (400 MHz, CDCl₃) δ 4.47 (s, 2H), 4.28 (s, 2H), 0.91-0.87 (m, 12H), 0.14-0.01 (m, 9H).

### Intermediate 113b: 3-((tert-butyldimethylsilyl)oxy)-2-oxopropyl (9Z,12Z)-octadeca-9,12-dienoate

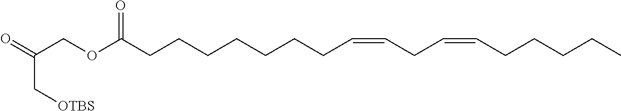 [SMILES: CCCCC/C=C\C/C=C\CCCCCCCC(=O)OCC(=O)CO[Si](C)(C)C(C)(C)C]

To a mixture of Intermediate 113a (5.5 g, 1.0 equiv.), EDCI (6.19 g, 1.2 equiv.), DMAP (658 mg, 0.2 equiv.), and DIPEA (14.06 mL, 3.0 equiv.) in DCM (55 mL) was added (9Z,12Z)-octadeca-9,12-dienoic acid (7.6 mL, 1.0 equiv.). The reaction was stirred at 15° C. for 12 h under N₂. Upon completion, the reaction mixture was poured into water and extracted 3× with DCM. The combined organic phase was dried with anhydrous Na₂SO₄, filtered and concentrated in vacuum. The residue was purified by column chromatography to afford product as a colorless oil (7 g, 56%). ¹H NMR (400 MHz, CDCl₃) δ 5.33-5.18 (m, 4H), 4.85 (s, 2H), 4.16 (s, 2H), 2.67 (t, J=6.5 Hz, 2H), 2.32 (t, J=7.5 Hz, 2H), 1.94 (q, J=6.9 Hz, 4H), 1.57 (q, J=7.3 Hz, 2H), 1.32-1.13 (m, 14H), 0.87-0.74 (m, 12H).

### p Intermediate 113c: 3-hydroxy-2-oxopropyl (9Z,12Z)-octadeca-9,12-dienoate

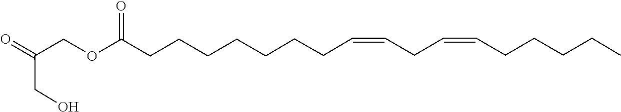 [SMILES: CCCCC/C=C\C/C=C\CCCCCCCC(=O)OCC(=O)CO]

To a solution of Intermediate 113b (7.0 g, 1.0 equiv.) in THF (70 mL) was added HF-pyridine (6.76 mL, 5.0 equiv.) dropwise at 0-15° C. The reaction mixture was stirred for 1 h at 15° C. The mixture was then poured into water and extracted 3× with EtOAc. The combined organic phase was washed with brine, dried with anhydrous Na₂SO₄, filtered, and concentrated in vacuum. The residue was purified by column chromatography to afford product as a colorless oil (3 g, 57%). 1H NMR (400 MHz, CDCl³) δ 5.35 (tp, J=11.2, 3.6, 3.2 Hz, 4H), 4.76 (s, 1H), 4.37 (s, 1H), 2.93 (s, 1H), 2.77 (t, J=6.4 Hz, 2H), 2.43 (t, J=7.5 Hz, 2H), 2.39-2.31 (m, 1H), 2.05 (p, J=7.1, 6.4 Hz, 4H), 1.65 (dp, J=11.8, 6.4, 5.4 Hz, 2H), 1.41-1.22 (m, 15H), 0.88 (t, J=6.7 Hz, 3H).

### Intermediate 113d: 3-((4,4-bis(octyloxy)butanoyl)oxy)-2-oxopropyl (9Z,12Z)-octadeca-9,12-dienoate

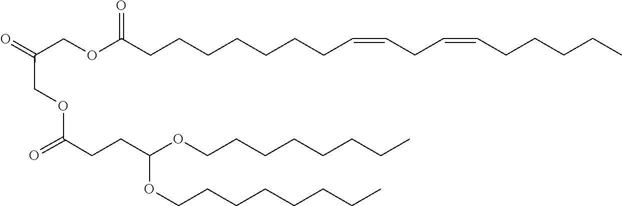 [SMILES: CCCCC/C=C\C/C=C\CCCCCCCC(=O)OCC(=O)COC(=O)CCC(OCCCCCCCC)OCCCCCCCC]

To a solution of Intermediate 113c (3 g, 1.0 equiv.), EDCI (1.96 g, 1.2 equiv.), DMAP (208 mg, 0.2 equiv.) and DIPEA (4.45 mL, 3.0 equiv.) in DCM (30 mL) was added Intermediate lc (2.93 g, 1.0 equiv.). Then the reaction mixture was stirred at 15° C. for 12 h under N₂. The residue was poured into water and extracted 3× with DCM. The combined organic phase was dried with anhydrous Na₂SO₄, filtered, and concentrated in vacuum. The residue was purified by column chromatography to afford product as a colorless oil (4 g, 69%). ¹H NMR (400 MHz, CDCl₃) δ 5.43-5.26 (m, 4H), 4.74 (d, J=1.9 Hz, 4H), 4.50 (t, J=5.5 Hz, 1H), 4.11 (q, J=7.1 Hz, 2H), 3.56 (dt, J=9.3, 6.6 Hz, 2H), 3.40 (dt, J=9.3, 6.7 Hz, 2H), 2.76 (t, J=6.4 Hz, 2H), 2.50 (t, J=7.5 Hz, 2H), 2.41 (t, J=7.5 Hz, 2H), 2.04 (d, J=5.9 Hz, 6H), 1.96 (dd, J=7.5, 5.5 Hz, 2H), 1.65 (p, J=7.2 Hz, 2H), 1.55 (q, J=6.9 Hz, 4H), 1.40-1.19 (m, 33H), 0.87 (td, J=6.8, 3.9 Hz, 8H).

### Intermediate 113e: 3-((4,4-bis(octyloxy)butanoyl)oxy)-2-hydroxypropyl (9Z,12Z)-octadeca-9,12-dienoate

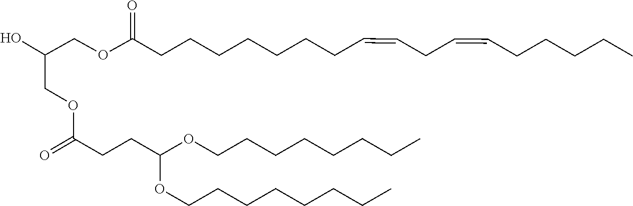 [SMILES: CCCCC/C=C\C/C=C\CCCCCCCC(=O)OCC(O)COC(=O)CCC(OCCCCCCCC)OCCCCCCCC]

To a solution of Intermediate 113d (4 g, 1.0 equiv.) in THF (80 mL), H₂O (40 mL, and toluene (20 mL) was added NaBH₄ (1.11 g, 5.0 equiv.) at 5° C. The mixture was stirred at 5° C. for 5 h. The reaction mixture was then poured into sat. NH₄Cl and extracted 2× with ethyl acetate. The combined organic phase was washed with brine, dried with anhydrous Na₂SO₄, filtered, and concentrated in vacuum. The residue was purified by column chromatography to afford product as a colorless oil (2.5 g, 62%).

### Intermediate 113f: 3-((4,4-bis(octyloxy)butanoyl)oxy)-2-(((4-nitrophenoxy)carbonyl)oxy)propyl (9Z,12Z)-octadeca-9,12-dienoate

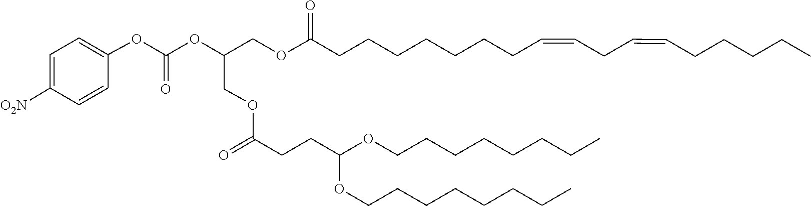 [SMILES: CCCCC/C=C\C/C=C\CCCCCCCC(=O)OCC(COC(=O)CCC(OCCCCCCCC)OCCCCCCCC)OC(=O)Oc1ccc([N+](=O)[O-])cc1]

To a solution of Intermediate 113e (2.5 g, 1.0 equiv.) and (4-nitrophenyl) carbonochloridate (1.48 g, 2.0 equiv.) in DCM (25 mL) was added DMAP (4 mg, 0.01 equiv.) and pyridine (5.93 mL, 20 equiv.). The reaction mixture was stirred for 5 h at 15° C. The reaction mixture was concentrated under reduced pressure to remove solvent. The crude product (colorless oil) was used directly in next step with additional purification (2.4 g, 79%).

### Example 113

### 3-((4,4-bis(octyloxy)butanoyl)oxy)-2-(((2-(diethylamino)ethyl)carbamoyl)oxy)propyl (9Z,12Z)-octadeca-9,12-dienoate

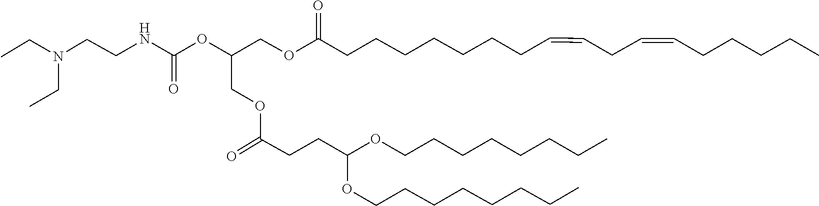 [SMILES: CCCCC/C=C\C/C=C\CCCCCCCC(=O)OCC(COC(=O)CCC(OCCCCCCCC)OCCCCCCCC)OC(=O)NCCN(CC)CC]

To a solution of Intermediate 113f (800 mg, 1.0 equiv.) and N′,N′-diethylethane-1,2-diamine (664 uL, 5.0 equiv.) in MeCN (10 mL) was added pyridine (1.53 mL, 20 equiv.)

and DMAP (12 mg, 0.1 equiv.). The reaction mixture was stirred at 15° C. for 12 h. The mixture was poured into water and extracted 3× with EtOAc. The combined organic phase was dried with anhydrous Na₂SO₄, filtered and concentrated in vacuum. The residue was purified by column chromatography to afford product as a colorless oil (320 mg, 41%). ¹H NMR (400 MHz, CDCl₃) δ 5.33 (ddt, J=19.7, 13.2, 7.4 Hz, 4H), 5.19-5.09 (m, 1H), 4.47 (t, J=5.6 Hz, 1H), 4.25 (dt, J=11.5, 3.8 Hz, 2H), 4.17 (dd, J=11.9, 5.7 Hz, 2H), 3.55 (dt, J=9.4, 6.7 Hz, 2H), 3.38 (dt, J=9.2, 6.7 Hz, 2H), 3.21 (q, J=5.7 Hz, 2H), 2.76 (t, J=6.5 Hz, 2H), 2.51 (q, J=7.3 Hz, 5H), 2.39 (t, J=7.6 Hz, 2H), 2.30 (t, J=7.6 Hz, 2H), 2.03 (q, J=6.9 Hz, 3H), 1.91 (td, J=7.6, 5.5 Hz, 2H), 1.57 (dt, J=21.9, 7.1 Hz, 5H), 1.29 (q, J=10.3, 5.5 Hz, 25H), 0.99 (t, J=7.1 Hz, 4H), 0.87 (td, J=6.7, 3.9 Hz, 6H). MS: 825.4 m/z [M+H].

### Synthesis of Example 114

### Intermediate 114a: 3-hydroxy-2-(hydroxymethyl)propyl stearate

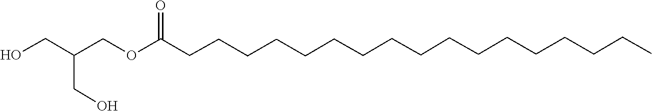 [SMILES: CCCCCCCCCCCCCCCCCC(=O)OCC(CO)CO]

To a solution (2,2-dimethyl-1,3-dioxan-5-yl)methanol (1.53 g, 10.5 mmol), stearic acid (3 g, 10.5 mmol), DMAP (256 mg, 2.1 mmol), and DIPEA (4.39 mL, 25.2 mmol) in DCM (25 mL) was added EDC-HCl (2.41 g, 12.6 mmol) at rt. The reaction mixture was stirred at rt for 24 h then diluted with water (25 mL). The organic layer was collected, washed with 1 HCl (10 mL), saturated NaHCO₃ solution (10 mL), then water (10 mL). Organic layer was collected, dried over anhydrous sodium sulfate, then concentrated in vacuo. The crude residue was dissolved in methanol, followed by hexanes until sample was fully solubilized and Dowex® 50×8 resin was added. The resulting reaction mixture was stirred for 24 h at rt, filtered, and concentrated in vacuo to provide 2.74 g (7.35 mmol, 70% yield) of the desired product as a white solid. ¹H NMR (CDCl₃, 400 MHz) δ 4.27 (d, J=6.3 Hz, 2H), 3.77 (m, 4H), 2.33 (m, 2H), 2.10 (s, 2H), 2.03 (m, 1H), 1.62 (m, 2H), 1.25 (m, 29H), 0.87 (t, J=6.7 Hz, 3H); MS: 373.32 m/z [M+H].

### Intermediate 114b: 3-((4,4-bis(octyloxy)butanoyl)oxy)-2-(hydroxymethyl)propyl stearate

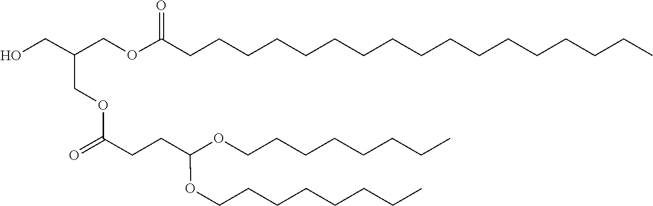 [SMILES: CCCCCCCCCCCCCCCCCC(=O)OCC(CO)COC(=O)CCC(OCCCCCCCC)OCCCCCCCC]

Intermediate 114b was synthesized in 43% yield from Intermediate 114a and Intermediate 1c using the method employed for Intermediate 1d. ¹H NMR (CDCl₃, 400 MHz) δ 4.49 (t, J=5.5 Hz, 1H), 4.17 (m, 4H), 3.62 (t, J=5.5 Hz, 2H), 3.56 (m, 2H), 3.40 (m, 2H), 2.41 (t, J=7.5 Hz, 2H), 2.32 (t, J=7.6 Hz, 2H), 2.20 (m, 2H), 1.62 (m, 2H), 1.55 (m, 6H), 1.27 (m, 49H), 0.88 (t, J=6.8 Hz, 9H); MS: 721.78 m/z [M+Na].

### Example 114

### 3-((4,4-bis(octyloxy)butanoyl)oxy)-2-((((3-(diethylamino)propoxy)carbonyl)oxy)methyl)propyl stearate

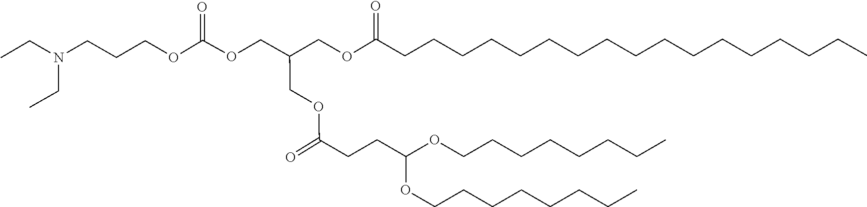 [SMILES: CCCCCCCCCCCCCCCCCC(=O)OCC(COC(=O)CCC(OCCCCCCCC)OCCCCCCCC)COC(=O)OCCCN(CC)CC]

Example 114 was synthesized in 30% yield from Intermediate 114b and 3-(diethylamino)propan-1-ol using the method employed for Example 1. ¹H NMR (CDCl₃, 400 MHz) δ 4.48 (t, J=5.5 Hz, 1H), 4.17 (m, 8H), 3.56 (m, 2H), 3.40 (m, 2H), 2.52 (s, 5H), 2.40 (m, 3H), 2.30 (t, J=7.6 Hz, 2H), 1.92 (m, 2H), 1.82 (s, 2H), 1.57 (m, 7H), 1.26 (m, 48H), 1.02 (t, J=6.8 Hz, 6H), 0.88 (m, 9H) ppm; MS: 857.14 m/z [M+H].

### Synthesis of Example 115

### Intermediate 115a: 3-hydroxy-2-(hydroxymethyl)propyl oleate

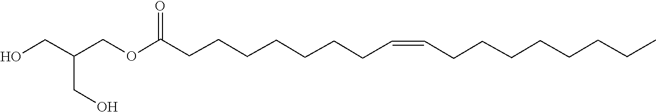 [SMILES: CCCCCCCC/C=C\CCCCCCCC(=O)OCC(CO)CO]

To a solution (2,2-dimethyl-1,3-dioxan-5-yl)methanol (1.53 g, 10.5 mmol), oleic acid (3.37 mL, 10.5 mmol), DMAP (256 mg, 2.1 mmol), and DIPEA (4.39 mL, 25.2 mmol) in DCM (25 mL) was added EDC-HCl (2.41 g, 12.6 mmol) at rt. The reaction mixture was stirred at rt for 24 h then diluted with water (25 mL). The organic layer was collected, washed with 1 HCl (10 mL), saturated NaHCO₃ solution (10 mL), then water (10 mL). Organic layer was collected, dried over anhydrous sodium sulfate, then concentrated in vacuo. The crude residue was dissolved in methanol and Dowex® 50×8 resin was added. The resulting reaction mixture was stirred for 24 h at rt, filtered, and concentrated in vacuo to provide 2.76 g (7.44 mmol, 70% yield) of the desired product as a clear oil. ¹H NMR (CDCl₃, 400 MHz) δ 5.35 (m, 2H), 4.27 (d, J=6.3 Hz, 2H), 3.77 (m, 4H), 2.33 (t, 7.5 Hz, 2H), 2.02 (m, 6H), 1.62 (m, 2H), 1.29 (m, 20H), 0.88 (t, J=6.8 Hz, 3H); MS: 371.29 m/z [M+H].

### Intermediate 115b: 3-((4,4-bis(octyloxy)butanoyl)oxy)-2-(hydroxymethyl)propyl oleate

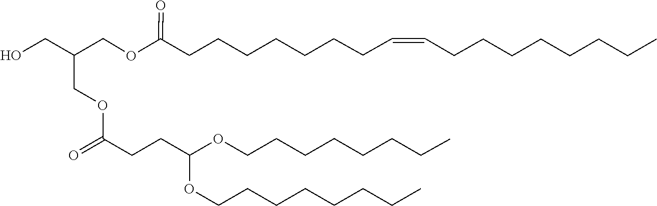 [SMILES: CCCCCCCC/C=C\CCCCCCCC(=O)OCC(CO)COC(=O)CCC(OCCCCCCCC)OCCCCCCCC]

Intermediate 115b was synthesized in 45% yield from Intermediate 115a and Intermediate 1c using the method employed for Intermediate 1d.¹HNMR (CDCl₃, 400 MHz) δ 5.34 (m, 2H), 4.49 (t, J=5.5 Hz, 1H) 4.18 (m, 4H), 3.62 (t, J=5.6 Hz, 2H), 3.56 (m, 2H), 3.40 (m, 2H), 2.41 (t, J=7.5 Hz, 2H), 2.32 (m, 2H), 2.20 (m, 2H), 2.01 (q, J=6.2 Hz, 4H), 1.94 (m, 2H), 1.61 (q, J=7.3 Hz, 2H), 1.56 (m, 6H), 1.29 (m, 42H), 0.87 (m, 9H); MS: 720.52 m/z [M+Na].

### Example 115

### 3-((4,4-bis(octyloxy)butanoyl)oxy)-2-((((3-(diethylamino)propoxy)carbonyl)oxy)methyl)propyl oleate

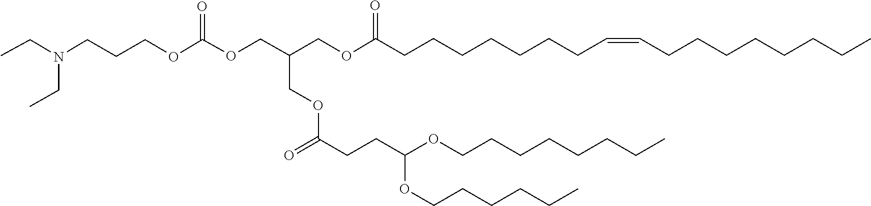

Example 115 was synthesized in 40% yield from Intermediate 115b and 3-(diethylamino)propan-1-ol using the method employed for Example 1. ¹H NMR (CDCl₃, 400 MHz) δ 5.34 (m, 2H), 4.48 (t, J=5.5 Hz, 1H), 4.17 (m, 8H), 3.56 (m, 2H), 3.40 (m, 2H), 2.52 (s, 5H), 2.40 (m, 3H), 2.30 (t, J=7.6 Hz, 2H), 2.00 (m, 4H), 1.92 (m, 2H), 1.82 (m, 2H), 1.57 (m, 7H), 1.26 (m, 40H), 1.02 (t, J=7.1 Hz, 6H), 0.88 (m, 9H) ppm; MS: 855.37 m/z [M+H].

### Synthesis of Example 116

### Intermediate 116a: 3-hydroxy-2-(hydroxymethyl)propyl (9Z,12Z,15Z)-octadeca-9,12,15-trienoate

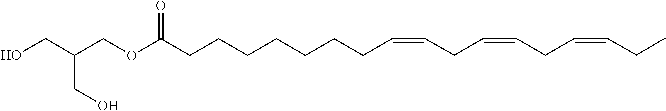 [SMILES: CC/C=C\C/C=C\C/C=C\CCCCCCCC(=O)OCC(CO)CO]

To a solution (2,2-dimethyl-1,3-dioxan-5-yl)methanol (1.53 g, 10.5 mmol), linolenic acid (3.28 mL, 10.5 mmol), DMAP (256 mg, 2.1 mmol), and DIPEA (4.39 mL, 25.2 mmol) in DCM (25 mL) was added EDC-HCl (2.41 g, 12.6 mmol) at rt. The reaction mixture was stirred at rt for 24 h then diluted with water (25 mL). The organic layer was collected, washed with 1 HCl (10 mL), saturated NaHCO₃ solution (10 mL), then water (10 mL). Organic layer was collected, dried over anhydrous sodium sulfate, then concentrated in vacuo. The crude residue was dissolved in methanol and Dowex® 50×8 resin was added. The resulting reaction mixture was stirred for 24 h at rt, filtered, and concentrated in vacuo to provide 2.36 g (6.43 mmol, 60% yield) of the desired product as a clear oil. ¹H NMR (CDCl₃, 400 MHz) δ 5.36 (m, 6H), 4.27 (d, J=6.3 Hz, 2H), 3.77 (m, 4H), 2.81 (m, 4H), 2.33 (t, J=7.5 Hz, 2H), 2.06 (m, 6H), 1.62 (m, 2H), 1.31 (m, 9H), 0.98 (t, J=7.5 Hz, 3H); MS: 367.29 m/z [M+H].

### Intermediate 116b: 3-((4,4-bis(octyloxy)butanoyl)oxy)-2-(hydroxymethyl)propyl (9Z,12Z,15Z)-octadeca-9,12,15-trienoate

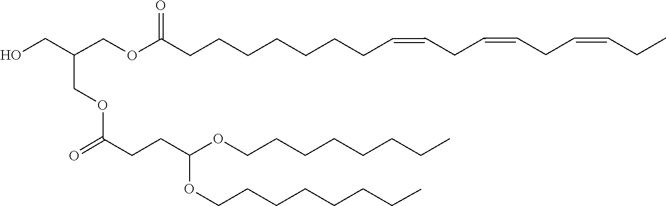 [SMILES: CC/C=C\C/C=C\C/C=C\CCCCCCCC(=O)OCC(CO)COC(=O)CCC(OCCCCCCCC)OCCCCCCCC]

Intermediate 116b was synthesized in 45% yield from Intermediate 116a and Intermediate lc using the method employed for Intermediate 1d. ¹H NMR (CDCl₃, 400 MHz) δ 5.36 (m, 6H), 4.49 (t, J=5.5 Hz, 1H), 4.18 (m, 4H), 3.62 (t, J=5.6 Hz, 2H), 3.56 (m, 2H), 3.40 (m, 2H), 2.81 (m, 4H), 2.41 (t, J=7.5 Hz, 2H), 2.32 (t, J=7.6 Hz, 2H), 2.20 (m, 2H), 2.07 (m, 4H), 1.93 (m, 2H), 1.62 (m, 2H), 1.56 (m, 6H), 1.31 (m, 30H), 0.98 (t, J=7.5 Hz, 3H), 0.88 (t, J=6.9 Hz, 6H); MS: 715.93 m/z [M+Na].

### Example 116

### 3-((4,4-bis(octyloxy)butanoyl)oxy)-2-((((3-(diethylamino)propoxy)carbonyl)oxy)methyl)propyl (9Z,12Z,15Z)-octadeca-9,12,15-trienoate

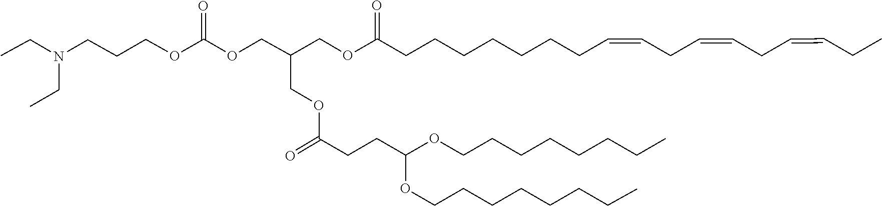 [SMILES: CC/C=C\C/C=C\C/C=C\CCCCCCCC(=O)OCC(COC(=O)CCC(OCCCCCCCC)OCCCCCCCC)COC(=O)OCCCN(CC)CC]

Example 116 was synthesized in 31% yield from Intermediate 116b and 3-(diethylamino)propan-1-ol using the method employed for Example 1. ¹H NMR (CDCl₃, 400 MHz) δ 5.34 (m, 6H), 4.48 (t, J=5.6 Hz, 1H), 4.17 (m, 8H), 3.56 (m, 2H), 3.40 (m, 2H), 2.81 (m, 4H) 2.52 (s, 5H), 2.40 (m, 3H), 2.30 (t, J=7.6 Hz, 2H), 2.08 (m, 4H), 1.92 (m, 2H), 1.82 (m, 2H), 1.57 (m, 8H), 1.29 (m, 28H), 0.99 (m, 8H), 0.88 (m, 6H) ppm; MS: 851.32 m/z [M+H].

### Synthesis of Example 117

### Intermediate 117a: heptadecan-9-yl (3-(((4-nitrophenoxy)carbonyl)oxy)-2-((((9Z,12Z)-octadeca-9,12-dienoyl)oxy)methyl)propyl) glutarate

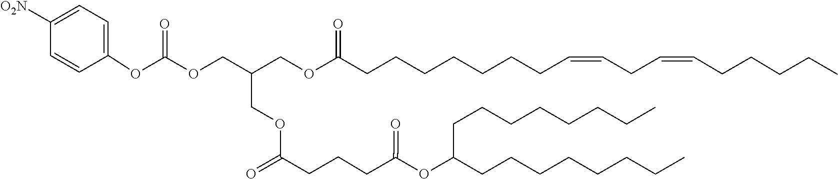 [SMILES: CCCCC/C=C\C/C=C\CCCCCCCC(=O)OCC(COC(=O)CCCC(=O)OC(CCCCCCCC)CCCCCCCC)COC(=O)Oc1ccc([N+](=O)[O-])cc1]

To a mixture of Intermediate 63a (1.0 equiv.) and (4-nitrophenyl) carbonochloridate (2.0 equiv.) in DCM (0.05-0.2 M) was added pyridine (2.0 equiv.). The mixture was stirred at 20° C. for 5 h under inert atmosphere. Upon completion, the mixture was concentrated in vacuo, and the resulting residue was diluted with hexanes and filtered. The filtrate was concentrated to afford a color residue that was used directly in the next step without further purification (47%).

### Example 117

### 3-(((2-(diethylamino)ethyl)carbamoyl)oxy)-2-((((9Z,12Z)-octadeca-9,12-dienoyl)oxy)methyl)propyl heptadecan-9-yl glutarate

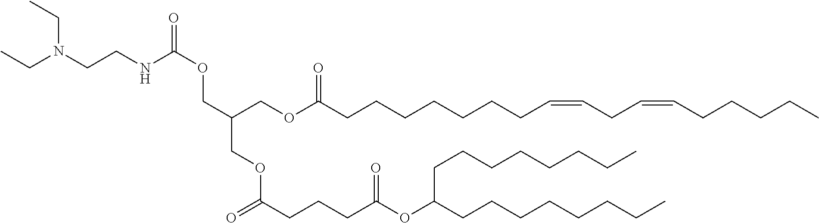 [SMILES: CCCCC/C=C\C/C=C\CCCCCCCC(=O)OCC(COC(=O)CCCC(=O)OC(CCCCCCCC)CCCCCCCC)COC(=O)NCCN(CC)CC]

To a mixture of Intermediate 117a (1.0 equiv.) in MeCN (0.1 M) was added N′,N′-diethylethane-1,2-diamine (2.0 equiv.), pyridine (2.0 equiv.), and DMAP (1.0 equiv.) under inert atmosphere. The mixture was stirred at 20° C. for 12 h under inert atmosphere, after which point the mixture was concentrated in vacuo. The resulting residue was diluted with EtOAc and washed 5× with 1 N NaHCO₃ and 3× with H₂O. The organic layer was dried over Na₂SO₄, filtered, concentrated in vacuo, and purified by column chromatography to afford product as a pale yellow oil (36%). ¹H NMR (400 MHz, CDCl₃) δ 5.36-5.21 (m, 5H), 4.79 (p, J=6.2 Hz, 1H), 4.06 (t, J=5.9 Hz, 6H), 3.20 (s, 2H), 2.70 (t, J=6.4 Hz, 2H), 2.49 (d, J=25.8 Hz, 6H), 2.36-2.20 (m, 8H), 1.97 (dd, J=7.8, 5.9 Hz, 5H), 1.87 (p, J=7.5 Hz, 3H), 1.54 (t, J=7.2 Hz, 2H), 1.43 (d, J=6.5 Hz, 4H), 1.35-1.06 (m, 42H), 0.98 (s, 6H), 0.81 (td, J=6.8, 4.9 Hz, 9H). MS: 863.7 m/z [M+H].

### Synthesis of Example 118

### Intermediate 118a: 1-(heptadecan-9-yl) 7-(3-(((4-nitrophenoxy)carbonyl)oxy)-2-((((9Z,12Z)-octadeca-9,12-dienoyl)oxy)methyl)propyl) heptanedioate

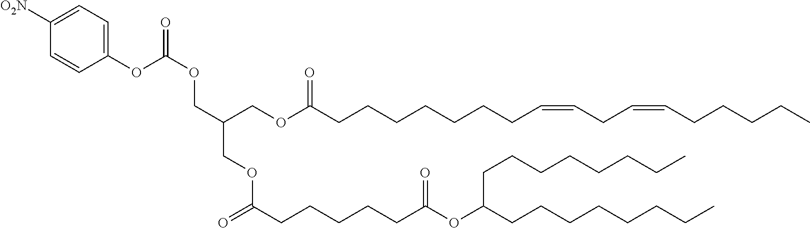 [SMILES: CCCCC/C=C\C/C=C\CCCCCCCC(=O)OCC(COC(=O)CCCCCC(=O)OC(CCCCCCCC)CCCCCCCC)COC(=O)Oc1ccc([N+](=O)[O-])cc1]

Intermediate 118a was synthesized in 61% yield from Intermediate 64b using the method employed for Intermediate 117a.

### Example 118

### 1-(3-(((2-(diethylamino)ethyl)carbamoyl)oxy)-2-((((9Z,12Z)-octadeca-9,12-dienoyl)oxy)methyl)propyl) 7-(heptadecan-9-yl) heptanedioate

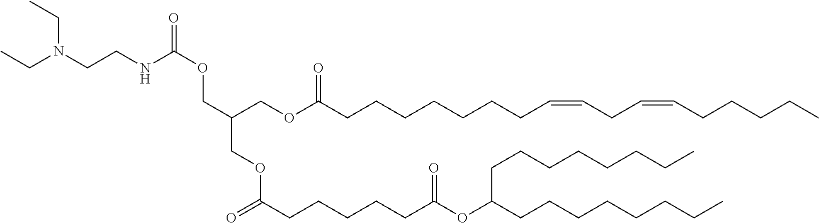 [SMILES: CCCCC/C=C\C/C=C\CCCCCCCC(=O)OCC(COC(=O)CCCCCC(=O)OC(CCCCCCCC)CCCCCCCC)COC(=O)NCCN(CC)CC]

Example 118 was synthesized in 96% yield from Intermediate 118a using the method employed for Example 117. ¹H NMR (400 MHz, CDCl₃) δ 5.28 (dtt, J=17.4, 10.8, 6.0 Hz, 4H), 5.15 (s, 1H), 4.79 (p, J=6.2 Hz, 1H), 4.05 (d, J=6.1 Hz, 6H), 3.14 (q, J=5.9 Hz, 2H), 2.70 (t, J=6.5 Hz, 2H), 2.45 (q, J=6.8 Hz, 6H), 2.37-2.18 (m, 7H), 1.98 (q, J=7.0 Hz, 4H), 1.56 (q, J=7.9 Hz, 6H), 1.43 (q, J=6.3 Hz, 4H), 1.21 (d, J=18.3 Hz, 40H), 0.93 (t, J=7.1 Hz, 6H), 0.81 (q, J=6.4 Hz, 9H). MS: 891.8 m/z [M+H].

### Example 119

### pKa Measurements

The pKa of each amine lipid was determined according to the method in Jayaraman, et al. (Angewandte Chemie, 2012) with the following adaptations. The pKa was determined for unformulated amine lipid in ethanol at a concentration of 2.94 mM. Lipid was diluted to 100 μM in 0.1 M phosphate buffer (Boston Bioproducts) where the pH ranged from 2.0-12.0. Fluorescence intensity was measured using excitation and emission wavelengths of 321 nm and 448 nm. Table 1 shows pKa measurements for listed compounds.

TABLE 1
pKa values

| Compound | pKa |
|---|---|
| Compound 1 | 6.4 |
| Compound 25 | 7.65 |
| Compound 26 | 7.39 |
| Compound 27 | 5.35 |
| Compound 28 | 7.3 |
| Compound 29 | 6.84 |
| Compound 30 | 6.06 |
| Compound 31 | 7.31 |
| Compound 32 | 7.32 |
| Compound 33 | 6.03 |
| Compound 34 | 7.84 |
| Compound 35 | 7.39 |
| Compound 36 | 6.58 |
| Compound 37 | 7.84 |
| Compound 38 | 7.17 |
| Compound 39 | 7.34 |
| Compound 40 | 7.33 |
| Compound 41 | 7.82 |
| Compound 42 | 6.9 |
| Compound 43 | 8.6 |
| Compound 44 | 6.1 |
| Compound 45 | 7.3 |
| Compound 46 | 8.9 |
| Compound 47 | 5.9 |
| Compound 48 | 5.3 |
| Compound 49 | 8.4 |
| Compound 50 | 5.2 |
| Compound 51 | 7.3 |
| Compound 52 | 7.8 |
| Compound 53 | 6.85 |
| Compound 54 | 6.4 |
| Compound 55 | 6.84 |
| Compound 56 | 6.85 |
| Compound 57 | 6.586 |
| Compound 58 | 6.4 |
| Compound 59 | 6.396 |
| Compound 60 | 6.382 |
| Compound 61 | 6.295 |
| Compound 62 | 6.03 |
| Compound 63 | 6.09 |
| Compound 64 | 6.08 |
| Compound 65 | 5.88 |
| Compound 66 | 6.06 |
| Compound 67 | 6.19 |
| Compound 68 | 6.1 |
| Compound 69 | 6.18 |
| Compound 70 | 6.02 |
| Compound 71 | 6.44 |
| Compound 72 | 6.07 |
| Compound 73 | 6.64 |
| Compound 74 | 6.39 |
| Compound 75 | 6.45 |
| Compound 76 | 6.17 |
| Compound 77 | 6.16 |
| Compound 78 | 6.11 |
| Compound 79 | 6.13 |
| Compound 80 | 6.21 |
| Compound 81 | 6.65 |
| Compound 82 | 6.31 |
| Compound 83 | 6.16 |
| Compound 84 | 6.16 |
| Compound 85 | 6.16 |
| Compound 86 | 6.16 |
| Compound 87 | 6.52 |
| Compound 88 | 6.84 |
| Compound 89 | 6.79 |
| Compound 90 | 6.14 |
| Compound 91 | 6.08 |
| Compound 92 | 6.16 |
| Compound 93 | 6.15 |
| Compound 94 | 6.14 |
| Compound 95 | 6.14 |
| Compound 96 | 5.76 |
| Compound 97 | 6.09 |
| Compound 98 | 6.4 |
| Compound 99 | 7.1 |
| Compound 100 | 7.2 |
| Compound 101 | 6.4 |
| Compound 102 | 5.7 |
| Compound 103 | 7.0 |
| Compound 107 | 6.43 |
| Compound 113 | NA |
| Compound 114 | 6.134 |
| Compound 115 | 5.957 |
| Compound 116 | 6.039 |
| Compound 117 | NA |
| Compound 118 | NA |

### Example 120

### Materials and Methods

LNP Compositions for In Vivo Editing

LNPs were prepared using various amine lipids in a 4-component lipid system consisting of an ionizable lipid (e.g. an amine lipid), DSPC, cholesterol and PEG-2k-DMG. Molar concentrations of lipids in the lipid component of the LNPs are used at mol % amine lipid/DSPC/cholesterol/PEG-2k-DMG of 50/9/38/3. In assays for percent liver editing in mice, Cas9 mRNA and chemically modified sgRNA targeting a mouse sequence were formulated in LNPs, at either a 1:1 w/w ratio, 1:2 w/w ratio, or 1:1.3 w/w ratio.

LNP Formulation

The lipid components were dissolved in 100% ethanol with the lipid component molar ratios described below. The chemically modified sgRNA and Cas9 mRNA were combined and dissolved in 25 mM citrate, 100 mM NaCl, pH 5.0, resulting in a concentration of total RNA cargo of approximately 0.45 mg/mL. The LNPs were formulated with an N/P ratio of about 6, with the ratio of chemically modified sgRNA: Cas9 mRNA at either a 1:1 w/w ratio, 1:2 w/w ratio, or 1:1.3 w/w ratio as described below.

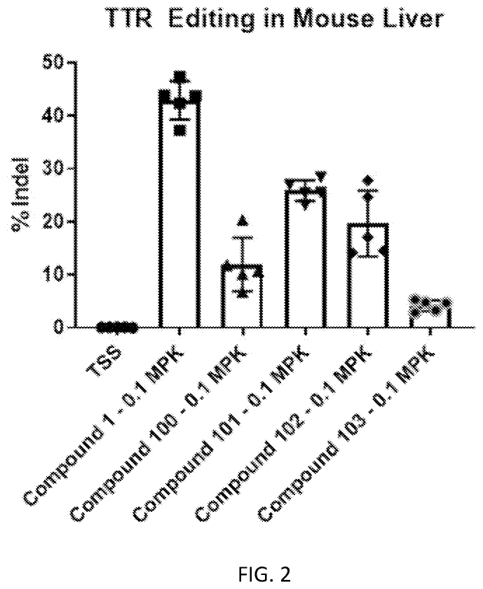

The LNPs were formed by an impinging jet mixing of the lipid in ethanol with two volumes of RNA solution and one volume of water. The lipid in ethanol was mixed through a mixing cross with the two volumes of RNA solution. A fourth stream of water is mixed with the outlet stream of the cross through an inline tee. (See, e.g., WO2016010840, FIG. 2.) Alternatively, the LNPs were formed by microfluidic mixing of the lipid and RNA solutions using a Precision Nanosystems NanoAssemblr™ Benchtop Instrument, according to the manufacturer's protocol. A 2:1 ratio of aqueous to organic solvent was maintained during mixing using differential flow rates. The LNPs were held for 1 hour at room temperature, and further diluted with water (approximately 1:1 v/v). Diluted LNPs were concentrated using tangential flow filtration on a flat sheet cartridge (Sartorius, 100 kD MWCO) and then buffer exchanged by diafiltration into 50 mM Tris, 45 mM NaCl, 5% (w/v) sucrose, pH 7.5 (TSS). Alternatively, the final buffer exchange into TSS was completed with PD-10 desalting columns (GE). If required, compositions were concentrated by centrifugation with Amicon 100 kDa centrifugal filters (Millipore). The resulting mixture was then filtered using a 0.2 μm sterile filter. The final LNP was stored at 4° C. or −80° C. until further use.

LNP Composition Analytics

Dynamic Light Scattering (“DLS”) is used to characterize the polydispersity index (“PDI”) and size of the LNPs of the present disclosure. DLS measures the scattering of light that results from subjecting a sample to a light source. PDI, as determined from DLS measurements, represents the distribution of particle size (around the mean particle size) in a population, with a perfectly uniform population having a PDI of zero.

Electropheretic light scattering is used to characterize the surface charge of the LNP at a specified pH. The surface charge, or the zeta potential, is a measure of the magnitude of electrostatic repulsion/attraction between particles in the LNP suspension.

Asymetric-Flow Field Flow Fractionation—Multi-Angle Light Scattering (AF4-MALS) is used to separate particles in the composition by hydrodynamic radius and then measure the molecular weights, hydrodynamic radii and root mean square radii of the fractionated particles. This allows the ability to assess molecular weight and size distributions as well as secondary characteristics such as the Burchard-Stockmeyer Plot (ratio of root mean square (“rms”) radius to hydrodynamic radius over time suggesting the internal core density of a particle) and the rms conformation plot (log of rms radius vs log of molecular weight where the slope of the resulting linear fit gives a degree of compactness vs elongation).

Nanoparticle tracking analysis (NTA, Malvern Nanosight) can be used to determine particle size distribution as well as particle concentration. LNP samples are diluted appropriately and injected onto a microscope slide. A camera records the scattered light as the particles are slowly infused through field of view. After the movie is captured, the Nanoparticle Tracking Analysis processes the movie by tracking pixels and calculating a diffusion coefficient. This diffusion coefficient can be translated into the hydrodynamic radius of the particle. The instrument also counts the number of individual particles counted in the analysis to give particle concentration.

Cryo-electron microscopy (“cryo-EM”) can be used to determine the particle size, morphology, and structural characteristics of an LNP.

Lipid compositional analysis of the LNPs can be determined from liquid chromotography followed by charged aerosol detection (LC-CAD). This analysis can provide a comparison of the actual lipid content versus the theoretical lipid content.

LNP compositions are analyzed for average particle size, polydispersity index (PDI), total RNA content, encapsulation efficiency of RNA, and zeta potential. LNP compositions may be further characterized by lipid analysis, AF4-MALS, NTA, and/or cryo-EM. Average particle size and polydispersity are measured by dynamic light scattering (DLS) using a Malvern Zetasizer DLS instrument or Wyatt NanoStar. LNP samples were diluted with PBS buffer prior to being measured by DLS. Z-average diameter which is an intensity-based measurement of average particle size is reported along with number average diameter and PDI. A Malvern Zetasizer or Wyatt NanoStar instrument is also used to measure the zeta potential of the LNP. Samples are diluted 1:17 (50 μL into 800 μL) in 0.1× PBS, pH 7.4 prior to measurement.

A fluorescence-based assay (Ribogreen®, ThermoFisher Scientific) is used to determine total RNA concentration and free RNA. Encapsulation efficiency is calculated as (Total RNA-Free RNA)/Total RNA. LNP samples are diluted appropriately with 1× TE buffer containing 0.2% Triton-X 100 to determine total RNA or 1× TE buffer to determine free RNA. Standard curves are prepared by utilizing the starting RNA solution used to make the compositions and diluted in 1× TE buffer +/− 0.2% Triton-X 100. Diluted RiboGreen® dye (according to the manufacturer's instructions) is then added to each of the standards and samples and allowed to incubate for approximately 10 minutes at room temperature, in the absence of light. A SpectraMax M5 Microplate Reader (Molecular Devices) is used to read the samples with excitation, auto cutoff and emission wavelengths set to 488 nm, 515 nm, and 525 nm respectively. Total RNA and free RNA are determined from the appropriate standard curves.

Encapsulation efficiency is calculated as (Total RNA-Free RNA)/Total RNA. The same procedure may be used for determining the encapsulation efficiency of a DNA-based cargo component. In a fluorescence-based assay, for single-strand DNA Oligreen Dye may be used, and for double-strand DNA, Picogreen Dye. Alternatively, the total RNA concentration can be determined by a reverse-phase ion-pairing (RP-IP) HPLC method. Triton X-100 is used to disrupt the LNPs, releasing the RNA. The RNA is then separated from the lipid components chromatographically by RP-IP HPLC and quantified against a standard curve using UV absorbance at 260 nm.

AF4-MALS is used to look at molecular weight and size distributions as well as secondary statistics from those calculations. LNPs are diluted as appropriate and injected into a AF4 separation channel using an HPLC autosampler where they are focused and then eluted with an exponential gradient in cross in cross flow across the channel. All fluid is driven by an HPLC pump and Wyatt Eclipse Instrument. Particles eluting from the AF4 channel flow through a UV detector, multi-angle light scattering detector, quasi-elastic light scattering detector and differential refractive index detector. Raw data is processed by using a Debye model to determine molecular weight and rms radius from the detector signals.

Lipid components in LNPs are analyzed quantitatively by HPLC coupled to a charged aerosol detector (CAD). Chromatographic separation of 4 lipid components is achieved by reverse phase HPLC. CAD is a destructive mass-based detector which detects all non-volatile compounds and the signal is consistent regardless of analyte structure.

Cas9 mRNA and gRNA Cargos

The Cas9 mRNA (e.g. SEQ ID NO: 3) cargo was prepared by in vitro transcription. Capped and polyadenylated Cas9 mRNA comprising 1× NLS was generated by in vitro transcription using a linearized plasmid DNA template and T7 RNA polymerase using a method as follows. Plasmid DNA containing a T7 promoter and a 100 nt poly(A/T) region is linearized by incubating at 37° C. for 2 hours with XbaI with the following conditions: 200 ng/μL plasmid, 2 U/μL XbaI (NEB), and 1× reaction buffer. The XbaI is inactivated by heating the reaction at 65° C. for 20 min. The linearized plasmid is purified from enzyme and buffer salts using a silica maxi spin column (Epoch Life Sciences) and analyzed by agarose gel to confirm linearization. The IVT reaction to generate Cas9 modified mRNA is performed by incubating at 37° C. for 4 hours in the following conditions: 50 ng/μL linearized plasmid; 2 mM each of GTP, ATP, CTP, and N1-methyl pseudo-UTP (Trilink); 10 mM ARCA (Trilink); 5 U/μL T7 RNA polymerase (NEB); 1 U/μL Murine RNase inhibitor (NEB); 0.004 U/μL Inorganic E. coli pyrophosphatase (NEB); and 1× reaction buffer. After the 4 hr incubation, TURBO DNase (ThermoFisher) is added to a final concentration of 0.01 U/μL, and the reaction is incubated for an additional 30 minutes to remove the DNA template. The Cas9 mRNA was purified with TFF or an LiCl precipitation-containing method.

A capped and polyadenylated Cas9 mRNA comprising SEQ ID NO:6 and 1× NLS was generated by in vitro transcription using a linearized plasmid DNA template and T7

RNA polymerase. plasmid DNA containing a T7 promoter and a poly(A/T) region between 90-100 nt is linearized by incubating at 37° C. with XbaI to completion. The linearized plasmid is purified from enzyme and buffer salts. The IVT reaction to generate Cas9 modified mRNA is performed by incubating at 37° C. for 1.5 or 2 hours in the following conditions: 50 ng/μL linearized plasmid; 5 mM each of GTP, ATP, CTP, and N1-methyl pseudo-UTP (Trilink); 25 mM ARCA (Trilink); 5 U/μL T7 RNA polymerase; 1 U/μL Murine RNase inhibitor; 0.004 U/μL Inorganic E. coli pyrophosphatase ; and 1× reaction buffer. TURBO DNase (ThermoFisher) is then added to remove the DNA template.

mRNA was purified from enzyme and nucleotides using a RNeasy Maxi kit (Qiagen) according to the manufacturer's protocol. Alternately, mRNA was purified using a MEGAclear kit (Invitrogen) according to the manufacturer's protocol. Alternatively, mRNA is purified using LiCl precipitation, ammonium acetate precipitation and sodium acetate precipitation. Alternatively, mRNA is purified with a LiCl precipitation method followed by further purification by tangential flow filtration. Alternatively, RNA was purified by LiCl precipitation in combination with tangential flow filtration. The transcript concentration was determined by measuring the light absorbance at 260 nm (Nanodrop), and the transcript was analyzed by capillary electrophoresis by Fragment Analyzer (Agilent).

The sgRNAs in the following examples were chemically synthesized by known methods using phosphoramidites.

LNP Delivery In Vivo

Mouse Studies

CD-1 female mice ranging from 6-10 weeks of age were used in each study. Animals were weighed and grouped according to body weight for preparing dosing solutions based on group average weight. LNPs were dosed via the lateral tail vein in a volume of 0.2 mL per animal (approximately 10 mL per kilogram body weight). The animals were periodically observed for adverse effects for at least 24 hours post dose. For studies measuring in vivo editing in liver, CD-1 female mice were dosed at 0.1 mg/kg unless otherwise noted. Animals were euthanized at 6 or 7 days by ex sanguination via cardiac puncture under isoflurane anesthesia. Liver tissue was collected from each animal for DNA extraction and analysis. Blood was collected into serum separator tubes or into tubes containing buffered sodium citrate for plasma as described herein. Cohorts of mice were measured for editing by Next-Generation Sequencing (NGS).

Rat Studies

Sprague Dawley female rats ranging from 6-7 weeks of age were used in each study. Each animal was weighed and dosing solutions were prepared based on body weight. LNPs were dosed via the lateral tail vein in a volume of 0.35 mL per animal (approximately 2 mL per kilogram body weight). The animals were periodically observed for adverse effects for at least 24 hours post dose.

For studies measuring in vivo editing in liver, animals were dosed at 0.1 mg/kg or 0.3 mg/kg. Animals were euthanized by CO₂ asphyxiation 6 days post dose. At necropsy, the liver was collected for editing analysis by NGS and blood was collected into serum separator tubes for serum TTR measurement.

NGS Sequencing

In brief, to quantitatively determine the efficiency of editing at the target location in the genome, genomic DNA was isolated and deep sequencing was utilized to identify the presence of insertions and deletions introduced by gene editing.

PCR primers were designed around the target site (e.g., within B2M), and the genomic area of interest was amplified. Additional PCR was performed according to the manufacturer's protocols (Illumina) to add the necessary chemistry for sequencing. The amplicons were sequenced on an Illumina MiSeq instrument. The reads were aligned to the mouse or rat reference genome (e.g., GRCm38) after eliminating those having low quality scores. The resulting files containing the reads were mapped to the reference genome (BAM files), where reads that overlapped the target region of interest were selected and the number of wild type reads versus the number of reads which contain an insertion, substitution, or deletion was calculated.

The editing percentage (e.g., the “indel efficiency” or “percent indels”) was defined as the total number of sequence reads with insertions or deletions over the total number of sequence reads, including wild type.

Transthyretin (TTR) ELISA Analysis

Blood was collected and the serum was isolated as indicated. The total mouse TTR serum levels were determined using a Mouse Prealbumin (Transthyretin) ELISA Kit (Aviva Systems Biology, Cat. OKIA00111). Briefly, sera were serial diluted with kit sample diluent to a final dilution of 10,000-fold for 0.1 mg/kg dose and final dilutions of 10,000-fold and of 2,500-fold for 0.3 mg/kg. This diluted sample was then added to the ELISA plates and the assay was carried out according to the manufacturer's directions.

### Example 121

### Assessing Lipid Efficacy by In Vivo Editing

We assessed in vivo editing efficiency for materials delivered with formulations including various amine lipid compounds. Editing was measured using either G282 (SEQ ID No: 1) which targets the mouse TTR gene or G650 (SEQ ID No. 2) which targets the mouse B2M gene. Lipids described above were assessed for efficacy through in vivo editing experiments. LNPs were formulated at a 1:1 or 1:1.3 w/w ratio of sgRNA to Cas9 mRNA, and at an N/P ratio of about 6. Molar concentrations of lipids in the lipid component of the LNPs are used at mol % amine lipid/DSPC/cholesterol/PEG-2k-DMG of 50/9/38/3. The final LNPs were characterized to determine the encapsulation efficiency, polydispersity index, and average particle size according to the analytical methods provided above. Analysis of average particle size, polydispersity (PDI), total RNA content and encapsulation efficiency of RNA are shown in Table 2.

TABLE 2
Composition Analytics

| | | Encapsulation | | | Number | |
|---|---|---|---|---|---|---|
| Compound | sg:mRNA | (%) | Z-avg (nm) | PDI | mean (nm) | Experiment |
| Compound 1 | 1:1.3 | 97 | 77.85 | 0.022 | 63.16 | 1 |
| Compound 2 | 1:1.3 | 96 | 75.64 | 0.046 | 59.17 | 1 |
| Compound 3 | 1:1.3 | 91 | 86.16 | 0.079 | 61.67 | 1 |
| Compound 4 | 1:1.3 | 100 | 72.11 | 0.129 | 44.92 | 1 |
| Compound 5 | 1:1.3 | 98 | 66.28 | 0.059 | 50.89 | 1 |
| Compound 6 | 1:1.3 | 100 | 56.86 | 0.066 | 41.82 | 1 |
| Compound 1 | 1:1 | 96 | 77.23 | 0.008 | 63.61 | 2 |
| Compound 1 | 1:1 | 97 | 75.7 | 0.026 | 62.04 | 2 |
| Compound 7 | 1:1 | 99 | 67.61 | 0.126 | 43.91 | 2 |
| Compound 8 | 1:1 | 100 | 53.88 | 0.093 | 37.17 | 2 |
| Compound 10 | 1:1 | 90 | 57.21 | 0.086 | 39.12 | 2 |
| Compound 11 | 1:1 | 97 | 85.35 | 0.022 | 68.35 | 2 |
| Compound 12 | 1:1 | 83 | 88.53 | 0.007 | 73.4 | 2 |
| Compound 13 | 1:1 | 97 | 91.12 | 0.013 | 74.6 | 2 |
| Compound 1 | 1:1 | 97 | 75.7 | 0.026 | 62.04 | 3 |
| Compound 1 | 1:1 | 97 | 75.55 | 0.043 | 60.41 | 3 |
| Compound 9 | 1:1 | 89 | 62.99 | 0.058 | 45.11 | 3 |
| Compound 23 | 1:1 | 95 | 74.78 | 0.068 | 56.62 | 3 |
| Compound 24 | 1:1 | 98 | 76.82 | 0.027 | 61.34 | 3 |
| Compound 1 | 1:1 | 97 | 75.55 | 0.043 | 60.41 | 4 |
| Compound 1 | 1:1 | 97 | 75.7 | 0.026 | 62.04 | 4 |
| Compound 14 | 1:1 | 93 | 64.69 | 0.091 | 45.18 | 4 |
| Compound 17 | 1:1 | 98 | 70.97 | 0.037 | 56.58 | 4 |
| Compound 18 | 1:1 | 97 | 68.72 | 0.090 | 48.22 | 4 |
| Compound 20 | 1:1 | 96 | 79.25 | 0.023 | 64.34 | 4 |
| Compound 21 | 1:1 | 94 | 75.41 | 0.039 | 59.75 | 4 |
| Compound 22 | 1:1 | 98 | 89.43 | 0.004 | 73.96 | 4 |
| Compound 1 | 1:1 | 99 | 76 | 0.040 | 59 | 5 |
| Compound 25 | 1:1 | 99 | 87 | 0.080 | 61 | 5 |
| Compound 26 | 1:1 | 98 | 88 | 0.000 | 73 | 5 |
| Compound 27 | 1:1 | 97 | 66 | 0.100 | 45 | 5 |
| Compound 28 | 1:1 | 98 | 83 | 0.010 | 68 | 5 |
| Compound 32 | 1:1 | 99 | 77 | 0.040 | 61 | 5 |
| Compound 1 | 1:1 | 97 | 79.18 | 0.047 | 63.19 | 6 |
| Compound 36 | 1:1 | 98 | 73.71 | 0.024 | 59.48 | 6 |
| Compound 37 | 1:1 | 100 | 65.01 | 0.091 | 44.73 | 6 |
| Compound 38 | 1:1 | 96 | 91.5 | 0.019 | 74.58 | 6 |
| Compound 39 | 1:1 | 97 | 79.81 | 0.024 | 64.79 | 6 |
| Compound 41 | 1:1 | 98 | 74.83 | 0.074 | 54.1 | 6 |
| Compound 1 | 1:1 | 97 | 79.18 | 0.047 | 63.19 | 7 |
| Compound 42 | 1:1 | 99 | 74.03 | 0.027 | 58.66 | 7 |
| Compound 44 | 1:1 | 91 | 81.19 | 0.025 | 64.78 | 7 |
| Compound 45 | 1:1 | 98 | 82.93 | 0.007 | 69.23 | 7 |
| Compound 47 | 1:1 | 97 | 58.93 | 0.086 | 40.95 | 7 |
| Compound 48 | 1:1 | 97 | 64.68 | 0.085 | 45.32 | 7 |
| Compound 51 | 1:1 | 98 | 86.15 | 0.018 | 72.23 | 7 |
| Compound 52 | 1:1 | 99 | 86.72 | 0.032 | 69.4 | 7 |
| Compound 1 | 1:1 | 97 | 79.18 | 0.047 | 63.19 | 8 |
| Compound 1 | 1:1 | 98 | 78.08 | 0.007 | 63.4 | 8 |
| Compound 53 | 1:1 | 99 | 76.24 | 0.040 | 60.35 | 8 |
| Compound 54 | 1:1 | 98 | 95.76 | 0.019 | 76.95 | 8 |
| Compound 55 | 1:1 | 96 | 101 | 0.048 | 79.46 | 8 |
| Compound 56 | 1:1 | 96 | 105.2 | 0.067 | 81.44 | 8 |
| Compound 1 | 1:1 | 99 | 86.06 | 0.033 | 71.23 | 9 |
| Compound 40 | 1:1 | 98 | 74.12 | 0.056 | 56.73 | 9 |
| Compound 1 | 1:1 | 99 | 86.06 | 0.033 | 71.23 | 10 |
| Compound 24 | 1:1 | 98 | 83.92 | 0.033 | 66.88 | 10 |
| Compound 29 | 1:1 | 98 | 82.41 | 0.009 | 66.92 | 10 |
| Compound 31 | 1:1 | 98 | 75.11 | 0.059 | 58.67 | 10 |
| Compound 1 | 1:1 | 98 | 74.98 | 0.042 | 60.38 | 11 |
| Compound 24 | 1:1 | 97 | 77.24 | 0.025 | 63.16 | 11 |
| Compound 29 | 1:1 | 98 | 78.63 | 0.002 | 64.63 | 11 |
| Compound 31 | 1:1 | 99 | 73.65 | 0.015 | 59.35 | 11 |
| Compound 1 | 1:1 | 98 | 75.34 | 0.052 | 59.17 | 12 |
| Compound 57 | 1:1 | 89 | 102 | 0.011 | 83.41 | 12 |
| Compound 58 | 1:1 | 95 | 89.07 | 0.029 | 72.42 | 12 |
| Compound 59 | 1:1 | 97 | 87.86 | 0.028 | 73.04 | 12 |
| Compound 97 | 1:1 | 98 | 68.66 | 0.037 | 55.14 | 12 |
| Compound 1 | 1:1 | 97 | 78.85 | 0.036 | 64.05 | 13 |
| Compound 60 | 1:1 | 97 | 104.2 | 0.022 | 84.46 | 13 |
| Compound 61 | 1:1 | 97 | 87.26 | 0.018 | 72 | 13 |
| Compound 1 | 1:1 | 98 | 82.71 | 0.056 | 64.97 | 14 |
| Compound 62 | 1:1 | 98 | 73.46 | 0.044 | 59.35 | 14 |
| Compound 63 | 1:1 | 98 | 79.37 | 0.019 | 64.05 | 14 |
| Compound 64 | 1:1 | 98 | 85.16 | 0.032 | 69.25 | 14 |
| Compound 65 | 1:1 | 98 | 70.19 | 0.034 | 55.76 | 14 |
| Compound 66 | 1:1 | 98 | 58.54 | 0.071 | 44.03 | 14 |
| Compound 67 | 1:1 | 99 | 69.85 | 0.025 | 56.56 | 14 |
| Compound 68 | 1:1 | 98 | 72.71 | 0.008 | 60.04 | 14 |
| Compound 54 | 1:1 | 98 | 89.8 | 0.037 | 71.8 | 15 |
| Compound 55 | 1:1 | 92 | 113.2 | 0.039 | 90.16 | 15 |
| Compound 69 | 1:1 | 98 | 85.11 | 0.026 | 68.12 | 15 |
| Compound 70 | 1:1 | 98 | 83.47 | 0.017 | 66.78 | 15 |
| Compound 1 | 1:1 | 96 | 83.65 | 0.031 | 67.22 | 16 |
| Compound 1 | 1:1 | 99 | 84.99 | 0.007 | 71.1 | 16 |
| Compound 54 | 1:1 | 98 | 99.71 | 0.058 | 78.39 | 16 |
| Compound 59 | 1:1 | 99 | 100.4 | 0.005 | 84.05 | 16 |
| Compound 76 | 1:1 | 99 | 111.8 | 0.097 | 80.2 | 16 |
| Compound 77 | 1:1 | 99 | 101.8 | 0.006 | 85.4 | 16 |
| Compound 79 | 1:1 | 99 | 99.24 | 0.019 | 83.64 | 16 |
| Compound 83 | 1:1 | 99 | 89.55 | 0.009 | 74.41 | 16 |
| Compound 84 | 1:1 | 95 | 114.2 | 0.086 | 86.08 | 16 |
| Compound 85 | 1:1 | 99 | 97.86 | 0.006 | 81.21 | 16 |
| Compound 86 | 1:1 | 98 | 99.01 | 0.016 | 81.23 | 16 |
| Compound 1 | 1:1 | 96 | 83.65 | 0.031 | 67.22 | 17 |
| Compound 1 | 1:1 | 99 | 85.79 | 0.002 | 68.64 | 17 |
| Compound 59 | 1:1 | 99 | 100.4 | 0.005 | 84.05 | 17 |
| Compound 80 | 1:1 | 98 | 105.8 | 0.016 | 87.3 | 17 |
| Compound 82 | 1:1 | 98 | 117.5 | 0.038 | 94.01 | 17 |
| Compound 87 | 1:1 | 98 | 98.47 | 0.006 | 81.57 | 17 |
| Compound 88 | 1:1 | 98 | 117.1 | 0.011 | 99.51 | 17 |
| Compound 89 | 1:1 | 95 | 107.3 | 0.008 | 89.4 | 17 |
| Compound 90 | 1:1 | 99 | 99.62 | 0.017 | 81.15 | 17 |
| Compound 91 | 1:1 | 99 | 93.13 | 0.028 | 75.67 | 17 |
| Compound 92 | 1:1 | 99 | 93.69 | 0.022 | 77.35 | 17 |
| Compound 93 | 1:1 | 98 | 95.19 | 0.004 | 78.73 | 17 |
| Compound 94 | 1:1 | 99 | 96.35 | 0.018 | 78.22 | 17 |
| Compound 95 | 1:1 | 99 | 91.1 | 0.029 | 74.5 | 17 |
| Compound 1 | 1:1 | 99 | 84.48 | 0.041 | 66.21 | 18 |
| Compound 59 | 1:1 | 99 | 100.4 | 0.005 | 84.05 | 18 |
| Compound 71 | 1:1 | 99 | 95.57 | 0.035 | 76.95 | 18 |
| Compound 72 | 1:1 | 99 | 91.65 | 0.002 | 75.22 | 18 |
| Compound 73 | 1:1 | 97 | 110.6 | 0.008 | 94.5 | 18 |
| Compound 74 | 1:1 | 99 | 105.7 | 0.021 | 87.14 | 18 |
| Compound 75 | 1:1 | 98 | 110.9 | 0.020 | 92.74 | 18 |
| Compound 1 | 1:1 | 96 | 83.65 | 0.031 | 67.22 | 19 |
| Compound 1 | 1:1 | 98 | 86.84 | 0.020 | 71.49 | 19 |
| Compound 96 | 1:1 | 98 | 81.55 | 0.019 | 65.94 | 19 |
| Compound 1 | 1:1 | 99 | 76 | 0.04 | 59 | 20 |
| Compound 98 | 1:1 | 99 | 70 | 0.02 | 57 | 20 |
| Compound 99 | 1:1 | 99 | 89 | 0.003 | 74 | 20 |

Table 3 shows editing percentages in mouse liver as measured by NGS.

TABLE 3
Editing efficiency in mouse liver, providing % indel formation for G282
and G650 experiments and serum TTR levels for G282 experiments.

| | Editing | | | Serum TTR | | | | | |
|---|---|---|---|---|---|---|---|---|---|
| | Mean | | | Serum | | | | | |
| | % | | | TTR | | | | | |
| Compound | Indel | SD | N | (ug/ml) | SD | N | % TSS | Guide | Experiment |
| TSS | 0.1 | 0.0 | 5 | 1235 | 279 | 5 | 100% | G282 | 1 |
| Compound 1 | 15.7 | 4.0 | 5 | 523 | 178 | 5 | 42% | G282 | 1 |
| Compound 1 | 18.7 | 12.8 | 5 | 773 | 255 | 5 | 63% | G282 | 1 |
| Compound 1 | 15.1 | 8.8 | 5 | 689 | 320 | 5 | 56% | G282 | 1 |
| Compound 2 | 6.3 | 2.8 | 5 | 1114 | 257 | 5 | 90% | G282 | 1 |
| Compound 3 | 8.8 | 3.1 | 5 | 1297 | 75 | 5 | 105% | G282 | 1 |
| Compound 4 | 0.1 | 0.0 | 5 | 1347 | 283 | 5 | 109% | G282 | 1 |
| Compound 5 | 7.0 | 5.2 | 5 | 737 | 229 | 5 | 60% | G282 | 1 |
| Compound 6 | 0.1 | 0.0 | 5 | 1107 | 251 | 5 | 90% | G282 | 1 |
| TSS | 0.1 | 0.0 | 5 | 905 | 230 | 5 | 100% | G282 | 2 |
| Compound 1 | 24.9 | 5.1 | 5 | 563 | 113 | 5 | 62% | G282 | 2 |
| Compound 1 | 21.5 | 6.8 | 5 | 658 | 99 | 5 | 73% | G282 | 2 |
| Compound 7 | 0.2 | 0.1 | 5 | 940 | 142 | 5 | 104% | G282 | 2 |
| Compound 8 | 0.1 | 0.0 | 5 | 962 | 138 | 5 | 106% | G282 | 2 |
| Compound 10 | 0.5 | 0.2 | 5 | 871 | 136 | 5 | 96% | G282 | 2 |
| Compound 11 | 16.8 | 3.1 | 5 | 706 | 91 | 5 | 78% | G282 | 2 |
| Compound 12 | 20.3 | 8.2 | 5 | 736 | 131 | 5 | 81% | G282 | 2 |
| Compound 13 | 12.0 | 1.7 | 5 | 837 | 173 | 5 | 93% | G282 | 2 |
| TSS | 0.0 | 0.1 | 5 | 865 | 81 | 5 | 100% | G282 | 3 |
| Compound 1 | 32.0 | 9.9 | 5 | 498 | 145 | 5 | 58% | G282 | 3 |
| Compound 9 | 0.1 | 0.1 | 5 | 865 | 117 | 5 | 100% | G282 | 3 |
| Compound 23 | 8.6 | 6.2 | 5 | 716 | 101 | 5 | 83% | G282 | 3 |
| Compound 24 | 34.2 | 8.0 | 5 | 397 | 50 | 5 | 46% | G282 | 3 |
| TSS | 0.1 | 0.1 | 5 | 1035 | 179 | 5 | 100% | G282 | 4 |
| Compound 1 | 20.5 | 6.4 | 5 | 742 | 172 | 5 | 72% | G282 | 4 |
| Compound 14 | 0.2 | 0.1 | 5 | 908 | 136 | 5 | 88% | G282 | 4 |
| Compound 17 | 12.0 | 3.6 | 5 | 727 | 75 | 5 | 70% | G282 | 4 |
| Compound 18 | 2.1 | 0.7 | 5 | 849 | 98 | 5 | 82% | G282 | 4 |
| Compound 20 | 24.5 | 12.3 | 5 | 476 | 164 | 5 | 46% | G282 | 4 |
| Compound 21 | 0.8 | 0.3 | 5 | 897 | 124 | 5 | 87% | G282 | 4 |
| Compound 22 | 11.5 | 2.9 | 5 | 634 | 60 | 5 | 61% | G282 | 4 |
| TSS | 0.1 | 0.0 | 5 | 904 | 150 | 5 | 100% | G282 | 5 |
| Compound 1 | 14.4 | 5.8 | 5 | 743 | 96 | 5 | 82% | G282 | 5 |
| Compound 25 | 3.9 | 1.5 | 5 | 1013 | 268 | 5 | 112% | G282 | 5 |
| Compound 26 | 8.2 | 2.1 | 5 | 1008 | 93 | 5 | 112% | G282 | 5 |
| Compound 27 | 0.4 | 0.1 | 5 | 841 | 166 | 5 | 93% | G282 | 5 |
| Compound 28 | 9.9 | 1.5 | 5 | 661 | 138 | 5 | 73% | G282 | 5 |
| Compound 32 | 28.8 | 8.2 | 5 | 388 | 150 | 5 | 43% | G282 | 5 |
| TSS | 0.1 | 0.0 | 5 | 801 | 115 | 5 | 100% | G282 | 6 |
| Compound 1 | 14.7 | 9.5 | 5 | 865 | 197 | 5 | 108% | G282 | 6 |
| Compound 36 | 0.2 | 0.1 | 5 | 795 | 136 | 5 | 99% | G282 | 6 |
| Compound 37 | 1.6 | 0.2 | 5 | 617 | 117 | 5 | 77% | G282 | 6 |
| Compound 38 | 0.2 | 0.1 | 5 | 481 | 137 | 5 | 60% | G282 | 6 |
| Compound 39 | 2.5 | 1.1 | 5 | 656 | 327 | 5 | 82% | G282 | 6 |
| Compound 41 | 9.0 | 3.7 | 5 | 758 | 33 | 5 | 95% | G282 | 6 |
| TSS | 0.1 | 0.0 | 5 | 1114 | 135 | 5 | 100% | G282 | 7 |
| Compound 1 | 18.3 | 6.4 | 4 | 784 | 175 | 5 | 70% | G282 | 7 |
| Compound 42 | 5.2 | 2.4 | 5 | 902 | 77 | 5 | 81% | G282 | 7 |
| Compound 44 | 3.8 | 1.6 | 5 | 910 | 219 | 5 | 82% | G282 | 7 |
| Compound 45 | 4.0 | 0.8 | 5 | 1096 | 155 | 5 | 98% | G282 | 7 |
| Compound 47 | 0.1 | 0.0 | 5 | 1075 | 185 | 5 | 96% | G282 | 7 |
| Compound 48 | 0.1 | 0.0 | 5 | 915 | 163 | 5 | 82% | G282 | 7 |
| Compound 51 | 13.5 | 5.3 | 5 | 843 | 95 | 5 | 76% | G282 | 7 |
| Compound 52 | 6.4 | 1.3 | 5 | 935 | 77 | 5 | 84% | G282 | 7 |
| TSS | 0.1 | 0.0 | 5 | 875 | 92 | 5 | 100% | G282 | 8 |
| Compound 1 | 22.6 | 9.7 | 5 | 687 | 137 | 5 | 78% | G282 | 8 |
| Compound 53 | 28.2 | 8.5 | 5 | 413 | 152 | 5 | 47% | G282 | 8 |
| Compound 54 | 46.7 | 10.9 | 5 | 270 | 142 | 5 | 31% | G282 | 8 |
| Compound 55 | 31.5 | 6.4 | 5 | 442 | 123 | 5 | 51% | G282 | 8 |
| Compound 56 | 15.5 | 4.4 | 4 | 576 | 67 | 4 | 66% | G282 | 8 |
| TSS | 0.1 | 0.1 | 4 | 884 | 113 | 5 | 100% | G282 | 9 |
| Compound 1 | 33.5 | 8.9 | 5 | 388 | 103 | 5 | 44% | G282 | 9 |
| Compound 40 | 42.1 | 15.7 | 4 | 393 | 262 | 5 | 44% | G282 | 9 |
| TSS | 0.2 | 0.0 | 5 | ND | ND | ND | ND | G282 | 10 |
| Compound 1 | 29.4 | 4.6 | 5 | ND | ND | ND | ND | G282 | 10 |
| Compound 24 | 43.2 | 9.1 | 5 | ND | ND | ND | ND | G282 | 10 |
| Compound 29 | 18.4 | 6.6 | 5 | ND | ND | ND | ND | G282 | 10 |
| Compound 31 | 26.7 | 14.5 | 5 | ND | ND | ND | ND | G282 | 10 |
| TSS | 0.0 | 0.1 | 5 | ND | ND | ND | ND | G650 | 11 |
| Compound 1 | 13.6 | 6.1 | 5 | ND | ND | ND | ND | G650 | 11 |
| Compound 24 | 25.8 | 8.1 | 5 | ND | ND | ND | ND | G650 | 11 |
| Compound 29 | 19.7 | 6.3 | 5 | ND | ND | ND | ND | G650 | 11 |
| Compound 31 | 24.5 | 5.6 | 5 | ND | ND | ND | ND | G650 | 11 |
| TSS | 0.1 | 0.1 | 5 | ND | ND | ND | ND | G650 | 12 |
| Compound 1 | 10.2 | 2.8 | 4 | ND | ND | ND | ND | G650 | 12 |
| Compound 57 | 10.0 | 2.2 | 5 | ND | ND | ND | ND | G650 | 12 |
| Compound 58 | 14.9 | 3.0 | 5 | ND | ND | ND | ND | G650 | 12 |
| Compound 59 | 19.7 | 2.8 | 5 | ND | ND | ND | ND | G650 | 12 |
| Compound 97 | 5.1 | 2 | 5 | ND | ND | ND | ND | G650 | 12 |
| TSS | 0.1 | 0.1 | 5 | ND | ND | ND | ND | G650 | 13 |
| Compound 1 | 15.7 | 5.5 | 5 | ND | ND | ND | ND | G650 | 13 |
| Compound 60 | 23.7 | 10.5 | 5 | ND | ND | ND | ND | G650 | 13 |
| Compound 61 | 20.2 | 9.5 | 5 | ND | ND | ND | ND | G650 | 13 |
| TSS | 0.1 | 0.0 | 5 | ND | ND | ND | ND | G650 | 14 |
| Compound 1 | 16.1 | 2.8 | 5 | ND | ND | ND | ND | G650 | 14 |
| Compound 62 | 6.5 | 1.1 | 5 | ND | ND | ND | ND | G650 | 14 |
| Compound 63 | 15.0 | 1.6 | 5 | ND | ND | ND | ND | G650 | 14 |
| Compound 64 | 19.3 | 4.8 | 5 | ND | ND | ND | ND | G650 | 14 |
| Compound 65 | 4.7 | 2.0 | 5 | ND | ND | ND | ND | G650 | 14 |
| Compound 66 | 0.8 | 0.3 | 5 | ND | ND | ND | ND | G650 | 14 |
| Compound 67 | 3.1 | 1.0 | 5 | ND | ND | ND | ND | G650 | 14 |
| Compound 68 | 2.9 | 0.8 | 5 | ND | ND | ND | ND | G650 | 14 |
| TSS | 0.7 | 1.3 | 5 | ND | ND | ND | ND | G650 | 15 |
| Compound 1 | 14.8 | 4.1 | 5 | ND | ND | ND | ND | G650 | 15 |
| Compound 54 | 27.6 | 9.5 | 5 | ND | ND | ND | ND | G650 | 15 |
| Compound 55 | 30.2 | 13.4 | 5 | ND | ND | ND | ND | G650 | 15 |
| Compound 69 | 21.3 | 3.0 | 5 | ND | ND | ND | ND | G650 | 15 |
| Compound 70 | 9.7 | 2.5 | 5 | ND | ND | ND | ND | G650 | 15 |
| TSS | 0.1 | 0.1 | 5 | 907 | 92 | 5 | 100% | G282 | 16 |
| Compound 1 | 35.4 | 6.1 | 5 | 446 | 63 | 5 | 49% | G282 | 16 |
| Compound 1 | 14.9 | 3.0 | 5 | 607 | 85 | 5 | 67% | G282 | 16 |
| Compound 54 | 27.0 | 5.9 | 5 | 574 | 144 | 5 | 63% | G282 | 16 |
| Compound 59 | 39.0 | 12.0 | 5 | 367 | 168 | 5 | 40% | G282 | 16 |
| Compound 76 | 7.0 | 3.3 | 5 | 735 | 134 | 5 | 81% | G282 | 16 |
| Compound 77 | 30.4 | 9.5 | 5 | 478 | 157 | 5 | 53% | G282 | 16 |
| Compound 79 | 40.3 | 11.6 | 5 | 351 | 140 | 5 | 39% | G282 | 16 |
| Compound 83 | 26.4 | 8.2 | 5 | 488 | 87 | 5 | 54% | G282 | 16 |
| Compound 84 | 13.3 | 5.3 | 5 | 648 | 124 | 5 | 71% | G282 | 16 |
| Compound 85 | 3.5 | 1.3 | 5 | 762 | 42 | 5 | 84% | G282 | 16 |
| Compound 86 | 22.6 | 13.6 | 5 | 582 | 172 | 5 | 64% | G282 | 16 |
| TSS | 0.1 | 0.0 | 5 | 756 | 169 | 5 | 100% | G282 | 17 |
| Compound 1 | 27.2 | 5.7 | 5 | 490 | 90 | 5 | 65% | G282 | 17 |
| Compound 1 | 25.0 | 5.2 | 5 | 505 | 113 | 5 | 67% | G282 | 17 |
| Compound 59 | 31.8 | 8.2 | 5 | 431 | 105 | 5 | 57% | G282 | 17 |
| Compound 80 | 10.3 | 2.2 | 5 | 873 | 220 | 5 | 116% | G282 | 17 |
| Compound 82 | 5.0 | 1.5 | 5 | 953 | 185 | 5 | 126% | G282 | 17 |
| Compound 87 | 26.2 | 8.7 | 5 | 511 | 107 | 5 | 68% | G282 | 17 |
| Compound 88 | 32.1 | 5.5 | 5 | 582 | 145 | 5 | 77% | G282 | 17 |
| Compound 89 | 9.2 | 3.2 | 5 | 774 | 76 | 5 | 102% | G282 | 17 |
| Compound 90 | 26.0 | 6.7 | 5 | 504 | 100 | 5 | 67% | G282 | 17 |
| Compound 91 | 13.9 | 4.4 | 5 | 633 | 54 | 5 | 84% | G282 | 17 |
| Compound 92 | 26.6 | 5.6 | 5 | 470 | 102 | 5 | 62% | G282 | 17 |
| Compound 93 | 15.0 | 2.3 | 5 | 669 | 154 | 5 | 88% | G282 | 17 |
| Compound 94 | 39.4 | 6.6 | 5 | 286 | 95 | 5 | 38% | G282 | 17 |
| Compound 95 | 30.3 | 7.7 | 5 | 408 | 88 | 5 | 54% | G282 | 17 |
| TSS | 0.1 | 0.0 | 5 | 919 | 230 | 5 | 100% | G282 | 18 |
| Compound 1 | 41.6 | 9.0 | 5 | 431 | 270 | 5 | 47% | G282 | 18 |
| Compound 1 | 32.0 | 9.9 | 4 | 587 | 121 | 4 | 64% | G282 | 18 |
| Compound 59 | 35.6 | 8.4 | 5 | 604 | 229 | 5 | 66% | G282 | 18 |
| Compound 71 | 52.4 | 4.0 | 4 | 462 | 156 | 5 | 50% | G282 | 18 |
| Compound 72 | 42.4 | 7.4 | 5 | 479 | 188 | 5 | 52% | G282 | 18 |
| Compound 73 | 41.6 | 8.8 | 4 | 550 | 187 | 3 | 60% | G282 | 18 |
| Compound 74 | 42.8 | 12.9 | 5 | 406 | 144 | 5 | 44% | G282 | 18 |
| Compound 75 | 25.4 | 4.3 | 4 | 623 | 201 | 5 | 68% | G282 | 18 |
| TSS | 0.1 | 0.0 | 5 | 777 | 89 | 5 | 100% | G282 | 19 |
| Compound 1 | 30.4 | 5.1 | 5 | 311 | 76 | 5 | 40% | G282 | 19 |
| Compound 1 | 38.6 | 8.0 | 5 | 247 | 82 | 5 | 32% | G282 | 19 |
| Compound 96 | 15.3 | 8.2 | 5 | 449 | 95 | 5 | 58% | G282 | 19 |
| TSS | 0.08 | 0.04 | 5 | ND | ND | ND | ND | G282 | 20 |
| Compound 1 | 20.5 | 10.6 | 5 | ND | ND | ND | ND | G282 | 20 |
| Compound 98 | 10.56 | 5.37 | 5 | ND | ND | ND | ND | G282 | 20 |
| Compound 99 | 26.38 | 15.2 | 5 | ND | ND | ND | ND | G282 | 20 |

### Example 122

### Dose Response of Editing in Liver

To assess whether the editing was dose responsive, experiments were performed in vivo at various LNP dose levels. Cas9 mRNA of Example 120 was formulated as LNPs with an sgRNA targeting TTR (G282, SEQ ID NO: 1; or G502, SEQ ID NO: 4).

Alternatively, Cas9 mRNA of Example 120 was formulated as LNPs with an sgRNA targeting B2M (G650, SEQ ID NO: 2) These LNPs were formulated at a w/w ratio of a sgRNA and Cas9 mRNA as indicated in Table 4. The LNPs were formulated using the cross flow procedure with lipid molar compositions of mol % amine lipid/DSPC/cholesterol/PEG-2k-DMG of 50/9/38/3 and an N/P ratio of 6.0.

LNP compositions were analyzed for average particle size, polydispersity (PDI), total RNA content and encapsulation efficiency of RNA as described in Example 120.

Analysis of average particle size, polydispersity (PDI), total RNA content and encapsulation efficiency of RNA are shown in Table 4.

TABLE 4
Composition Analytics

| | | | | | Number | |
|---|---|---|---|---|---|---|
| | | | Z-avg | | mean | |
| Compound | sg:mRNA | % E | (nm) | PDI | (nm) | Experiment |
| Compound 1 | 1:1 | 97 | 79.18 | 0.047 | 63.19 | 101 |
| Compound 29 | 1:1 | 99 | 84 | 0.003 | 70 | 101 |
| Compound 31 | 1:1 | 99 | 76 | 0.04 | 61 | 101 |
| Compound 1 | 1:1 | 98 | 84.93 | 0.007 | 69.51 | 102 |
| Compound 40 | 1:1 | 95 | 83.69 | 0.014 | 66.92 | 102 |
| Compound 53 | 1:1 | 99 | 70.47 | 0.019 | 56.05 | 102 |
| Compound 54 | 1:1 | 98 | 96.98 | 0.032 | 77.96 | 102 |
| Compound 1 | 1:1 | 99 | 79.83 | 0.015 | 62.86 | 103 |
| Compound 59 | 1:1 | 98 | 87.31 | 0.012 | 72.60 | 103 |
| Compound 1 | 1:1 | 98 | 82.71 | 0.056 | 64.97 | 104 |
| Compound 59 | 1:1 | 96 | 83.42 | 0.024 | 67.12 | 104 |
| Compound 1 | 1:2 | 99 | 88.60 | 0.033 | 73.15 | 105 |
| Compound 24 | 1:2 | 99 | 88.39 | 0.029 | 72.95 | 105 |
| Compound 59 | 1:2 | 99 | 96.76 | 0.041 | 79.11 | 105 |
| Compound 61 | 1:2 | 98 | 99.25 | 0.033 | 79.73 | 105 |
| Compound 94 | 1:2 | 99 | 98.90 | 0.032 | 80.04 | 105 |

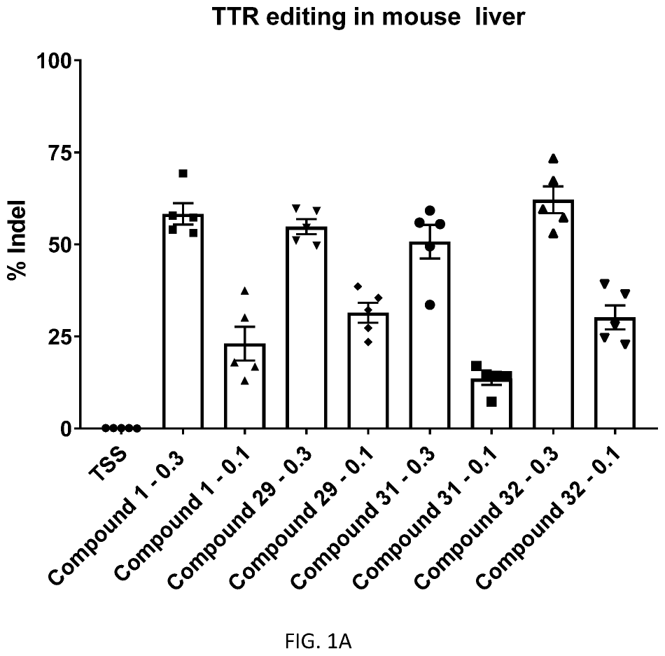

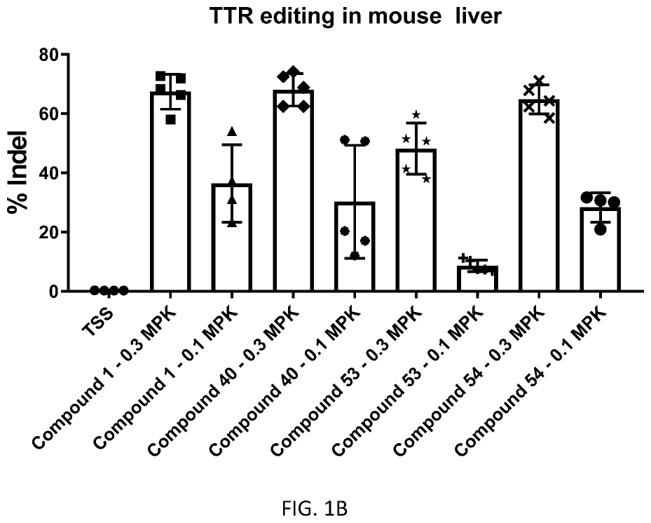

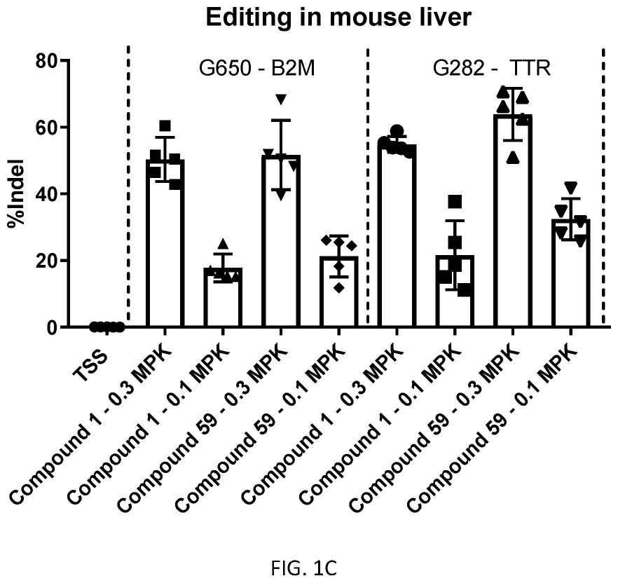

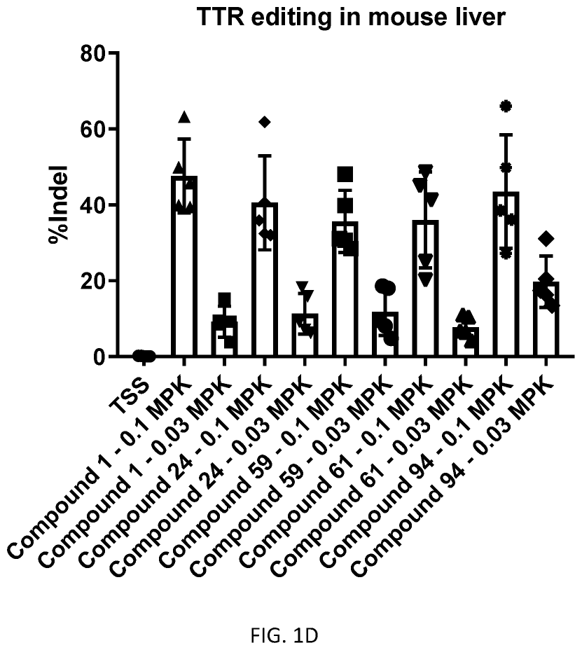

CD-1 female mice were dosed i.v. as specified in Table 5 and assessed for editing in liver. Results are shown in FIGS. 1A-1D and Table 5.

TABLE 5
TTR liver editing and serum TTR levels for dose response providing % indel formation for
G282, G502 and G650 experiments and serum TTR levels for G282 and G502 experiments.

| | Editing | | | | Serum TTR | | | | | |
|---|---|---|---|---|---|---|---|---|---|---|
| | | Mean | | | Serum | | | | | |
| | Dose | % | | | TTR | | | | | |
| Compound | (mg/kg) | Indel | SD | N | (ug/ml) | SD | N | % TSS | Guide | Experiment |
| TSS | TSS | 0.1 | 0.0 | 5 | 1369 | 387 | 5 | 100% | G282 | 101 |
| Compound 1 | 0.1 | 23.1 | 10.3 | 5 | 1083 | 665 | 5 | 79% | G282 | 101 |
| Compound 1 | 0.3 | 58.3 | 6.5 | 5 | 271 | 135 | 5 | 20% | G282 | 101 |
| Compound 29 | 0.1 | 31.4 | 6.1 | 5 | 663 | 348 | 5 | 48% | G282 | 101 |
| Compound 29 | 0.3 | 54.8 | 4.5 | 5 | 205 | 122 | 5 | 15% | G282 | 101 |
| Compound 31 | 0.1 | 13.5 | 3.6 | 5 | 976 | 457 | 5 | 71% | G282 | 101 |
| Compound 31 | 0.3 | 50.7 | 10.2 | 5 | 226 | 99 | 5 | 17% | G282 | 101 |
| TSS | TSS | 0.2 | 0.1 | 4 | 918 | 100 | 5 | 100% | G282 | 102 |
| Compound 1 | 0.1 | 36.4 | 13.0 | 4 | 500 | 263 | 4 | 54% | G282 | 102 |
| Compound 1 | 0.3 | 67.4 | 5.9 | 5 | 123 | 61 | 5 | 13% | G282 | 102 |
| Compound 40 | 0.1 | 30.2 | 19.1 | 5 | 443 | 164 | 5 | 48% | G282 | 102 |
| Compound 40 | 0.3 | 68.0 | 5.5 | 5 | 80 | 46 | 5 | 9% | G282 | 102 |
| Compound 53 | 0.1 | 8.6 | 2.0 | 5 | 619 | 133 | 5 | 67% | G282 | 102 |
| Compound 53 | 0.3 | 48.2 | 8.7 | 5 | 231 | 69 | 5 | 25% | G282 | 102 |
| Compound 54 | 0.1 | 28.3 | 5.0 | 4 | 449 | 12 | 4 | 49% | G282 | 102 |
| Compound 54 | 0.3 | 64.8 | 4.9 | 5 | 97 | 51 | 5 | 11% | G282 | 102 |
| TSS | TSS | 0.1 | 0.1 | 5 | 608 | 118 | 5 | 100% | G282 | 103 |
| Compound 1 | 0.1 | 21.6 | 10.4 | 5 | 462 | 181 | 5 | 76% | G282 | 103 |
| Compound 1 | 0.3 | 54.9 | 2.4 | 5 | 82 | 24 | 5 | 13% | G282 | 103 |
| Compound 59 | 0.1 | 32.4 | 6.2 | 5 | 300 | 76 | 5 | 49% | G282 | 103 |
| Compound 59 | 0.3 | 63.9 | 7.8 | 5 | 43 | 50 | 3 | 7% | G282 | 103 |
| Compound 1 | 0.1 | 17.8 | 4.2 | 5 | ND | ND | 0 | ND | G650 | 104 |
| Compound 1 | 0.3 | 50.3 | 6.6 | 5 | ND | ND | 0 | ND | G650 | 104 |
| Compound 59 | 0.1 | 21.2 | 6.1 | 5 | ND | ND | 0 | ND | G650 | 104 |
| Compound 59 | 0.3 | 51.6 | 10.4 | 5 | ND | ND | 0 | ND | G650 | 104 |
| TSS | TSS | 0.14 | 0.1 | 5 | 1098 | 84 | 5 | 100% | G502 | 105 |
| Compound 1 | 0.03 | 9.24 | 4.1 | 5 | 931 | 41 | 5 | 85% | G502 | 105 |
| Compound 1 | 0.1 | 47.62 | 9.7 | 5 | 294 | 135 | 5 | 27% | G502 | 105 |
| Compound 24 | 0.03 | 11.34 | 5.4 | 5 | 772 | 126 | 5 | 70% | G502 | 105 |
| Compound 24 | 0.1 | 40.54 | 12.4 | 5 | 420 | 178 | 5 | 38% | G502 | 105 |
| Compound 59 | 0.03 | 11.8 | 6.2 | 5 | 909 | 115 | 5 | 83% | G502 | 105 |
| Compound 59 | 0.1 | 35.64 | 8.2 | 5 | 467 | 166 | 5 | 42% | G502 | 105 |
| Compound 61 | 0.03 | 7.78 | 2.9 | 5 | 711 | 66 | 5 | 65% | G502 | 105 |
| Compound 61 | 0.1 | 36.06 | 12.6 | 5 | 472 | 143 | 5 | 43% | G502 | 105 |
| Compound 94 | 0.03 | 19.76 | 6.8 | 5 | 605 | 93 | 5 | 55% | G502 | 105 |
| Compound 94 | 0.1 | 43.52 | 14.9 | 5 | 352 | 201 | 5 | 32% | G502 | 105 |

### Example 123

### TTR Liver Editing and Serum TTR Levels Providing % Indel Formation for G502 Experiments

We assessed in vivo delivery by measuring editing efficiency for materials delivered with formulations that included various amine lipid compounds. Cas9 mRNA (SEQ ID NO: 6) was formulated as LNPs with an sgRNA targetting the TTR mouse gene (SEQ ID NO: 4). LNPs were formulated at a 1:2 w/w ratio of sgRNA to Cas9 mRNA, and at an N/P ratio of about 6. Molar concentrations of lipids in the lipid component of the LNPs are used at mol % amine lipid/DSPC/cholesterol/PEG-2k-DMG of 50/9/38/3. The final LNPs compositions were characterized and analyzed as described in Example 120. Analysis of average particle size, polydispersity (PDI), total RNA content and encapsulation efficiency of RNA are shown in Table 6.

TABLE 6
Composition Analytics

| | | | | | Number | |
|---|---|---|---|---|---|---|
| | | | Z-avg | | mean | |
| Compound | sg:mRNA | % E | (nm) | PDI | (nm) | Experiment |
| Compound 1 | 1:2 | 99 | 88 | 0.04 | 71 | 106 |
| Compound 100 | 1:2 | 99 | 89 | 0.04 | 70 | 106 |
| Compound 101 | 1:2 | 99 | 73 | 0.04 | 58 | 106 |
| Compound 102 | 1:2 | 98 | 71 | 0.02 | 57 | 106 |
| Compound 103 | 1:2 | 99 | 65 | 0.05 | 50 | 106 |
| Compound 1 | 1:2 | 99 | 91 | 0.002 | 76 | 107 |
| Compound 114 | 1:2 | 96 | 87 | 0.02 | 72 | 107 |
| Compound 115 | 1:2 | 97 | 93 | 0.01 | 76 | 107 |
| Compound 116 | 1:2 | 99 | 90 | 0 | 74 | 107 |

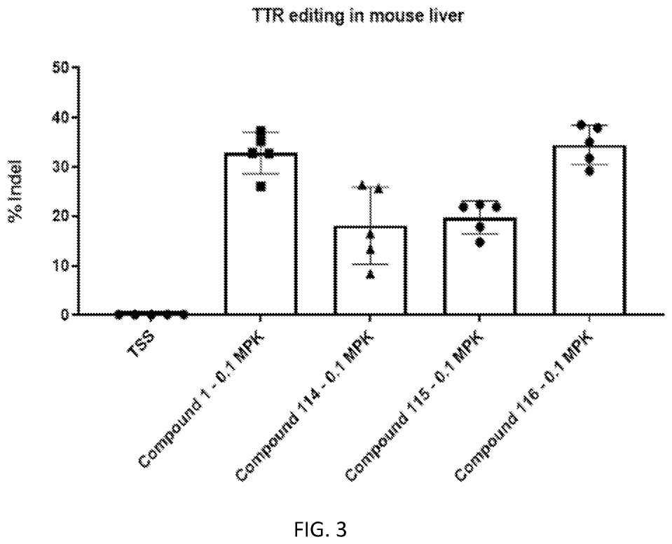

CD-1 female mice were dosed i.v. at 0.1 mg/kg, assessed for editing in liver, and circulating TTR levels were measured. Results are shown in FIGS. 2, 3 and Table 7.

TABLE 7
Editing efficiency in mouse, providing % indel formation
for G502 experiments and serum TTR levels.

| | Editing | | Serum TTR | | | | | | |
|---|---|---|---|---|---|---|---|---|---|
| | Mean % | | | Serum TTR | | Guide | Experiment | | |
| Compound | Indel | SD | N | (ug/ml) | SD | N | % TSS | | |
| TSS | 0.1 | 0.0 | 5 | 1168.5 | 326.6 | 5 | 100 | G502 | 106 |
| Compound 1 | 42.9 | 3.6 | 5 | 442 | 107.9 | 5 | 37.8 | G502 | 106 |
| Compound 100 | 12 | 5.1 | 5 | 1017.4 | 187.4 | 5 | 81.3 | G502 | 106 |
| Compound 101 | 25.9 | 1.9 | 5 | 655.8 | 118.7 | 5 | 56.1 | G502 | 106 |
| Compound 102 | 19.6 | 6.2 | 5 | 728.1 | 170.4 | 5 | 62.3 | G502 | 106 |
| Compound 103 | 4.2 | 1.0 | 5 | 1043.8 | 154.9 | 5 | 89.3 | G502 | 106 |
| TSS | 0.1 | 0.0 | 5 | 1105.5 | 229.9 | 5 | 100 | G502 | 107 |
| Compound 1 | 32.8 | 4.2 | 5 | 593.76 | 174.5 | 5 | 53.7 | G502 | 107 |
| Compound 114 | 18.04 | 7.8 | 5 | 978.01 | 276.8 | 5 | 88.4 | G502 | 107 |
| Compound 115 | 19.68 | 3.3 | 5 | 816.3 | 128.2 | 5 | 73.8 | G502 | 107 |
| Compound 116 | 34.48 | 4.0 | 5 | 567.09 | 68.1 | 5 | 51.3 | G502 | 107 |

### Example 124

### TTR Liver Editing and Serum TTR Levels Providing % Indel Formation for G650 Experiments

The in vivo delivery to liver, spleen, and bone marrow was assessed by measuring editing efficiency for materials delivered with formulations including various amine lipid compounds. Cas9 mRNA (SEQ ID NO: 6) was formulated as LNPs with an sgRNA targetting the TTR mouse gene (G650, SEQ ID: 2). LNPs were formulated at a 1:2 w/w ratio of sgRNA to Cas9 mRNA and at an N/P ratio of about 6. Molar concentrations of lipids in the lipid component of the LNPs are used at mol % amine lipid/DSPC/cholesterol/PEG-2k-DMG of 50/9/38/3. The final LNPs compositions were characterized and analyzed as described in Example 120. Analysis of average particle size, polydispersity (PDI), total RNA content and encapsulation efficiency of RNA are shown in Table 8.

TABLE 8
Composition Analytics

| | | | | | Number | |
|---|---|---|---|---|---|---|
| | | | Z-avg | | mean | |
| Compound | sg:mRNA | % E | (nm) | PDI | (nm) | Experiment |
| Compound 1 | 1:2 | 98 | 86 | 0.03 | 70 | 108 |
| Compound 107 | 1:2 | 94 | 84 | 0.07 | 65 | 108 |

CD-1 female mice were dosed i.v. at 0.1, 0.3, or 1 mg/kg and assessed for editing in liver, spleen, and bone marrow. Results are shown in Table 9.

TABLE 9
Editing efficiency in mouse, providing %
indel formation for G650 experiments

| | | Liver | | | |
|---|---|---|---|---|---|
| | Dose | editing | % Editing | % Editing | |
| Compound | (mg/kg) | (%) | in Spleen | in BM | Experiment |
| TSS | — | 0.1 | 0.1 | NA | 108 |
| Compound 1 | 0.1 | 23.74 | 0.62 | NA | 108 |
| | 0.3 | 58.48 | 2.2 | NA | 108 |
| | 1 | 73.6 | 6.26 | 2.66 | 108 |
| Compound | 0.1 | 0.52 | 0.08 | NA | 108 |
| 107 | 0.3 | 1.04 | 0.12 | NA | 108 |
| | 1 | 5.92 | 0.3 | 0.32 | 108 |

### Example 125

### Delivery in Rat Studies

In vivo delivery in a rat model was assessed by measuring editing efficiency for materials delivered with formulations including various amine lipid compounds. Cas9 mRNA (SEQ ID NO: 6) was formulated as LNPs with an sgRNA targetting the TTR rat gene (G534, SEQ ID NO: 5). LNPs were formulated and characterized as described in Example 123. Analysis of average particle size, polydispersity (PDI), total RNA content and encapsulation efficiency of RNA are shown in Table 10.

TABLE 10
Composition Analytics

| | | | | | Number | |
|---|---|---|---|---|---|---|
| | | | Z-avg | | mean | |
| Compound | sg:mRNA | % E | (nm) | PDI | (nm) | Experiment |
| Compound 1 | 1:2 | 98 | 88 | 0.01 | 73 | 109 |
| Compound 12 | 1:2 | 97 | 80 | 0.06 | 62 | 109 |
| Compound 59 | 1:2 | 98 | 91 | 0.04 | 75 | 109 |
| Compound 94 | 1:2 | 97 | 95 | 0.02 | 77 | 109 |

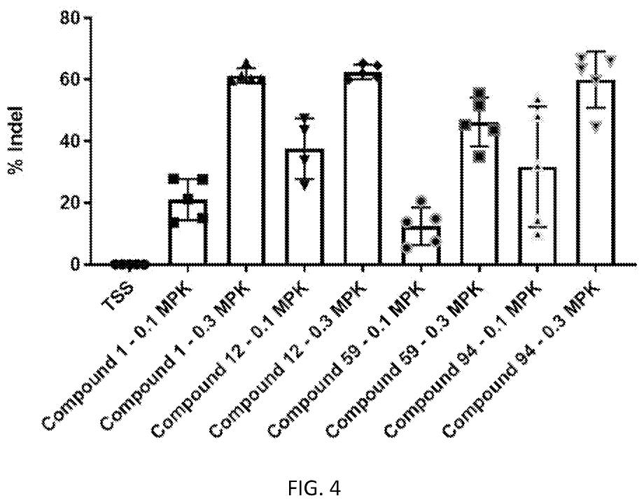

Sprague Dawley female rats were dosed i.v. at 0.1 or 0.3 mg/kg and assessed for editing in liver. Results are shown in FIG. 4 and Table 11.

TABLE 11
Editing efficiency in rat, providing % indel formation
for G534 experiments and serum TTR levels.

| | | Editing | | Serum TTR | | | | | | |
|---|---|---|---|---|---|---|---|---|---|---|
| | Dose | Mean % | | | Serum TTR | | Guide | | | |
| Compound | (mg/kg) | Indel | SD | N | (ug/ml) | SD | N | % TSS | Experiment | |
| TSS | 0 | 0.1 | 0.0 | 5 | 1620.7 | 165.6 | 5 | 100.0 | G534 | 109 |
| Compound 1 | 0.1 | 21.1 | 6.7 | 5 | 916.9 | 141.1 | 5 | 56.6 | G534 | 109 |
| Compound 1 | 0.3 | 61.3 | 2.3 | 5 | 114.3 | 27.4 | 5 | 7.1 | G534 | 109 |
| Compound 12 | 0.1 | 37.6 | 9.8 | 4 | 744.2 | 196.4 | 4 | 45.9 | G534 | 109 |
| Compound 12 | 0.3 | 62.5 | 2.4 | 5 | 89.1 | 25.5 | 5 | 5.5 | G534 | 109 |
| Compound 59 | 0.1 | 12.4 | 6.1 | 5 | 1124.0 | 225.9 | 5 | 69.4 | G534 | 109 |
| Compound 59 | 0.3 | 46.2 | 7.9 | 5 | 337.9 | 95.2 | 5 | 20.9 | G534 | 109 |
| Compound 94 | 0.1 | 31.7 | 19.6 | 5 | 639.5 | 264.1 | 5 | 39.5 | G534 | 109 |
| Compound 94 | 0.3 | 60.0 | 9.1 | 5 | 132.9 | 115.3 | 5 | 8.2 | G534 | 109 |

SEQUENCE TABLE

| | | SEQ |
|---|---|---|
| | | ID |
| Description | Sequence | No. |
| G000282 | mU*mU*mA*CAGCCACGUCUACAGCAGUUUUAGAmG | 1 |
| sgRNA targeting | mCmUmAmGmAmAmAmUmAmGmCAAGUUAAAAUAA | |
| mouse TTR | GGCUAGUCCGUUAUCAmAmCmUmUmGmAmAmAmA | |
| | mAmGmUmGmGmCmAmCmCmGmAmGmUmCmGmGmU | |
| | mGmCmU*mU*mU*mU | |
| G000650 | mG*mA*mC*AAGCACCAGAAAGACCAGUUUUAGAmG | 2 |
| sgRNA targeting | mCmUmAmGmAmAmAmUmAmGmCAAGUUAAAAUAA | |
| human B2M | GGCUAGUCCGUUAUCAmAmCmUmUmGmAmAmAmA | |
| | mAmGmUmGmGmCmAmCmCmGmAmGmUmCmGmGmU | |
| | mGmCmU*mU*mU*mU | |
| mRNA encoding | GGGUCCCGCAGUCGGCGUCCAGCGGCUCUGCUUGUU | 3 |
| Cas9 | CGUGUGUGUGUCGUUGCAGGCCUUAUUCGGAUCCG | |
| | CCACCAUGGACAAGAAGUACAGCAUCGGACUGGAC | |
| | AUCGGAACAAACAGCGUCGGAUGGGCAGUCAUCAC | |
| | AGACGAAUACAAGGUCCCGAGCAAGAAGUUCAAGG | |
| | UCCUGGGAAACACAGACAGACACAGCAUCAAGAAG | |
| | AACCUGAUCGGAGCACUGCUGUUCGACAGCGGAGA | |
| | AACAGCAGAAGCAACAAGACUGAAGAGAACAGCAA | |
| | GAAGAAGAUACACAAGAAGAAAGAACAGAAUCUGC | |
| | UACCUGCAGGAAAUCUUCAGCAACGAAAUGGCAAA | |
| | GGUCGACGACAGCUUCUUCCACAGACUGGAAGAAA | |
| | GCUUCCUGGUCGAAGAAGACAAGAAGCACGAAAGA | |
| | CACCCGAUCUUCGGAAACAUCGUCGACGAAGUCGCA | |
| | UACCACGAAAAGUACCCGACAAUCUACCACCUGAGA | |
| | AAGAAGCUGGUCGACAGCACAGACAAGGCAGACCU | |
| | GAGACUGAUCUACCUGGCACUGGCACACAUGAUCA | |
| | AGUUCAGAGGACACUUCCUGAUCGAAGGAGACCUG | |
| | AACCCGGACAACAGCGACGUCGACAAGCUGUUCAUC | |
| | CAGCUGGUCCAGACAUACAACCAGCUGUUCGAAGA | |
| | AAACCCGAUCAACGCAAGCGGAGUCGACGCAAAGG | |
| | CAAUCCUGAGCGCAAGACUGAGCAAGAGCAGAAGA | |
| | CUGGAAAACCUGAUCGCACAGCUGCCGGGAGAAAA | |
| | GAAGAACGGACUGUUCGGAAACCUGAUCGCACUGA | |
| | GCCUGGGACUGACACCGAACUUCAAGAGCAACUUC | |
| | GACCUGGCAGAAGACGCAAAGCUGCAGCUGAGCAA | |
| | GGACACAUACGACGACGACCUGGACAACCUGCUGGC | |
| | ACAGAUCGGAGACCAGUACGCAGACCUGUUCCUGG | |
| | CAGCAAAGAACCUGAGCGACGCAAUCCUGCUGAGC | |
| | GACAUCCUGAGAGUCAACACAGAAAUCACAAAGGC | |
| | ACCGCUGAGCGCAAGCAUGAUCAAGAGAUACGACG | |
| | AACACCACCAGGACCUGACACUGCUGAAGGCACUGG | |
| | UCAGACAGCAGCUGCCGGAAAAGUACAAGGAAAUC | |
| | UUCUUCGACCAGAGCAAGAACGGAUACGCAGGAUA | |
| | CAUCGACGGAGGAGCAAGCCAGGAAGAAUUCUACA | |
| | AGUUCAUCAAGCCGAUCCUGGAAAAGAUGGACGGA | |
| | ACAGAAGAACUGCUGGUCAAGCUGAACAGAGAAGA | |
| | CCUGCUGAGAAAGCAGAGAACAUUCGACAACGGAA | |
| | GCAUCCCGCACCAGAUCCACCUGGGAGAACUGCACG | |
| | CAAUCCUGAGAAGACAGGAAGACUUCUACCCGUUC | |
| | CUGAAGGACAACAGAGAAAAGAUCGAAAAGAUCCU | |
| | GACAUUCAGAAUCCCGUACUACGUCGGACCGCUGGC | |
| | AAGAGGAAACAGCAGAUUCGCAUGGAUGACAAGAA | |
| | AGAGCGAAGAAACAAUCACACCGUGGAACUUCGAA | |
| | GAAGUCGUCGACAAGGGAGCAAGCGCACAGAGCUU | |
| | CAUCGAAAGAAUGACAAACUUCGACAAGAACCUGC | |
| | CGAACGAAAAGGUCCUGCCGAAGCACAGCCUGCUG | |
| | UACGAAUACUUCACAGUCUACAACGAACUGACAAA | |
| | GGUCAAGUACGUCACAGAAGGAAUGAGAAAGCCGG | |
| | CAUUCCUGAGCGGAGAACAGAAGAAGGCAAUCGUC | |
| | GACCUGCUGUUCAAGACAAACAGAAAGGUCACAGU | |
| | CAAGCAGCUGAAGGAAGACUACUUCAAGAAGAUCG | |
| | AAUGCUUCGACAGCGUCGAAAUCAGCGGAGUCGAA | |
| | GACAGAUUCAACGCAAGCCUGGGAACAUACCACGA | |
| | CCUGCUGAAGAUCAUCAAGGACAAGGACUUCCUGG | |
| | ACAACGAAGAAAACGAAGACAUCCUGGAAGACAUC | |
| | GUCCUGACACUGACACUGUUCGAAGACAGAGAAAU | |
| | GAUCGAAGAAAGACUGAAGACAUACGCACACCUGU | |
| | UCGACGACAAGGUCAUGAAGCAGCUGAAGAGAAGA | |
| | AGAUACACAGGAUGGGGAAGACUGAGCAGAAAGCU | |
| | GAUCAACGGAAUCAGAGACAAGCAGAGCGGAAAGA | |
| | CAAUCCUGGACUUCCUGAAGAGCGACGGAUUCGCA | |
| | AACAGAAACUUCAUGCAGCUGAUCCACGACGACAG | |
| | CCUGACAUUCAAGGAAGACAUCCAGAAGGCACAGG | |
| | UCAGCGGACAGGGAGACAGCCUGCACGAACACAUC | |
| | GCAAACCUGGCAGGAAGCCCGGCAAUCAAGAAGGG | |
| | AAUCCUGCAGACAGUCAAGGUCGUCGACGAACUGG | |
| | UCAAGGUCAUGGGAAGACACAAGCCGGAAAACAUC | |
| | GUCAUCGAAAUGGCAAGAGAAAACCAGACAACACA | |
| | GAAGGGACAGAAGAACAGCAGAGAAAGAAUGAAGA | |
| | GAAUCGAAGAAGGAAUCAAGGAACUGGGAAGCCAG | |
| | AUCCUGAAGGAACACCCGGUCGAAAACACACAGCU | |
| | GCAGAACGAAAAGCUGUACCUGUACUACCUGCAGA | |
| | ACGGAAGAGACAUGUACGUCGACCAGGAACUGGAC | |
| | AUCAACAGACUGAGCGACUACGACGUCGACCACAUC | |
| | GUCCCGCAGAGCUUCCUGAAGGACGACAGCAUCGAC | |
| | AACAAGGUCCUGACAAGAAGCGACAAGAACAGAGG | |
| | AAAGAGCGACAACGUCCCGAGCGAAGAAGUCGUCA | |
| | AGAAGAUGAAGAACUACUGGAGACAGCUGCUGAAC | |
| | GCAAAGCUGAUCACACAGAGAAAGUUCGACAACCU | |
| | GACAAAGGCAGAGAGAGGAGGACUGAGCGAACUGG | |
| | ACAAGGCAGGAUUCAUCAAGAGACAGCUGGUCGAA | |
| | ACAAGACAGAUCACAAAGCACGUCGCACAGAUCCU | |
| | GGACAGCAGAAUGAACACAAAGUACGACGAAAACG | |
| | ACAAGCUGAUCAGAGAAGUCAAGGUCAUCACACUG | |
| | AAGAGCAAGCUGGUCAGCGACUUCAGAAAGGACUU | |
| | CCAGUUCUACAAGGUCAGAGAAAUCAACAACUACC | |
| | ACCACGCACACGACGCAUACCUGAACGCAGUCGUCG | |
| | GAACAGCACUGAUCAAGAAGUACCCGAAGCUGGAA | |
| | AGCGAAUUCGUCUACGGAGACUACAAGGUCUACGA | |
| | CGUCAGAAAGAUGAUCGCAAAGAGCGAACAGGAAA | |
| | UCGGAAAGGCAACAGCAAAGUACUUCUUCUACAGC | |
| | AACAUCAUGAACUUCUUCAAGACAGAAAUCACACU | |
| | GGCAAACGGAGAAAUCAGAAAGAGACCGCUGAUCG | |
| | AAACAAACGGAGAAACAGGAGAAAUCGUCUGGGAC | |
| | AAGGGAAGAGACUUCGCAACAGUCAGAAAGGUCCU | |
| | GAGCAUGCCGCAGGUCAACAUCGUCAAGAAGACAG | |
| | AAGUCCAGACAGGAGGAUUCAGCAAGGAAAGCAUC | |
| | CUGCCGAAGAGAAACAGCGACAAGCUGAUCGCAAG | |
| | AAAGAAGGACUGGGACCCGAAGAAGUACGGAGGAU | |
| | UCGACAGCCCGACAGUCGCAUACAGCGUCCUGGUCG | |
| | UCGCAAAGGUCGAAAAGGGAAAGAGCAAGAAGCUG | |
| | AAGAGCGUCAAGGAACUGCUGGGAAUCACAAUCAU | |
| | GGAAAGAAGCAGCUUCGAAAAGAACCCGAUCGACU | |
| | UCCUGGAAGCAAAGGGAUACAAGGAAGUCAAGAAG | |
| | GACCUGAUCAUCAAGCUGCCGAAGUACAGCCUGUU | |
| | CGAACUGGAAAACGGAAGAAAGAGAAUGCUGGCAA | |
| | GCGCAGGAGAACUGCAGAAGGGAAACGAACUGGCA | |
| | CUGCCGAGCAAGUACGUCAACUUCCUGUACCUGGCA | |
| | AGCCACUACGAAAAGCUGAAGGGAAGCCCGGAAGA | |
| | CAACGAACAGAAGCAGCUGUUCGUCGAACAGCACA | |
| | AGCACUACCUGGACGAAAUCAUCGAACAGAUCAGC | |
| | GAAUUCAGCAAGAGAGUCAUCCUGGCAGACGCAAA | |
| | CCUGGACAAGGUCCUGAGCGCAUACAACAAGCACA | |
| | GAGACAAGCCGAUCAGAGAACAGGCAGAAAACAUC | |
| | AUCCACCUGUUCACACUGACAAACCUGGGAGCACCG | |
| | GCAGCAUUCAAGUACUUCGACACAACAAUCGACAG | |
| | AAAGAGAUACACAAGCACAAAGGAAGUCCUGGACG | |
| | CAACACUGAUCCACCAGAGCAUCACAGGACUGUACG | |
| | AAACAAGAAUCGACCUGAGCCAGCUGGGAGGAGAC | |
| | GGAGGAGGAAGCCCGAAGAAGAAGAGAAAGGUCUA | |
| | GCUAGCCAUCACAUUUAAAAGCAUCUCAGCCUACCA | |
| | UGAGAAUAAGAGAAAGAAAAUGAAGAUCAAUAGCU | |
| | UAUUCAUCUCUUUUUCUUUUUCGUUGGUGUAAAGC | |
| | CAACACCCUGUCUAAAAAACAUAAAUUUCUUUAAU | |
| | CAUUUUGCCUCUUUUCUCUGUGCUUCAAUUAAUAA | |
| | AAAAUGGAAAGAACCUCGAGAAAAAAAAAAAAAAA | |
| | AAAAAAAAAAAAAAAAAAAAAAAAAAAAAAAAAAA | |
| | AAAAAAAAAAAAAAAAAAAAAAAAAAAAAAAAAAA | |
| | AAAAAAAAAAAAAAAUCUAG | |
| G000502 | mA*mC*mA*CAAAUACCAGUCCAGCGGUUUUAGAmG | 4 |
| sgRNA targeting | mCmUmAmGmAmAmAmUmAmGmCAAGUUAAAAUAA | |
| mouse TTR | GGCUAGUCCGUUAUCAmAmCmUmUmGmAmAmAmA | |
| | mAmGmUmGmGmCmAmCmCmGmAmGmUmCmGmGmU | |
| | mGmCmU*mU*mU*mU | |
| G000534 | mA*mC*mG*CAAAUAUCAGUCCAGCGGUUUUAGAmG | 5 |
| sgRNA targeting | mCmUmAmGmAmAmAmUmAmGmCAAGUUAAAAUAA | |
| rat TTR | GGCUAGUCCGUUAUCAmAmCmUmUmGmAmAmAmA | |
| | mAmGmUmGmGmCmAmCmCmGmAmGmUmCmGmGmU | |
| | mGmCmU*mU*mU*mU | |
| Cas9 mRNA | AUGGACAAGAAGUACUCCAUCGGCCUGGACAUCGG | 6 |
| | CACCAACUCCGUGGGCUGGGCCGUGAUCACCGACGA | |
| | GUACAAGGUGCCCUCCAAGAAGUUCAAGGUGCUGG | |
| | GCAACACCGACCGGCACUCCAUCAAGAAGAACCUGA | |
| | UCGGCGCCCUGCUGUUCGACUCCGGCGAGACCGCCG | |
| | AGGCCACCCGGCUGAAGCGGACCGCCCGGCGGCGGU | |
| | ACACCCGGCGGAAGAACCGGAUCUGCUACCUGCAGG | |
| | AGAUCUUCUCCAACGAGAUGGCCAAGGUGGACGAC | |
| | UCCUUCUUCCACCGGCUGGAGGAGUCCUUCCUGGUG | |
| | GAGGAGGACAAGAAGCACGAGCGGCACCCCAUCUU | |
| | CGGCAACAUCGUGGACGAGGUGGCCUACCACGAGA | |
| | AGUACCCCACCAUCUACCACCUGCGGAAGAAGCUGG | |
| | UGGACUCCACCGACAAGGCCGACCUGCGGCUGAUCU | |
| | ACCUGGCCCUGGCCCACAUGAUCAAGUUCCGGGGCC | |
| | ACUUCCUGAUCGAGGGCGACCUGAACCCCGACAACU | |
| | CCGACGUGGACAAGCUGUUCAUCCAGCUGGUGCAG | |
| | ACCUACAACCAGCUGUUCGAGGAGAACCCCAUCAAC | |
| | GCCUCCGGCGUGGACGCCAAGGCCAUCCUGUCCGCC | |
| | CGGCUGUCCAAGUCCCGGCGGCUGGAGAACCUGAUC | |
| | GCCCAGCUGCCCGGCGAGAAGAAGAACGGCCUGUUC | |
| | GGCAACCUGAUCGCCCUGUCCCUGGGCCUGACCCCC | |
| | AACUUCAAGUCCAACUUCGACCUGGCCGAGGACGCC | |
| | AAGCUGCAGCUGUCCAAGGACACCUACGACGACGAC | |
| | CUGGACAACCUGCUGGCCCAGAUCGGCGACCAGUAC | |
| | GCCGACCUGUUCCUGGCCGCCAAGAACCUGUCCGAC | |
| | GCCAUCCUGCUGUCCGACAUCCUGCGGGUGAACACC | |
| | GAGAUCACCAAGGCCCCCCUGUCCGCCUCCAUGAUC | |
| | AAGCGGUACGACGAGCACCACCAGGACCUGACCCUG | |
| | CUGAAGGCCCUGGUGCGGCAGCAGCUGCCCGAGAA | |
| | GUACAAGGAGAUCUUCUUCGACCAGUCCAAGAACG | |
| | GCUACGCCGGCUACAUCGACGGCGGCGCCUCCCAGG | |
| | AGGAGUUCUACAAGUUCAUCAAGCCCAUCCUGGAG | |
| | AAGAUGGACGGCACCGAGGAGCUGCUGGUGAAGCU | |
| | GAACCGGGAGGACCUGCUGCGGAAGCAGCGGACCU | |
| | UCGACAACGGCUCCAUCCCCCACCAGAUCCACCUGG | |
| | GCGAGCUGCACGCCAUCCUGCGGCGGCAGGAGGACU | |
| | UCUACCCCUUCCUGAAGGACAACCGGGAGAAGAUC | |
| | GAGAAGAUCCUGACCUUCCGGAUCCCCUACUACGUG | |
| | GGCCCCCUGGCCCGGGGCAACUCCCGGUUCGCCUGG | |
| | AUGACCCGGAAGUCCGAGGAGACCAUCACCCCCUGG | |
| | AACUUCGAGGAGGUGGUGGACAAGGGCGCCUCCGC | |
| | CCAGUCCUUCAUCGAGCGGAUGACCAACUUCGACAA | |
| | GAACCUGCCCAACGAGAAGGUGCUGCCCAAGCACUC | |
| | CCUGCUGUACGAGUACUUCACCGUGUACAACGAGC | |
| | UGACCAAGGUGAAGUACGUGACCGAGGGCAUGCGG | |
| | AAGCCCGCCUUCCUGUCCGGCGAGCAGAAGAAGGCC | |
| | AUCGUGGACCUGCUGUUCAAGACCAACCGGAAGGU | |
| | GACCGUGAAGCAGCUGAAGGAGGACUACUUCAAGA | |
| | AGAUCGAGUGCUUCGACUCCGUGGAGAUCUCCGGC | |
| | GUGGAGGACCGGUUCAACGCCUCCCUGGGCACCUAC | |
| | CACGACCUGCUGAAGAUCAUCAAGGACAAGGACUU | |
| | CCUGGACAACGAGGAGAACGAGGACAUCCUGGAGG | |
| | ACAUCGUGCUGACCCUGACCCUGUUCGAGGACCGGG | |
| | AGAUGAUCGAGGAGCGGCUGAAGACCUACGCCCAC | |
| | CUGUUCGACGACAAGGUGAUGAAGCAGCUGAAGCG | |
| | GCGGCGGUACACCGGCUGGGGCCGGCUGUCCCGGAA | |
| | GCUGAUCAACGGCAUCCGGGACAAGCAGUCCGGCA | |
| | AGACCAUCCUGGACUUCCUGAAGUCCGACGGCUUCG | |
| | CCAACCGGAACUUCAUGCAGCUGAUCCACGACGACU | |
| | CCCUGACCUUCAAGGAGGACAUCCAGAAGGCCCAGG | |
| | UGUCCGGCCAGGGCGACUCCCUGCACGAGCACAUCG | |
| | CCAACCUGGCCGGCUCCCCCGCCAUCAAGAAGGGCA | |
| | UCCUGCAGACCGUGAAGGUGGUGGACGAGCUGGUG | |
| | AAGGUGAUGGGCCGGCACAAGCCCGAGAACAUCGU | |
| | GAUCGAGAUGGCCCGGGAGAACCAGACCACCCAGA | |
| | AGGGCCAGAAGAACUCCCGGGAGCGGAUGAAGCGG | |
| | AUCGAGGAGGGCAUCAAGGAGCUGGGCUCCCAGAU | |
| | CCUGAAGGAGCACCCCGUGGAGAACACCCAGCUGCA | |
| | GAACGAGAAGCUGUACCUGUACUACCUGCAGAACG | |
| | GCCGGGACAUGUACGUGGACCAGGAGCUGGACAUC | |
| | AACCGGCUGUCCGACUACGACGUGGACCACAUCGUG | |
| | CCCCAGUCCUUCCUGAAGGACGACUCCAUCGACAAC | |
| | AAGGUGCUGACCCGGUCCGACAAGAACCGGGGCAA | |
| | GUCCGACAACGUGCCCUCCGAGGAGGUGGUGAAGA | |
| | AGAUGAAGAACUACUGGCGGCAGCUGCUGAACGCC | |
| | AAGCUGAUCACCCAGCGGAAGUUCGACAACCUGACC | |
| | AAGGCCGAGCGGGGCGGCCUGUCCGAGCUGGACAA | |
| | GGCCGGCUUCAUCAAGCGGCAGCUGGUGGAGACCC | |
| | GGCAGAUCACCAAGCACGUGGCCCAGAUCCUGGACU | |
| | CCCGGAUGAACACCAAGUACGACGAGAACGACAAG | |
| | CUGAUCCGGGAGGUGAAGGUGAUCACCCUGAAGUC | |
| | CAAGCUGGUGUCCGACUUCCGGAAGGACUUCCAGU | |
| | UCUACAAGGUGCGGGAGAUCAACAACUACCACCAC | |
| | GCCCACGACGCCUACCUGAACGCCGUGGUGGGCACC | |
| | GCCCUGAUCAAGAAGUACCCCAAGCUGGAGUCCGA | |
| | GUUCGUGUACGGCGACUACAAGGUGUACGACGUGC | |
| | GGAAGAUGAUCGCCAAGUCCGAGCAGGAGAUCGGC | |
| | AAGGCCACCGCCAAGUACUUCUUCUACUCCAACAUC | |
| | AUGAACUUCUUCAAGACCGAGAUCACCCUGGCCAAC | |
| | GGCGAGAUCCGGAAGCGGCCCCUGAUCGAGACCAAC | |
| | GGCGAGACCGGCGAGAUCGUGUGGGACAAGGGCCG | |
| | GGACUUCGCCACCGUGCGGAAGGUGCUGUCCAUGCC | |
| | CCAGGUGAACAUCGUGAAGAAGACCGAGGUGCAGA | |
| | CCGGCGGCUUCUCCAAGGAGUCCAUCCUGCCCAAGC | |
| | GGAACUCCGACAAGCUGAUCGCCCGGAAGAAGGAC | |
| | UGGGACCCCAAGAAGUACGGCGGCUUCGACUCCCCC | |
| | ACCGUGGCCUACUCCGUGCUGGUGGUGGCCAAGGU | |
| | GGAGAAGGGCAAGUCCAAGAAGCUGAAGUCCGUGA | |
| | AGGAGCUGCUGGGCAUCACCAUCAUGGAGCGGUCC | |
| | UCCUUCGAGAAGAACCCCAUCGACUUCCUGGAGGCC | |
| | AAGGGCUACAAGGAGGUGAAGAAGGACCUGAUCAU | |
| | CAAGCUGCCCAAGUACUCCCUGUUCGAGCUGGAGA | |
| | ACGGCCGGAAGCGGAUGCUGGCCUCCGCCGGCGAGC | |
| | UGCAGAAGGGCAACGAGCUGGCCCUGCCCUCCAAGU | |
| | ACGUGAACUUCCUGUACCUGGCCUCCCACUACGAGA | |
| | AGCUGAAGGGCUCCCCCGAGGACAACGAGCAGAAG | |
| | CAGCUGUUCGUGGAGCAGCACAAGCACUACCUGGA | |
| | CGAGAUCAUCGAGCAGAUCUCCGAGUUCUCCAAGC | |
| | GGGUGAUCCUGGCCGACGCCAACCUGGACAAGGUG | |
| | CUGUCCGCCUACAACAAGCACCGGGACAAGCCCAUC | |
| | CGGGAGCAGGCCGAGAACAUCAUCCACCUGUUCACC | |
| | CUGACCAACCUGGGCGCCCCCGCCGCCUUCAAGUAC | |
| | UUCGACACCACCAUCGACCGGAAGCGGUACACCUCC | |
| | ACCAAGGAGGUGCUGGACGCCACCCUGAUCCACCAG | |
| | UCCAUCACCGGCCUGUACGAGACCCGGAUCGACCUG | |
| | UCCCAGCUGGGCGGCGACGGCGGCGGCUCCCCCAAG | |
| | AAGAAGCGGAAGGUGUGA | |

2′-O-methyl modifications and phosphorothioate linkages as represented below (m=2′-OMe; *=phosphorothioate).

1. A compound of Formula II 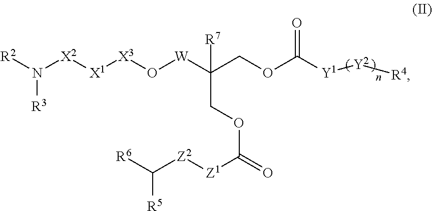
wherein
X² is C₂₋₅ alkylene,
X³ is C(═O) or a direct bond,
Y¹ is C₂₋₁₂ alkylene,
n is 0 to 3,
R⁴ is C₁₋₁₅ alkyl,
R⁵ is C₅₋₉ alkyl or C₆₋₁₀ alkoxy,
R⁶ is C₅₋₉ alkyl or C₆₋₁₀ alkoxy,
W is methylene or a direct bond,
R⁷ is H or Me, and
(i) X¹ is O, NR¹, or a direct bond,
R¹ is H or Me,
R² taken together with R³ and the nitrogen atom to which they are attached form a 5-, 6-, or 7-membered ring, or
R³ is C₁₋₃ alkyl and R² is C₁₋₃ alkyl, or
R² taken together with the nitrogen atom to which it is attached and 1-3 carbon atoms of X² form a 4-, 5-, or 6-membered ring, or
X¹ is NR¹, and R¹ and R² taken together with the nitrogen atoms to which they are attached form a 5- or 6-membered ring, and
Y² is selected from 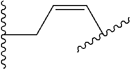 [SMILES: */C=C\C*]
(in either orientation), 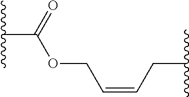 [SMILES: *C/C=C\COC(*)=O]
(in either orientation), and 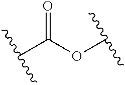 [SMILES: *OC(*)=O]
(in either orientation),
Z¹ is C₁₋₆ alkylene or a direct bond, and
Z² is 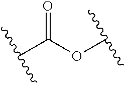 [SMILES: *OC(*)=O]
(in either orientation);
(ii) X¹ is O, NR¹, or a direct bond,
R¹ is H or Me,
R² taken together with R³ and the nitrogen atom to which they are attached form a 5-, 6-, or 7-membered ring, or
R³ is C₁₋₃ alkyl and R² is C₁₋₃ alkyl, or
R² taken together with the nitrogen atom to which it is attached and 1-3 carbon atoms of X² form a 4-, 5-, or 6-membered ring, or
X¹ is NR¹, and R¹ and R² taken together with the nitrogen atoms to which they are attached form a 5- or 6-membered ring, and
Z¹ is C₁₋₆ alkylene or a direct bond,
Z² is 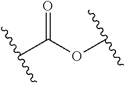 [SMILES: *OC(*)=O]
(in either orientation) or absent, provided that if Z¹ is a direct bond, Z² is absent, and
Y² is 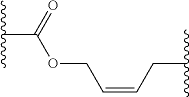 [SMILES: *C/C=C\COC(*)=O]
(in either orientation), or
(iii) X¹ is NR¹,
R¹ is H or Me,
R² taken together with R³ and the nitrogen atom to which they are attached form a 5-, 6-, or 7-membered ring, or
R³ is C₂₋₃ alkyl and R² is C₂₋₃ alkyl, or
R² taken together with the nitrogen atom to which it is attached and 1-3 carbon atoms of X² form a 4-, 5-, or 6-membered ring, or
X¹ is NR¹, and R¹ and R² taken together with the nitrogen atoms to which they are attached form a 5- or 6-membered ring,
Z¹ is C₁₋₆ alkylene or a direct bond, and
Z² is 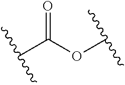 [SMILES: *OC(*)=O]
(in either orientation) or absent, provided that if Z¹ is a direct bond, Z² is absent, and
Y² is selected from 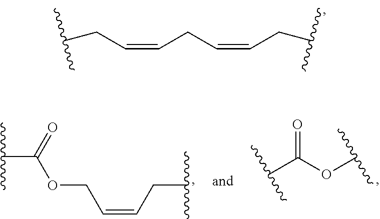 [SMILES: *C/C=C\C/C=C\C* ; *C/C=C\COC(*)=O ; *OC(*)=O]
or a salt thereof.

2. (canceled)

3. The compound of claim 1, wherein X² is linear C₂ alkylene, C₃ alkylene, or C₄ alkylene.

4.-5. (canceled)

6. The compound of claim 1, wherein R³ is C₁ alkyl or C₂ alkyl.

7. (canceled)

8. The compound of claim 1, wherein R² is C₁ alkyl or C₂ alkyl.

9. (canceled)

10. The compound of claim 1, wherein R² taken together with the nitrogen atom and 1-3 carbon atoms of X² form a 5- to 6-membered ring or wherein R² and R³ taken together with the nitrogen atom form a 5-membered ring.

11.-16. (canceled)

17. The compound of claim 1, wherein Y¹ is linear C₅₋₇ alkylene.

18. (canceled)

19. The compound of claim 1, wherein R⁴ is linear C₆₋₁₂ alkyl.

20. The compound of claim 1, wherein Z¹ is linear C₂₋₄ alkylene.

21.-22. (canceled)

23. The compound of any one of claim 1, wherein R⁵ and R⁶ are each independently linear C₇₋₉ alkoxy.

24. The compound of claim 1, wherein Y² is

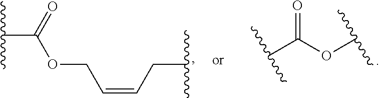 [SMILES: *C/C=C\COC(*)=O ; *OC(*)=O]

25. The compound of claim 1, wherein Y¹ is linear C₇ alkylene, Y² is 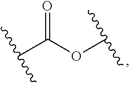 [SMILES: *OC(*)=O]
n=1, and R⁴ is linear C₁₀ alkyl.

26. The compound of claim 1, wherein Z² is

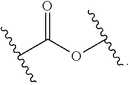 [SMILES: *OC(*)=O]

27. The compound of claim 1, wherein Z¹, Z², and R⁵ are selected to form a linear chain of 6-18 atoms, including the carbon and oxygen atoms of the ester and the acetal.

28. The compound of claim 1, wherein Y¹, Y², and R⁴ are selected to form a linear chain of 14-24 atoms, including the carbon and oxygen atoms of the ester(s), if present.

29. A compound is-selected from: 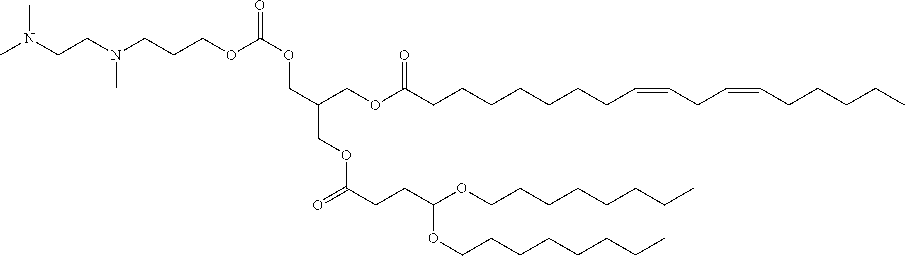 [SMILES: CCCCC/C=C\C/C=C\CCCCCCCC(=O)OCC(COC(=O)CCC(OCCCCCCCC)OCCCCCCCC)COC(=O)OCCCN(C)CCN(C)C] 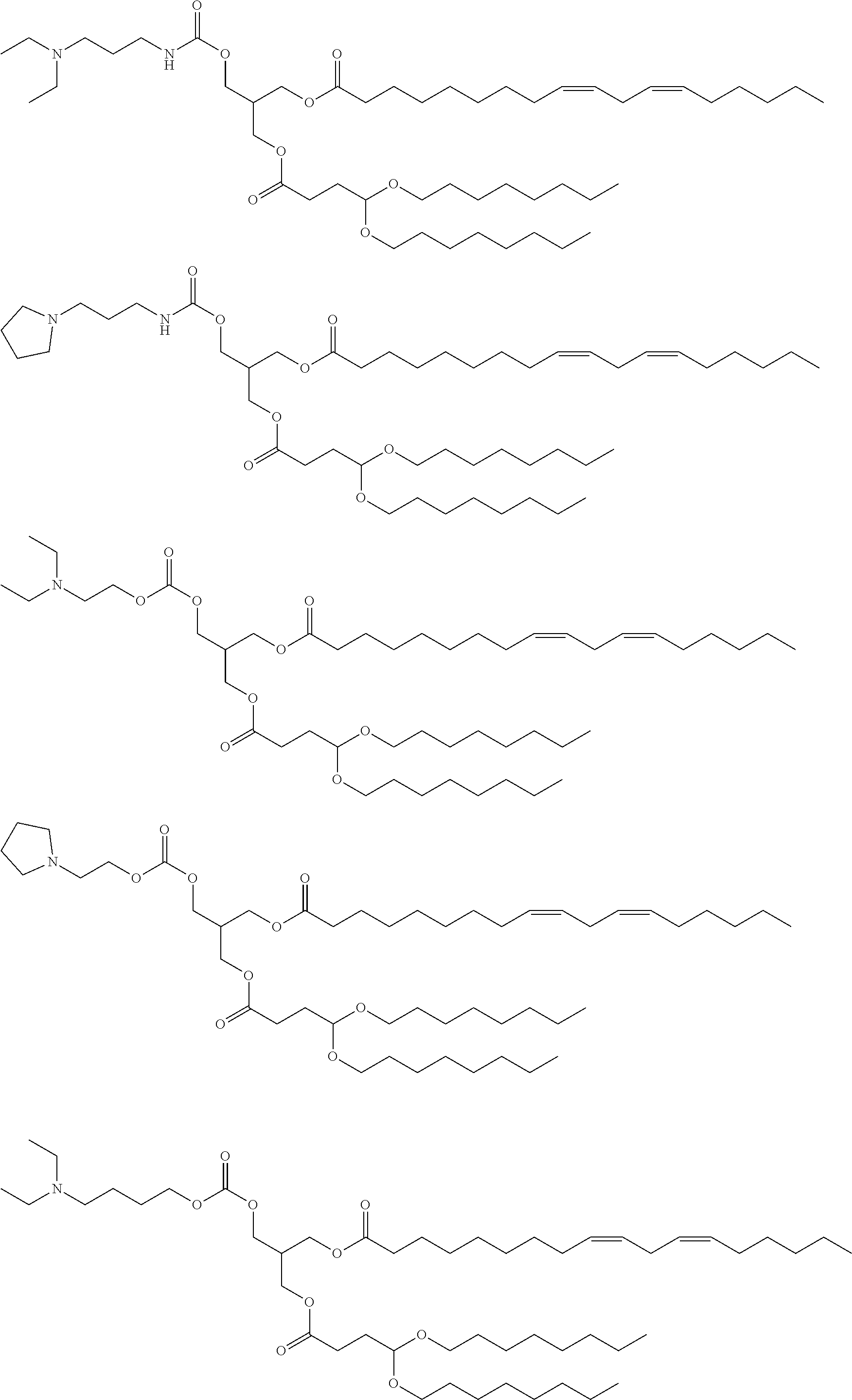 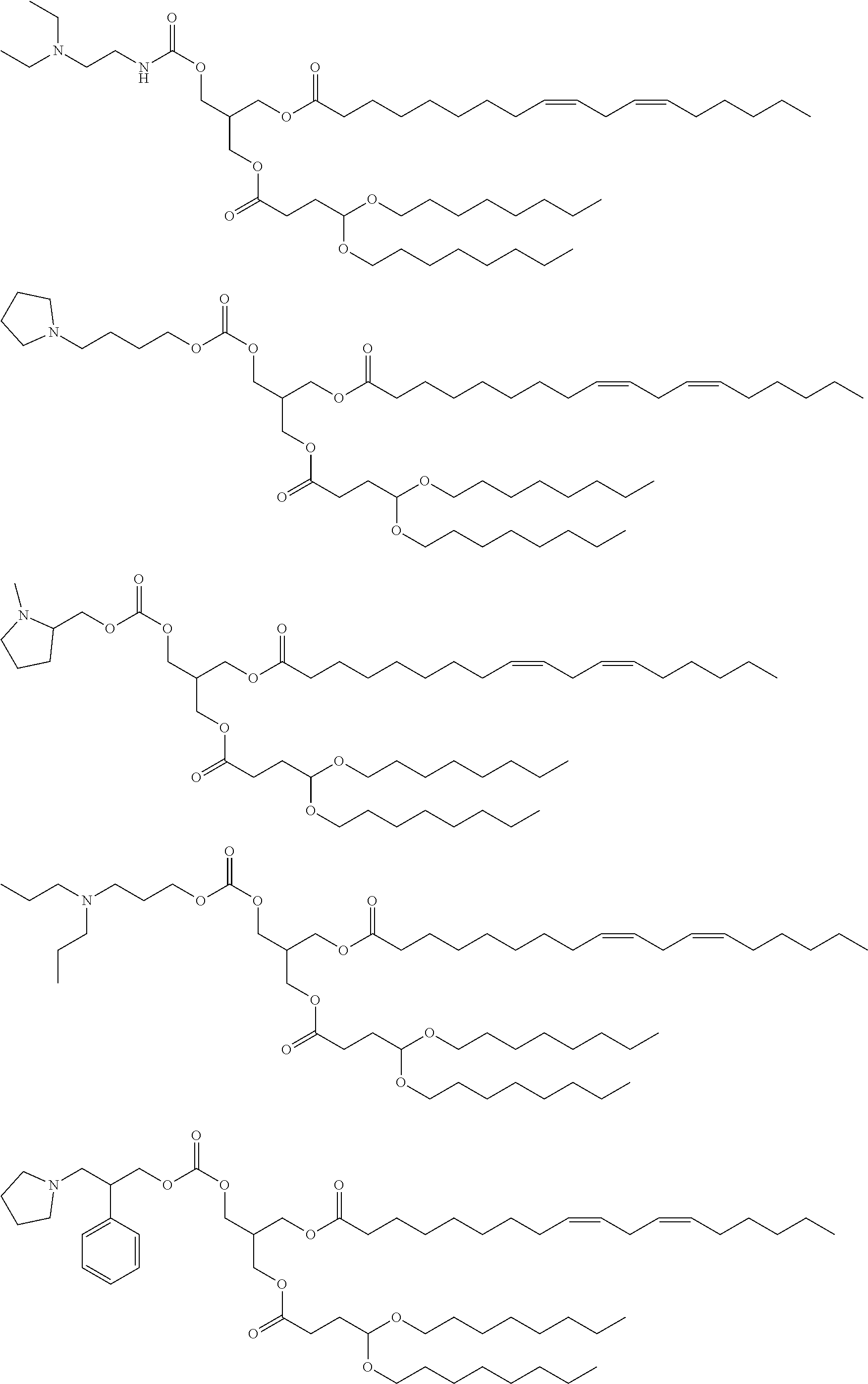 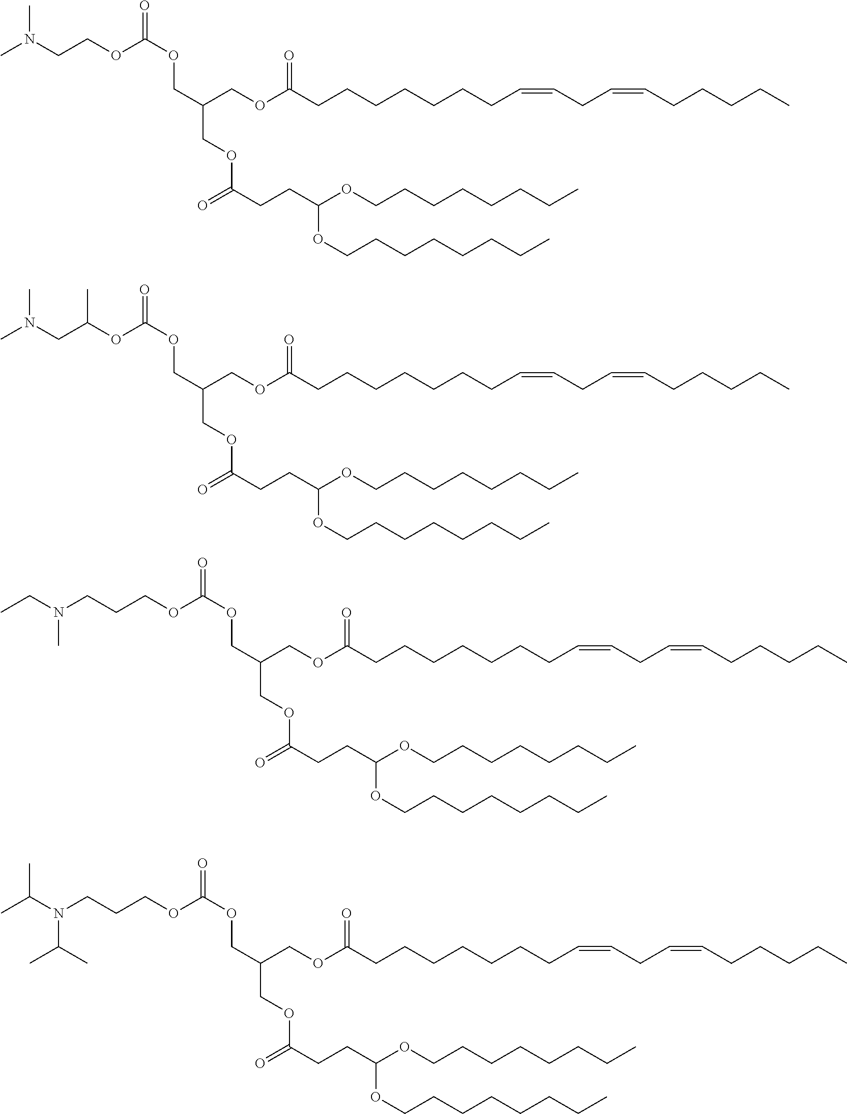 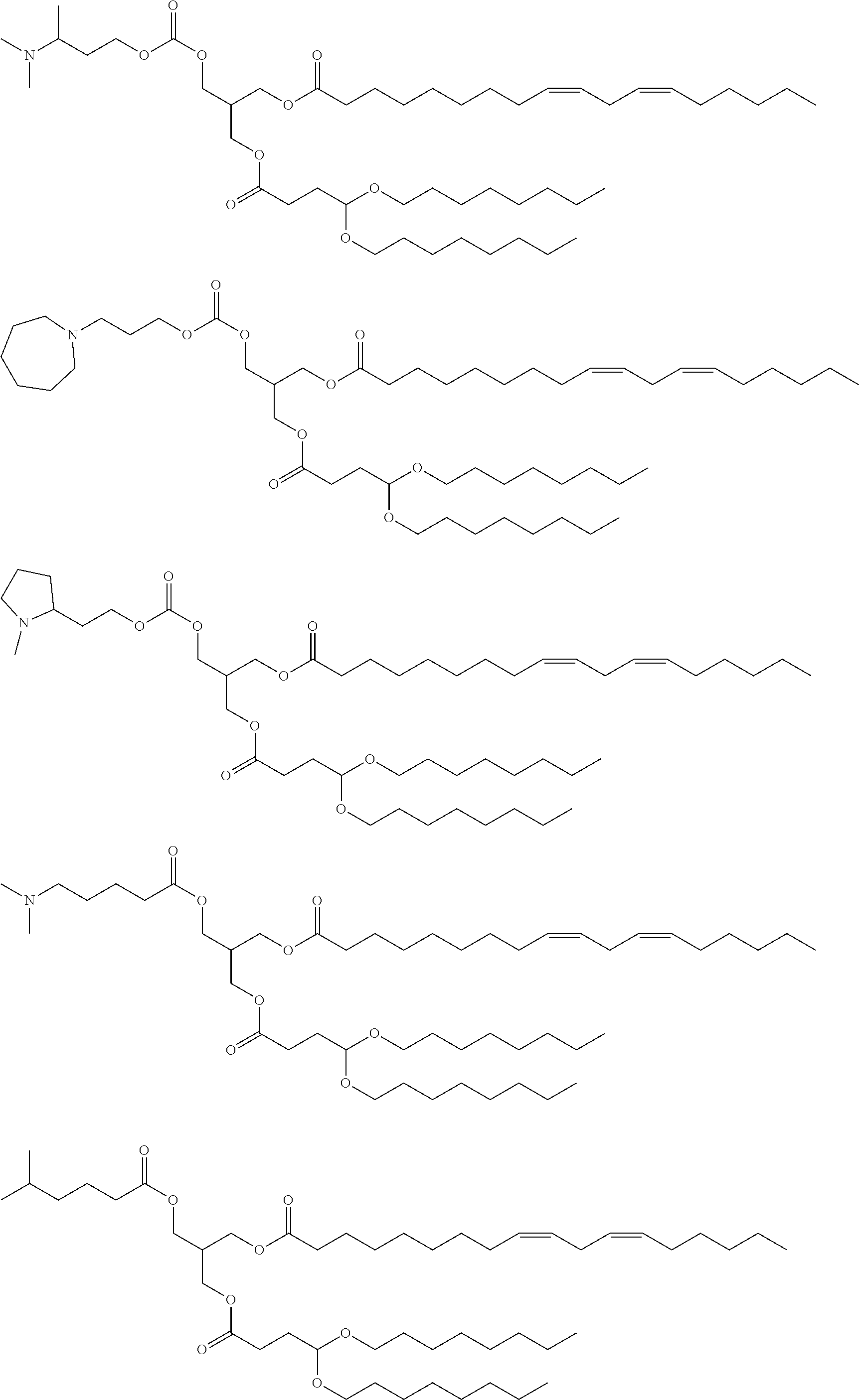 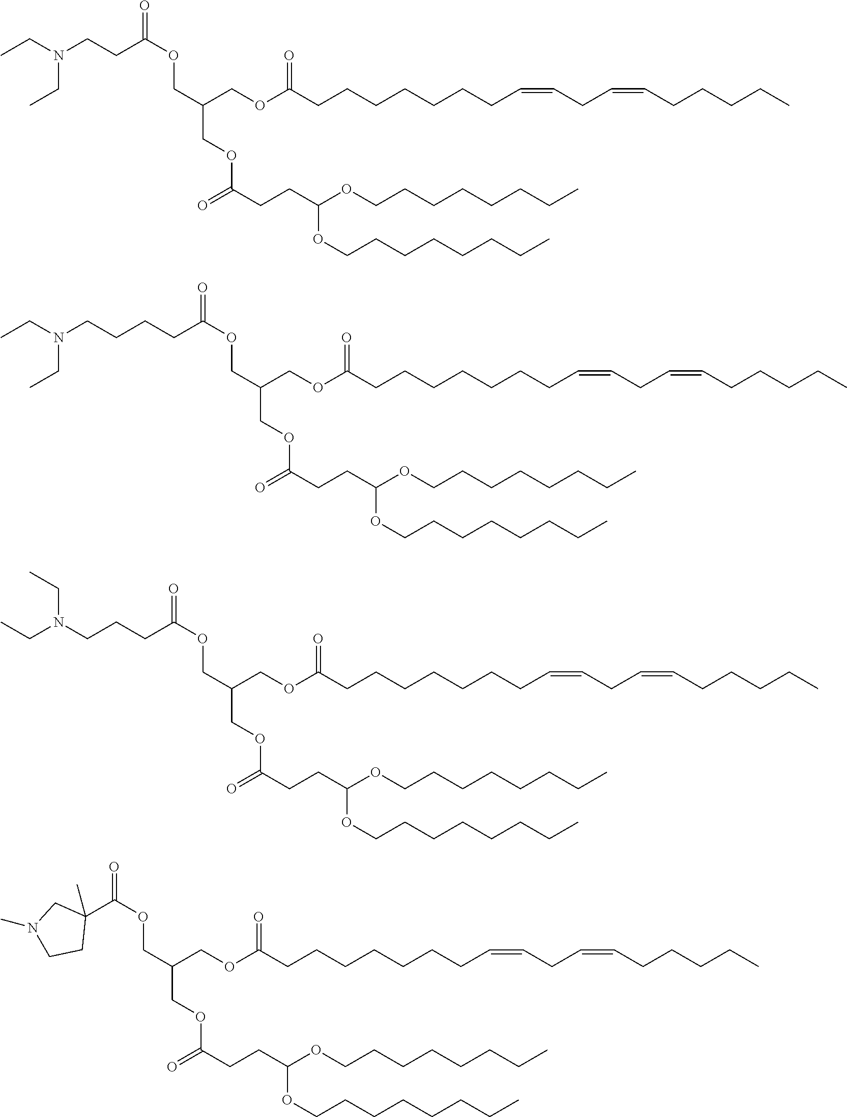 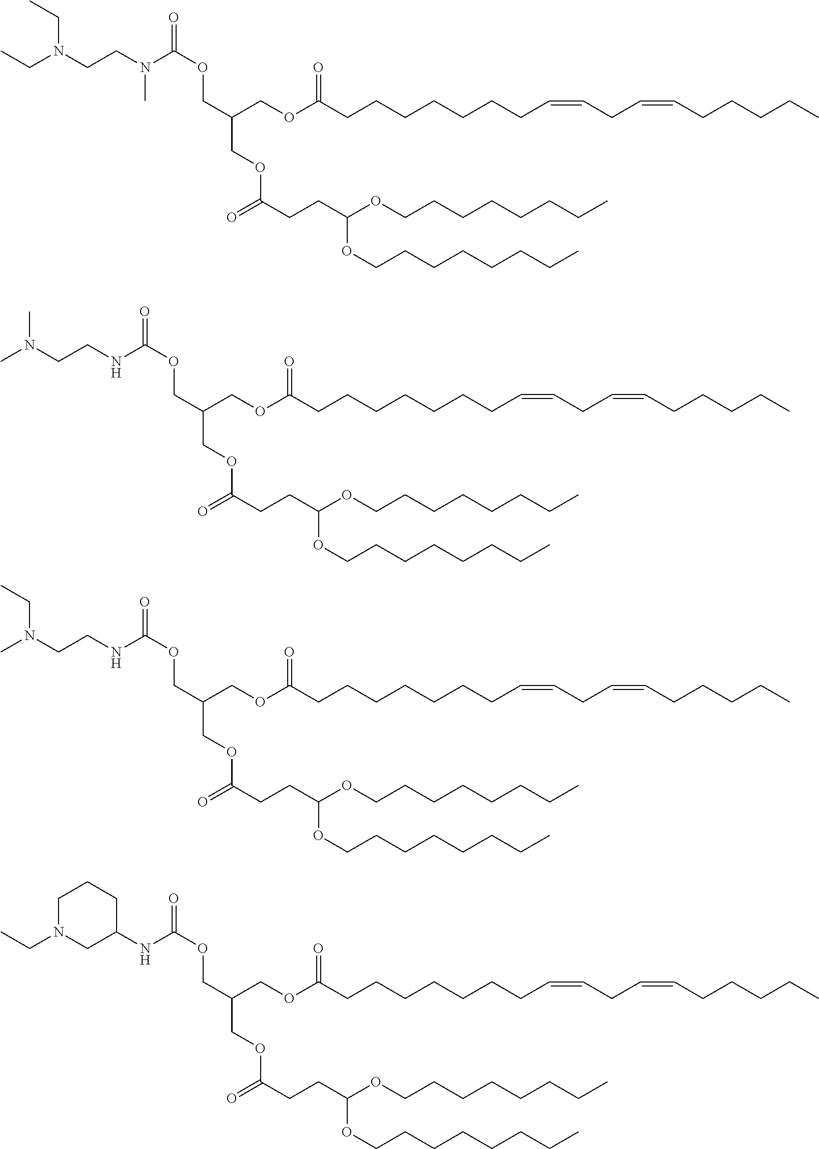 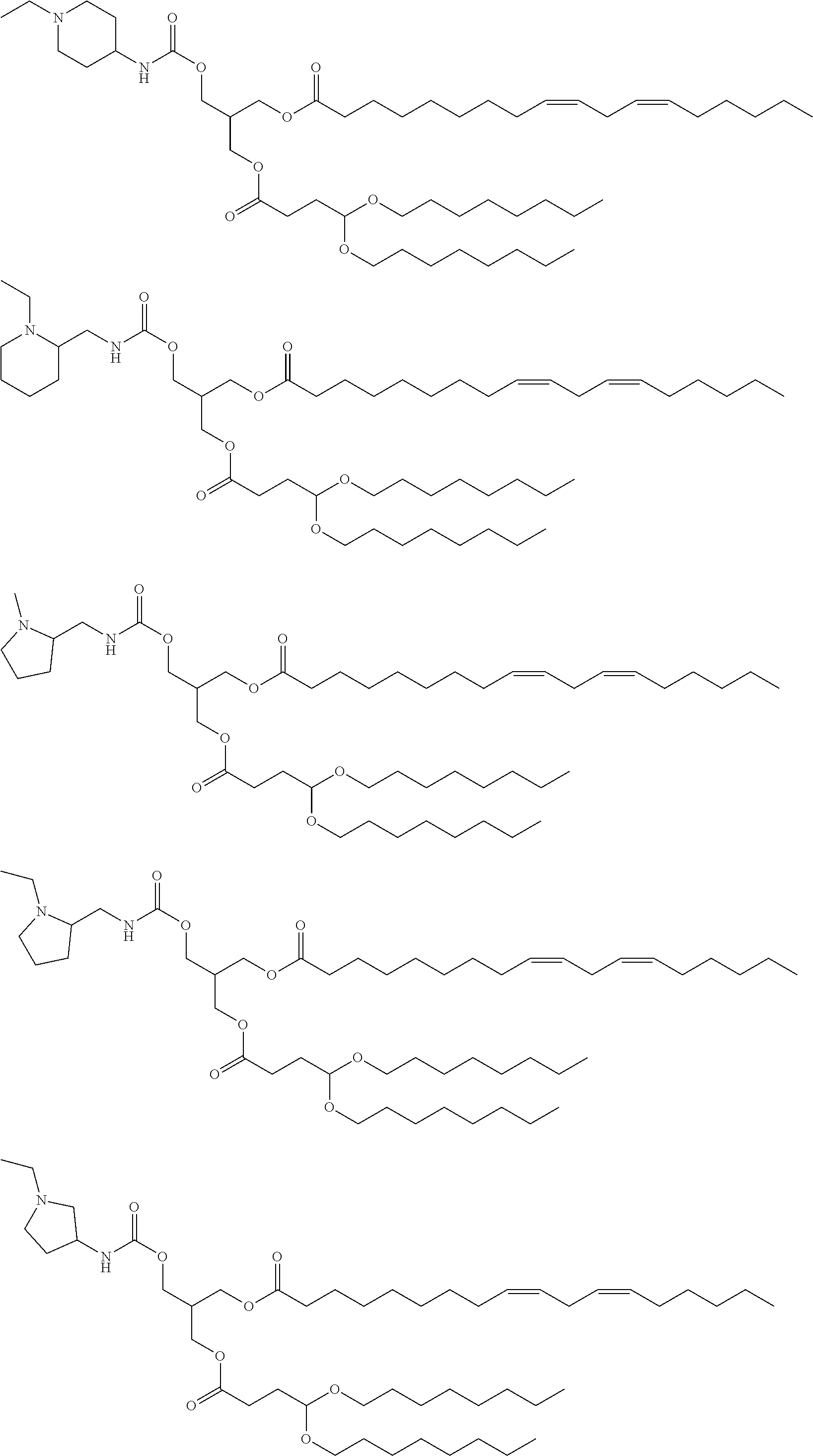 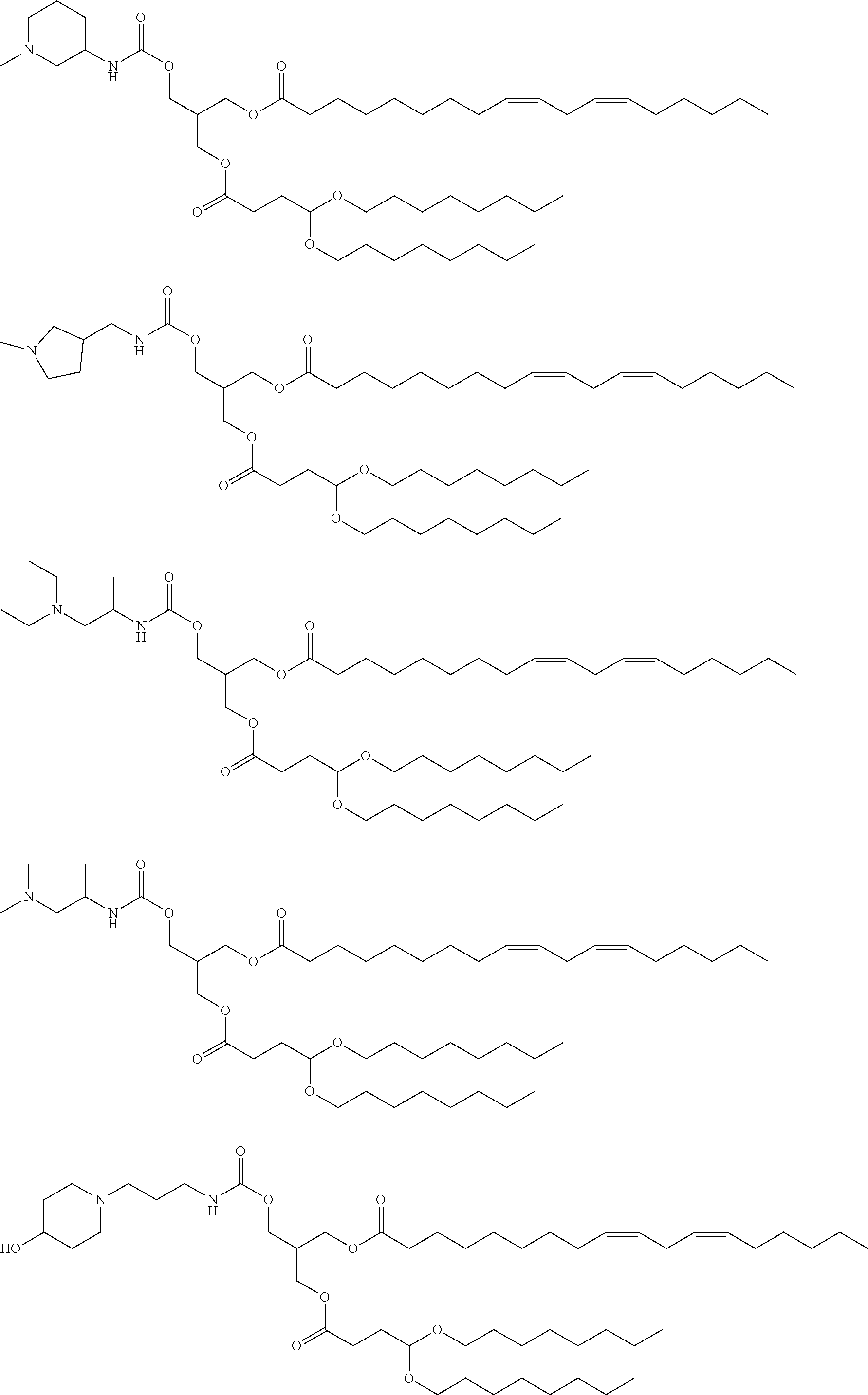 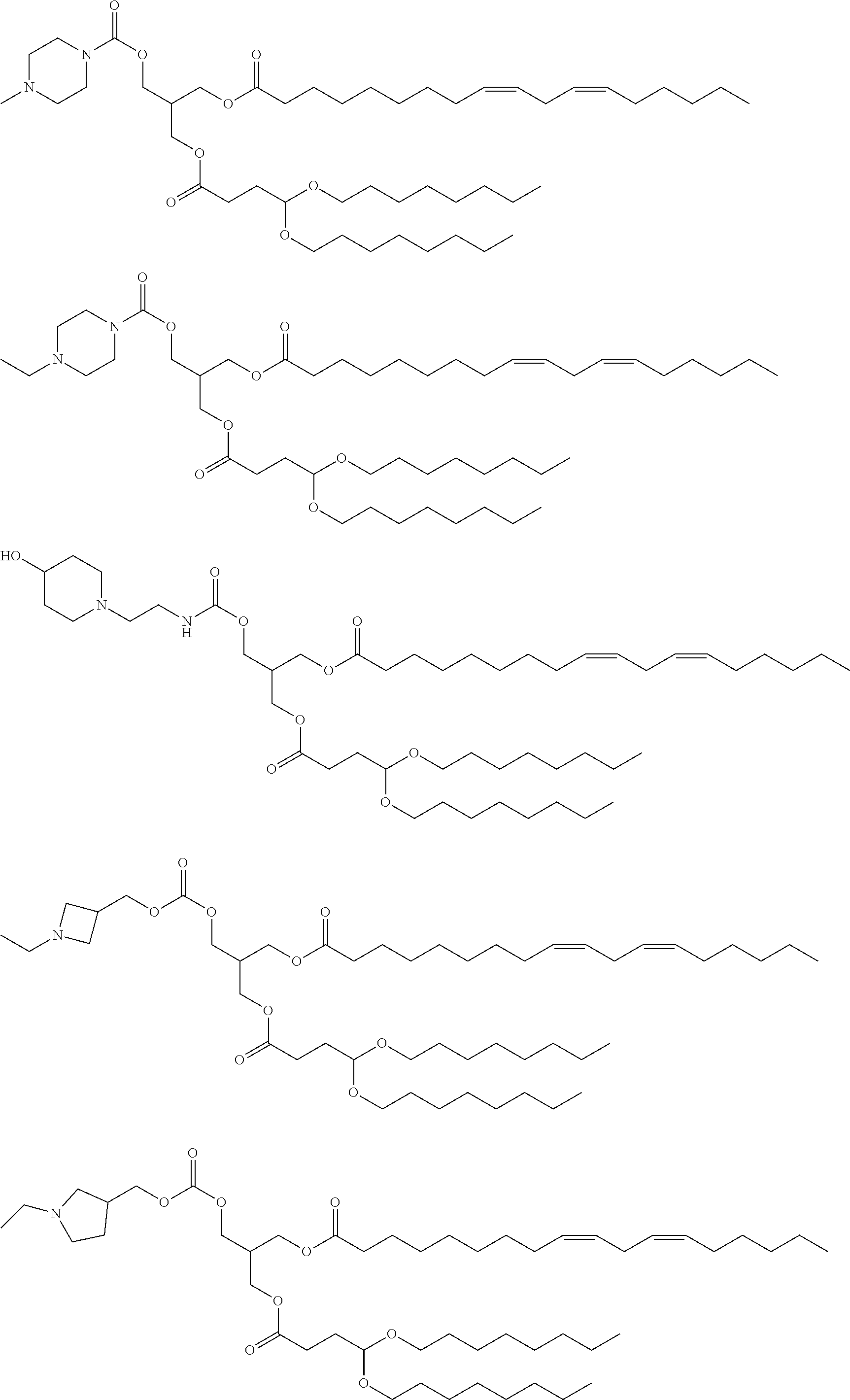 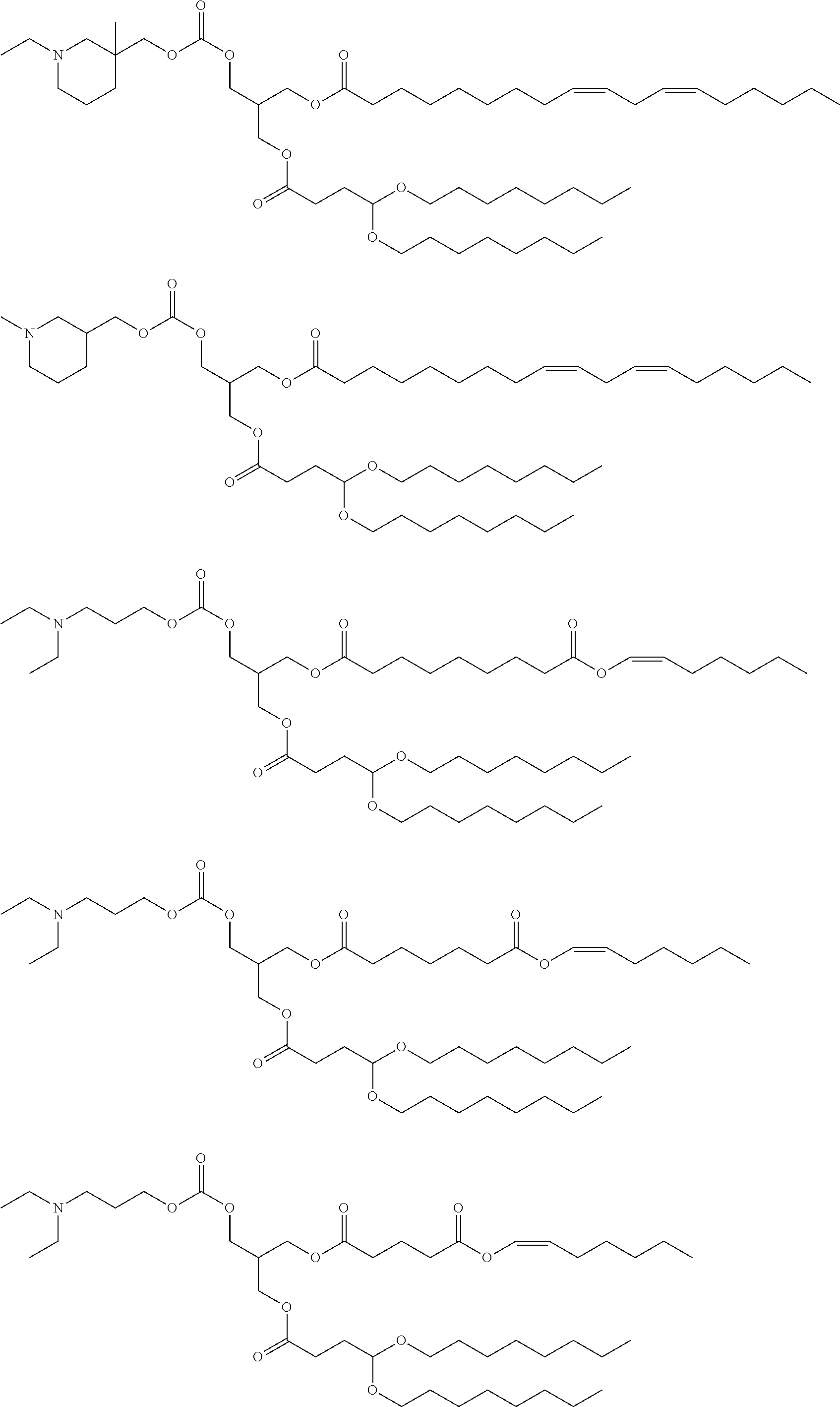 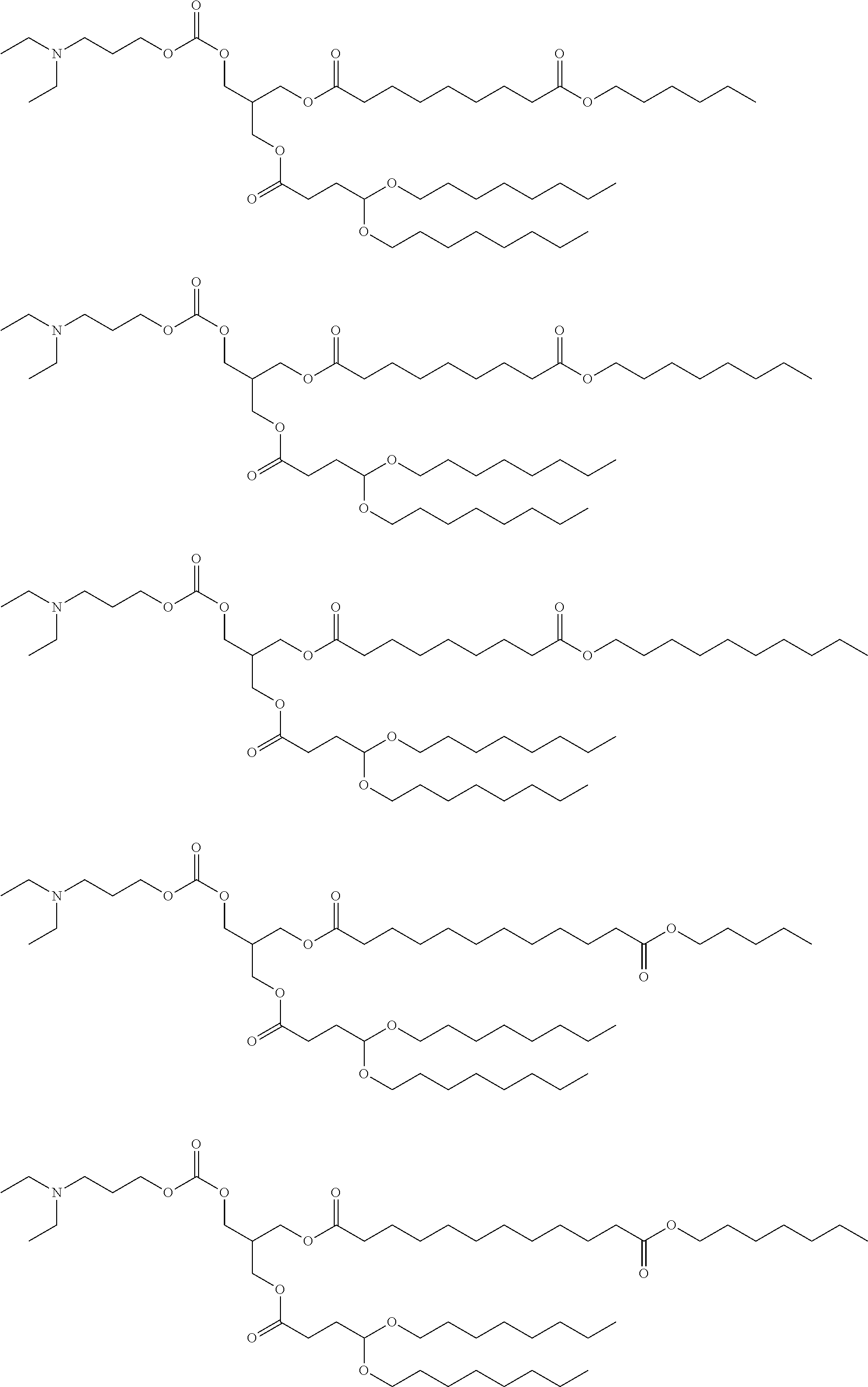 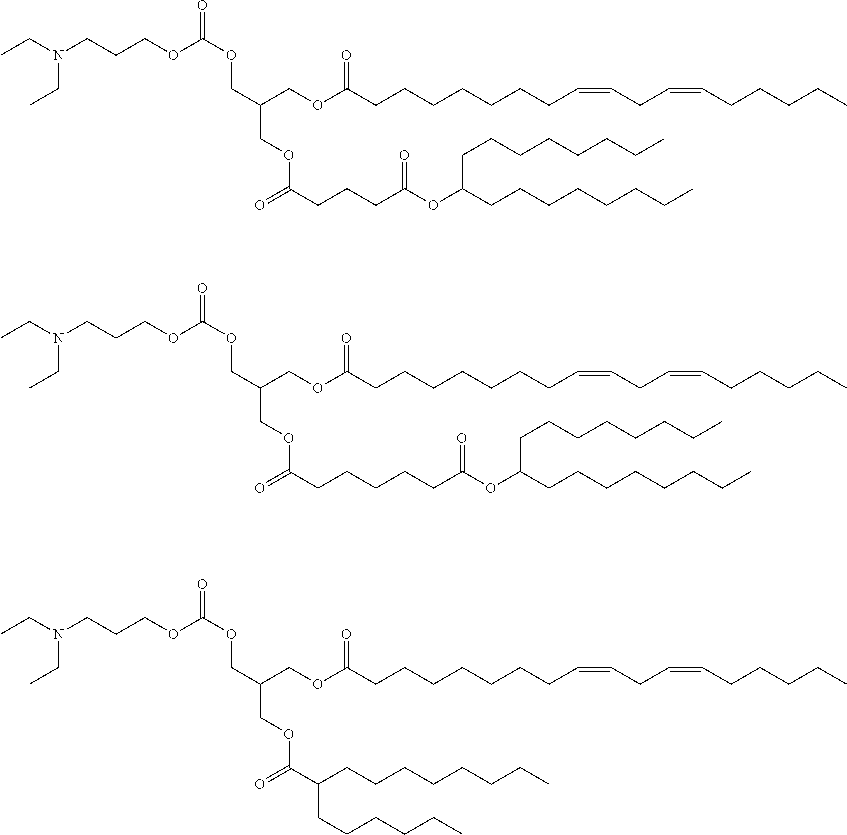 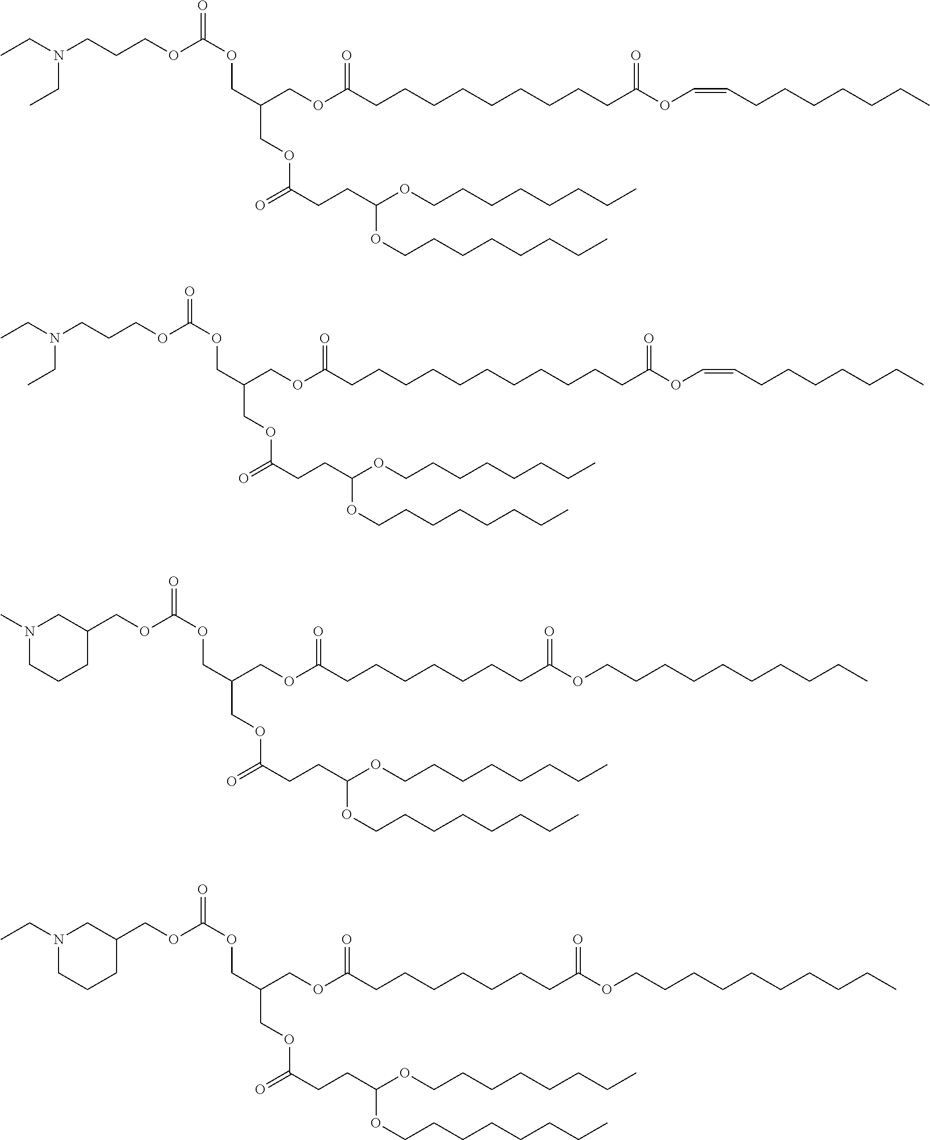 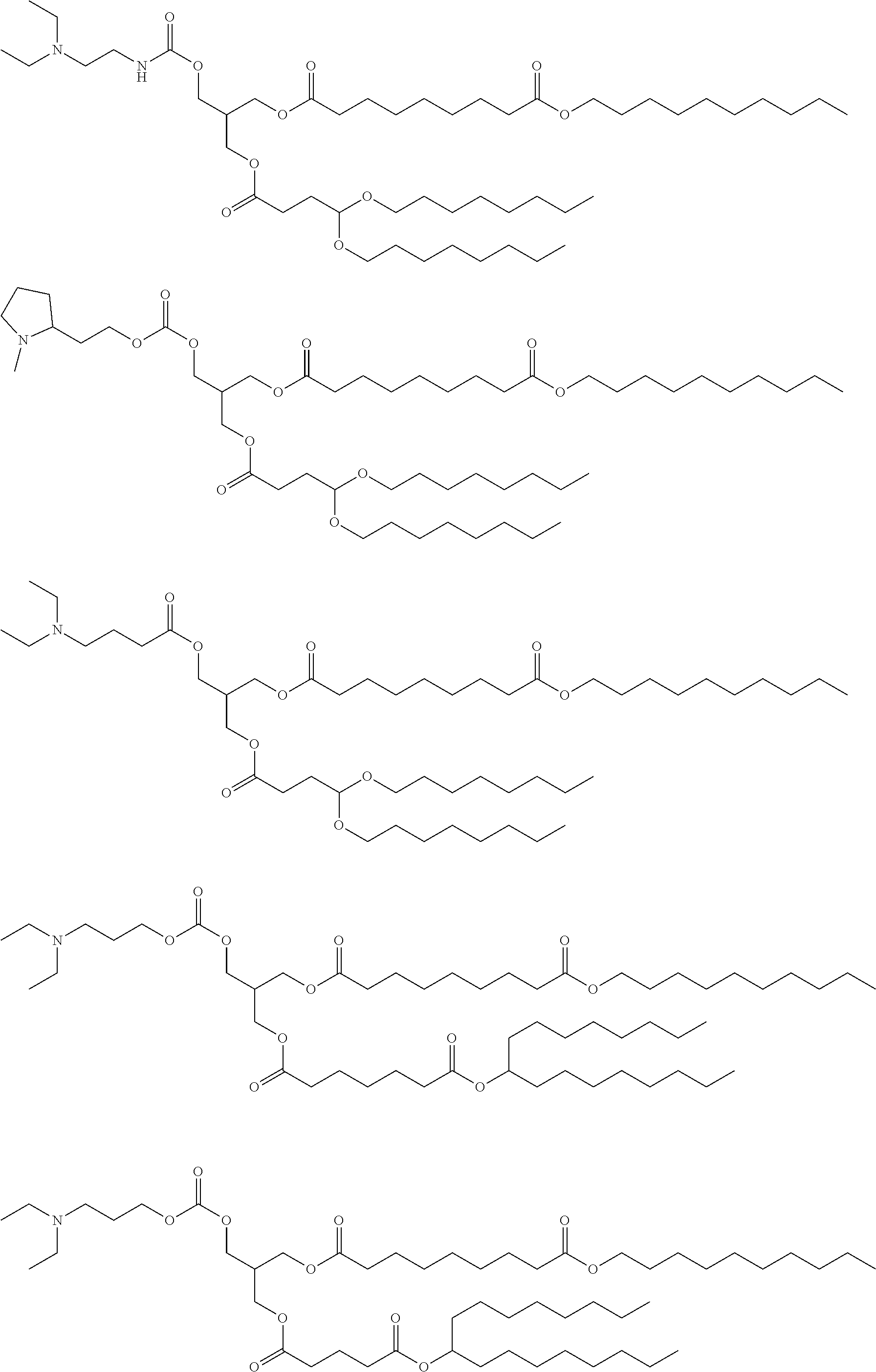 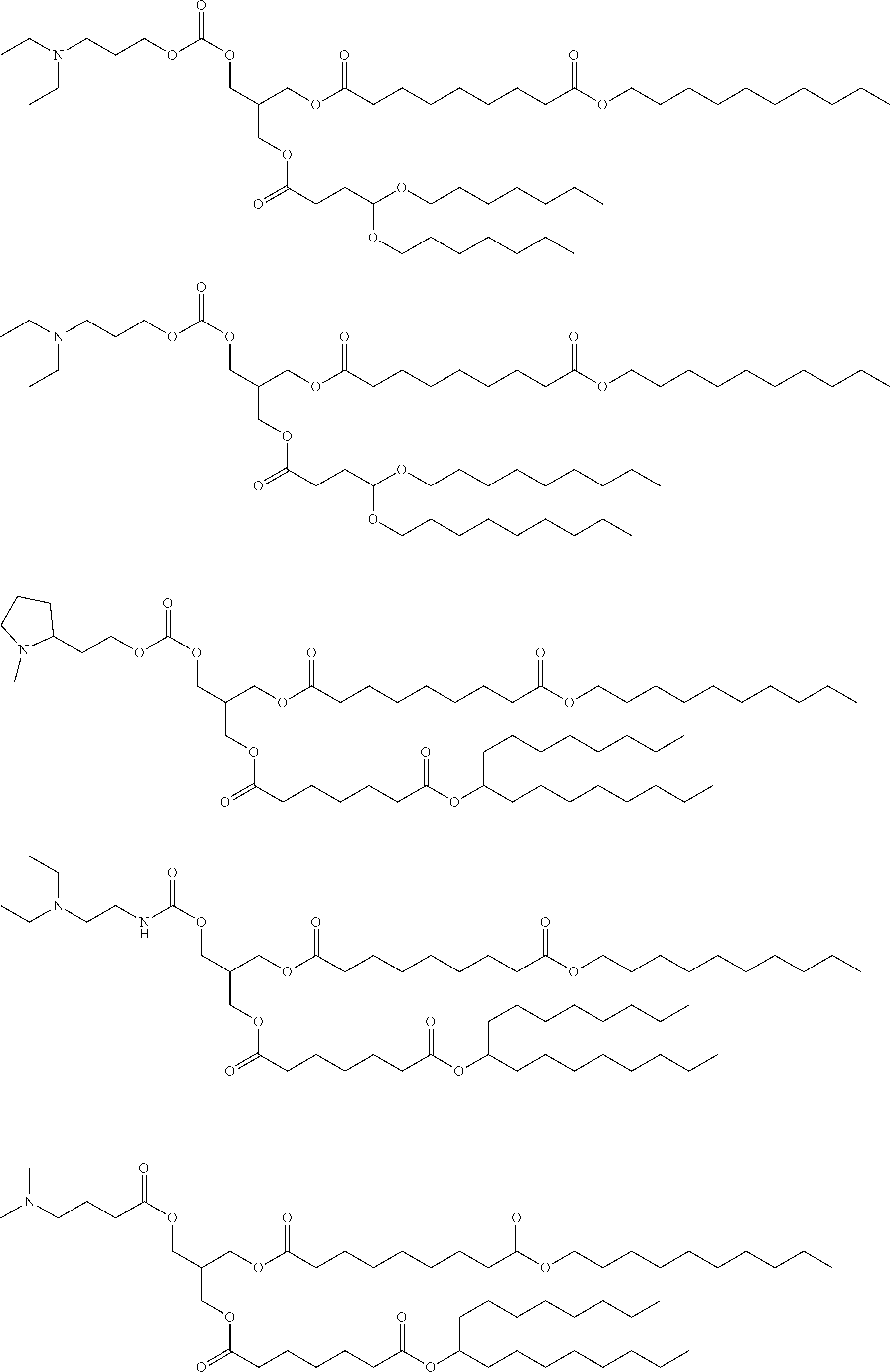 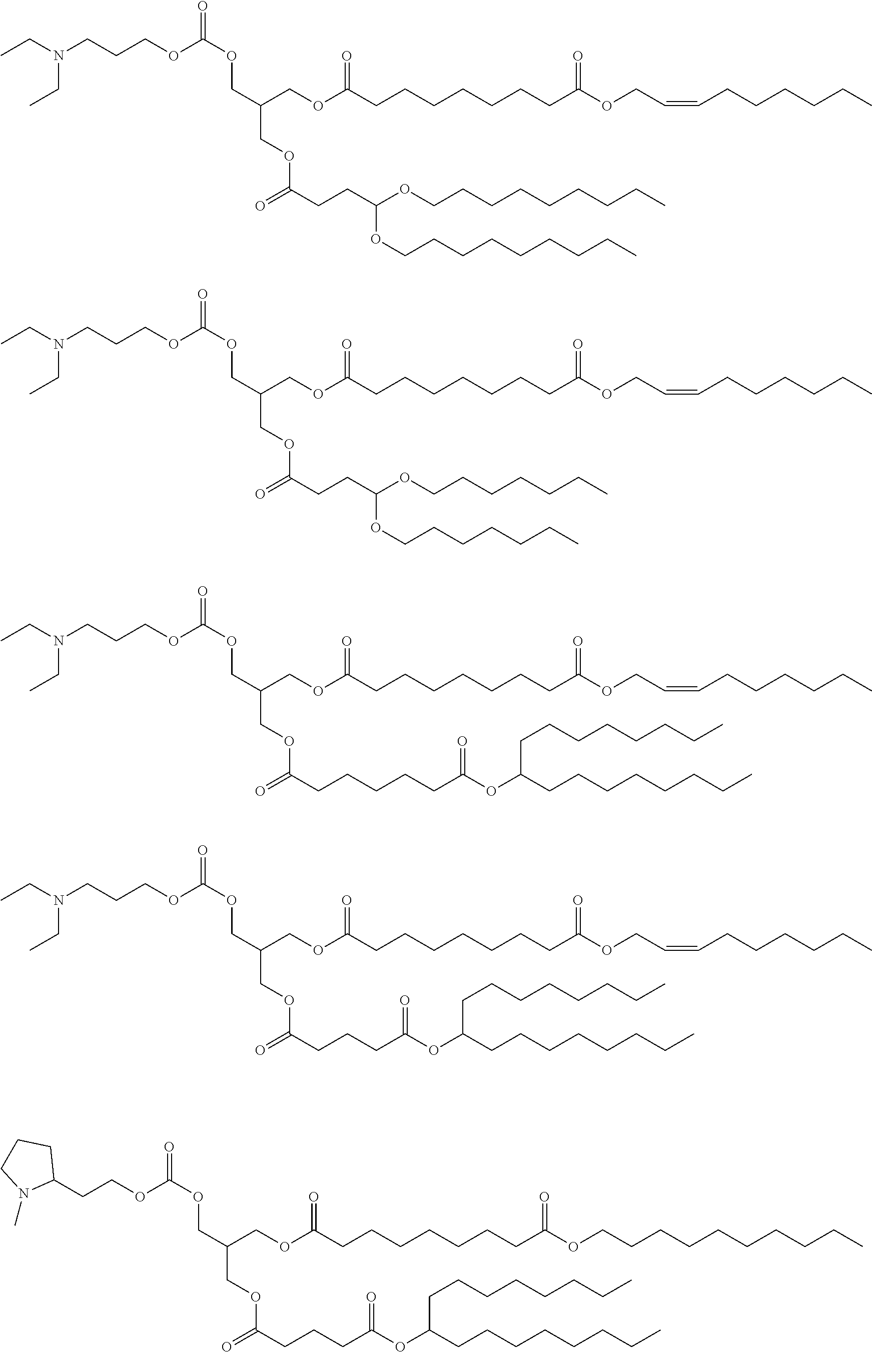 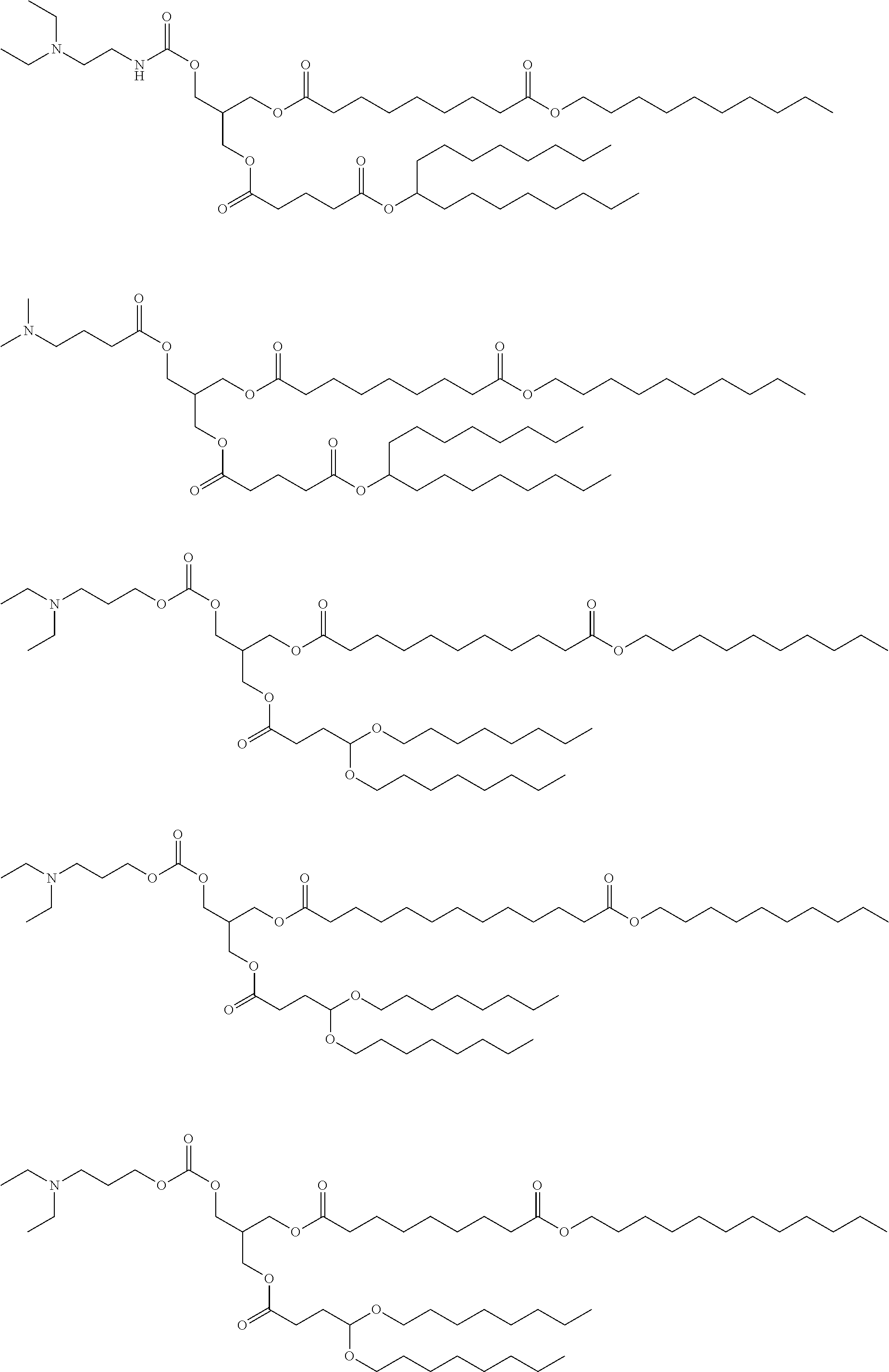 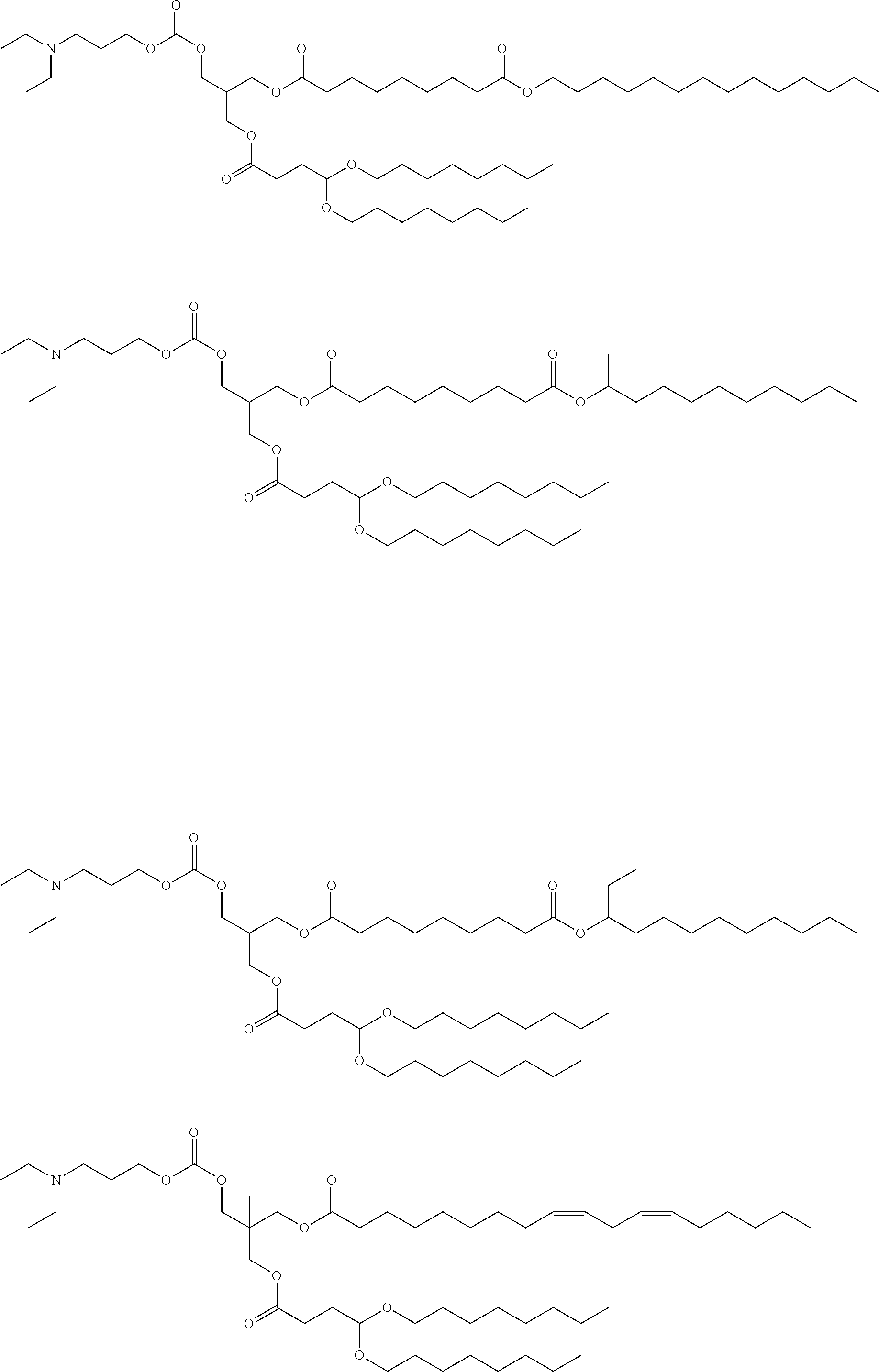 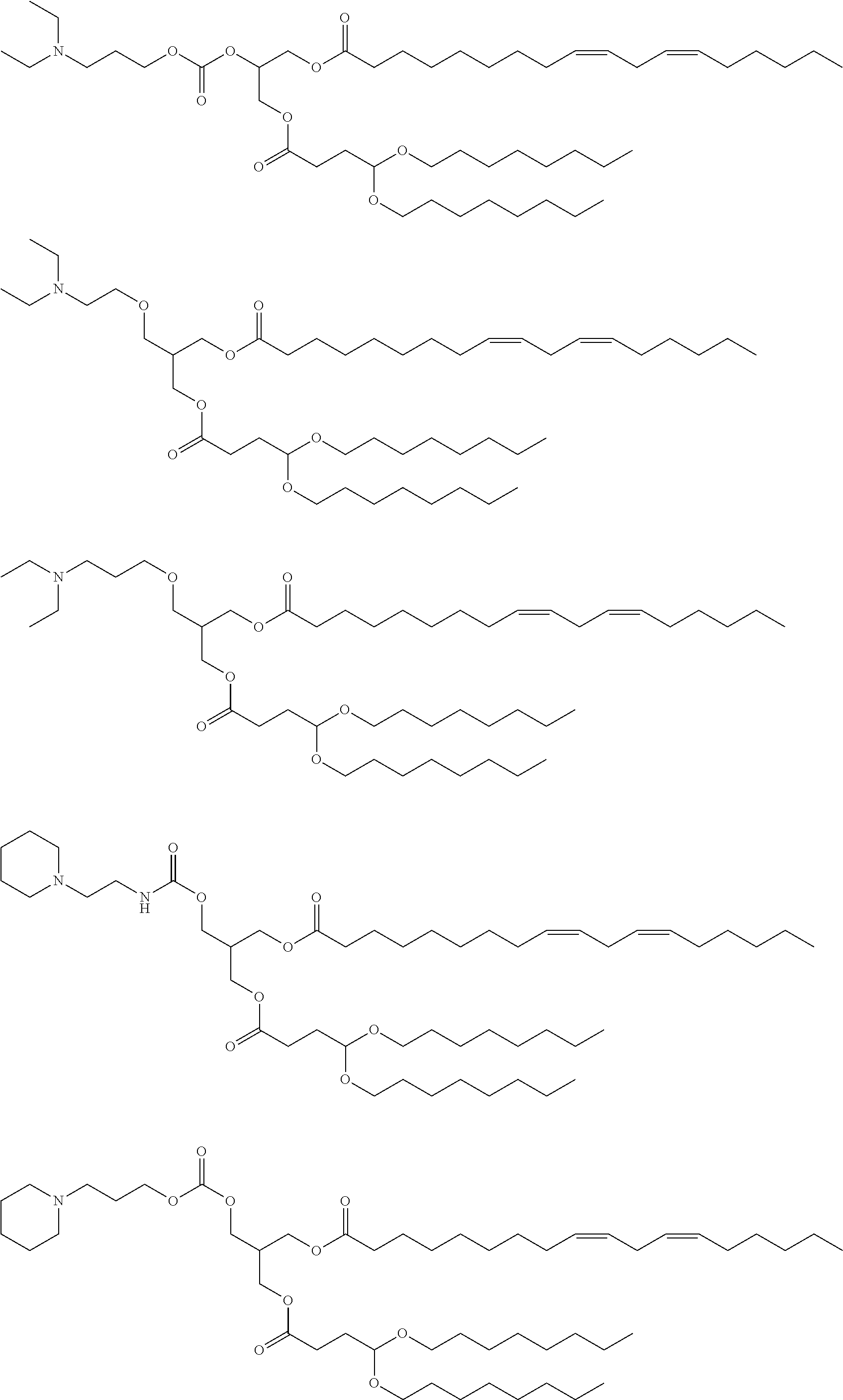 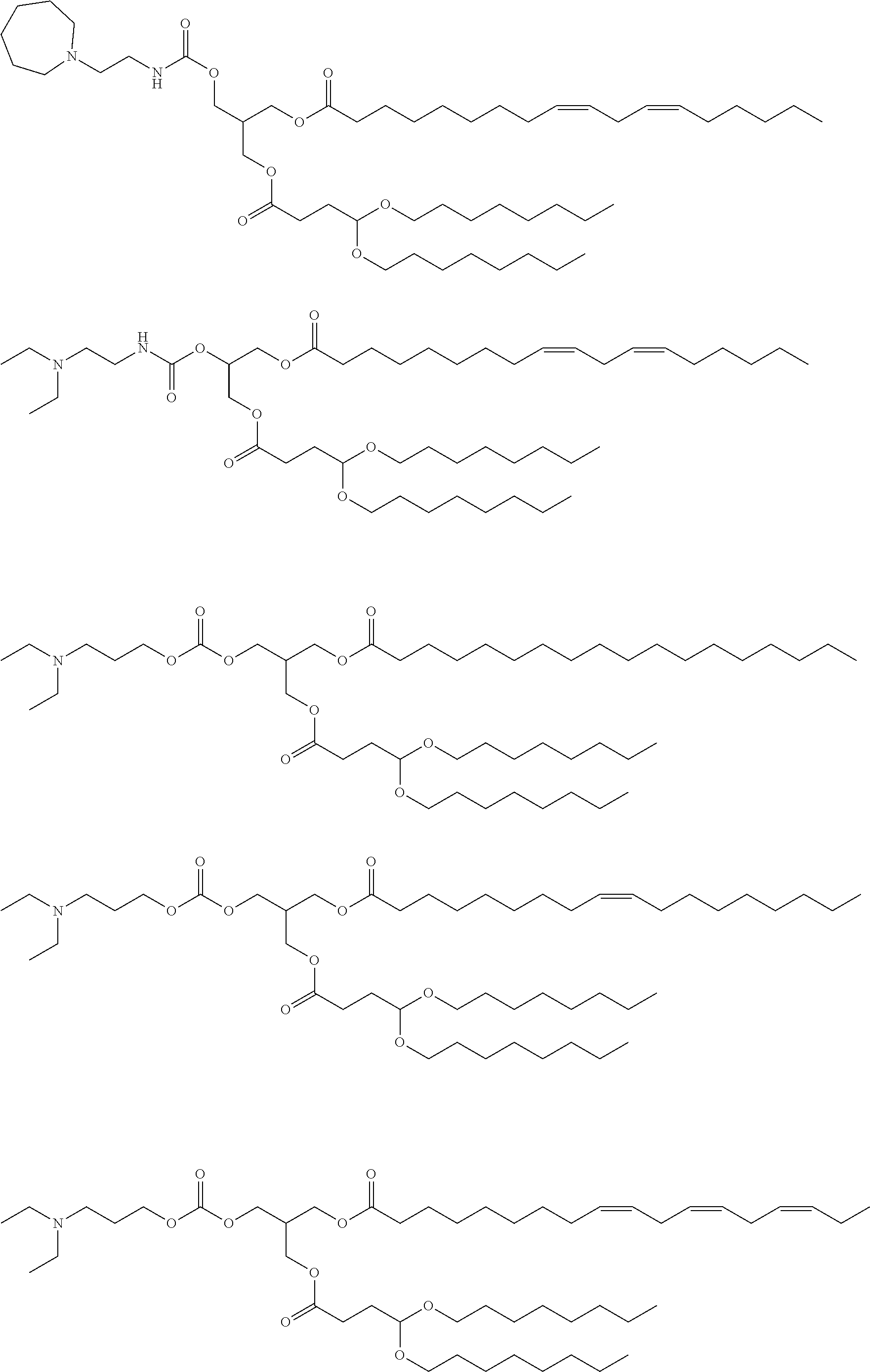 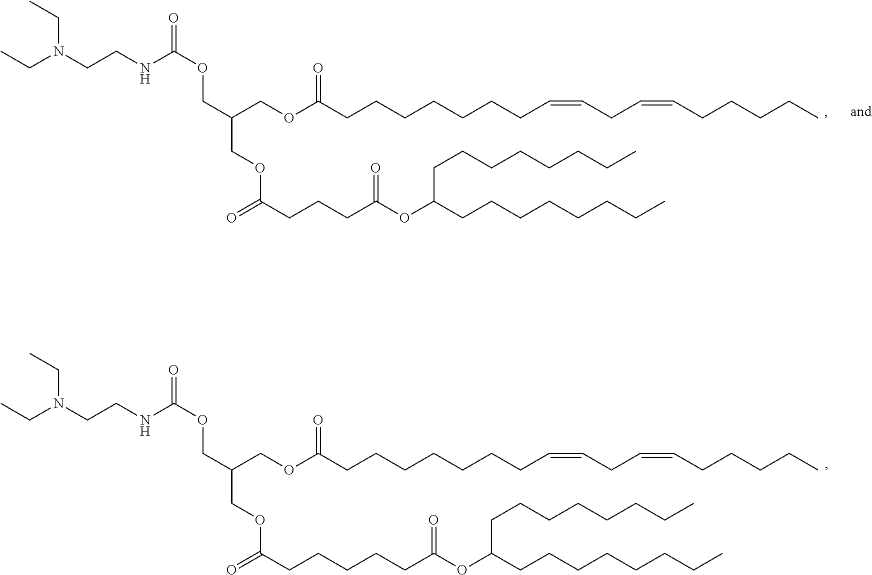
or a salt thereof.

30.-33. (canceled)

34. A lipid nanoparticle (LNP) composition comprising a lipid component, wherein the lipid component comprises a compound of claim 1; and wherein the lipid component further comprises a helper lipid, a PEG lipid, and a neutral lipid.

35.-40. (canceled)

41. The LNP composition of claim 34, wherein:
the composition comprises an aqueous component comprising a biologically active agent the biologically active agent comprises a nucleic acid, and
the LNP composition has an N/P ratio of about 6±1.

42.-48. (canceled)

49. The LNP composition of claim 34, further comprising an RNA component, wherein the RNA component comprises a sequence encoding an RNA-guided DNA-binding agent.

50. The LNP composition of claim 49, wherein the RNA component comprises a Cas nuclease mRNA.

51.-53. (canceled)

54. The LNP composition of claim 49, wherein the RNA component comprises a gRNA nucleic acid.

55.-61. (canceled)

62. The LNP composition of claim 49, further comprising at least one template nucleic acid.

63.-64. (canceled)

65. A method of gene editing, a method of delivering an RNA component to a cell, or a method of cleaving DNA, comprising contacting a cell with a composition of claim 54.

66.-84. (canceled)
